(19) United States
(12) Patent Application Publication
(10) Pub. No.: US 2008/0139449 A1
(43) Pub. Date: Jun. 12, 2008

(54) X-Ray Structure of Human Fpps and Use For Selecting Fpps Binding Compounds

(76) Inventors: Wolfgang Jahnke, Lorrach (DE); Jean-Michel Rondeau, Rixheim (FR); Martin Geiser, Therwil (CH); Paul Ramage, Oltingue (FR); Andre Strauss, Langnau im Emmental (CH)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 11/722,574

(22) PCT Filed: Jan. 2, 2006
(86) PCT No.: PCT/EP06/00002

§ 371 Date: Jun. 22, 2007

Related U.S. Application Data
(60) Provisional application No. 60/641,178, filed on Jan. 4, 2005.

(51) Int. Cl. A61K 38/00; C12N 9/10; C12P 21/04; A61P 43/00; C12Q 1/00; G06G 7/48

(52) U.S. Cl. 514/2; 435/183; 435/69.1; 435/4; 703/11

(57) ABSTRACT

The present invention relates to crystalline human farnesyl diphosphate synthase (FPPS), to the three-dimensional structure of free FPPS as well as the three-dimensional structures of FPPS in complex with substrates such as IPP (isopentenyl diphosphate) and/or with inhibitors such as Zometa® or Aredia®. Further, methods for preparing crystals of human FPPS are described. According to the invention the crystals can be used to determine the structures of FPPS homologs mutants, complexes with ligands, FPPS crystal forms and similar molecules of unknown structure. The invention further relates to the use of FPPS crystals to select new FPPS ligands, e.g. by X-ray screening and to design and/or identify inhibitors against FPPS. Furthermore, the invention relates to NMR methods for selecting and/or identifying new low molecular weight binders to FPPS, which represent new therapeutic agents.

The present invention relates to crystalline human farnesyl diphosphate synthase (FPPS), to the three-dimensional structure of free FPPS as well as the three-dimensional structures of FPPS in complex with ligands such as IPP (isopentenyl diphosphate) and/or with inhibitors such as Zometa® or Aredia®. Further, methods for preparing crystals of human FPPS are described. According to the invention the crystals can be used to determine the structures of FPPS homologs, mutants, complexes with ligands, FPPS crystal forms and similar molecules of unknown structure. The invention further relates to the use of FPPS crystals to select new FPPS ligands, e.g. by X-ray screening and to design and/or identify inhibitors against FPPS. Furthermore, the invention relates to NMR methods for selecting and/or identifying new low molecular weight binders to FPPS, which may be elaborated into new therapeutic agents.

Farnesyl diphosphate synthase (FPPS, E.C. 2.5.1.10), a homodimeric enzyme of the mevalonate/isoprene pathway, catalyses the two steps synthesis of farnesyl diphosphate (FPP), a precursor for the biosynthesis of steroids, ubiquinones, dolichols, heme a, and prenylated proteins. The FPPS reaction is Mg²⁺-dependent and involves the “head-to-tail” condensation between a homoallylic diphosphate, isopentenyl diphosphate (IPP), and an allylic diphosphate, dimethylallyl diphosphate (DMAPP) or geranyl diphosphate (GPP). The reaction proceeds through the formation of an allylic carbonium and leads to the formation of the next higher homologue of the substrate, with concomitant release of pyrophosphate (PPi) from the allylic substrate.

Early biochemical studies have indicated that FPPS possesses distinct allylic and homoallylic binding sites, with the binding of the allylic substrates requiring divalent metal ions (Mg²⁺ or Mn²⁺). The reaction follows an ordered mechanism, with the allylic substrate binding first to the enzyme. Moreover, the E-GPP-PPi complex formed upon condensation of IPP and DMAPP must undergo conformational changes to allow dissociation of pyrophosphate and translocation of GPP prior to the second condensation reaction with IPP to produce FPP.

Crystallographic analyses of avian FPPS have revealed the FPPS fold and provided information about allylic substrate binding. However, the location of the IPP binding site and the molecular mechanisms underlying the catalytic cycle have not been firmly established up to now. Recently, the structures of E. coli FPPS in complexes with IPP and a substrate analog or a biphosphonate were published (Hosfield et al., 2004, J Biol Chem, 279: 8526-8529). Because of the smaller size of E. coli FPPS, and low sequence identity, it is not clear to what extent these results can be applied to human FPPS.

FPPS was recently shown to be the molecular target of nitrogen-containing bisphosphonate drugs such as Aredia® (pamidronate, CGP023339A) and Zometa® (zoledronic acid, CGP042446). Bisphosphonates are an established and very effective class of drugs that inhibit bone resorption by osteoclasts and are thus used for the treatment of conditions involving abnormally increased bone turnover, e.g. osteoporosis, Paget's disease, hypercalcemia and bone metastases. Hence, FPPS is now recognized as an important drug target. It is anticipated that new FPPS inhibitors would have therapeutic potential not only for the treatment of bone diseases but also in oncology, for the treatment of elevated cholesterol levels, and as anti-infectives. In spite of its pharmaceutical relevance, structural information on human FPPS is still lacking. Structural models of inhibitor binding largely rely on the available crystallographic information on avian FPPS.

To be able to select and optimize inhibitors of human FPPS using structure-based approaches, in silico methods as well as high-throughput screening technologies it is necessary to determine the three-dimensional structure of human FPPS. According to the invention this demand is met by the provision of crystalline human farnesyl diphosphate synthase (FPPS).

The present invention describes the production of recombinant human FPPS for structural studies and lead finding and the first X-ray analyses of this enzyme.

According to the invention it has been found that the results obtained are at variance with previous data obtained with avian FPPS. Thus, the crystal structure data provided herein constitute new structural information towards the development of novel inhibitors of this important drug target.

According to the invention human FPPS (hFPPS) which e.g. can be expressed in E. coli can be purified to homogeneity and crystallized. The three-dimensional structure of the crystals can then be determined by X-ray crystallography. Both crystals of hFPPS in an unliganded state and in complex with ligands such as substrates, inhibitors and/or metal ions can be obtained. In a specific embodiment, crystals of hFPPS are in complex with pamidronate/Mn²⁺, zoledronate/Mg²⁺ and isopentenyl diphosphate/zoledronate/Mg²⁺.

In a specific embodiment, the crystalline hFPPS is present in an open conformation, in another specific embodiment in a closed conformation. The change from the open to the closed form involves mainly a large shift of one loop lining the active site, accompanied by a rigid body motion of the last 130 carboxy-terminal residues, which bring the two conserved DDXXD motifs in the enzyme active site closer to each other.

According to the invention it has further been found that nitrogen-containing biphosphonate inhibitors bind to the allylic substrate site and interact with both conserved DDXXD sequence motifs through a trinuclear metal center.

The present invention, in particular, relates to crystalline human FPPS which is in the form of a single crystal, in particular, in the form of a large single crystal having an edge length of at least 10 μm or preferably of at least 50 μm or preferably of at least 100 μm.

The crystals according to the invention preferably belong to space group P4₁2₁2. Crystals according to the invention which are present in open conformation (also referred to herein as crystal form II) preferably have cell dimensions of a=b=111 Å±20 Å, c=77 Å±20 Å or, more preferably, a=b=111 Å±10 Å and c=77 Å±10 Å.

Crystals according to the invention which are present in closed conformation (also referred to herein as crystal form I) preferably have a cell dimension of a=b=112 Å±20 Å and c=66 Å±20 Å and, more preferably, a=b=112 Å±10 Å and c=66 Å±10 Å.

By means of the purification method described herein it is possible, in particular, to provide crystals of such high purity that a resolution of the X-ray crystallography of ≦10 Å, more preferably ≦5 Å, even more preferably ≦3 Å and most preferably ≦2.6 Å can be achieved.

Furthermore, the structure determination of the hFPPS/ligand complexes allows to identify and determine hFPPS binding sites and, therefrom, to determine hFPPS ligands, in particular, inhibitors.

As used herein, the term “human FPPS” relates to any human enzyme having Farnesyl diphosphate synthase activity (FPPS, E.C. 2.5.1.10). In a specific embodiment, human FPPS is encoded by an amino acid sequence which matches that of Genbank entry BC010004, or a functional fragment of that sequence. In a preferred embodiment, a functional fragment of that sequence shares at least 80% identity, more preferably 90%, and even more preferably 95% identity with the corresponding fragment sequence of human FPPS of Genbank entry BC010004 when performing optimal alignment. Optimal alignment of sequences for determining a comparison window may be conducted by the local homology algorithm of Smith and Waterman (J. Theor. Biol., 91 (2) pgs. 370-380 (1981), by the homology alignment algorithm of Needleman and Wunsch, J. Miol. Biol., 48(3) pgs. 443-453 (1972), by the search for similarity via the method of Pearson and Lipman, PNAS, USA, 85(5) pgs. 2444-2448 (1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection.

The best alignment (i.e., resulting in the highest percentage of identity over the comparison window) generated by the various methods is selected for determining percentage identity.

As used in the present invention, human FPPS mutant is human FPPS having an amino acid sequence of human FPPS sharing at least 90% identity, more preferably 95%, and even more preferably 99% identity with the corresponding fragment sequence of human FPPS of Genbank entry BC010004 when performing optimal alignment. Preferably, a mutant of hFPPS is a single mutant of human FPPS, and more preferably, a deficient or non functional mutant. In a specific embodiment of the invention, human FPPS mutant is a human FPPS having a mutation in one or more of the amino acids of the binding pocket as defined below.

The term “ligand” according to the invention, refers to a molecule or group of molecules that bind to one or more specific sites of human FPPS, preferably to the binding pocket of human FPPS and most preferably one of the three identified binding sites of human FPPS. Ligands according to the invention are preferably low molecular weight molecules.

The term “low molecular weight compound” according to the invention refers to preferably organic compounds generally having a molecular weight less than about 1000 daltons, more preferably less than about 600 daltons. Most preferably, said low molecular weight compounds or ligands inhibit human FPPS activity.

As used herein, the term “binding pocket” refers to the region of human FPPS that, as a result of its shape and physico-chemical properties, favorably associates with another chemical entity or compound. Preferably, it refers to the binding pocket, consisting of the three binding sites identified by the present invention:
1) the binding site of the homoallylic substrate (IPP), lined by at least the following amino acids Gly56, Lys57, Arg60, Gln96, Arg113, Thr201, Tyr204, Phe239, Gln240 and Asp243,
2) the binding site of the allylic substrate (DMAPP or GPP) and of bisphosphonate-based inhibitors. This binding site features a trinuclear metal center involving both DDXXD motifs; it is lined by at least the following amino acids: Phe99, Leu100, Asp103, Asp107, Arg112, Thr167, Gln171, Lys200, Thr201, Tyr204, Glu240, Asp243 and Lys257, and in particular, the following amino acids: Leu100, Asp103, Asp107, Arg112, Gln171, Lys200, Thr201, Tyr204, Glu240, Asp243 and Lys257;
3) a novel binding site identified in the present invention, hereafter referred as “the novel binding site” and lined by at least the following amino acids: Tyr10, Lys57, Asn59, Arg60, Thr63, Ser205, Phe206, Phe239, Gln242, Leu246, Leu344, Lys347 and Ile348.

As used herein, the numbering of the residues is in agreement with the SwissProt entry P14324.

In particular, by the structure determination of an IPP/zoledronate/Mg²⁺/hFPPS complex the location of the homoallylic substrate binding site, i.e. the IPP binding site was achieved and conserved residues involved in IPP recognition were identified. Moreover, the biphosphonate inhibitor zoledronate was found to bind to the allylic substrate site through a trinuclear metal center.

According to the invention, it is preferred to use the information concerning the binding pocket for the selection and/or the design of new ligands, in particular new inhibitors for human FPPS, whereby here the ligand preferably interacts with one or more amino acids of the binding pocket, selected from the group consisting of Tyr10, Gly56, Lys57, Asn59, Arg60, Thr63, Gln96, Phe99, Leu100, Asp103, Asp107, Arg112, Arg113, Thr167, Gln171, Lys200, Thr201, Tyr204, Ser205, Phe206, Phe239, Gln240, Gln242, Asp243, Leu246, Lys257, Leu344, Lys347 and Ile348. More specifically, it interacts with one or more amino acids of the binding pocket, selected from the group consisting of Tyr10, Gly56, Lys57, Asn59, Arg60, Thr63, Gln96, Leu100, Asp103, Asp107, Arg112, Arg113, Gln171, Lys200, Thr201, Tyr204, Ser205, Phe206, Phe239, Gln240, Gln242, Asp243, Leu246, Lys257, Leu344, Lys347 and Ile348. More preferably, it is preferred to use the information concerning a novel binding site of human FPPS identified in the present invention, wherein said novel binding site comprises at least the following amino acids Tyr10, Lys57, Asn59, Arg60, Thr63 Ser205, Phe206, Phe239, Gln242, Leu246, Leu344, Lys347, Ile348. Thus it is a preferred object of the present invention to provide means for the design and/or identification of a novel ligand, especially a non biphosphonate ligand that interacts with one or more of the following amino acids comprised in the novel binding site selected among the group consisting of: Tyr10, Lys57, Asn59, Arg60, Thr63, Ser205, Phe206, Phe239, Gln242, Leu246, Leu344, Lys347 and Ile348.

In another specific embodiment, the hFPPS crystals of the invention comprise three metal cations, per FPPS monomer, in particular, Mg²⁺ or/and Mn²⁺.

The invention further relates to a method for producing a crystalline human FPPS preparation comprising the steps of:
(i) expressing recombinant human FPPS in E. coli, wherein said recombinant human FPPS comprises amino acid residues 6 to 353,
(ii) purifying expressed human FPPS, and
(iii) crystallizing the purified human FPPS.

Preferably, recombinant expression is achieved by using a plasmid encoding amino acid residues 6 to 353 from a sequence which matches that of Genbank entry BC010004.

In a specific embodiment, purification in step (ii) comprises purification via anion exchange column and size exclusion chromatography.

In another embodiment, crystallizing in step (iii) comprises crystallizing by vapor diffusion, free interface diffusion, microdialysis or microbatch under oil.

More preferably, the method for producing human FPPS is done according to the purification method 1, described later in the specification.

The invention also relates to a crystalline human FPPS obtainable by the above method.

The present invention thus provides a crystal structure of human FPPS defined by all or a selected portion of the structural coordinates shown in FIG. 14, FIG. 15, FIG. 16, FIG. 17 and/or FIG. 18, and similar structures thereof.

By “selected portion”, it is meant the structural coordinates of at least 10 amino acids shown in FIGS. 14, 15, 16, 17 and/or 18 and preferably at least 20 amino acids. In a preferred embodiment, a selected portion corresponds to the structural coordinates of the amino acids forming at least one of the three binding sites of the binding pocket as defined above.

By “similar structures”, it is meant structures of human FPPS having structural coordinates with variations when compared to the structural coordinates shown in FIG. 14, FIG. 15, FIG. 16, FIG. 17 and/or FIG. 18 within the range of the X-ray resolution of the crystal structure performed in the present examples, and preferably within the range of a resolution of the X-ray crystallography of ≦10 Å, more preferably ≦5 Å, even more preferably ≦3 Å and most preferably ≦2.6 Å.

It was found that human FPPS exists in two conformations, namely a closed conformation as well as an open conformation.

Indeed, during the refinement of the structure, it was surprisingly found that the human enzyme was, in comparison to avian FPPS, in a different conformational state to which reference is made as the closed state (FIG. 1). The structural overlay between the two conformational states have shown that only the last 130 carboxy-terminal residues are affected by the conformational switch, with a rigid body movement of helices H, I, J and α-1, α-2 and α-3 (FIG. 3). This conformational change brings helix H closer to helix D, thereby shortening the distance between the two DDXXD motifs and closing the active site. In the open conformation, the Oδ2 atoms of Asp103 and Asp243 are 12 Å apart, in the closed state this distance is reduced to 10 Å. This rigid body movement is accompanied by a large shift of the H-I loop, which behaves as a lid that clamps down over the active site in the closed state (FIG. 9). The position of the Cα atom of Gly256, at the tip of the H-I loop, is shifted by 6.7 Å. Several conserved residues lining the enzyme active site and involved in the binding of substrates are also affected by the conformational switch, notably Phe239, Gln240, Leu100, Lys257 and Lys266. Furthermore, some structural elements become better ordered in the closed conformation. This is the case for the H-I loop which has less well-defined electron density and higher B-factors in the open state, as well as for the last three carboxy-terminal residues of FPPS, Arg351, Arg352 and Lys353, which are disordered in the open state but interact with the bound substrates in the closed state. The dimer interface is, however, not affected by the conformational switch. The two FPPS subunits interact through helices A, B, D and E, which remain fixed and constitute a rigid core centered on the dimer two-fold axis.

In order to further investigate the structure of human FPPS and, in particular, to obtain information about the FPPS/ligand interactions, which are of interest, crystals containing human FPPS in complex with ligand molecules or/and inhibitor molecules can be produced.

The hFPPS crystals and crystal structure data, respectively, provided by the invention can be used, in particular, for determining binding sites of hFPPS as well as for selecting, designing, identifying and/or providing novel FPPS ligands.

In particular, starting out from the crystal structure data, ligand molecules can be easily obtained using computer-aided modeling programs. To this end, for example, first a three-dimensional representation of FPPS and the binding sites, respectively, is generated by means of the crystal structure data, the three-dimensional representation e.g. being an electron density map, a wire-frame model, a chicken-wire model, a ball- and stick-model, a space-filling model, a stick model, a ribbon model, a snake model, an arrow- and cylinder model, a molecular surface model or a combination thereof. Suitable ligands are selected by means of their three-dimensional structure, whereby said structure should be complementary to the interaction site of FPPS. To this end, for example, a three-dimensional representation of FPPS and a three-dimensional representation of a potential ligand compound is prepared and then it is tested, optionally computer-aided, whether the three-dimensional representation of the potential compound fits into the binding pocket of the three-dimensional representation of FPPS. This procedure is particularly suitable for rational drug design.

In one embodiment of the invention, a computer-based method is provided for the selection, identification and/or design of a ligand capable of binding to human FPPS, comprising the steps of:
a) providing a three-dimensional representation of human FPPS according to structure coordinates of human FPPS,
b) providing a three-dimensional representation of a candidate compound,
c) selecting a candidate compound whose three dimensional representation is complementary to the binding pocket of human FPPS, and,
d) optionally modifying said compound selected at step c) to maximize physical properties such as solubility, affinity, specificity and/or potency.

Ligands can be selected from screening compound databases or libraries and using a computational means to perform a fitting operation to a binding site of the binding pocket of human FPPS. The three dimensional structure of the binding pocket as provided in the present invention in whole or in part by the structural coordinates of the tables shown in FIG. 14, FIG. 15, FIG. 16, FIG. 17 and/or FIG. 18 can be used together with various docking programs.

The potential inhibitory or binding effect of a compound on human FPPS may be analysed prior to its actual synthesis and testing by the use of computer-modeling techniques. If the theoretical structure of the given chemical entity suggests insufficient interaction and association between it and human FPPS, the need for synthesis and testing of the compound is obviated. However, if computer modeling indicates a strong interaction, the molecule may then be synthesized and tested for its ability to bind to human FPPS. Thus, expensive and time-consuming synthesis of inoperative compounds may be avoided.

An inhibitory or other binding compound of human FPPS may be computationally evaluated and designed by means of a series of steps in which compounds are screened and selected for their ability to associate with the individual binding sites of human FPPS. Thus, one skilled in the art may use one of several methods to screen compounds for their ability to associate with human FPPS. This process may begin by visual inspection of, for example, the binding site on a computer screen based on the structural coordinates in whole or in part. Selected compounds may then be positioned in a variety of orientations, or “docked,” within the pocket binding site of human FPPS. Docking may be accomplished using software such as Quanta and SYBYL, followed by energy minimization and molecular dynamics with standard molecular mechanics force fields, such as CHARMM and AMBER. Specialized computer programs may be of use for selecting interesting compounds. These programs include, for example, GRID, available from Oxford University, Oxford, UK; MCSS or CATALYST, available from Molecular Simulations, Burlington, Mass.; AUTODOCK, available from Scripps Research Institute, La Jolla, Calif.; DOCK, available from University of California, San Francisco, Calif., and XSITE, available from University College of London, UK.

In a preferred embodiment, the structure coordinates of the closed conformation of human FPPS will be used in the above computer-based method.

Preferably, said compound is selected among those that interact with one or more amino acids of the binding pocket selected from the group consisting of Tyr10, Gly56, Lys57, Asn59, Arg60, Thr63, Gln96, Phe99, Leu100, Asp103, Asp107, Arg112, Arg113, Thr167, Gln171, Lys200, Thr201, Tyr204, Ser205, Phe206, Phe239, Gln240, Gln242, Asp243, Leu246, Lys257, Leu344, Lys347 and Ile348. More specifically, it interacts with one or more amino acids of the binding pocket selected from the group consisting of Tyr10, Gly56, Lys57, Asn59, Arg60, Thr63, Gln96, Leu100, Asp103, Asp107, Arg112, Arg113, Gln171, Lys200, Thr201, Tyr204, Ser205, Phe206, Phe239, Gln240, Gln242, Asp243, Leu246, Lys257, Leu344, Lys347 and Ile348.

In another preferred embodiment, said compound is selected from among the ligands that fit into the novel binding site. Preferably, said compound is selected among those that interact with one or more amino acids selected from the group consisting of Tyr10, Lys57, Asn59, Arg60, Thr63, Ser205, Phe206, Phe239, Gln242, Leu246, Leu344, Lys347 and Ile348.

In another preferred embodiment, the method is provided to design ligands by modifying said compound selected at step c) to maximize physical properties such as solubility, affinity, specificity and/or potency.

The designed compound must be capable of physically interacting with one or more of the amino acids of the binding pocket. The association may be chemical association, such as for example, covalent or non covalent binding, or van der Waals, hydrophobic, or electrostatic interactions. Second, the compound must be able to assume a conformation that allows it to associate with human FPPS, preferably the binding pocket of human FPPS. Although not all portions of the compound will necessarily participate in the association with human FPPS, those non participating portions may still influence the overall conformation of the molecule. Such conformational requirements include the overall three-dimensional structure and orientation of the chemical entity in relation to all or a portion of the binding site.

The structural coordinates shown in FIGS. 14, 15, 16, 17 and/or 18 are especially preferably stored on a computer-readable storage medium comprising a data storage medium with computer-readable data. The computer-readable storage medium can be part of a computer system.

The invention further relates to a method for selecting a ligand capable of binding to human FPPS, comprising:
a. co-crystallizing or incubating a candidate compound with human FPPS,
b. determining by X-ray or NMR methods the amino acids of human FPPS which interact with the candidate compound,
c. selecting a compound which interacts with one or more amino acids of the binding pocket selected among the group consisting of Tyr10, Gly56, Lys57, Asn59, Arg60, Thr63, Gln96, Phe99, Leu100, Asp103, Asp107, Arg112, Arg113, Thr167, Gln171, Lys200, Thr201, Tyr204, Ser205, Phe206, Phe239, Gln240, Gln242, Asp243, Leu246, Lys257, Leu344, Lys347 and Ile348, based on the results of step b, in particular from the group consisting of Tyr10, Gly56, Lys57, Asn59, Arg60, Thr63, Gln96, Leu100, Asp103, Asp107, Arg112, Arg113, Gln171, Lys200, Thr200, Tyr204, Ser205, Phe206, Phe239, Gln240, Gln242, Asp243, Leu246, Lys257, Leu344, Lys347 and Ile348

For carrying out step b), mapping of the binding site of a ligand is usually performed by recording NMR spectra with and without the candidate compound, and identifying those resonances of the protein that are affected by ligand binding. This requires assignment of the protein resonance prior to the analysis, or comparison with the pattern of chemical shift changes that occur upon binding of ligands with known binding sites. Alternatively, competition experiments using said ligands with known binding sites can yield equivalent information.

The invention therefore also relates to an NMR method for selecting improved binders to FPPS, in particular, low molecular weight binders. This method is based preferably on assigning selected resonances in an indirect manner. In particular, resonances which experience chemical shift changes upon displacement of one ligand, e.g. pamidronate, by another ligand, e.g. zoledronate, can be located in close vicinity to the location of the second ligand. Thus, those chemical shift changes indicate the ligand binding site. This approach can be further assisted by a paramagnetic relaxation enhancement which can be caused by displacement of diamagnetic metal ions, e.g. Mg²⁺ with paramagnetic metal ions, e.g. Mn²⁺. Residues unaffected by such a paramagnetic relaxation enhancement are ≧2 nm away from the paramagnetic center, whereas residues which are affected by such a paramagnetic relaxation enhancement are within 1.0 to 1.5 nm distance to the metal ions.

In a preferred embodiment, prior to step a), said candidate compound is selected according to a computer-based method of the invention as described above.

In another specific embodiment, the method of the invention further comprises the steps of:
d. designing analogs of the compound obtained at step c) to maximize physical properties such as solubility, affinity, specificity and/or potency,
e. repeating step a. to c. of the above method with the corresponding analogs to select novel compounds capable of binding to human FPPS.

The present invention further provides methods to design novel ligands of human FPPS, using fragment linking approaches. Compounds binding to each binding site are first selected.

Then, the ligands are linked together based on the spatial orientation, so that the designed novel compound fits within the two binding sites.

The invention thus relates to a method to design ligand to human FPPS, wherein said method comprises the steps of
a) providing a first ligand that binds to one or more amino acids of a first binding site of human FPPS,
b) providing a second ligand that binds to one or more amino acids of a second binding site of human FPPS,
c) linking said first ligand to said second ligand to design a ligand that binds to the first and second binding sites of human FPPS.

In a specific embodiment, the method comprises the steps of providing a third ligand that binds to one or more residues of a third binding site, and linking said third ligand to the ligand obtained at step c) to form a ligand that binds to the first, second and third binding sites.

Preferably, a first ligand at step a) is selected among the ligands that fit within the novel binding site of human FPPS. Preferably, said first ligand is selected from among the ligands that interact with one or more amino acids selected among the group consisting of: Tyr10, Lys57, Asn59, Arg60, Thr63, Ser205, Phe206, Phe239, Gln242, Leu246, Leu344, Lys347 and Ile348

The selection of an appropriate linking group is made by maintaining the spatial orientation of the ligands to one another and to the human FPPS based upon principles of bond angle and bond length information well known in the organic chemical art.

More preferably, a second ligand at step b) is selected from among the ligands that fit within the binding site of the homoallylic substrate (IPP) and/or the binding site of the allylic substrate (DMAPP or GPP). For example, a second ligand at step b) is selected from among the ligands that interact with one or more amino acids selected among the group consisting of: Gly56, Lys57, Arg60, Gln96, Arg113, Thr201, Tyr204, Phe239, Gln240 and Asp243 and/or with one or more amino acids selected among the group consisting of: Phe99, Leu100, Asp103, Asp107, Arg112, Thr167, Gln171, Lys200, Thr201, Tyr204, Glu240, Asp243 and Lys257, in particular from the group consisting of Leu100, Asp103, Asp107, Arg112, Gln171, Lys200, Thr201, Tyr204, Glu240, Asp243 and Lys257.

The present invention, finally, also relates to ligands for human FPPS which are obtained using the information given herein. Those ligands preferably are inhibitors of human FPPS. Such ligands are preferably used in pharmaceutical compositions and, in particular, in pharmaceutical compositions for the treatment and/or prevention of tumor-induced hypercalcemia, Paget's disease of bone, osteolytic metastases, postmenopausal osteoporosis, hypocholesterolemia and/or soft tissue cancer.

### LEGENDS OF THE FIGURES

FIG. 1: Overall structure of the human FPPS homodimer (closed conformation)

FIG. 2: Residual electron density (3σ contour) revealing the presence of an unknown endogeneous ligand within the active site cleft of human FPPS. The electron density was partially interpreted with a phosphate group, shown here in ball-and-stick representation (stereo view).

FIG. 3: Overlay of the closed (magenta Cα trace) and open state (cyan Cα trace) of human FPPS. The two structures were superimposed using the first 150 amino-terminal residues (rmsd=0.34 Å). The overlay reveals that only the last 130 C-terminal residues are actually affected by the conformational switch, notably the H-I loop and the H, α-1, α-2, α-3, I and J helices, while the first 220 residues show an rmsd of only 0.44 Å.

FIG. 4: Close-up view of the closed conformation of human FPPS (magenta Cα trace) superimposed onto the open state (cyan Cα trace). Note the large shift of the H helix and of the H-I loop, affecting notably the position of residues F239, Q240, D243, D247, G256, K257 and K266.

FIG. 5: Close-up view of the human FPPS complex with Mn2+ and pamidronate. Potential polar/electrostatic interactions are indicated by thin black line. Pamidronate is shown in ball-and-stick representation with transparent van der Waals surface. The trinuclear Mn2+ center is shown as violet spheres, together with coordinating water molecules (small cyan spheres).

FIG. 6: Coordination spheres of the three Mg²⁺ ions (2 different orientations). Cyan spheres represent well-defined water molecules. Polar interactions involving the hydroxyl group of Zometa (zoledronic acid) are indicated with dashed lines.

FIG. 7: Close-up view of the human FPPS complex with Mg²⁺ and Zometa (zoledronic acid). Potential polar/electrostatic interactions are indicated by thin black line. Zometa (zoledronic acid) is shown in ball-and-stick representation with transparent van der Waals surface. The trinuclear Mg²⁺ center is shown as violet spheres, together with coordinating water molecules (small cyan spheres).

FIG. 8: Electron density (σ_{A}-weighted, (Fo-Fc, (φ_{calc}) annealed omit electron density map, 4.0σ contour) for the bound ligands Zometa (zoledronic acid) and isopentenyl diphosphate. Green spheres mark the position of Mg²⁺ cations, cyan spheres indicate the location of water molecules belonging to the coordination spheres of the magnesium ions.

FIG. 9: Close-up view of the binding interactions between isopentenyl diphosphate (IPP) and its binding site on human FPPS. Potential hydrogen-bonds are indicated by thin black lines with their length given in Angstroms. IPP is shown in ball-and-stick representation together with its van der Waals surface. Zometa (zoledronic acid) is shown in ball-and-stick. Cyan spheres represent water molecules, green spheres magnesium ions and the magenta sphere the position of residual density tentatively ascribed to a sodium ion.

FIG. 10: ¹⁵N,¹H-TROSY NMR spectra of the FPPS homodimer (80 kDa): Unliganded FPPS (FPPS dimer concentration: 60 μM; black spectrum) and FPPS complexed by pamidronate/Mg²⁺ (FPPS dimer concentration: 60 μM, Pamidronate concentration: 270 μM, Mg²⁺ concentration: 900 μM; blue spectrum).

FIG. 11: ¹⁵N,¹H-TROSY NMR spectra of FPPS complexed to pamidronate/Mg²⁺ (FPPS dimer concentration: 60 μM, pamidronate concentration: 270 μM, Mg²⁺ concentration: 900 μM; blue spectrum), and FPPS complexed to zoledronate/Mg²⁺ (FPPS dimer concentration: 60 μM, Zoledronate concentration: 270 μM, Mg²⁺ concentration: 900 μM; red spectrum). Some resonances that are perturbed by the displacement of pamidronate by Zometa (zoledronic acid) are circled red. The additional peaks appearing in the “random coil region” between 7.5 and 8.0 ppm come from degraded FPPS.

FIG. 12: ¹⁵N,¹H-TROSY NMR spectra of FPPS complexed to zoledronate/Mg²⁺ (FPPS dimer concentration: 60 μM, zoledronate concentration: 270 μM, Mg²⁺ concentration: 900 μM; red spectrum), and after addition of IPP (400 μM; green spectrum). Some resonances perturbed by IPP addition are circled green. The additional peaks appearing in the “random coil region” between 7.5 and 8.0 ppm come from degraded FPPS.

FIG. 13: 800 MHz ¹⁵N,¹H-TROSY NMR spectra of FPPS complexed to zoledronate/Mg²⁺ (FPPS dimer concentration: 60 μM, zoledronate concentration: 270 μM, Mg²⁺ concentration: 800 μM; black spectrum), and of FPPS complexed to zoledronate/Mn²⁺ (FPPS dimer concentration: 60 μM, zoledronate concentration: 270 μM, Mn²⁺ concentration: 400 μM; orange spectrum).

FIG. 14: X-ray structural coordinates of hFPPS unliganded in closed form.

FIG. 15: X-ray structural coordinates of hFPPS unliganded in open form.

FIG. 16: X-ray structural coordinates of hFPPS in complex with pamidronate and Mn²⁺.

FIG. 17: X-ray structural coordinates of hFPPS in complex with zoledronate, IPP and Mg²⁺.

FIG. 18: X-ray structural coordinates of hFPPS in complex with zoledronate and Mg²⁺.

FIG. 19: Close-up view of the human FPPS complex with Zn²⁺ and Ibandronate. Potential polar/electrostatic interactions are indicated by thin black line. Ibandronate is shown in ball-and-stick representation. Two alternate conformations of Ibandronate, originating from the inversion of its tertiary nitrogen, were modeled and refined. The trinuclear Zn²⁺ center is shown as grey spheres, together with coordinating water molecules (small cyan spheres).

FIG. 20: X-ray structural coordinates of hFPPS in complex with ibandronate and Zn²⁺.

### EXAMPLES

1. Cloning and E. coli Expression

The plasmid encoding human farnesyl diphosphate synthase was from the I.M.A.G.E cDNA clone library (clone MGC:15352, IMAGE:4132071). Its sequence matched that of Genbank entry BC010004. The DNA encoding the amino acid fragment 6 to 351 was cloned by PCR using the oligonucleotides MG1053 (5′-ctggaagttctgttccaggggccaaattcagatgtttatgcccaagaa-3′) and MG1054 (5′-gtcgacgtaggcctttgaattcactttctccgcttgtagattttg-3′). The PCR fragment was then integrated into the plasmid pXI341 following the method of Geiser et al. (Bio Techniques 31 (2001) 88-92). The resulting plasmid, called pXI478, corresponds to human FPPS (amino acid residues 6 to 351 with a hexahistidine tag followed by a PreScission protease cleavage site at the N-terminus.

E. coli BL21 (DE3) Tuner cells (Novagen) were transformed with the pXI478 plasmid and stored in liquid nitrogen until fermentation was started.

### 2. Fermentation

### 2.1 Batch 1

Recombinant E. coli was cultured with an ISF-100 fermenter in 5 liters TBmod medium containing 25 mg/l kanamycin, first at 37° C. until induction at OD600nm=3.7 by 1 mM IPTG and then further cultured for 4 hours at 28° C. and pH=7.0 with pO₂=97-98%. The harvested cells (68 g fresh weight) expressed FPPS at high levels, about 50% in soluble form as shown by SDS-PAGE analysis (dominant band on coomassie-stained gel at the expected molecular weight of 40 kDa).

High level expression (>50 mg/l) of human FPPS (residues 6 to 353) can be achieved in E. coli. About 10 to 50% of the protein was produced in soluble form. The identity and integrity of the purified enzyme was established by N-terminal sequencing and mass spectrometry. Electrospray ionization mass spectroscopy under native conditions confirmed that human FPPS is a homodimer of 80 kDa. The protein was well behaved, well soluble (>20 mg/ml), stable, and showed the expected enzymatic activity.

### 2.2 Batch 2

Recombinant E. coli was cultured in 5.5 liters auto-inducing medium ZYP-5052 containing 25 mg/l kanamycin in an ISF-100 fermenter, auto-induced and cultured for 14 hours at 28° C. at pH=7.1 and pO2=100-89%. The harvested cells (114 g fresh weight) expressed FPPS at high levels, however about 10% only in soluble form.

### 3. Purification

3.1 Purification method 1: batch 1

68 g E. coli wet cell pellet (batch 1) was suspended in 560 ml buffer A (50 mM Tris pH 8.0, containing 5 mM each DTT, benzamidine-HCl and EDTA) and lysed by passing twice through an Avestin C-50 microfluidiser before centrifugation for 30 min at 15,000 rpm in an SLA1500 rotor (Sorvall). The resulting supernatant was loaded onto an XK26/10 column of Q-Sepharose HP equilibrated with buffer A. The column was washed with buffer A until the baseline had returned to zero, after which the column was eluted by a 0 to 1M gradient of NaCl in buffer A (over 15 column volumes; 750 ml). 10 ml fractions were collected, peaks pooled and analysed using 4-20% Novex Tris-glycine SDS-PAGE. A sharply eluting peak early in the gradient was confirmed by LC-MS to be FPPS. Based on analytical RP-HPLC, this peak contained 193 mg FPPS. 964 units of PreScission protease were added directly and the mix was incubated overnight at 4° C. LC-MS confirmed complete removal of the N-terminal His-tag. The digested fraction was concentrated by ultrafiltration to about 10 ml prior to size-exclusion chromatography using an XK26/60 column of Superdex 75 equilibrated with 25 mM Tris pH 8.0, 2 mM DTT and 25 mM NaCl. A single peak containing 168 mg protein (by RP-HPLC) was eluted in 40 ml total volume.

3.2 Purification Method 2: batch 2

114 g E. coli wet cell pellet (batch 2) was lysed in 940 ml buffer A (50 mM Tris pH 8.0, 5 mM DTT, 5 mM EDTA) and centrifuged at 34,000 g. The supernatant was sterile filtered and then loaded on a Q-Sepharose HP anion exchange column equilibrated with buffer A. Elution was performed by a 0 to 1.0M NaCl gradient over 8 column volumes. FPPS was eluted early in the gradient at about 100-150 mM NaCl. The fractions were analyzed by SDS-PAGE, pooled, and glycerol, ammonium sulfate and sodium chloride were added to a final concentration of 10% (w/v), 1.5M and 1.0M respectively. The sample was then loaded on a Phenyl Sepharose HP column equilibrated with 50 mM Tris pH 8.0, 10% (w/v) glycerol, 5 mM DTT, 1.0M NaCl, 1.5M ammonium sulfate, and eluted by an inverse salt gradient over 8 column volumes to 0.0M NaCl and 0.0M ammonium sulfate. FPPS eluted toward the end of the gradient. The fractions were analyzed by SDS-PAGE, pooled and loaded on a Superdex 75 size exclusion chromatography run with 25 mM Tris pH 8.0, 2.0 mM DTT, 25 mM NaCl. The fractions were pooled according to SDS-PAGE analysis, concentrated by ultrafiltration and dialysed against 20 mM sodium phosphate pH 7.2, 0.3M NaCl, 10 mM imidazole (buffer B). The sample was then loaded on a metal chelation column (HiTrap 5 ml) equilibrated with buffer B and eluted by a 10 mM to 1.0M gradient of imidazole. 154 units of PreScission protease were added and the reaction mix was dialysed overnight against 50 mM Tris pH 7.0, 150 mM NaCl, 1 mM EDTA, and 1 mM DTT. The dialysis buffer was then replaced by buffer B and the sample was subsequently loaded on the metal chelation column. The flow-through was collected, concentrated by ultrafiltration to about 4 ml and loaded (in four runs) on a Superdex 200 size-exclusion column equilibrated with buffer C (10 mM Tris pH 7.4, 25 mM NaCl, 5 mM TCEP). Fractions corresponding to the main peak were pooled, concentrated by ultrafiltration to 16 mg/ml, aliquoted and stored at −80° C.

### 4. Analytics

The purified protein had 350 amino acid residues in total, corresponding to the human FPPS sequence from asparagine 6 to lysine 353 with at its N-terminus an additional glycine and proline residue from the engineered PreScission protease cleavage site. It had a theoretical molecular weight of 40,141 Da. No residues were mutated. LC-ESMS analysis showed the expected mass. N-terminal sequencing by Edman degradation was in agreement with the expected amino acid sequence.

### 5. Crystallization

Crystallization was performed by the vapor diffusion method. Both the sitting drop (in Corning 96 well plates) and the hanging drop techniques (in Linbro 24 well plates) were used. The crystals used in this study were grown at 19° C. from 1.2M Na/K phosphate pH 4.7, 25% (v/v) glycerol, except for the pamidronate/Mn2+ complex which was grown at pH 5.3 under otherwise identical experimental conditions.

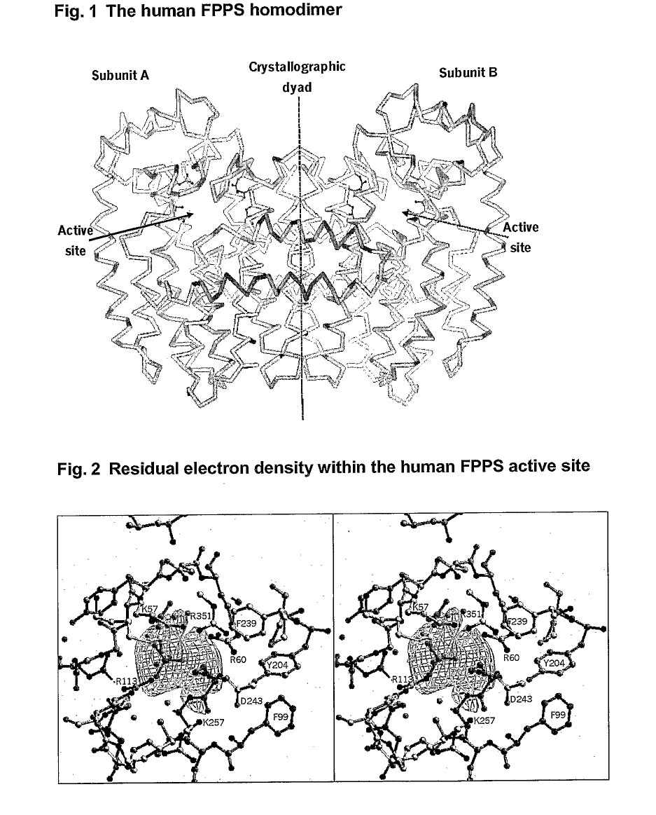

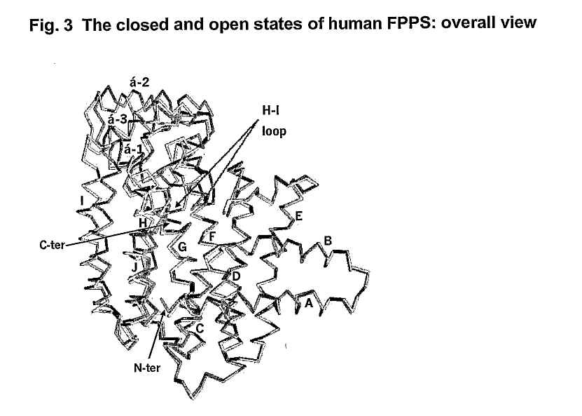

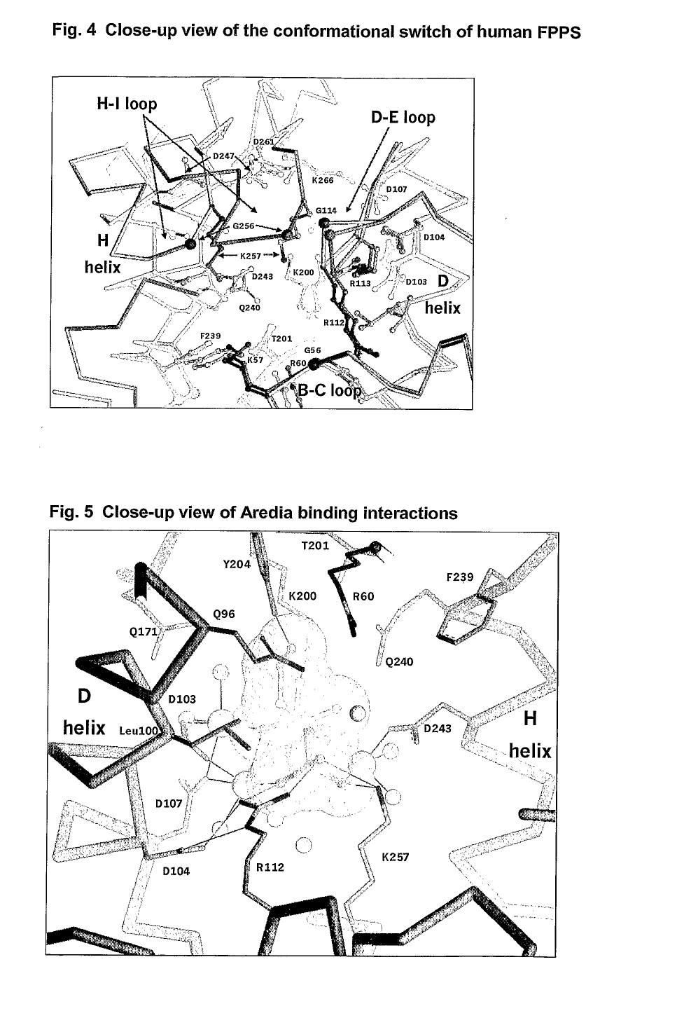

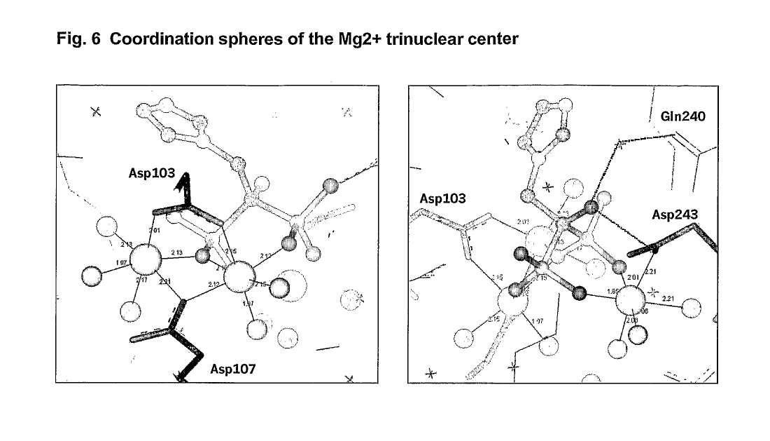

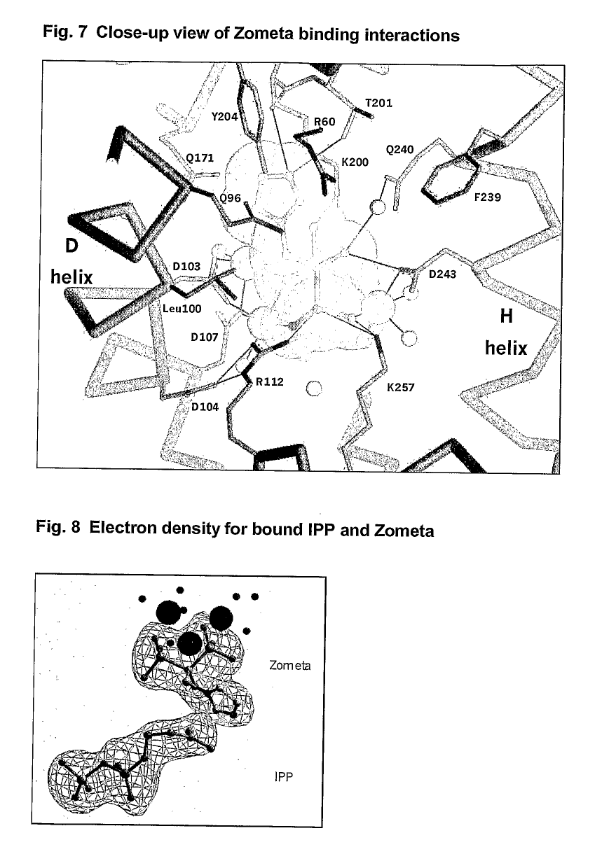

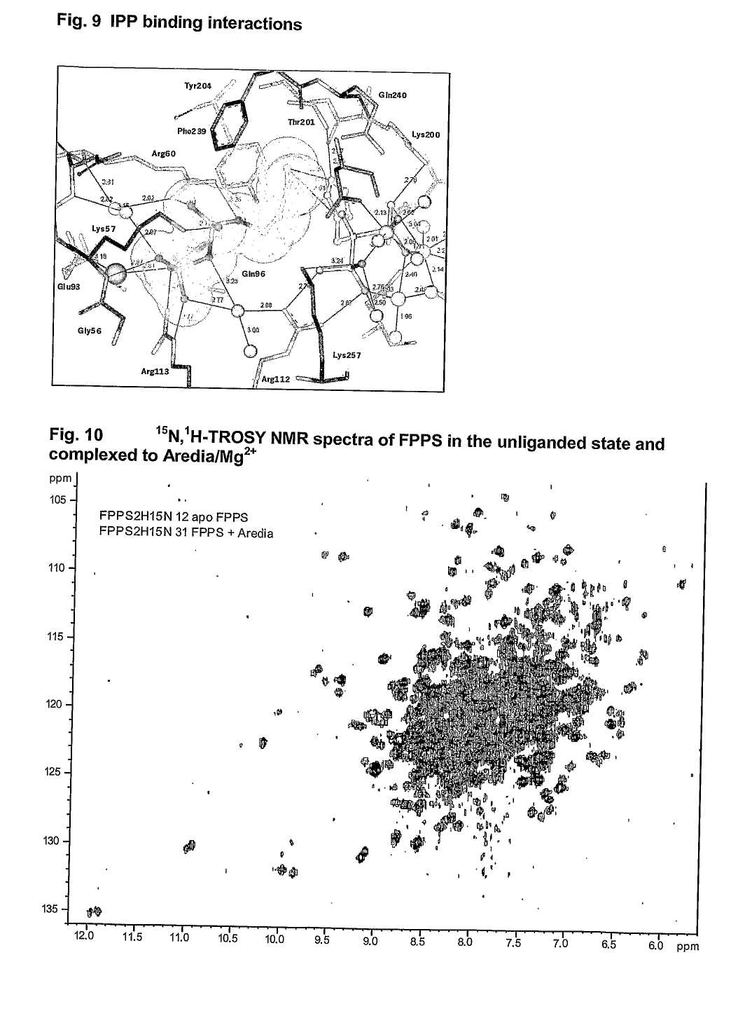

Large single crystals of human FPPS can be obtained under a variety of high salt conditions (1.0M ammonium citrate or 1.2M to 1.8M Na/K phosphate or ammonium phosphate) at pH 4.0 to 5.6 (FIG. 3-1). The crystals are fragile and are therefore preferably grown under conditions directly suitable for cryo-crystallography (1.2M Na/K phosphate, pH 4.7 to 5.6, 25% (v/v) glycerol). Two crystal forms can be observed, both in space group P4₁2₁2 with one FPPS subunit per asymmetric unit. Crystals of unliganded human FPPS in the open conformation have approximate cell dimensions of a=b=111 Å, c=77 Å (crystal form II) while those corresponding to the closed conformation exhibit cell dimensions of about a=b=112 Å, c=66 Å (crystal form I). Isomorphous crystals to the latter can be obtained under similar experimental conditions in the presence of pamidronate, zoledronate/IPP, and 5 mM MgCl₂ or MnCl₂.

Crystals of the unliganded enzyme in the open conformation were obtained with human FPPS isolated according to purification method 2. All other crystals were prepared with batch 1 (purification method 1). With both enzyme batches, the protein stock solutions were 16 mg/ml human FPPS (6-353) in 25 mM Tris-HCl pH 8.0, 25 mM NaCl, 2 mM DTT. Apo crystals were prepared by mixing equal volumes of the crystallization solution and protein stock. The complexes with pamidronate, zoledronate and IPP were prepared by co-crystallization at a reduced protein concentration (4.2 mg/ml) in presence of 5 mM MgCl₂ or MnCl₂. Stock solution of pamidronate (50 mM) and zoledronate (10 mM) were prepared in plain water and added to the enzyme to a final concentration of 2.5 mM and 0.5 mM, respectively. IPP was purchased from Sigma as a 1 mg/ml solution in 70% methanol, 30% 10 mM ammonium hydroxide, and diluted 1:50 with protein (3-fold molar excess of substrate).

5b. Crystallization of the FPPS Complex with Ibandronate

Crystals of the FPPS complex with Ibandronate were grown at 19° C. from 0.1M zinc acetate, 0.1M Na acetate, 12% PEG 4000 pH 4.4 by the vapour diffusion in sitting drop technique. Protein stock was 13.8 mg/ml human FPPS (6-353) in 10 mM Tris pH 7.4, 25 mM NaCl, 5 mM MgCl₂ and 1.0 mM Ibandronate.

### 6. X-ray Data Collection

X-ray data were collected at 95K using a MARCCD 165 mm detector and synchrotron radiation (Swiss Light Source, beam line XS06A). The crystals were mounted in cryo-loops and directly flash-frozen in the cold nitrogen stream. Diffraction data were recorded as 1.0° oscillation images which were processed and scaled with the HKL program suite version 1.96.6 (Otwinowski and Minor, 1997) or XDS/XSCALE (Kabsch, 1993). All crystals were in space group P4₁2₁2 with one FPPS monomer per asymmetric unit. Crystal data and data collection statistics are shown in Table 1.

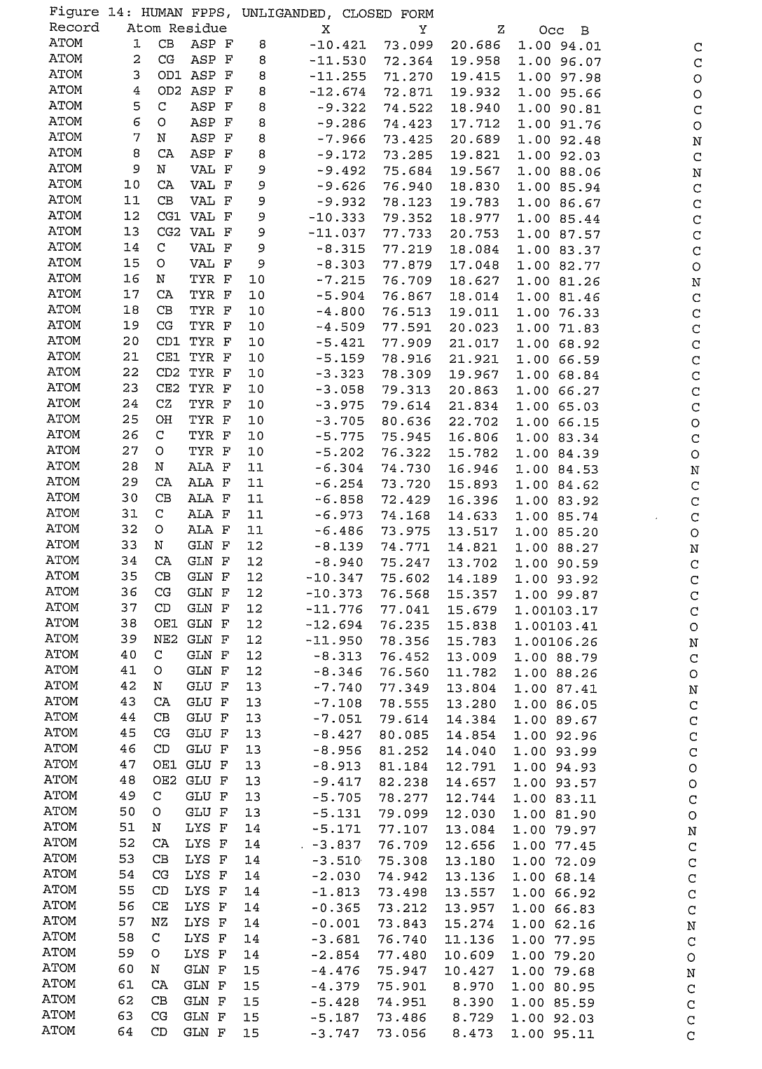

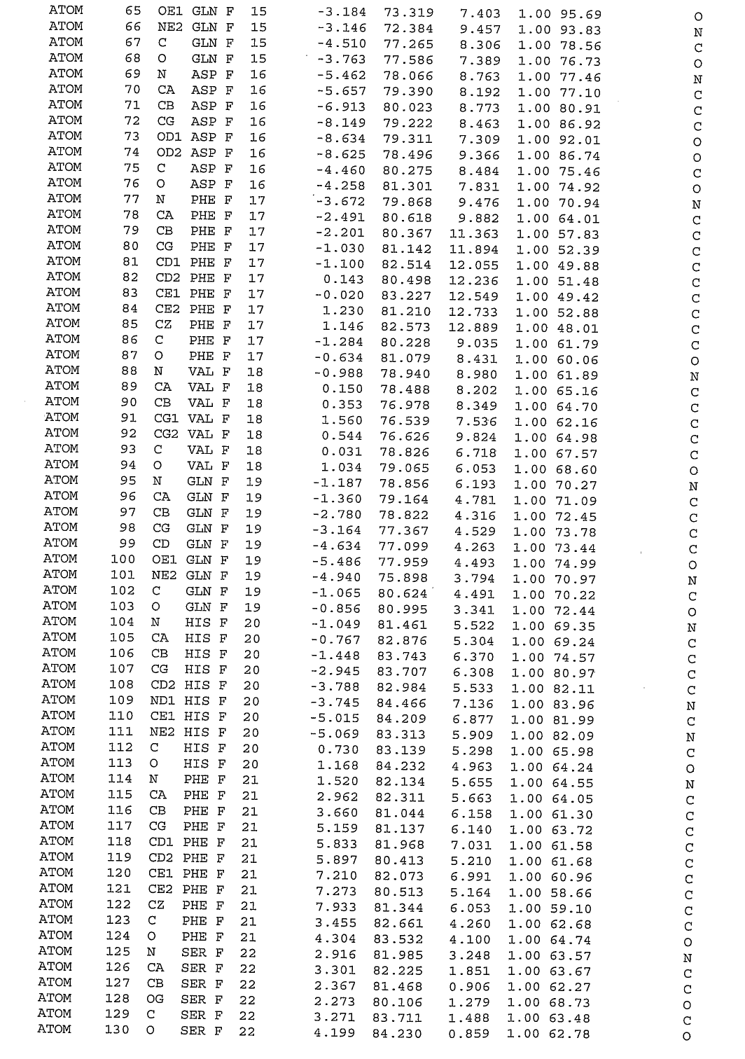

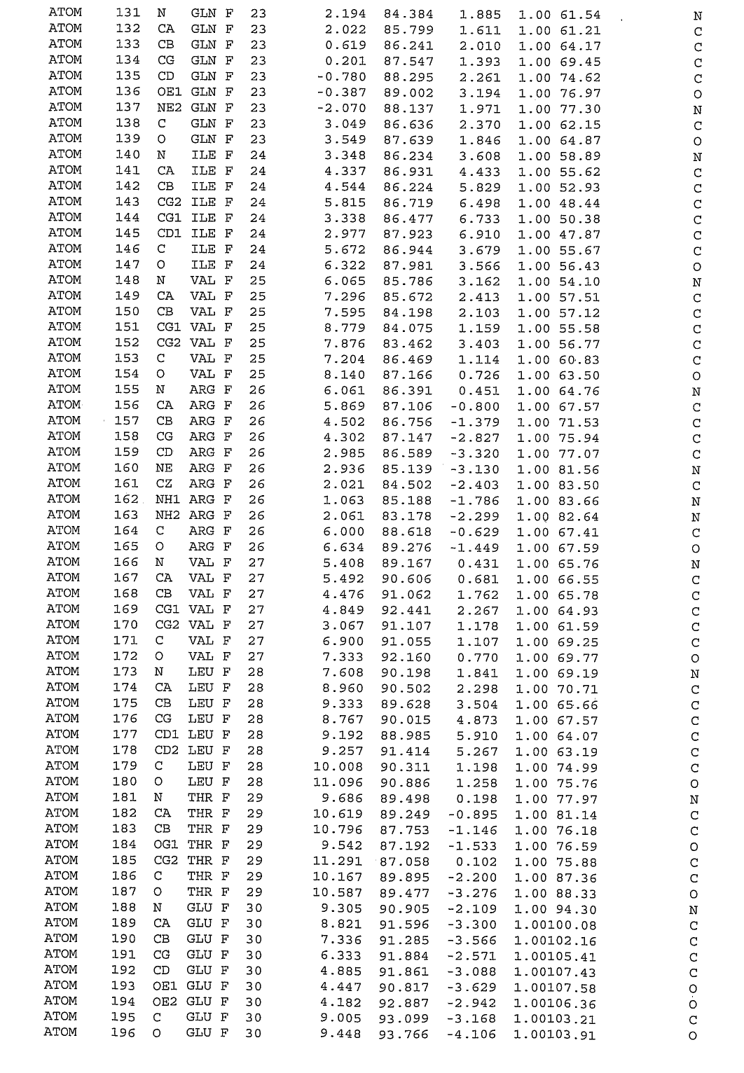

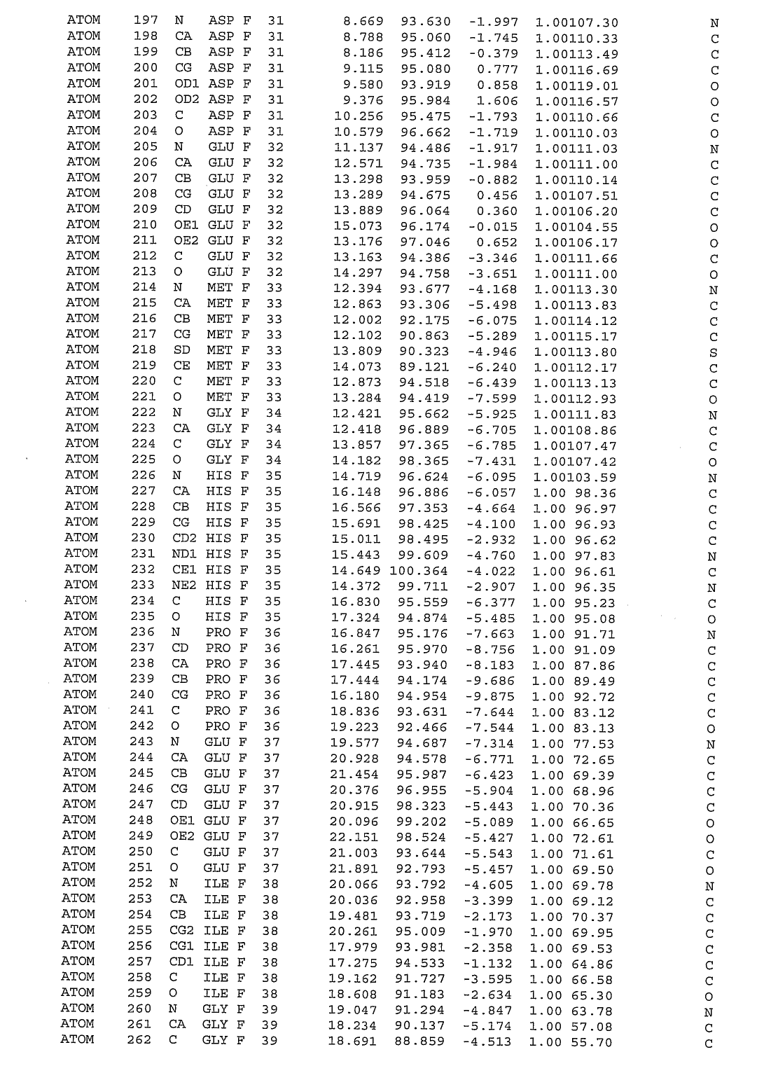

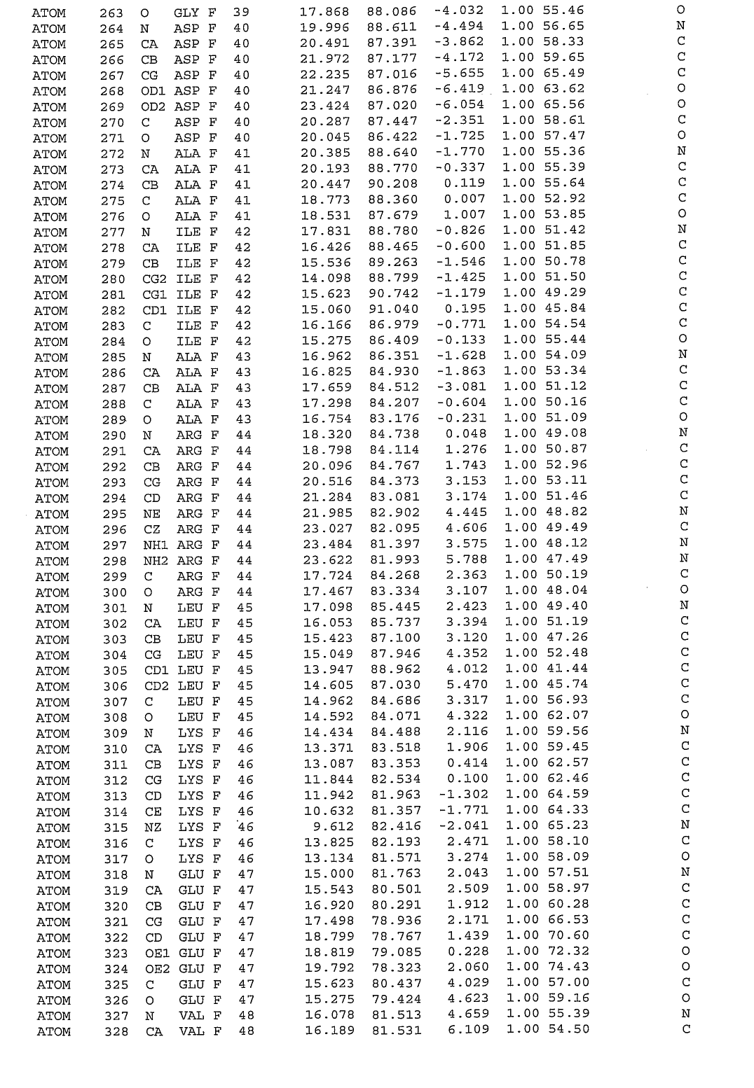

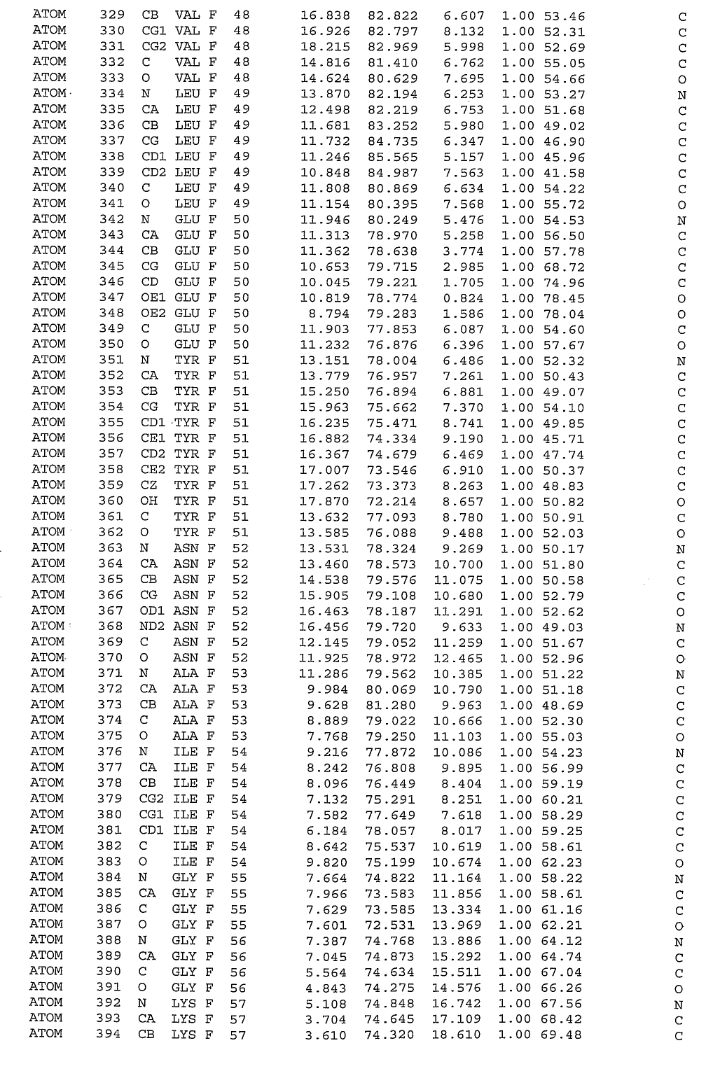

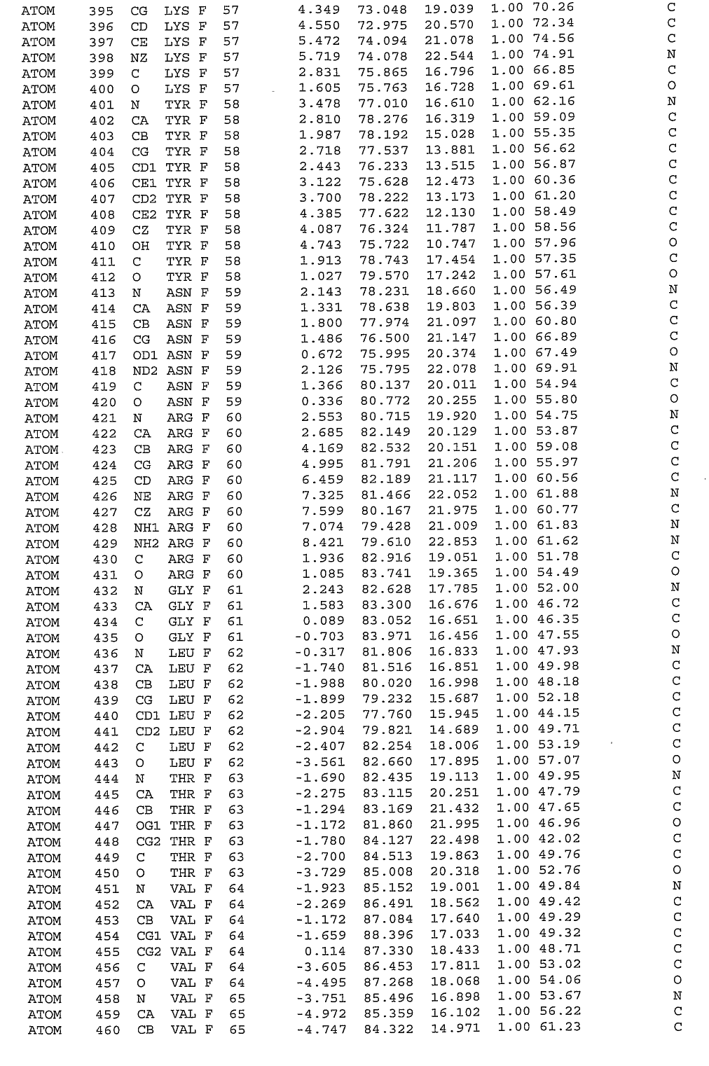

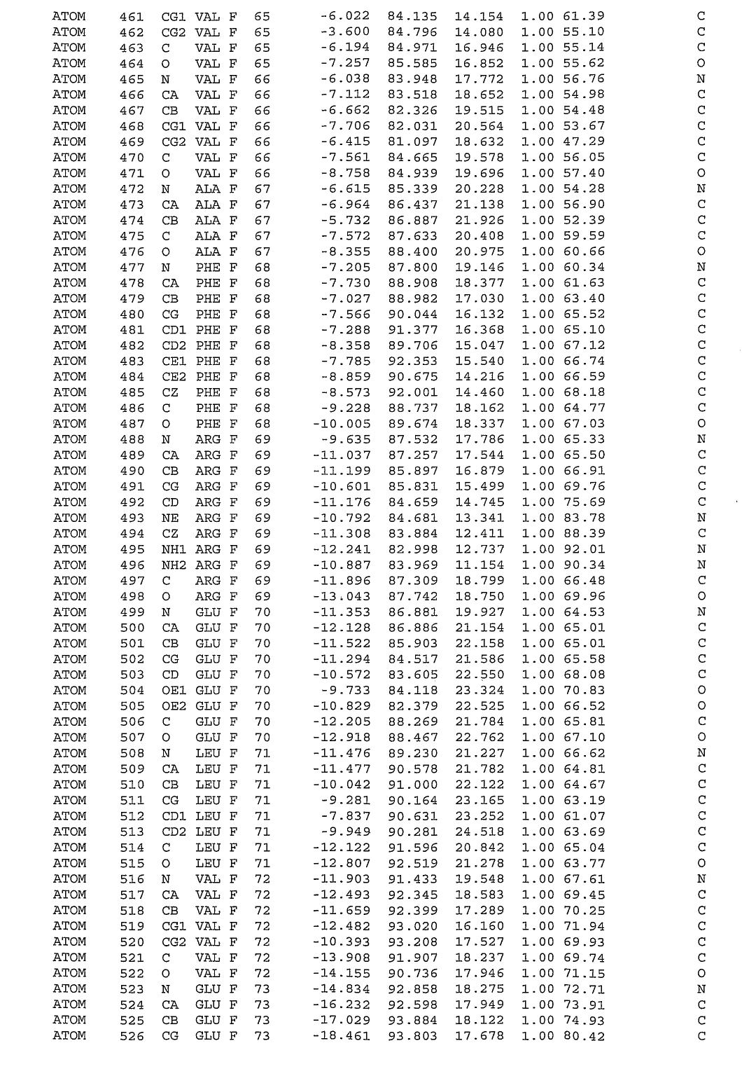

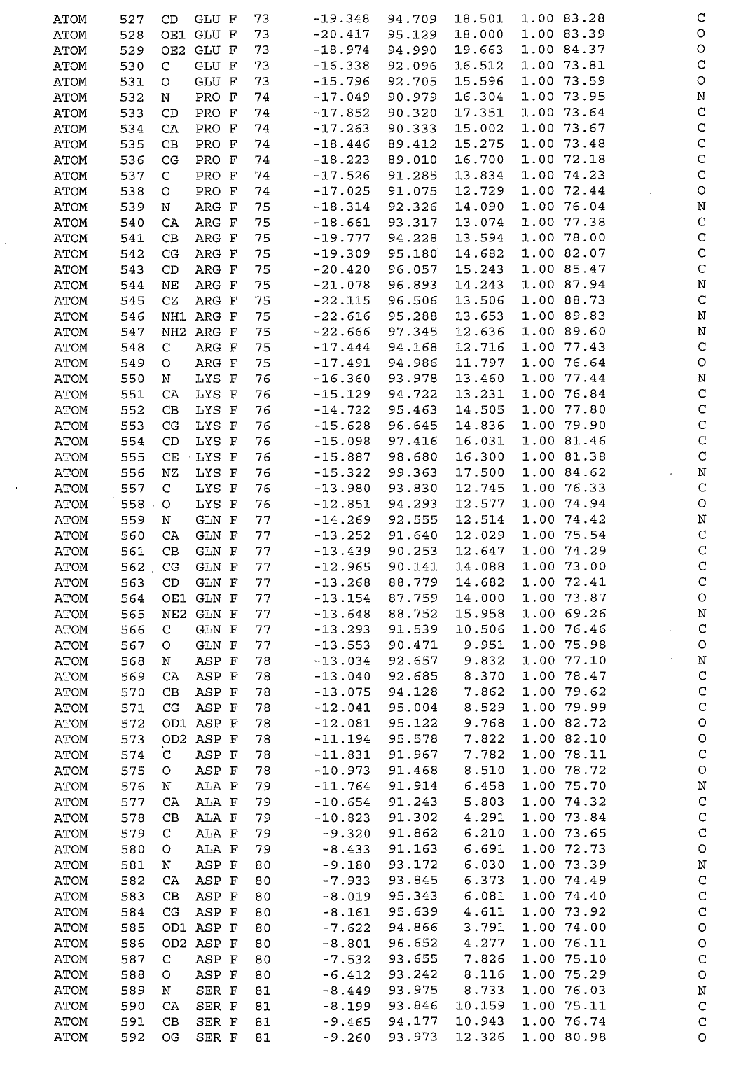

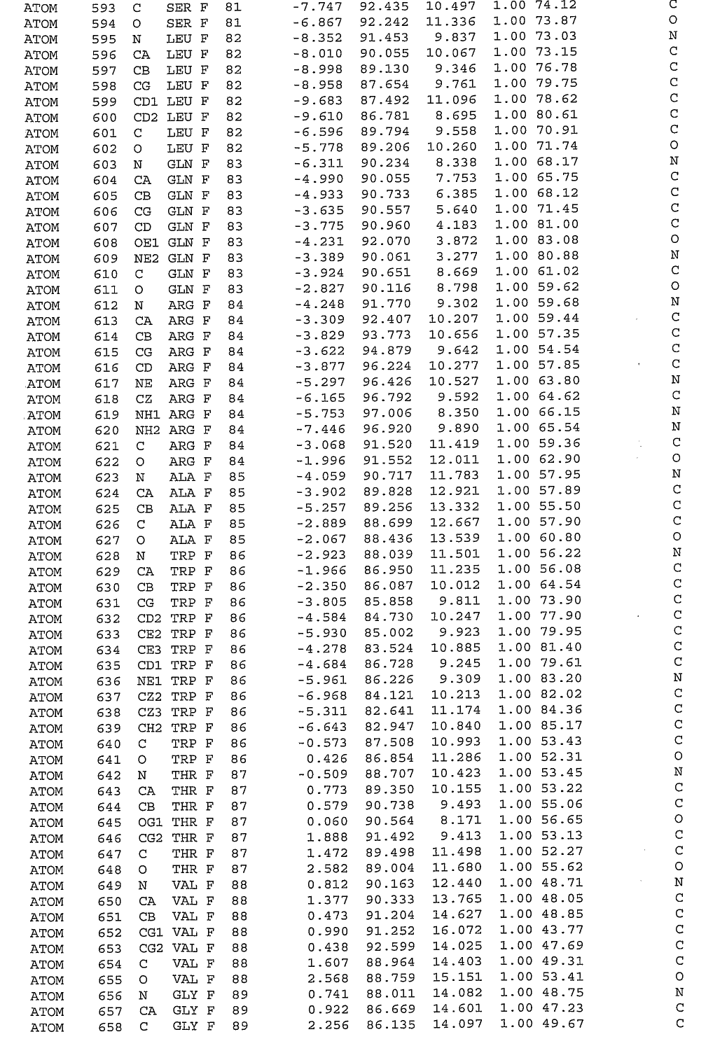

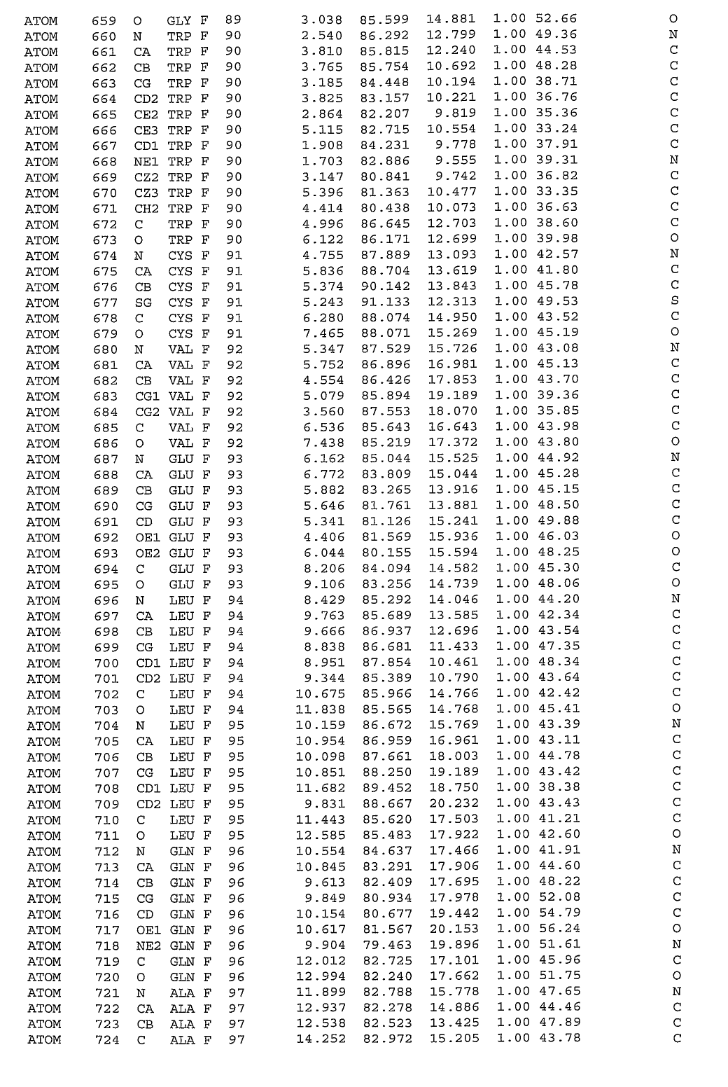

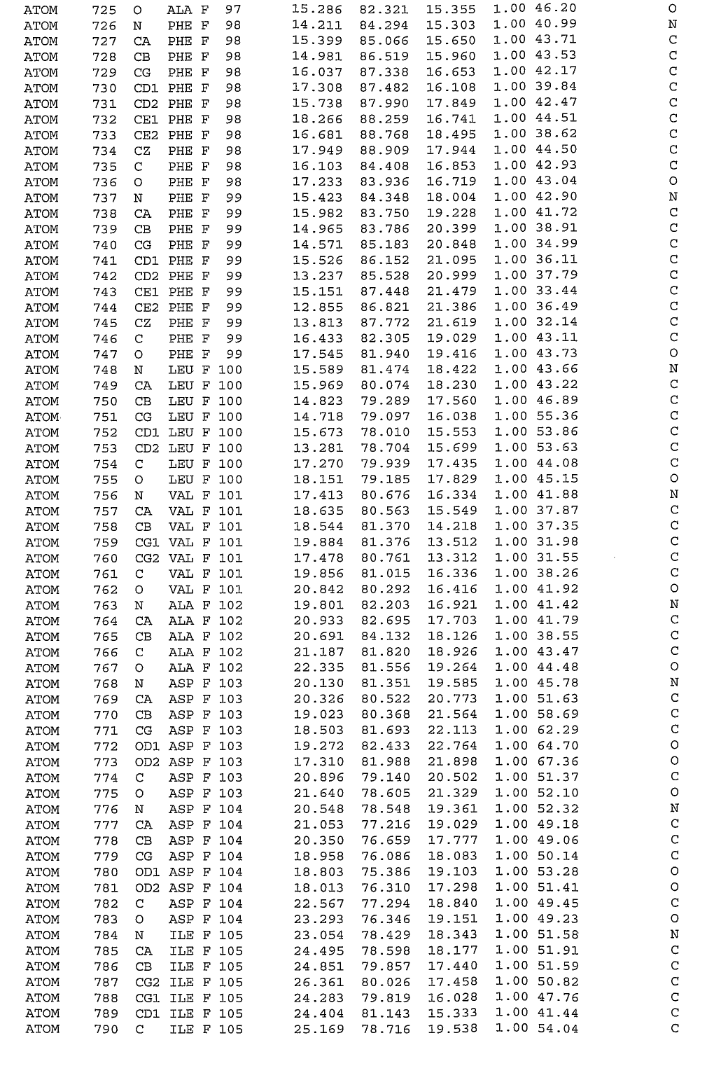

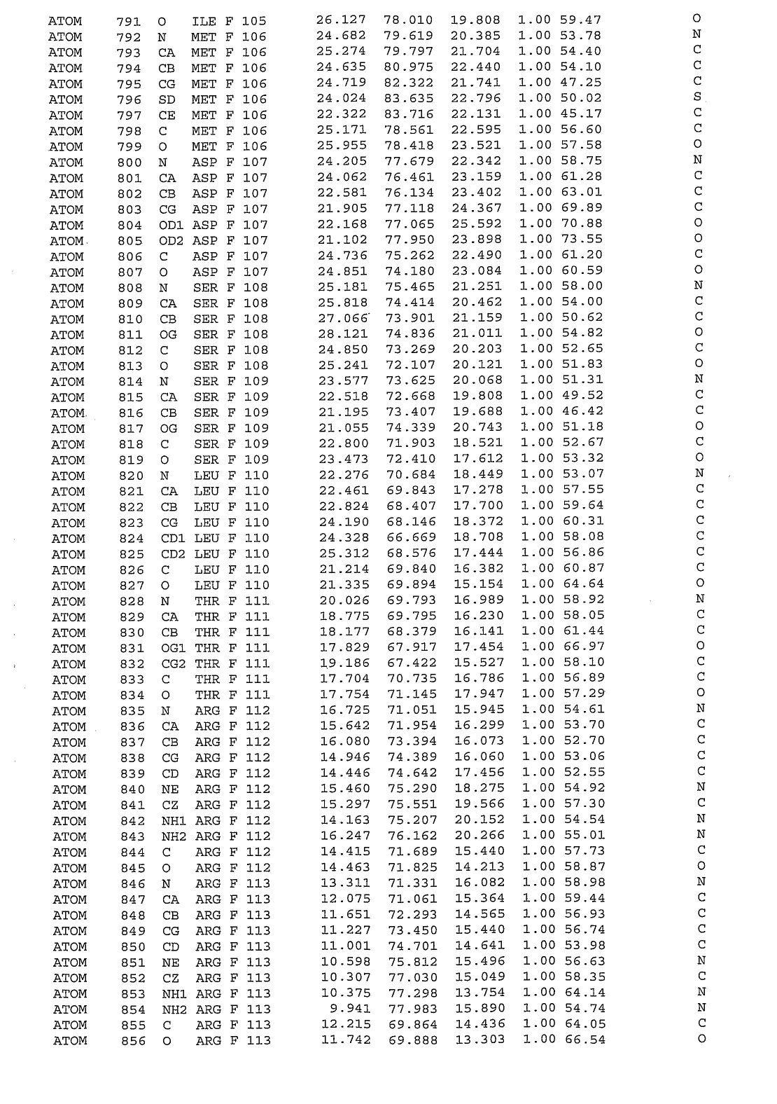

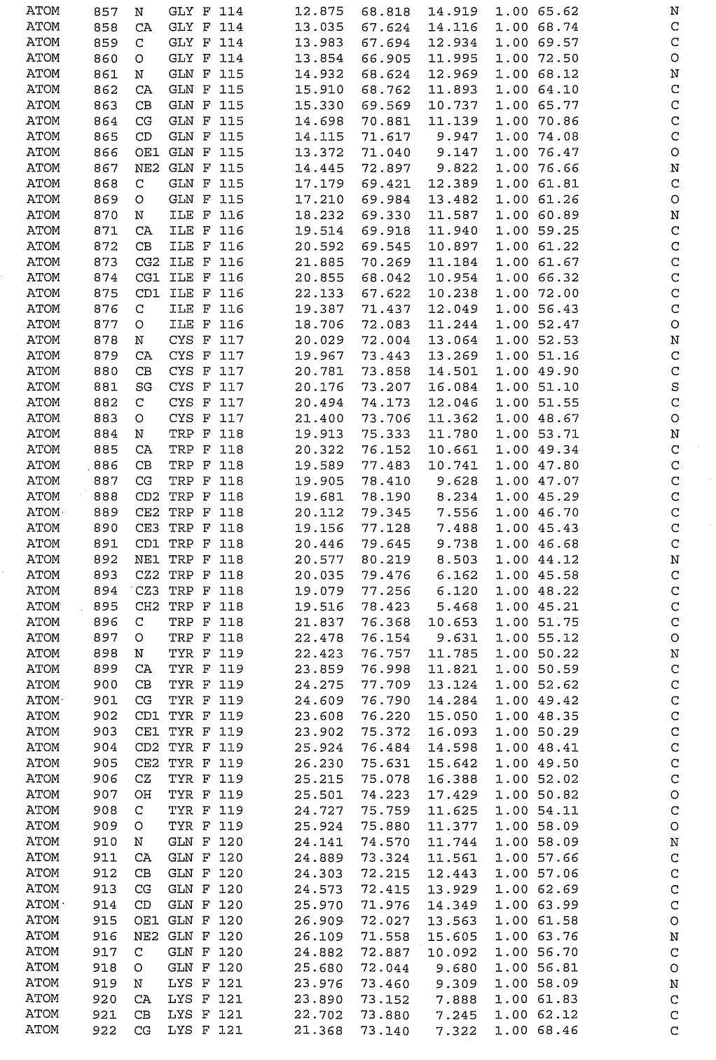

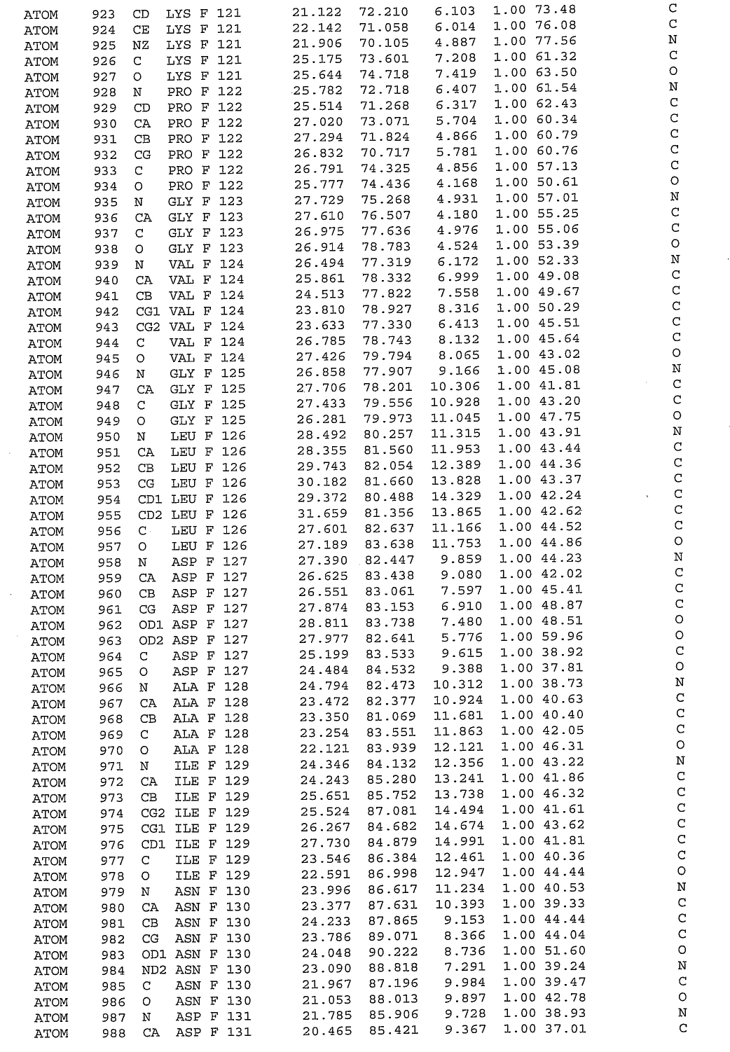

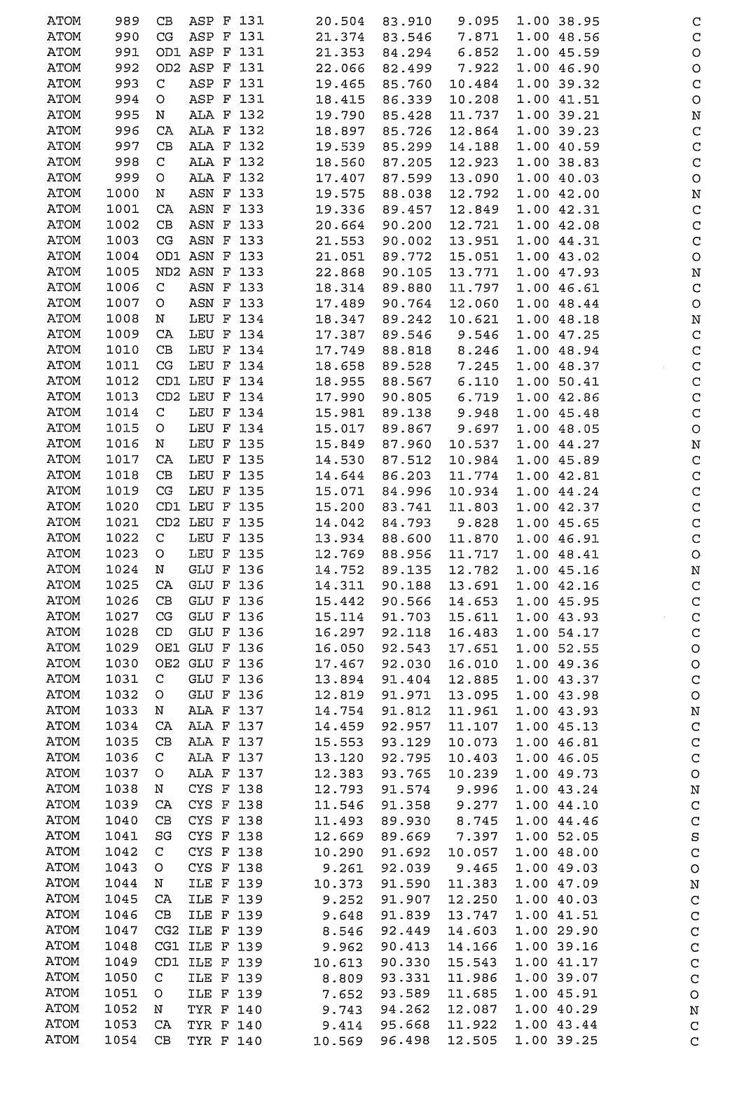

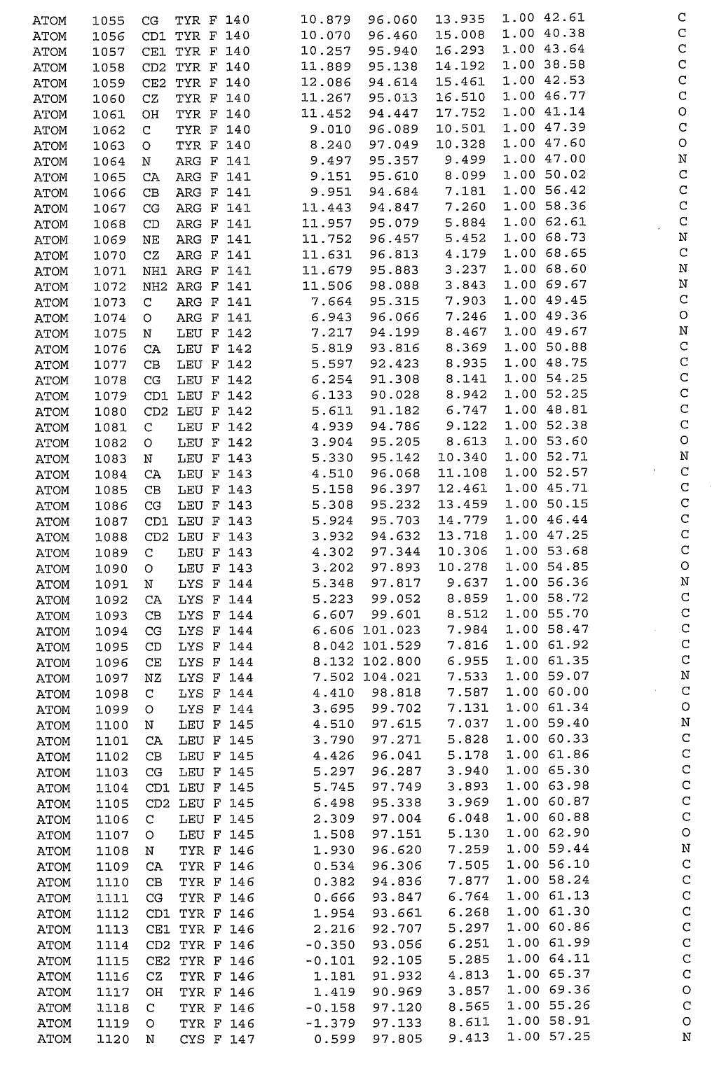

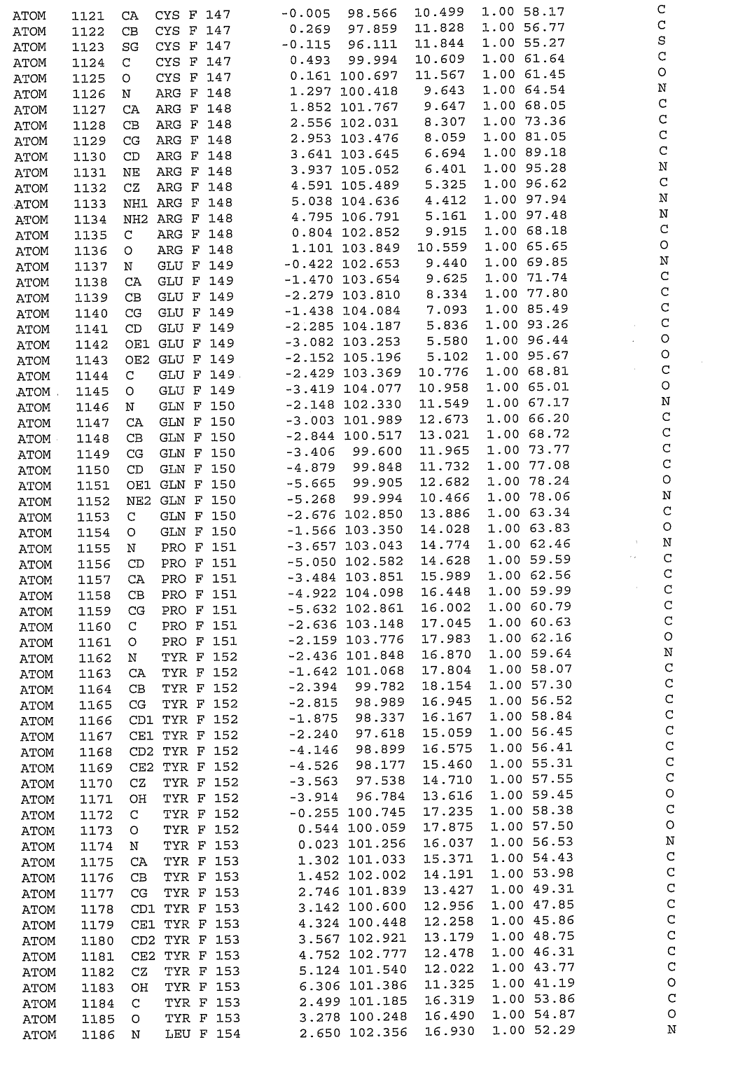

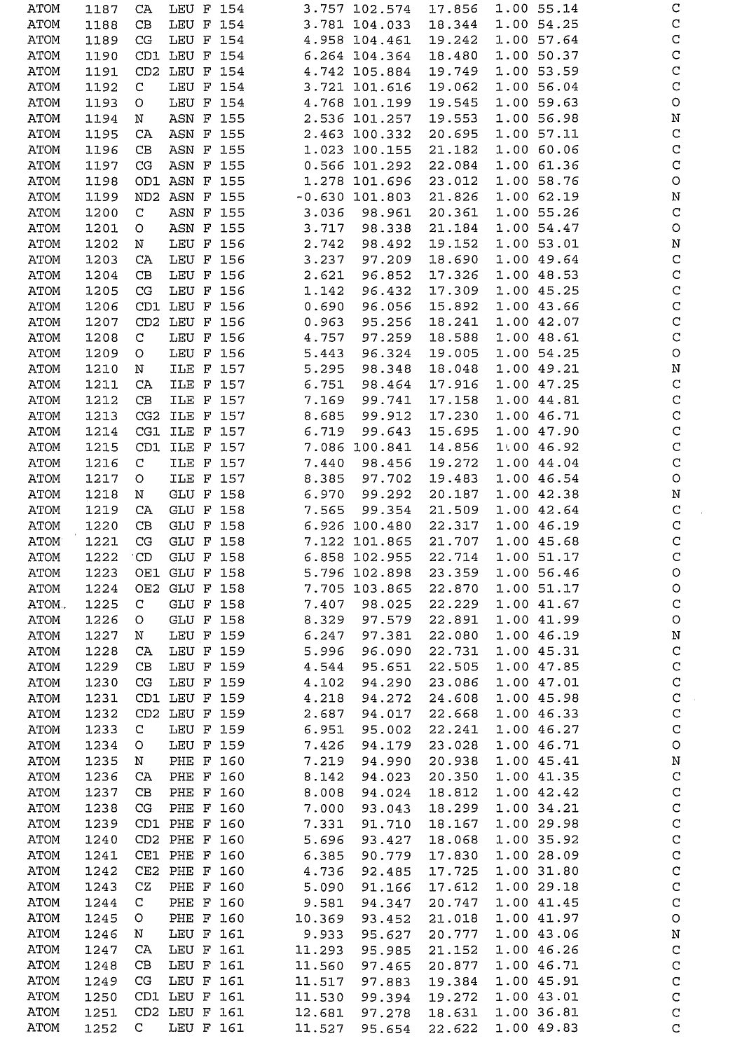

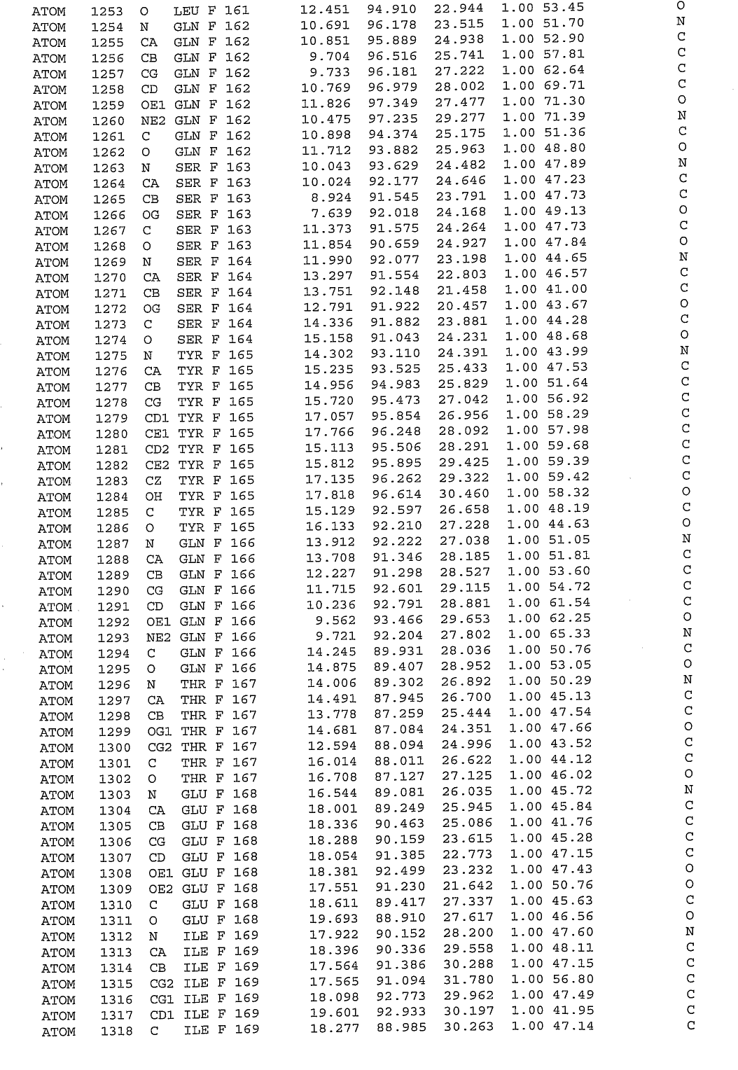

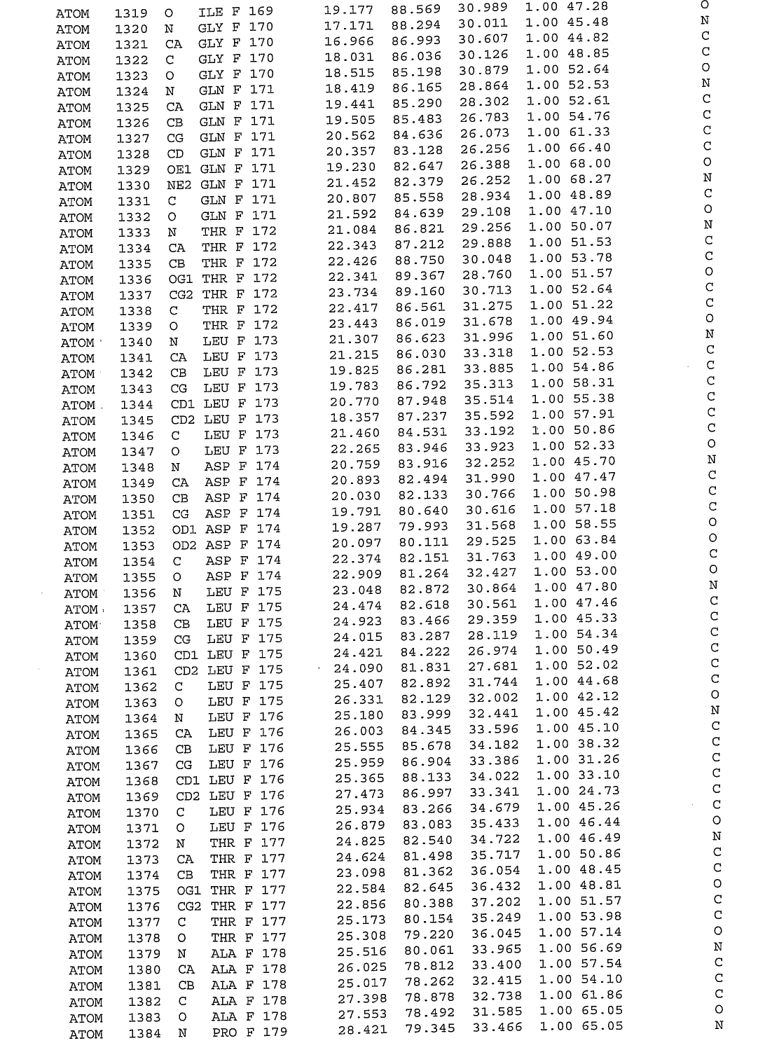

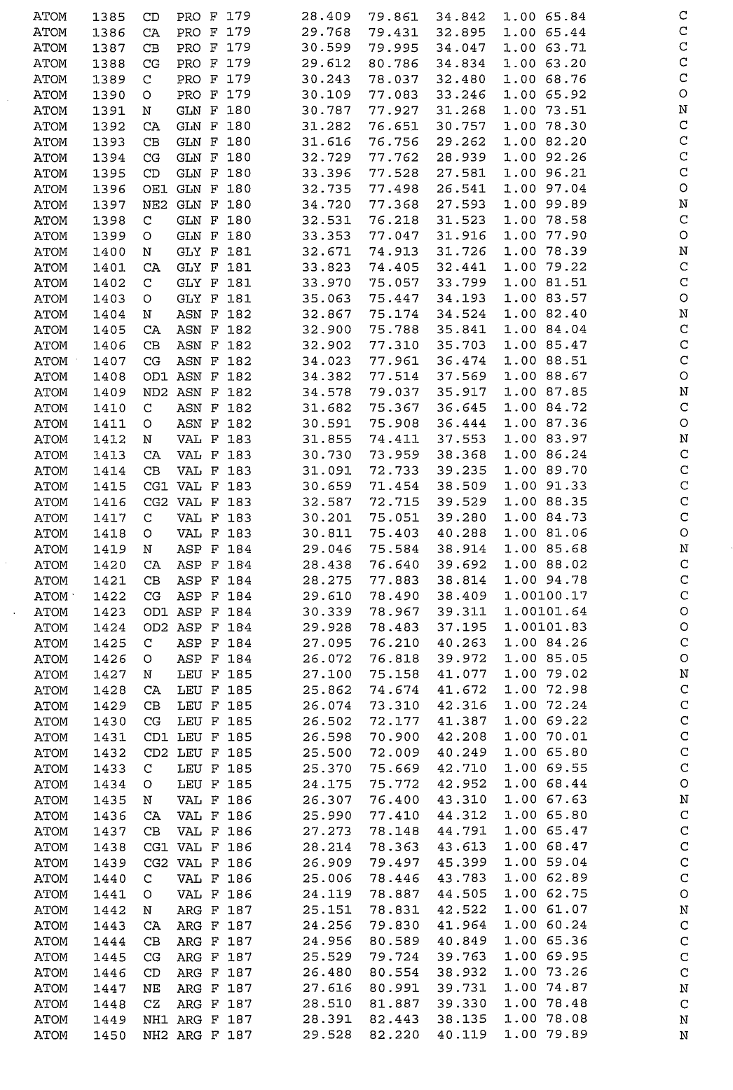

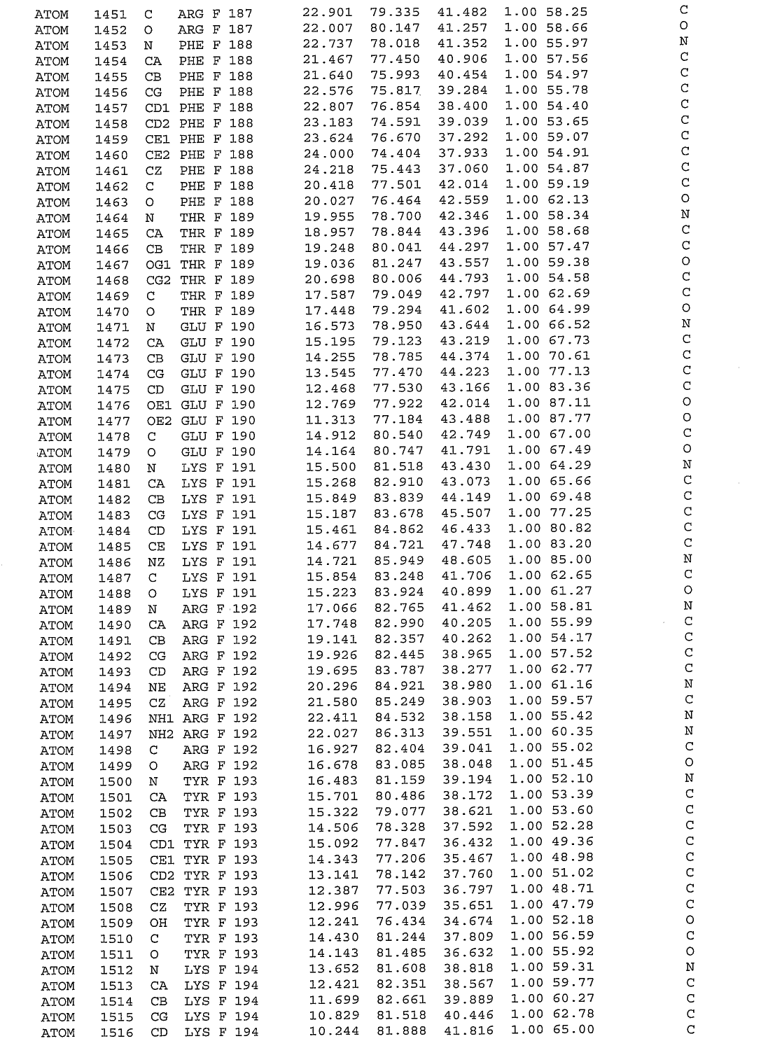

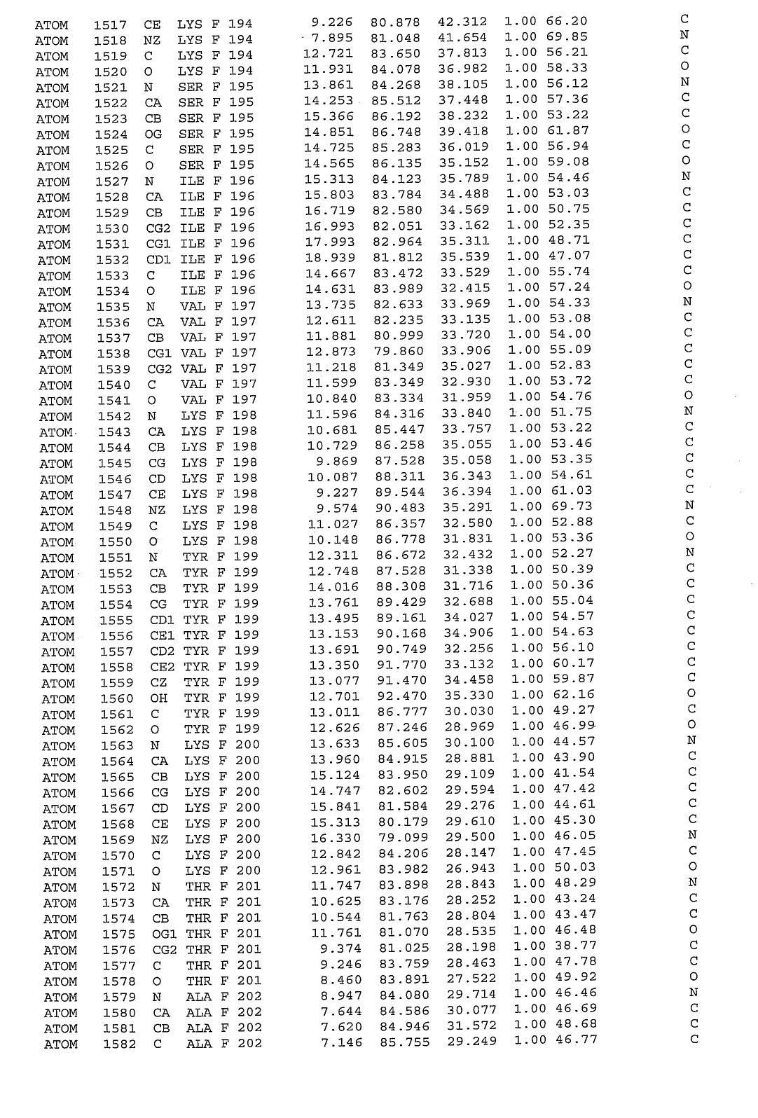

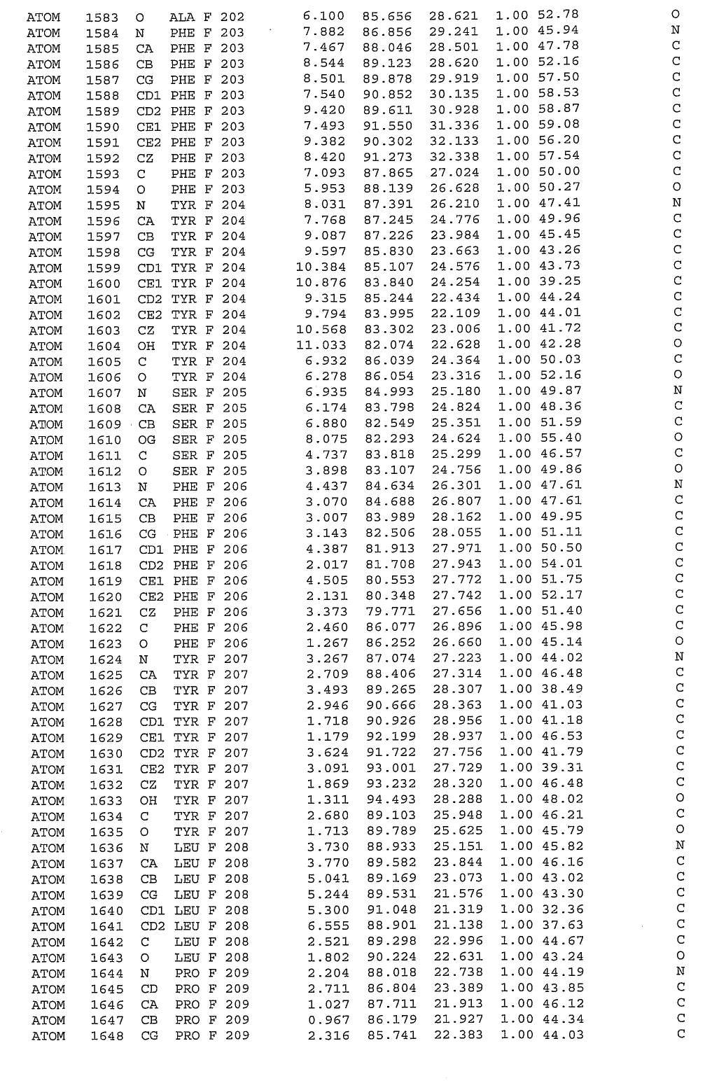

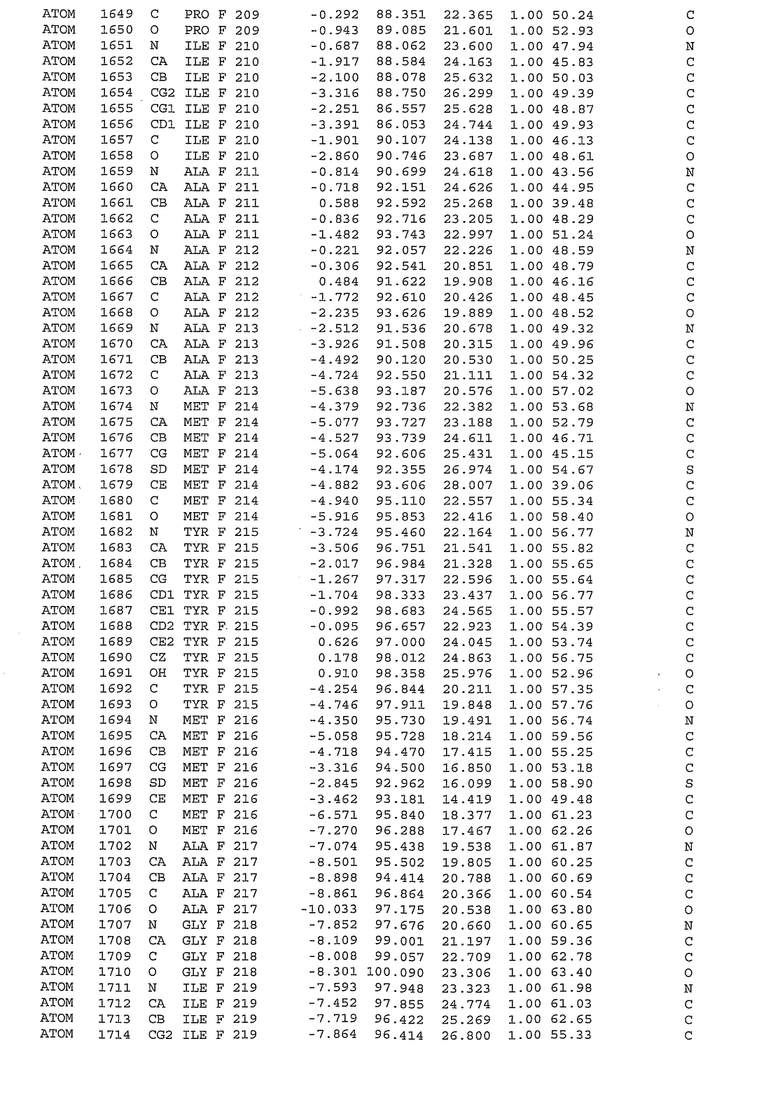

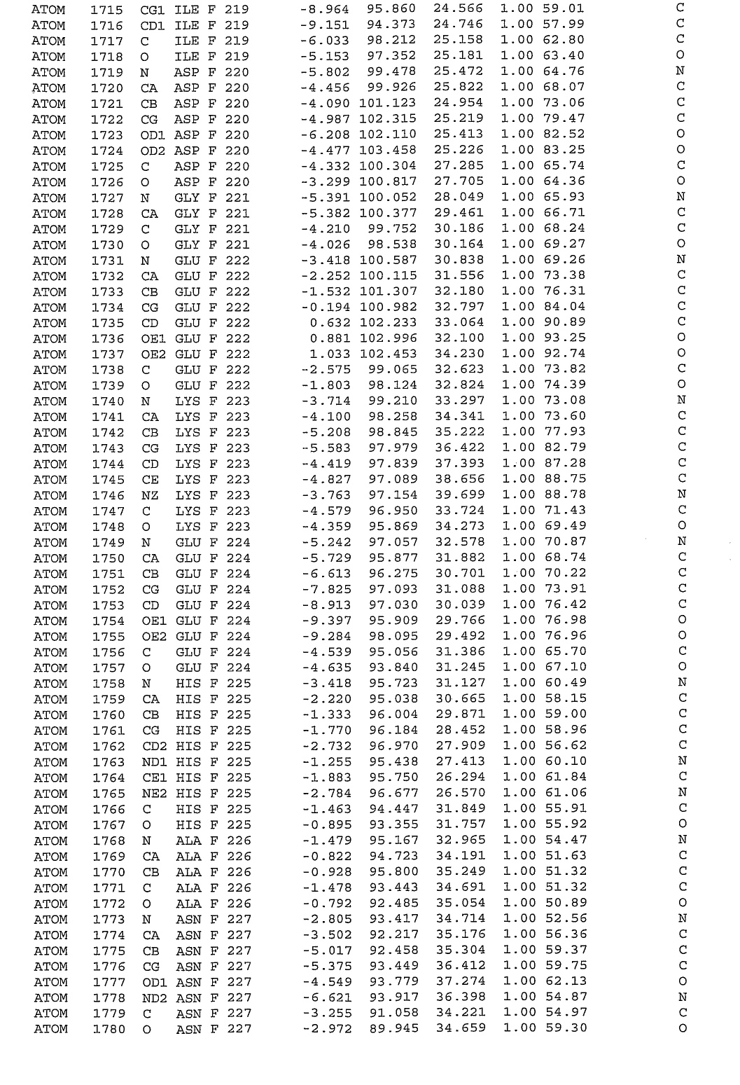

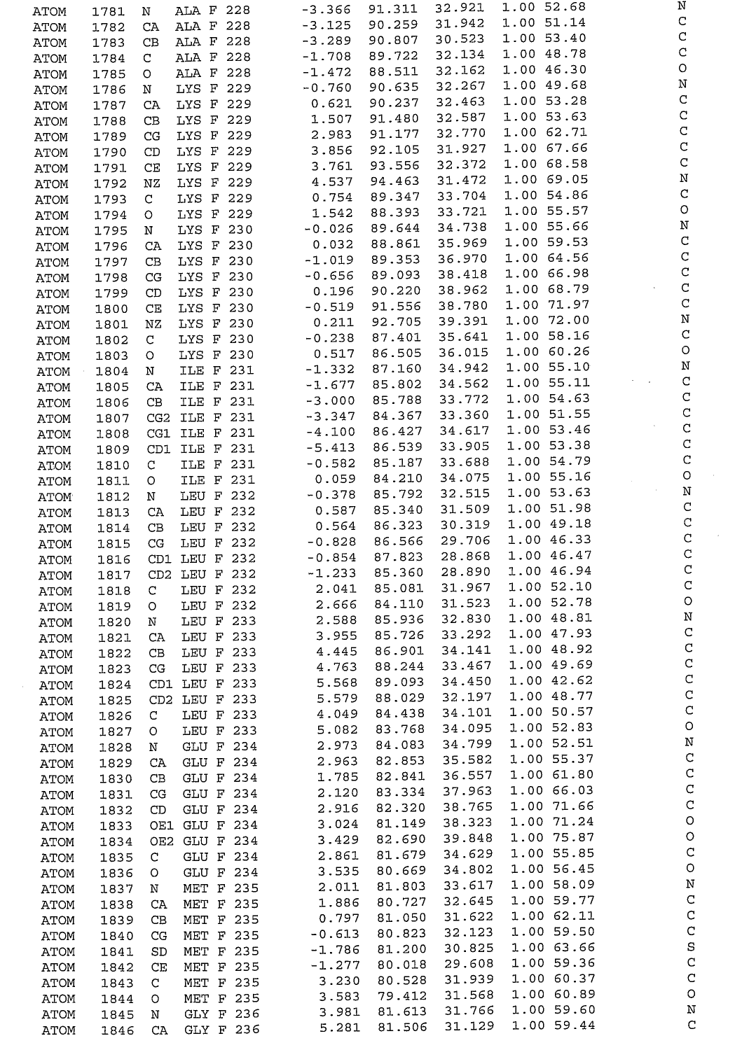

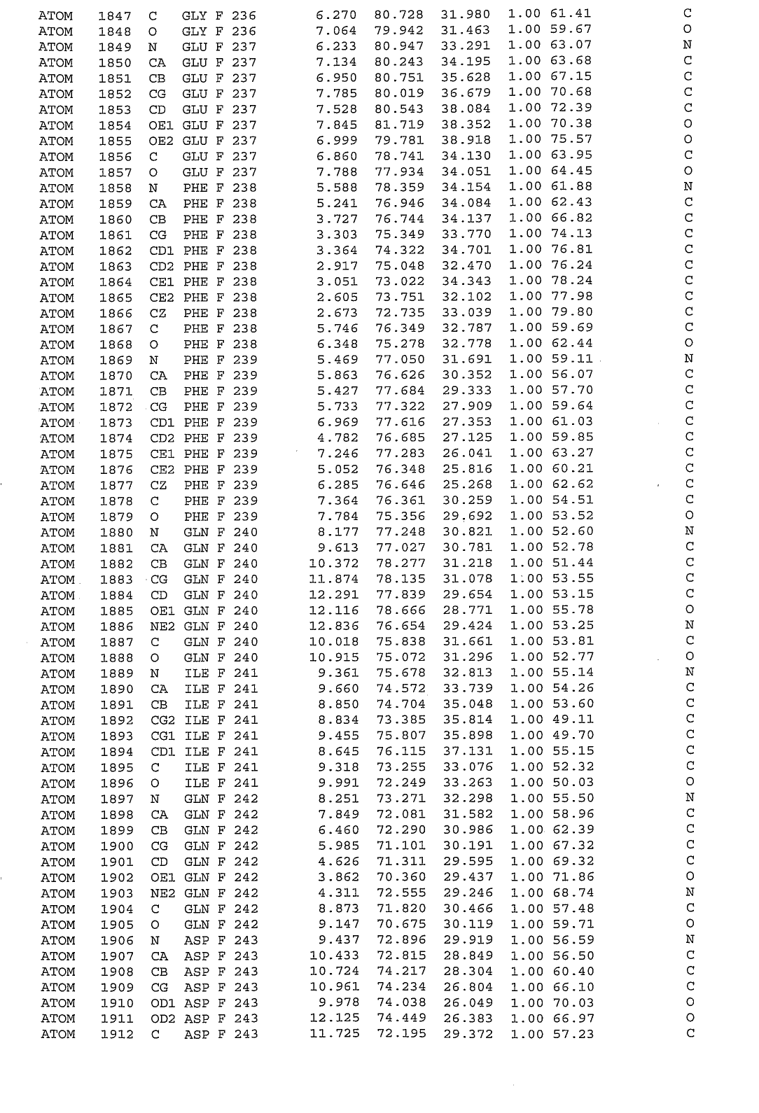

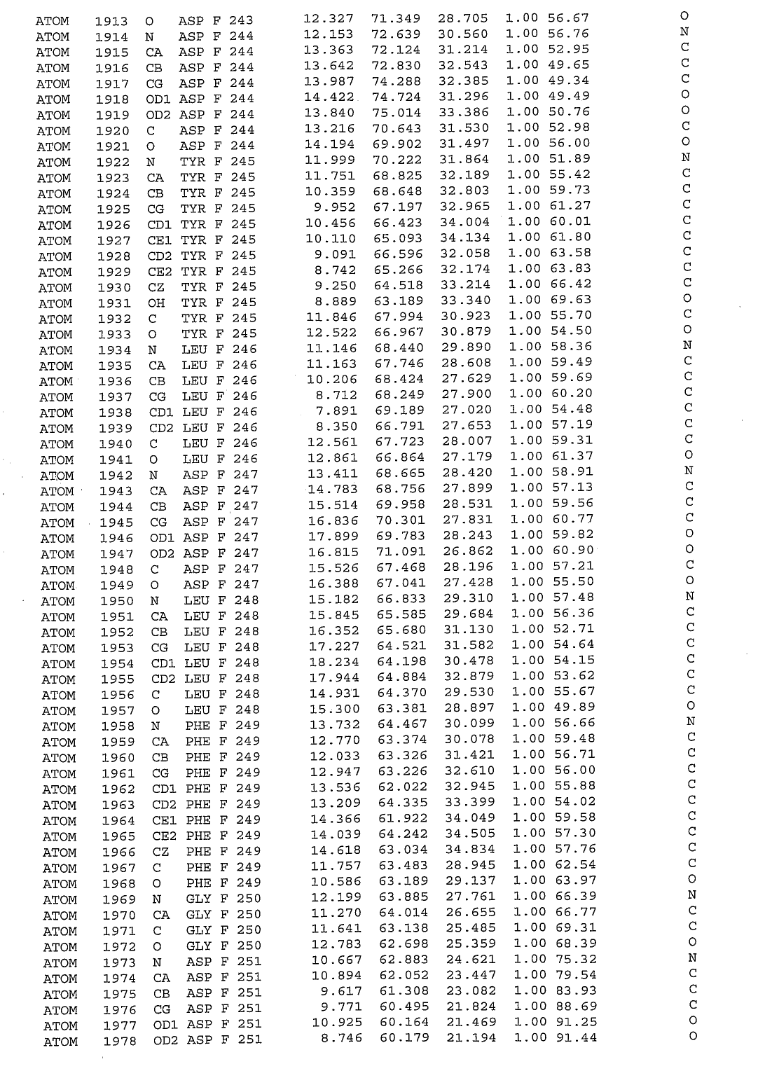

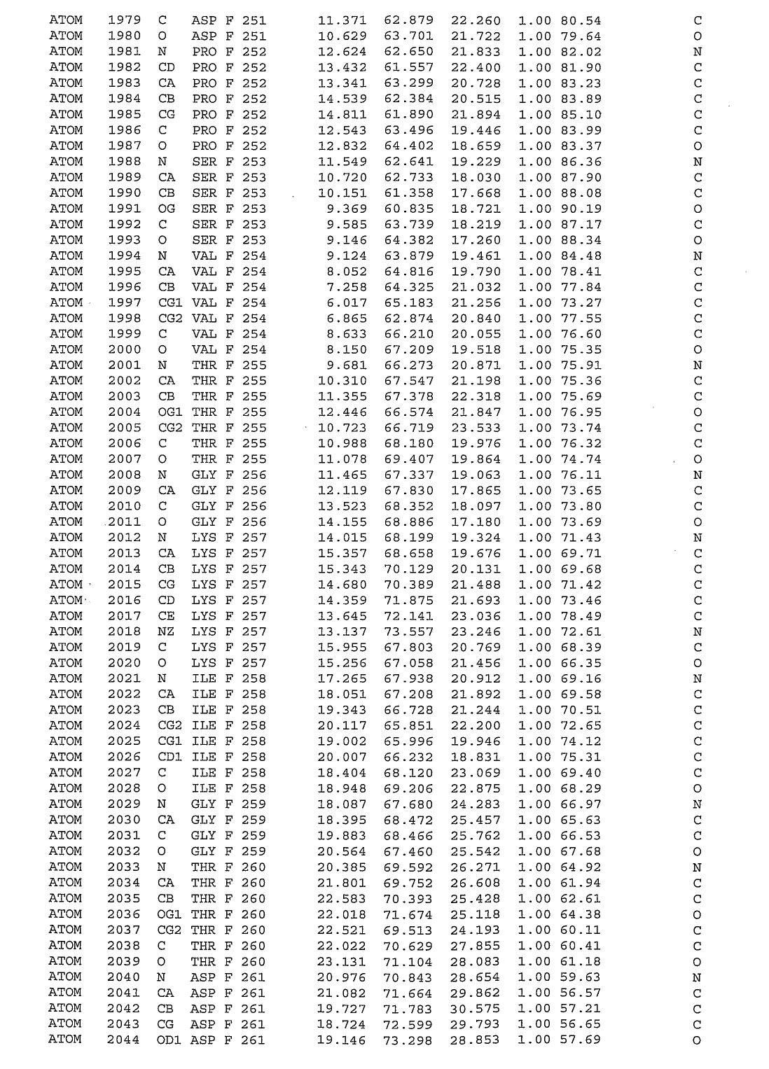

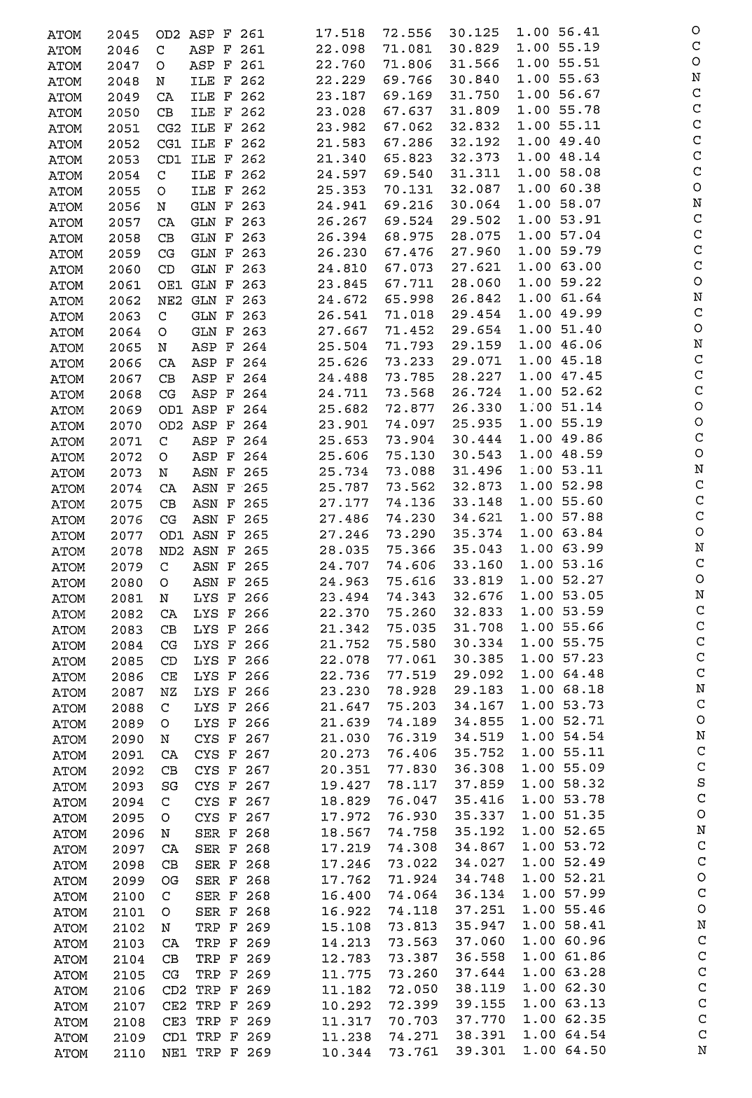

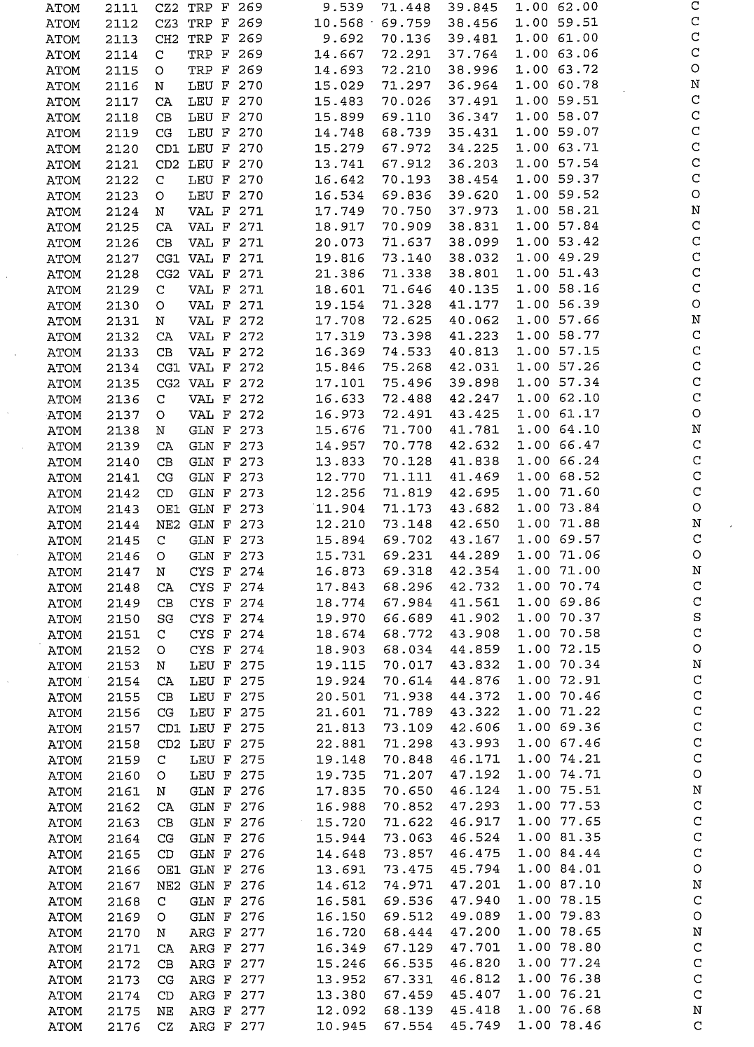

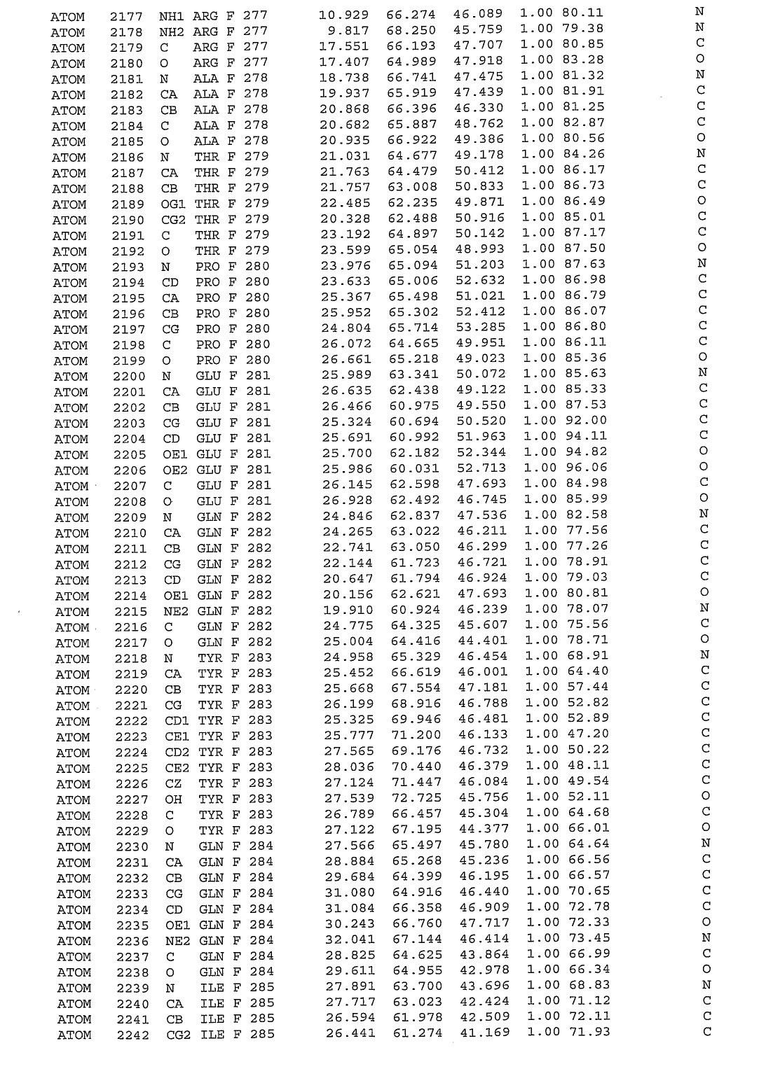

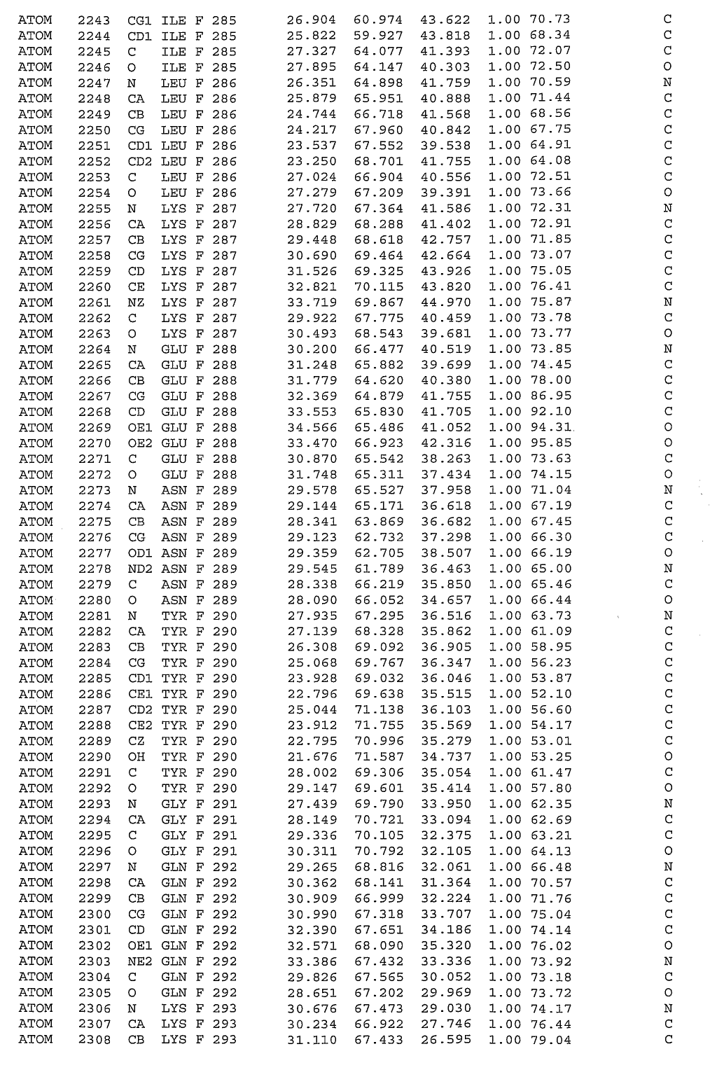

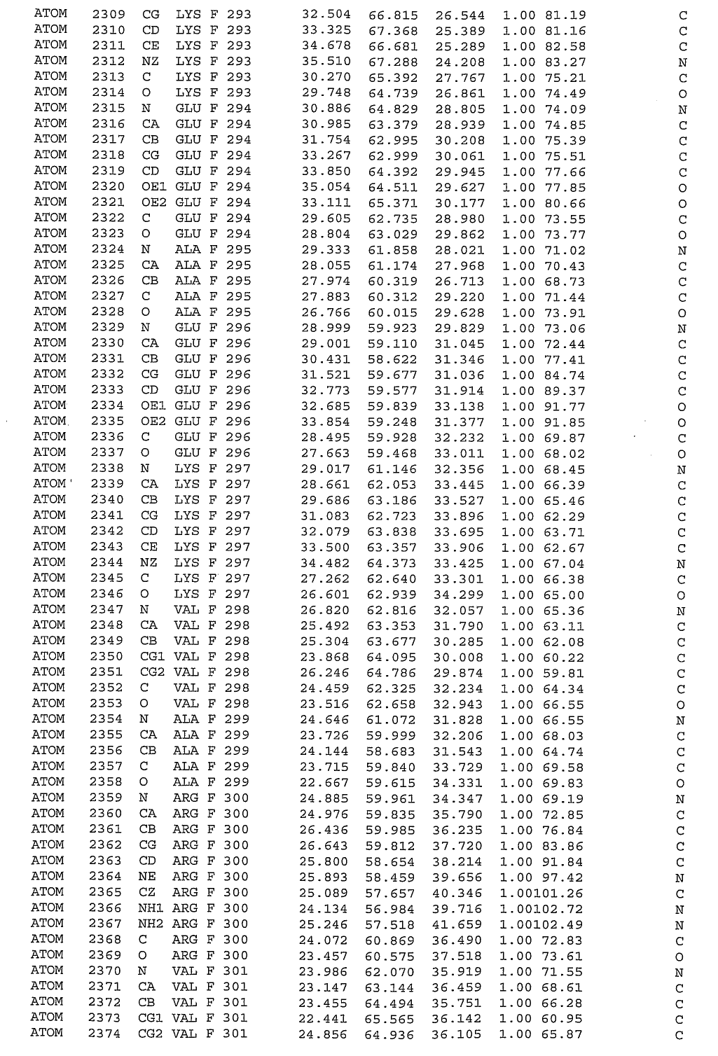

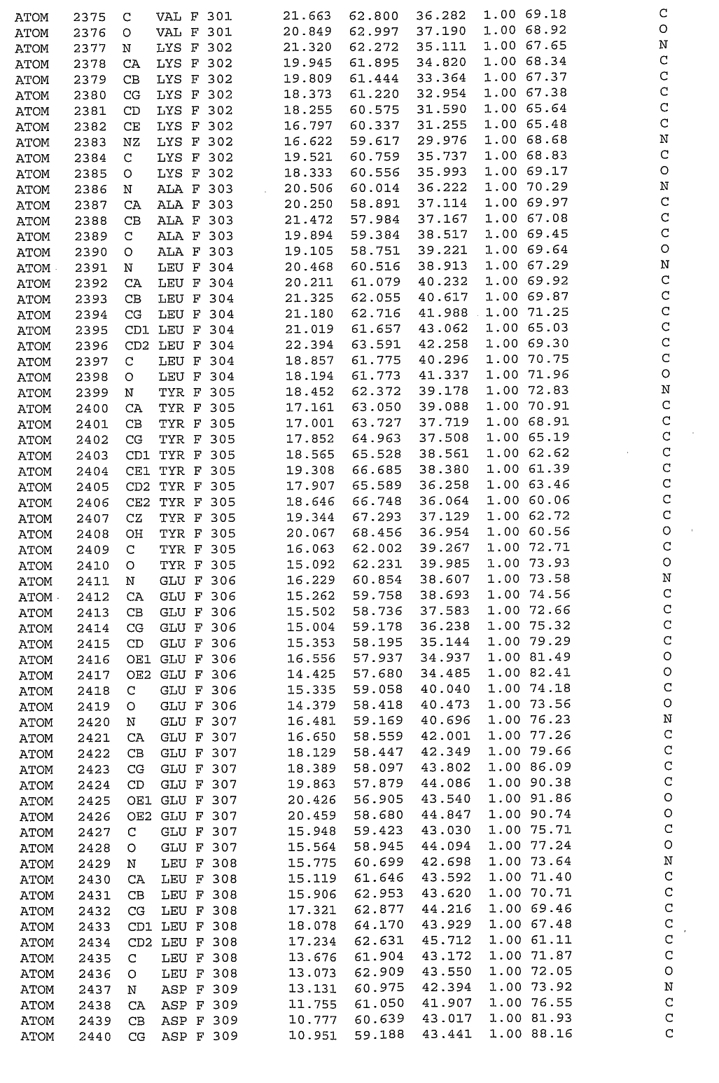

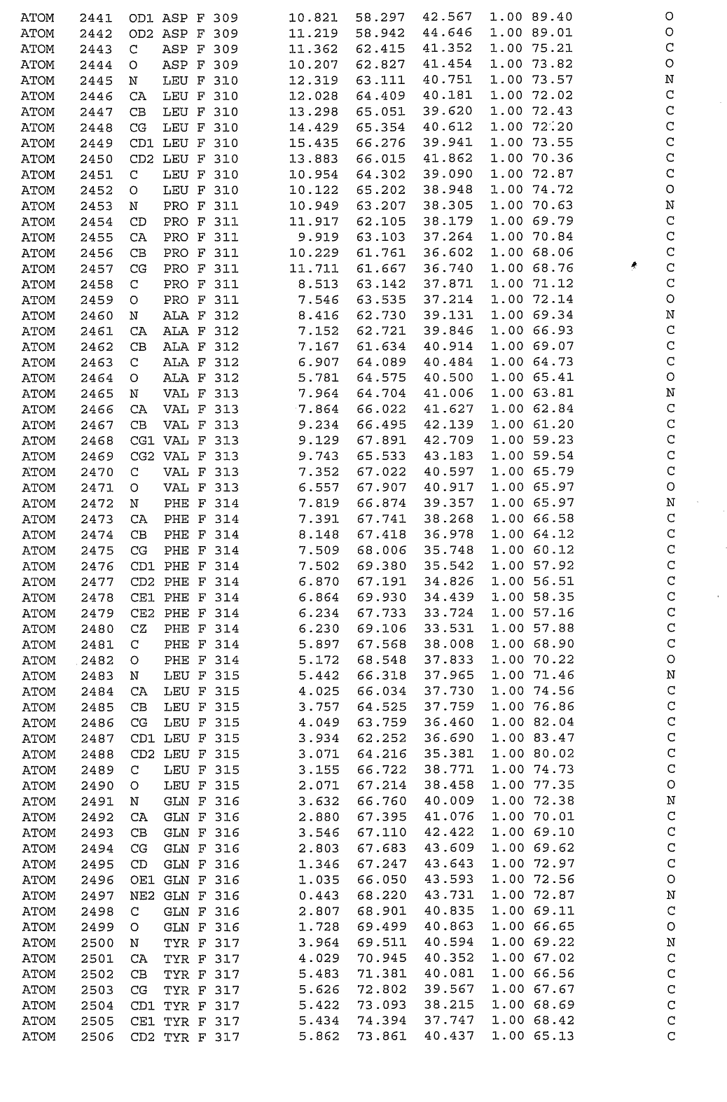

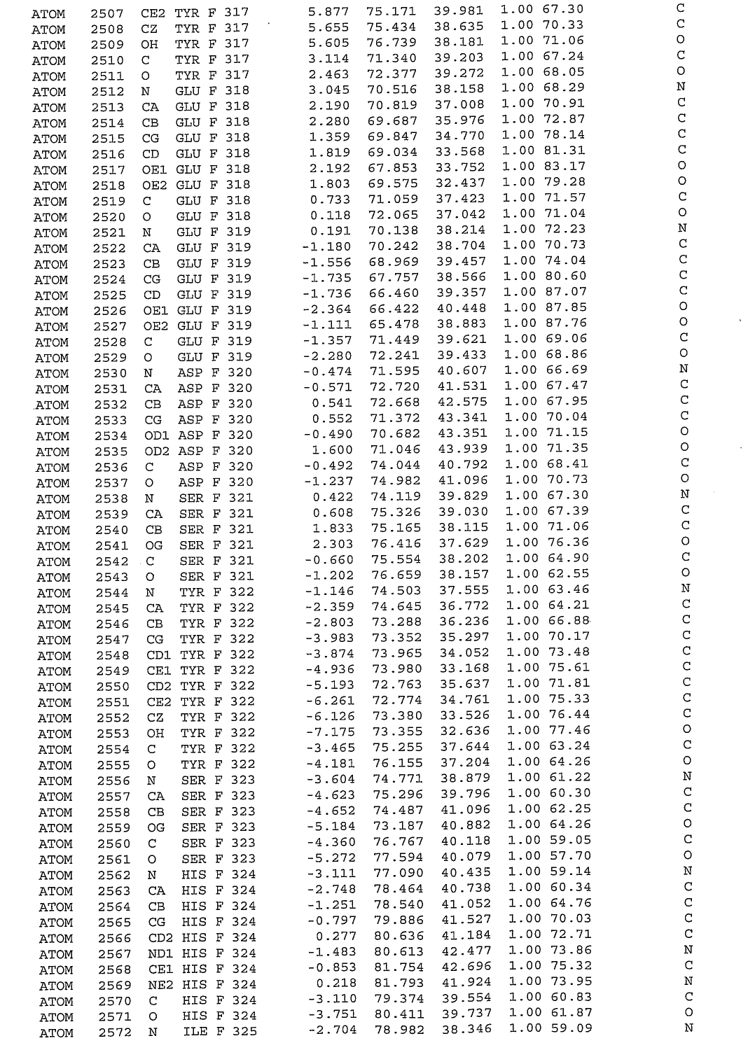

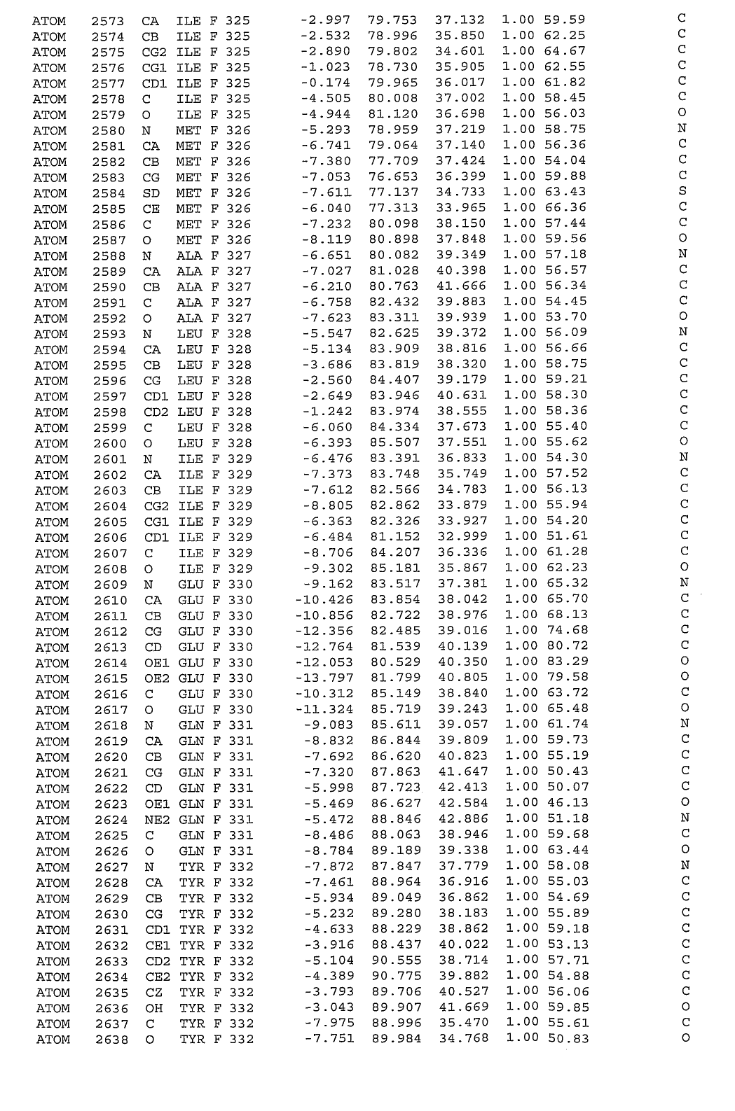

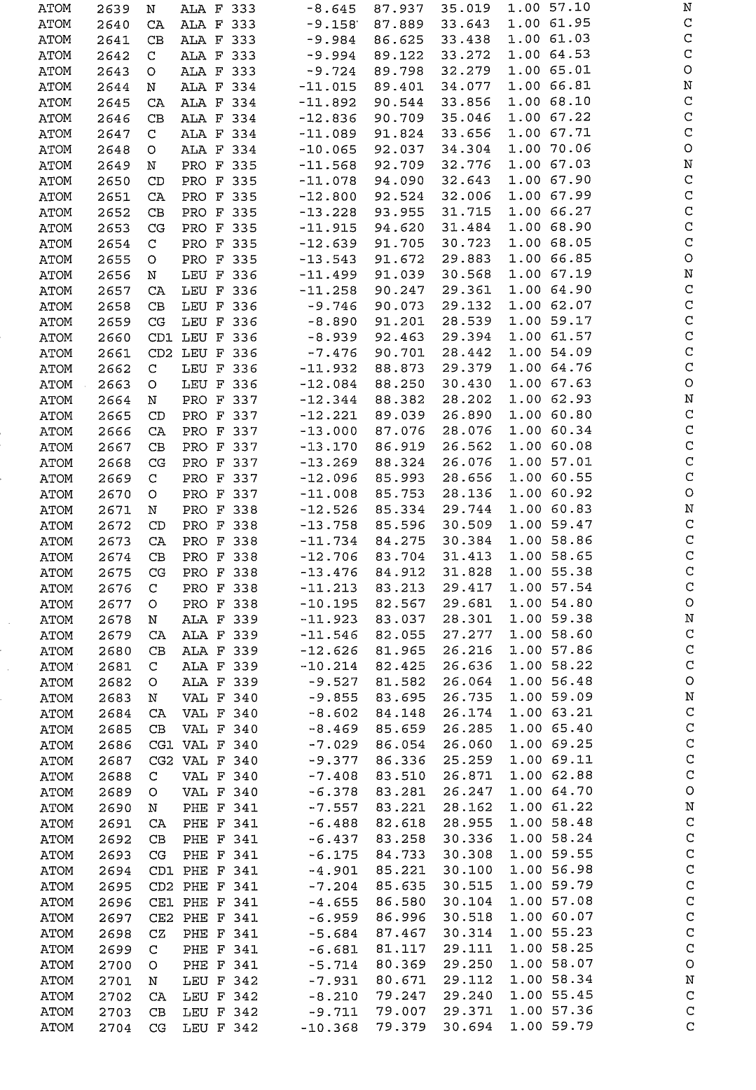

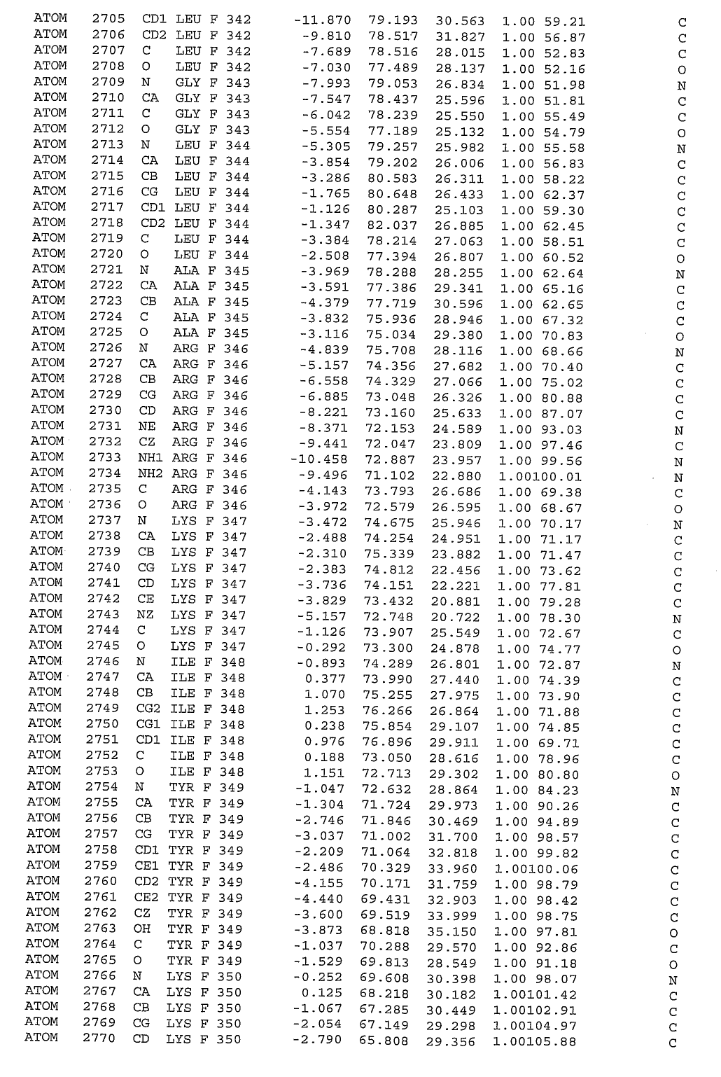

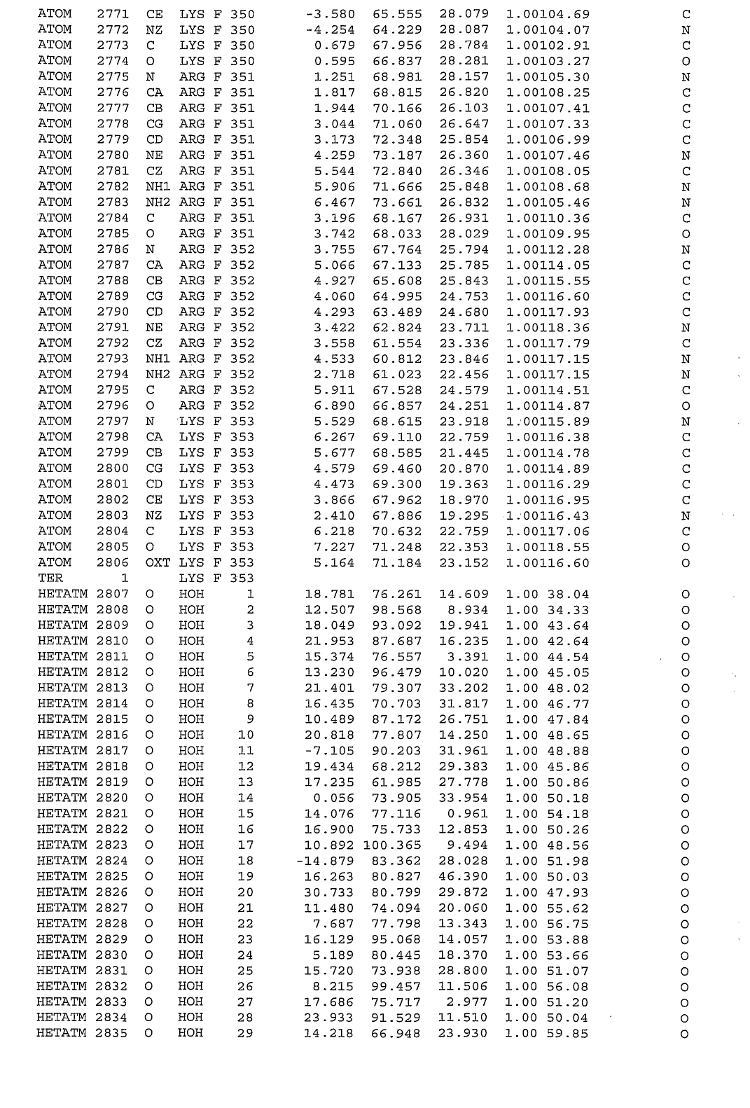

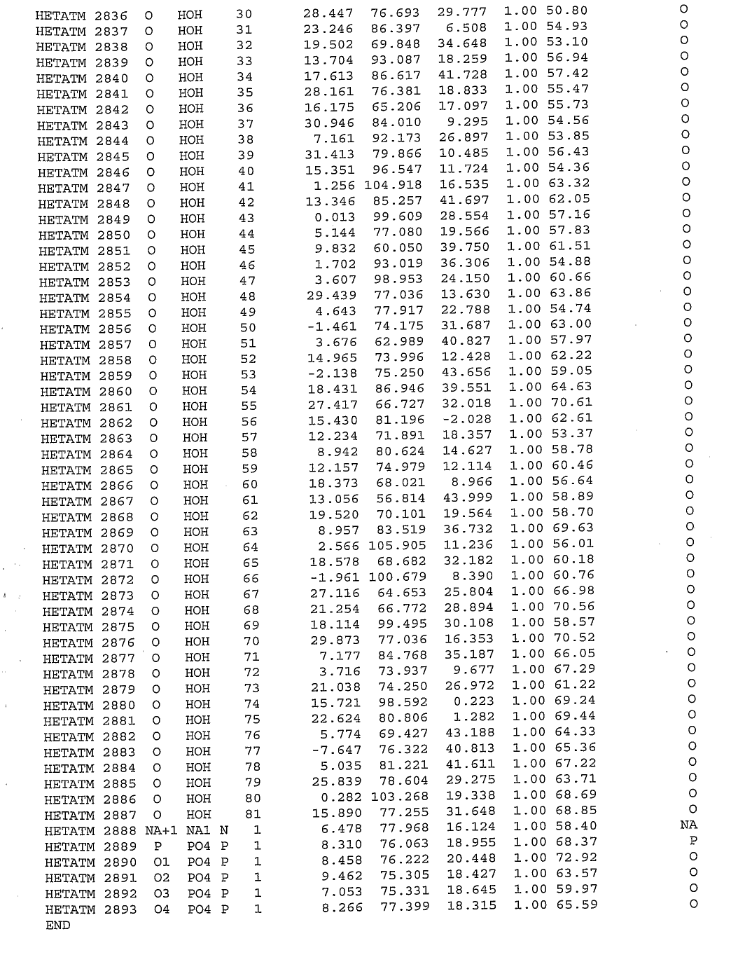

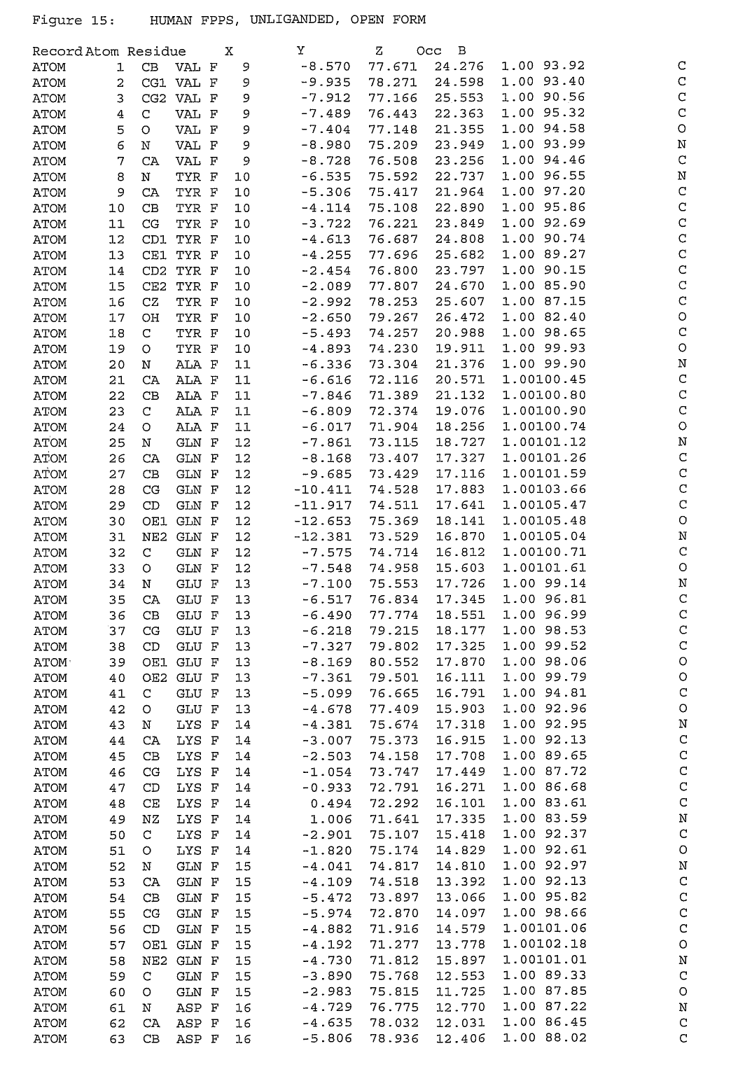

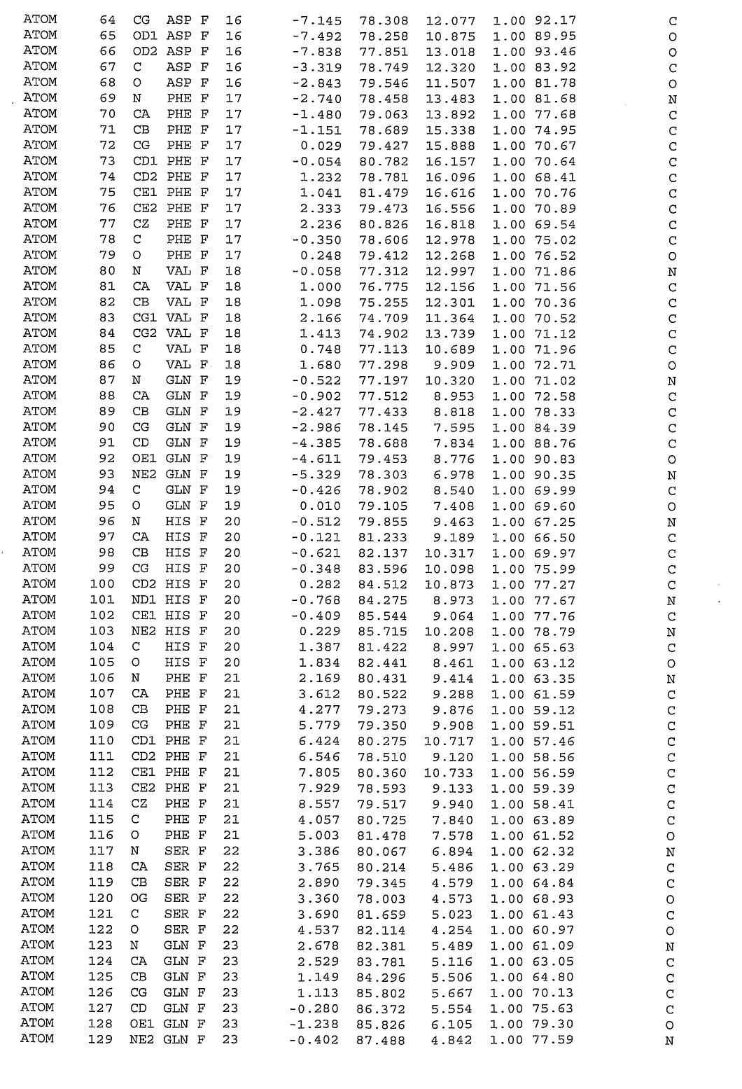

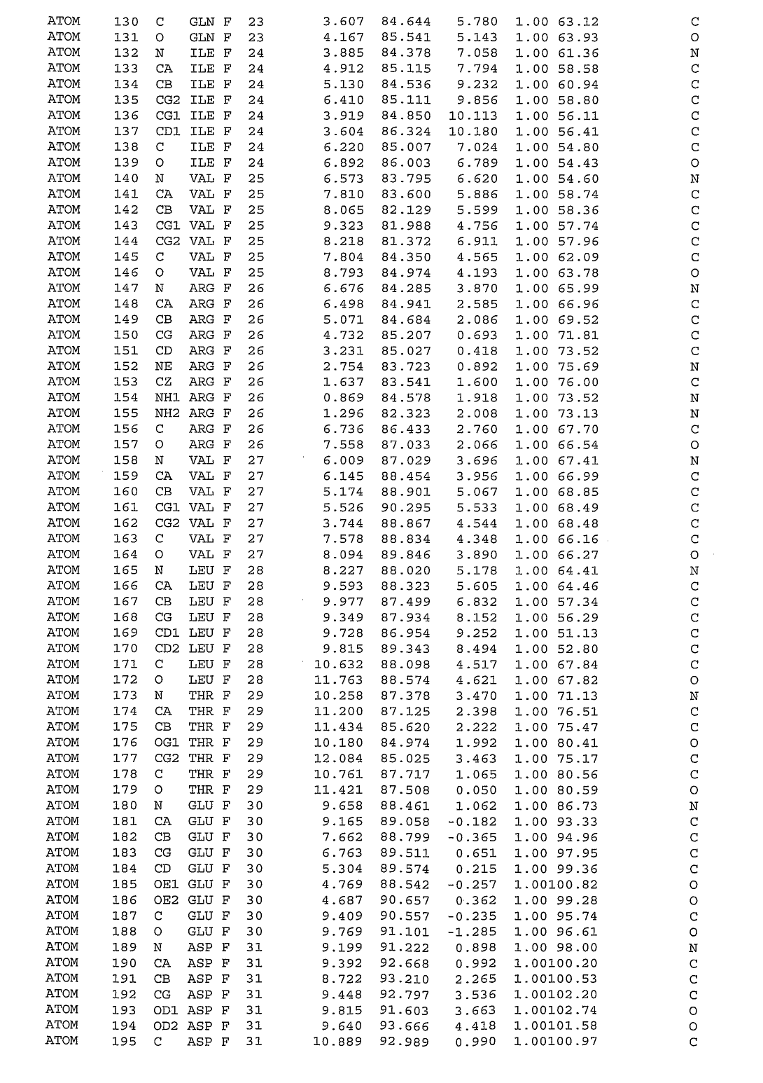

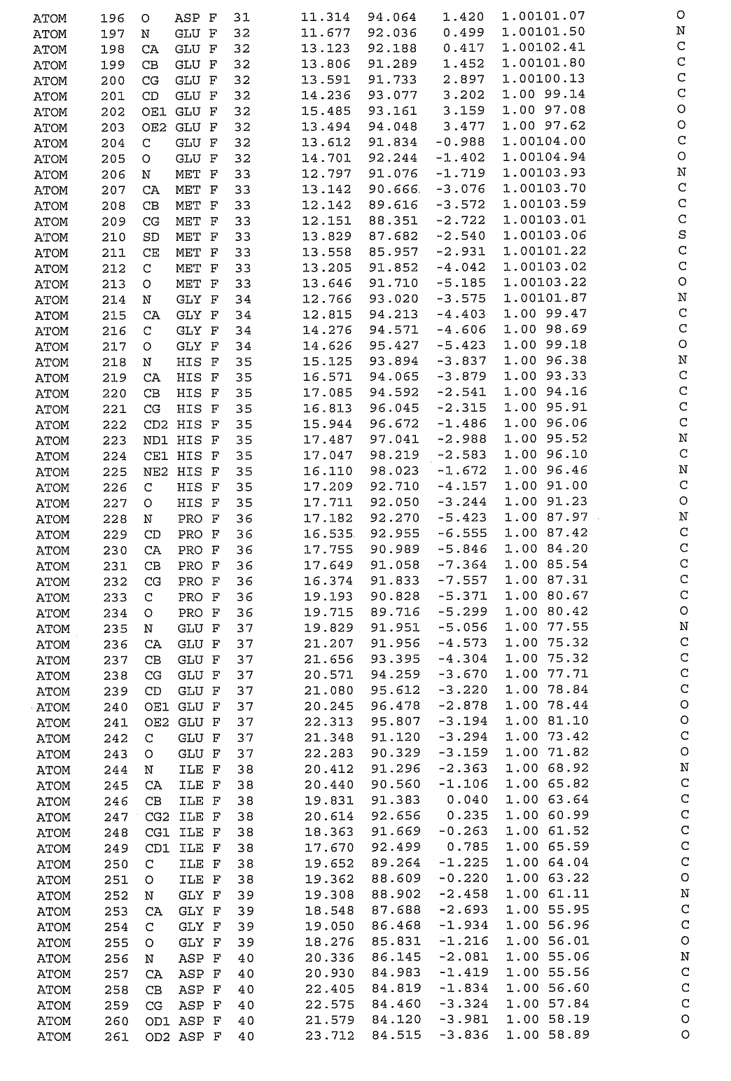

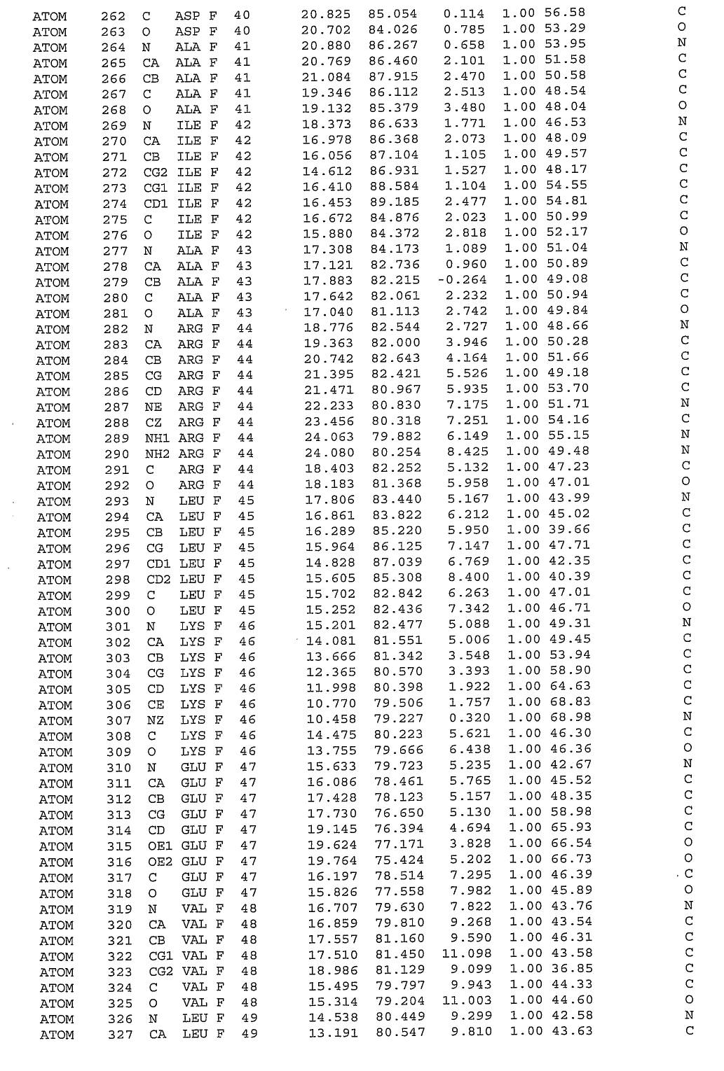

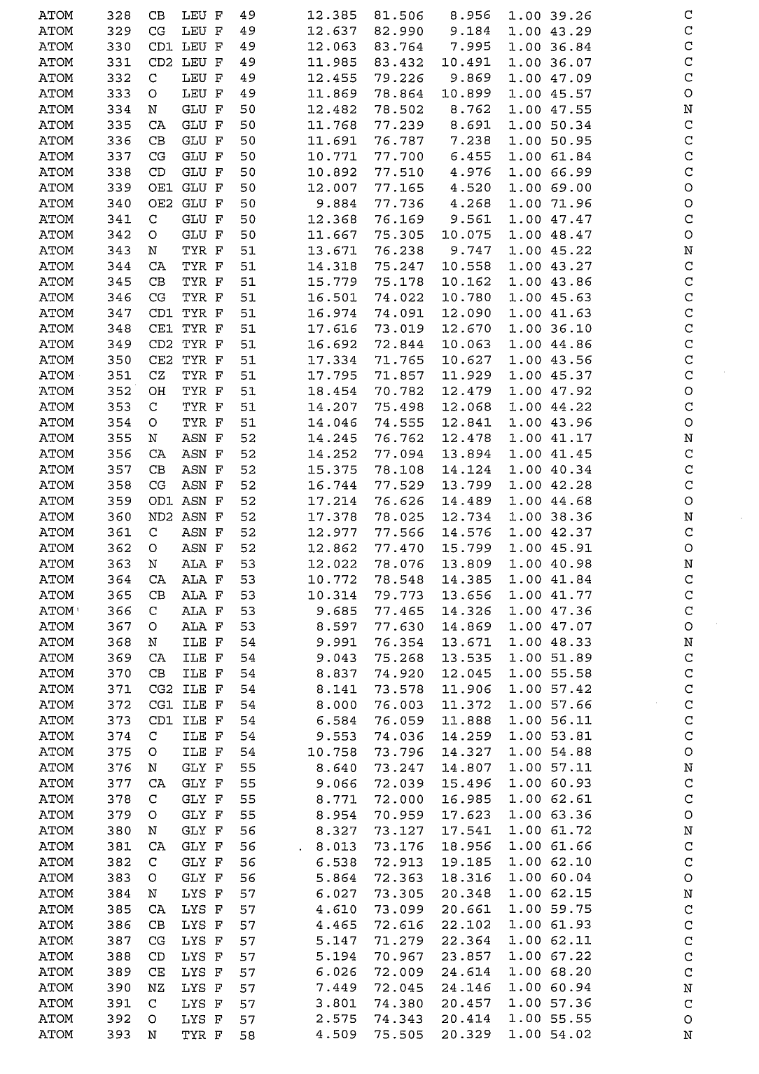

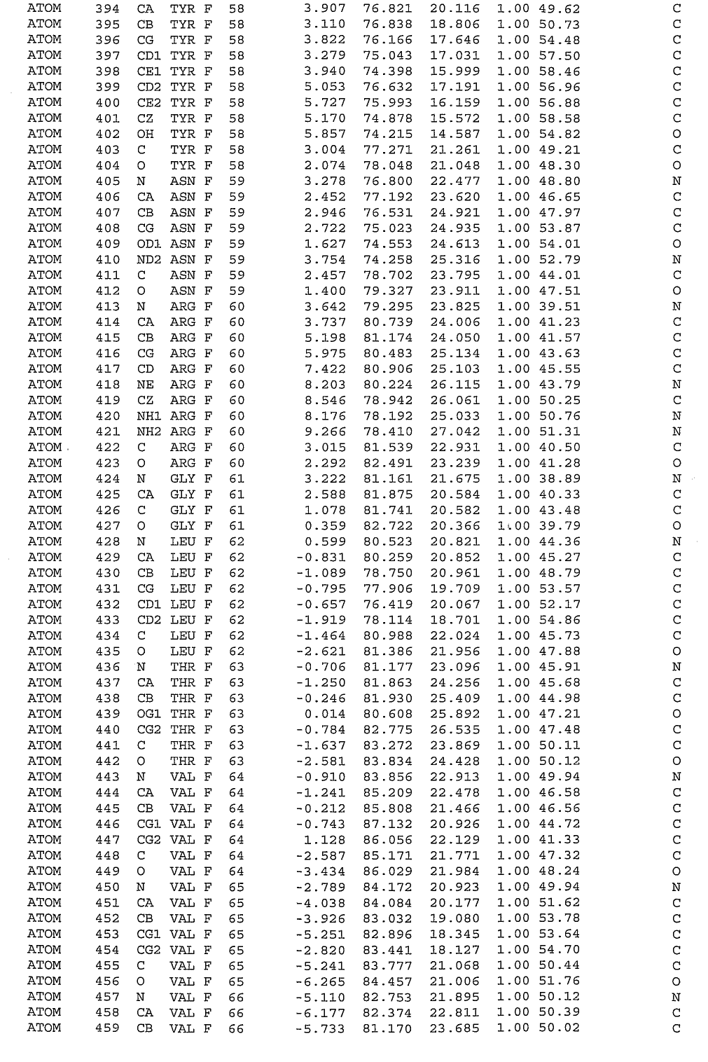

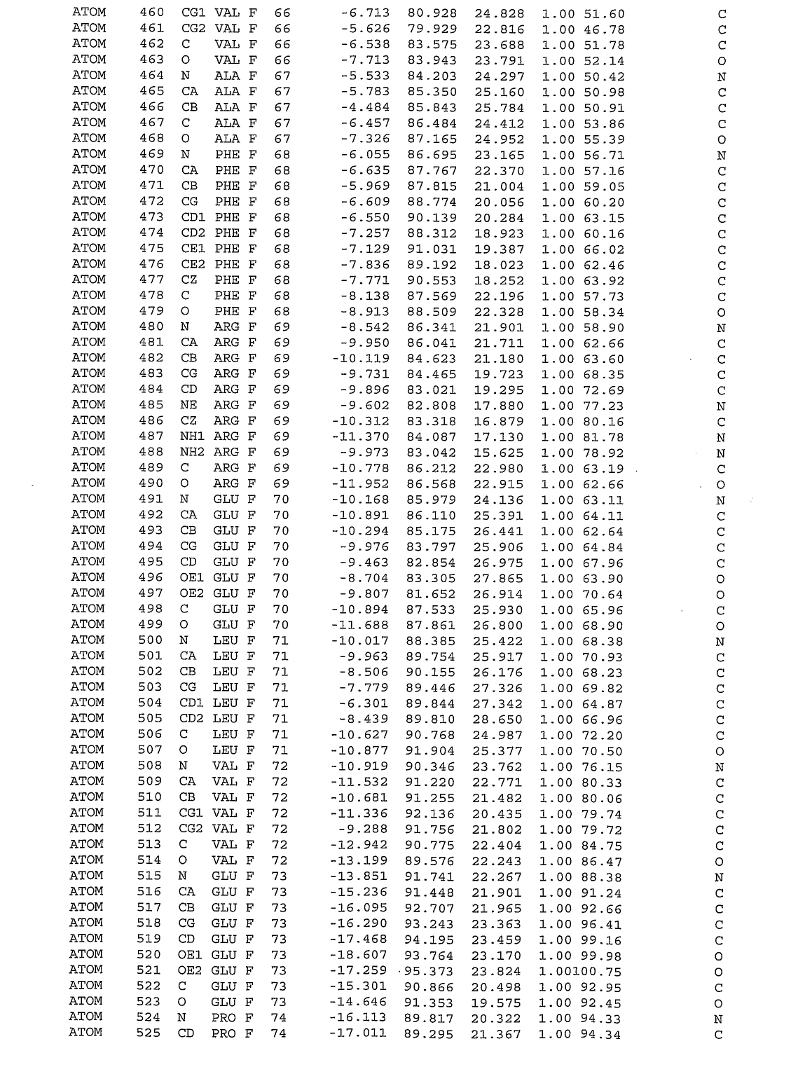

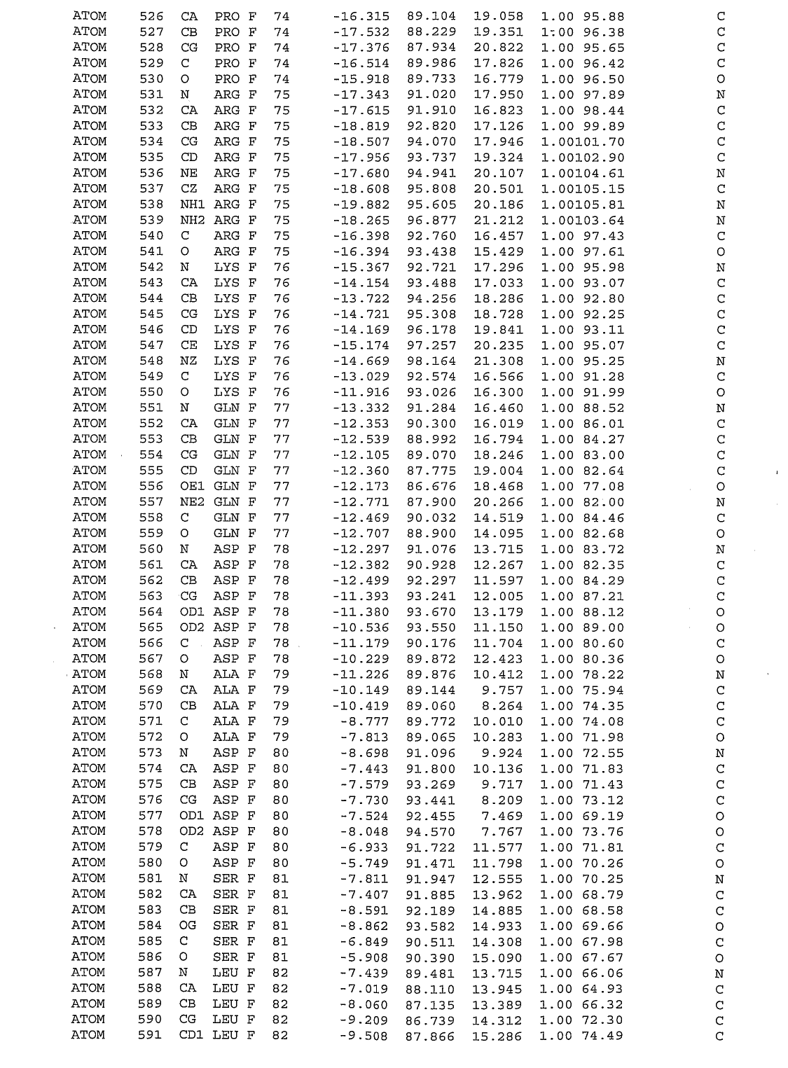

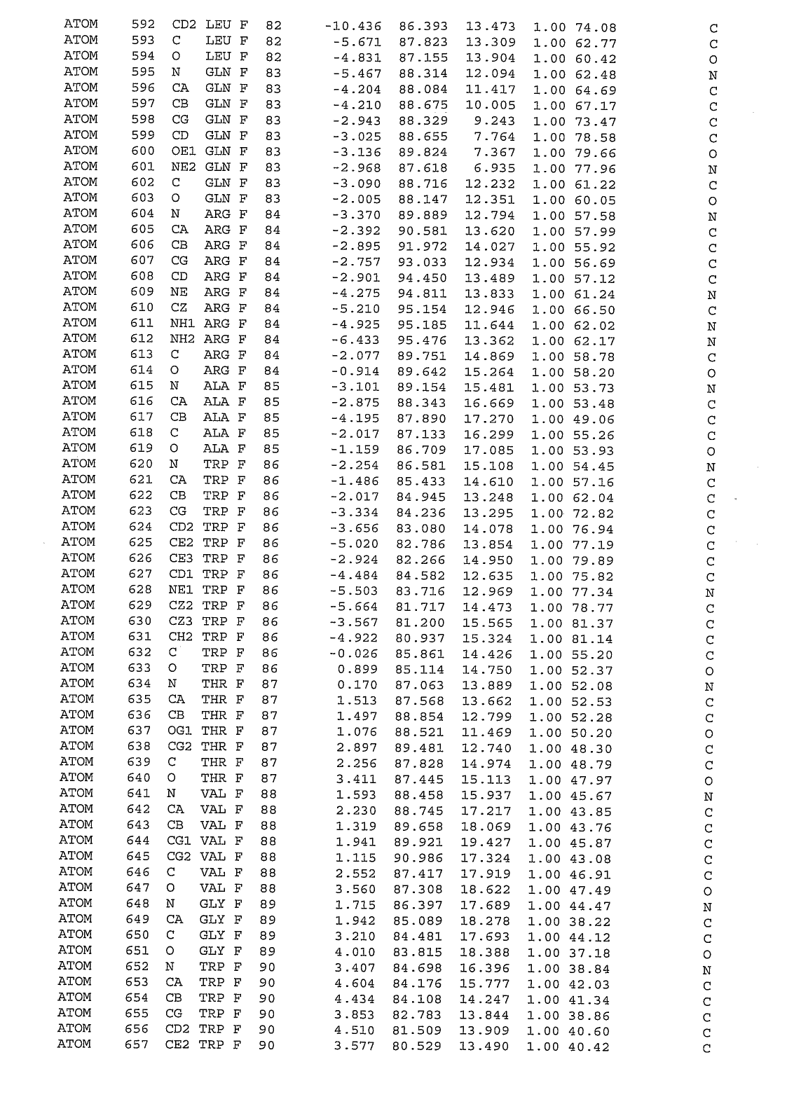

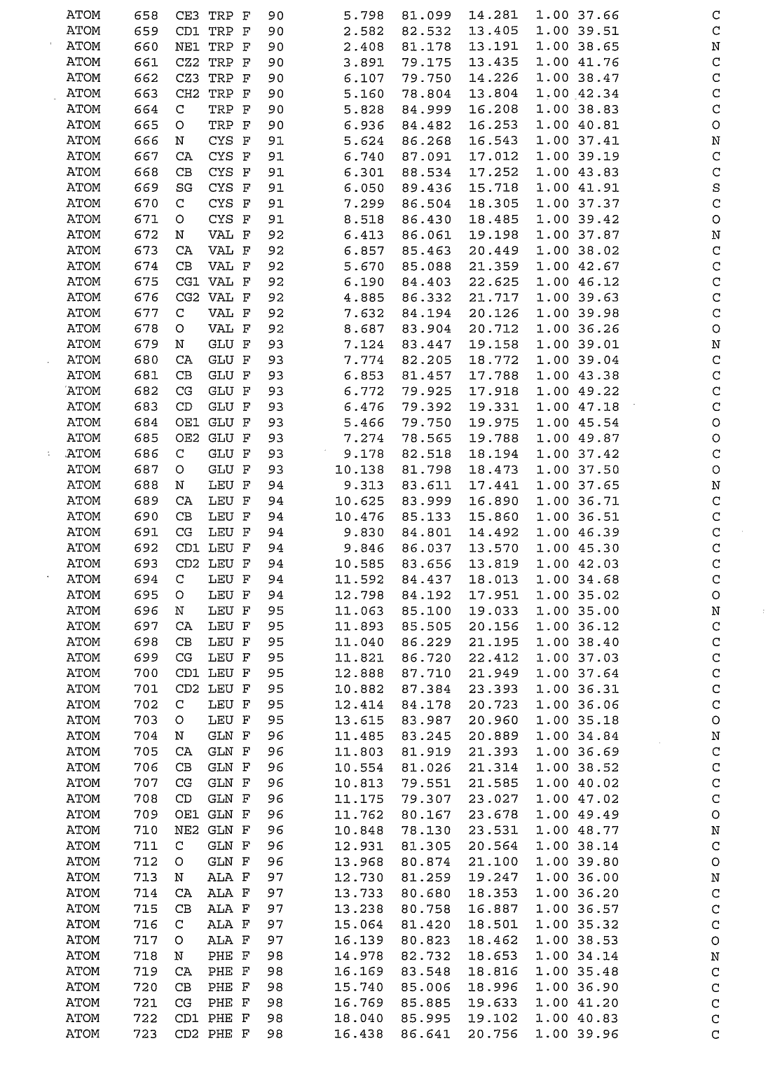

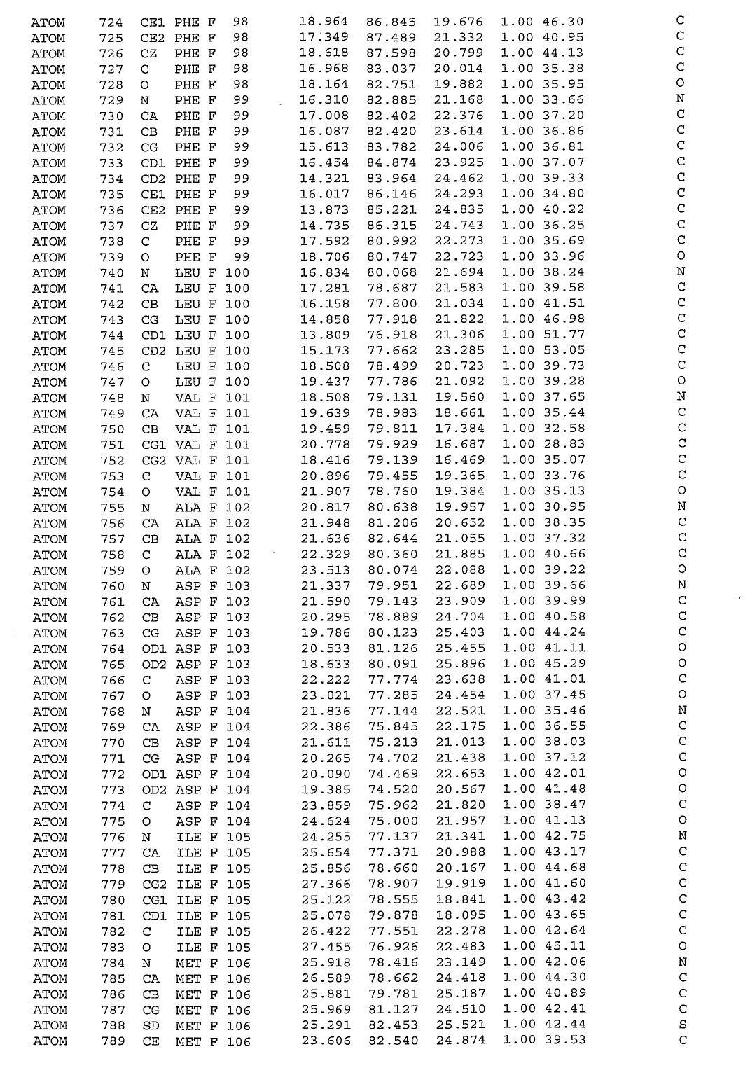

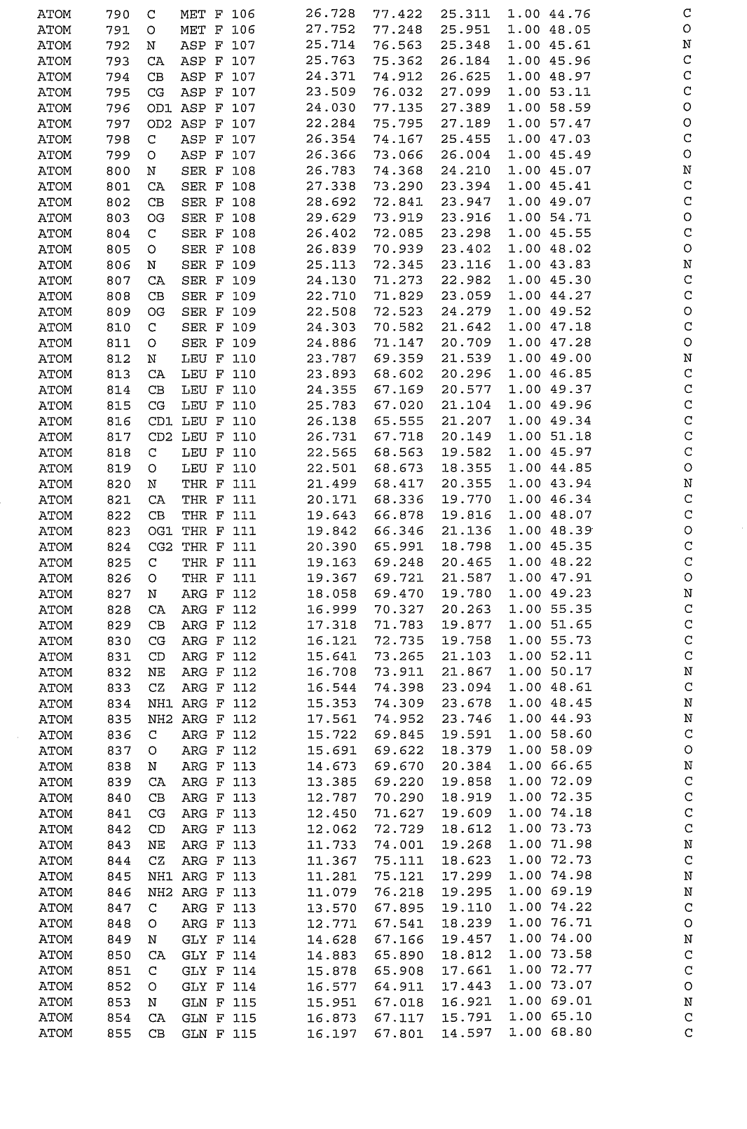

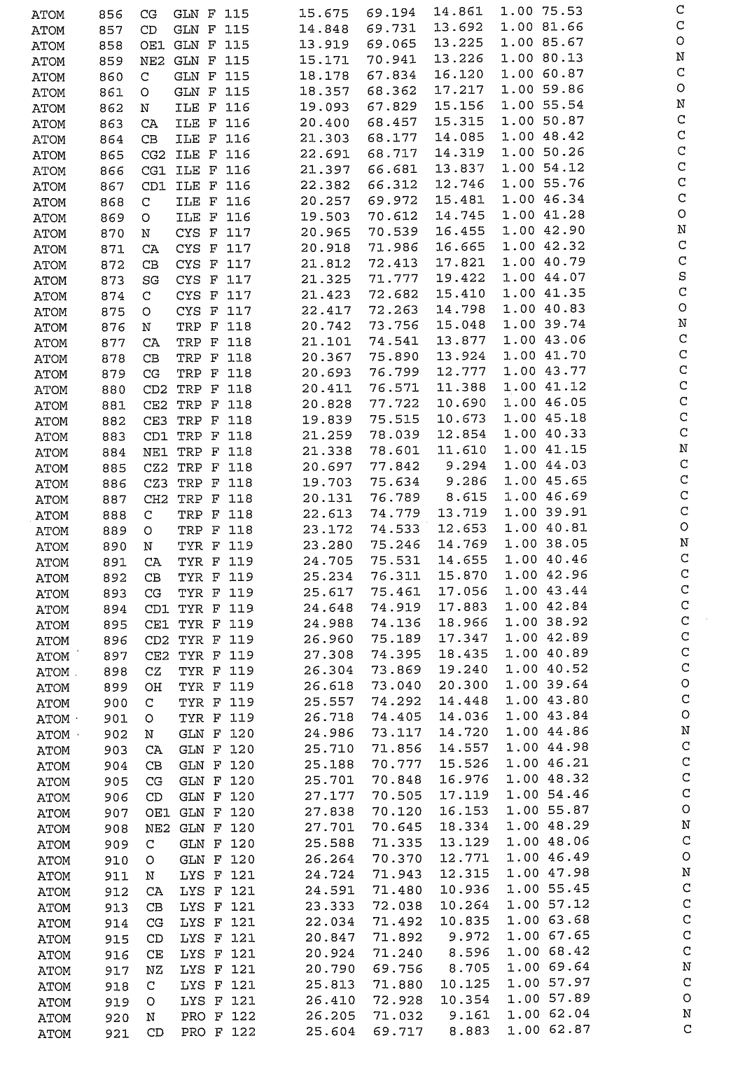

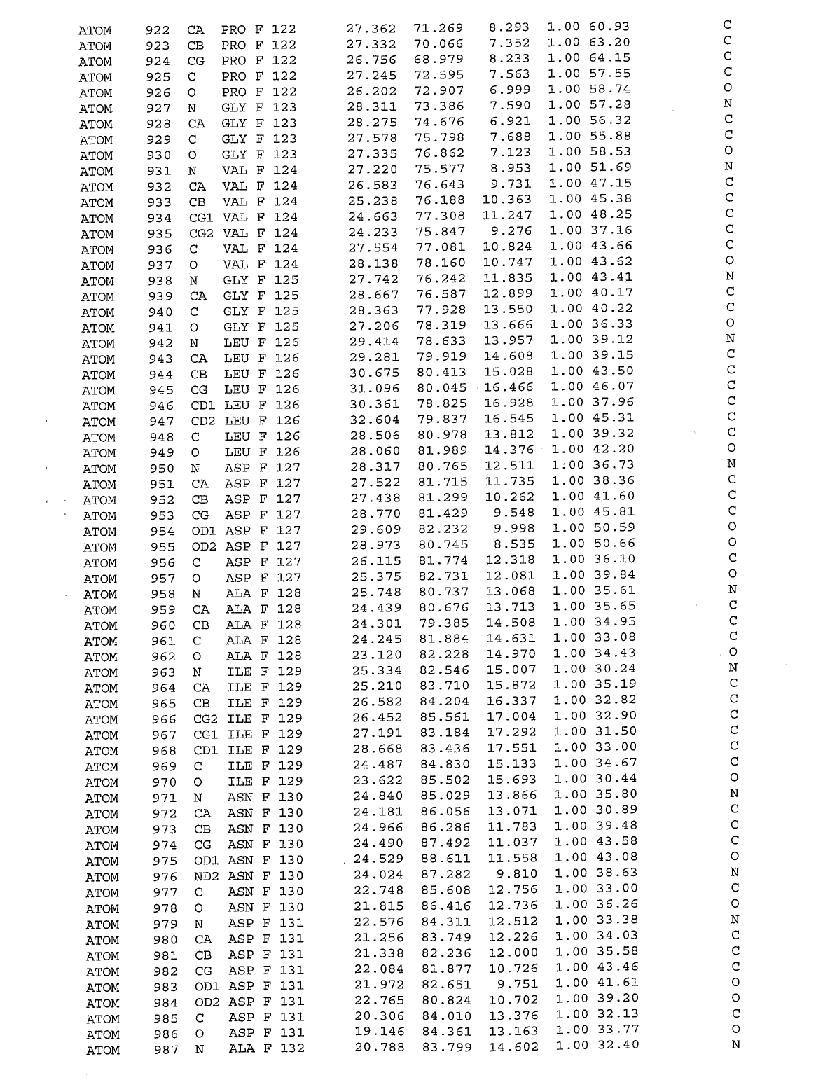

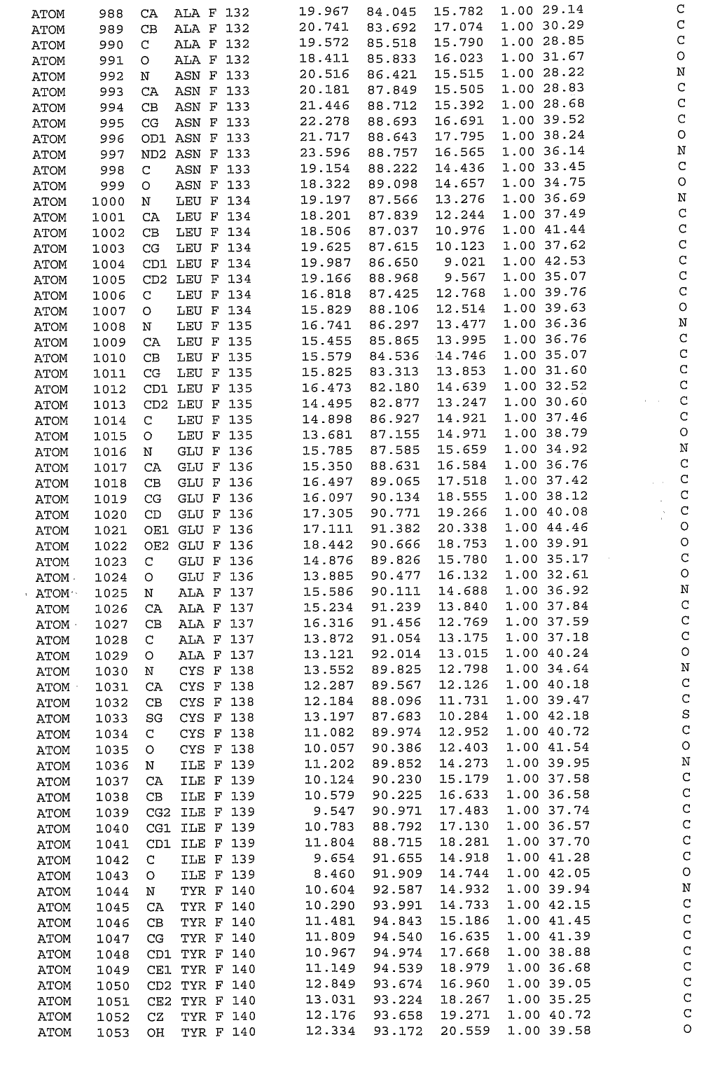

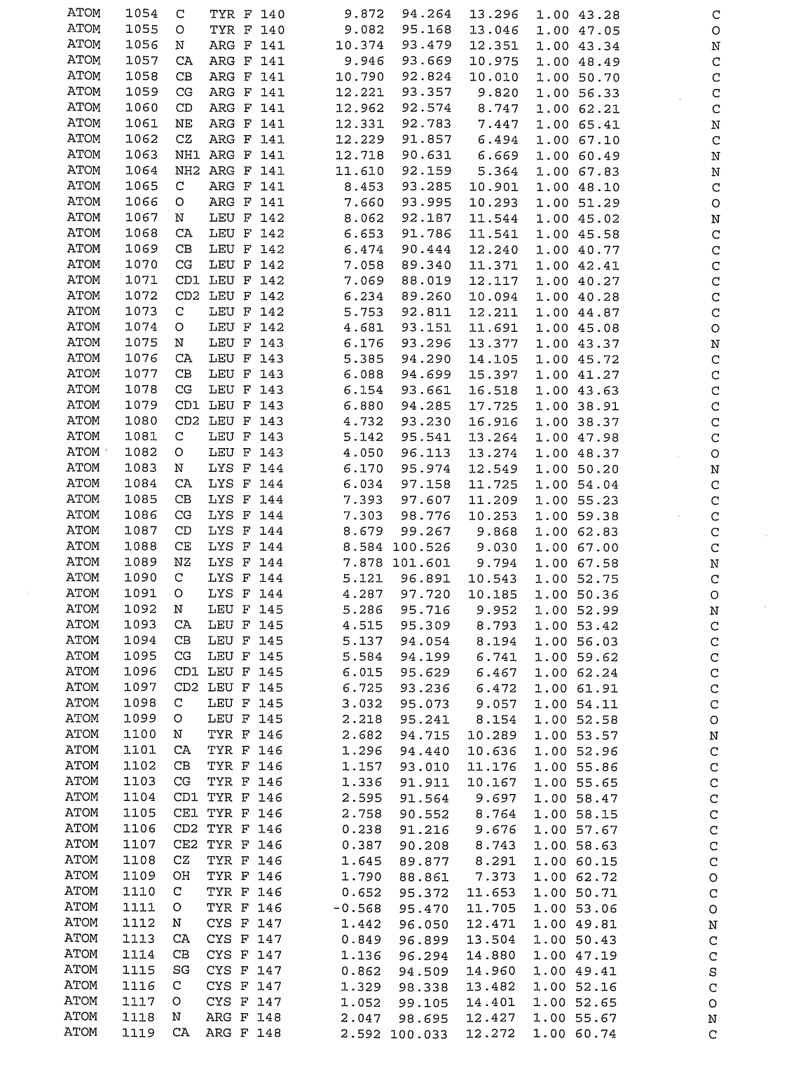

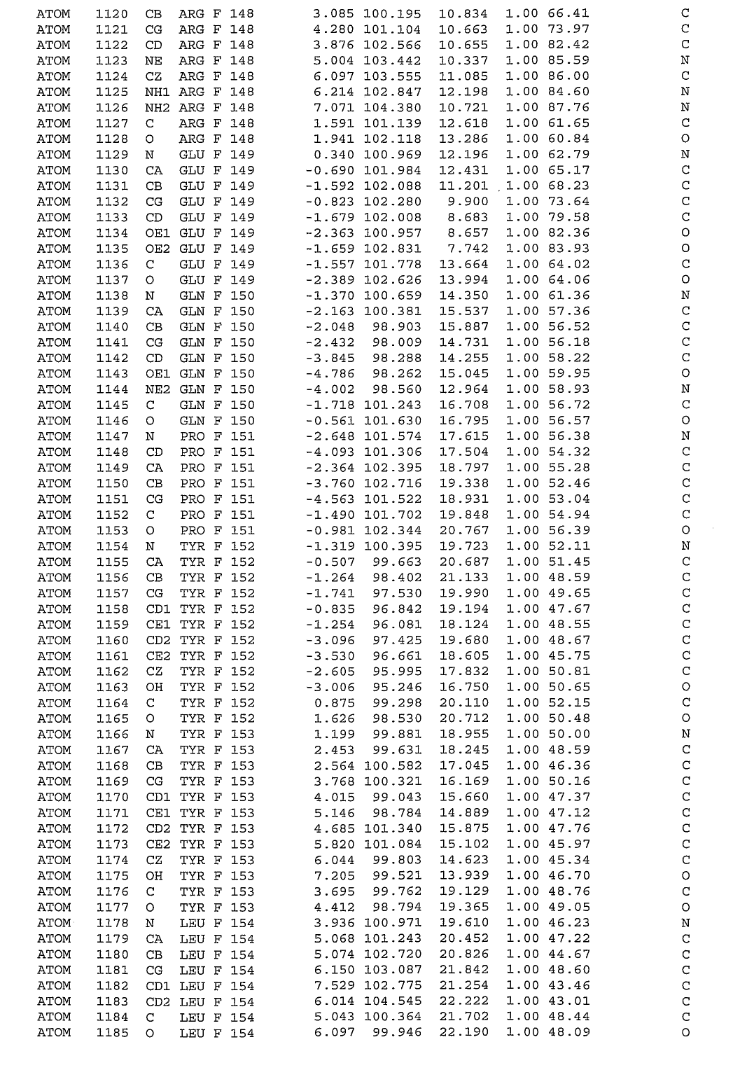

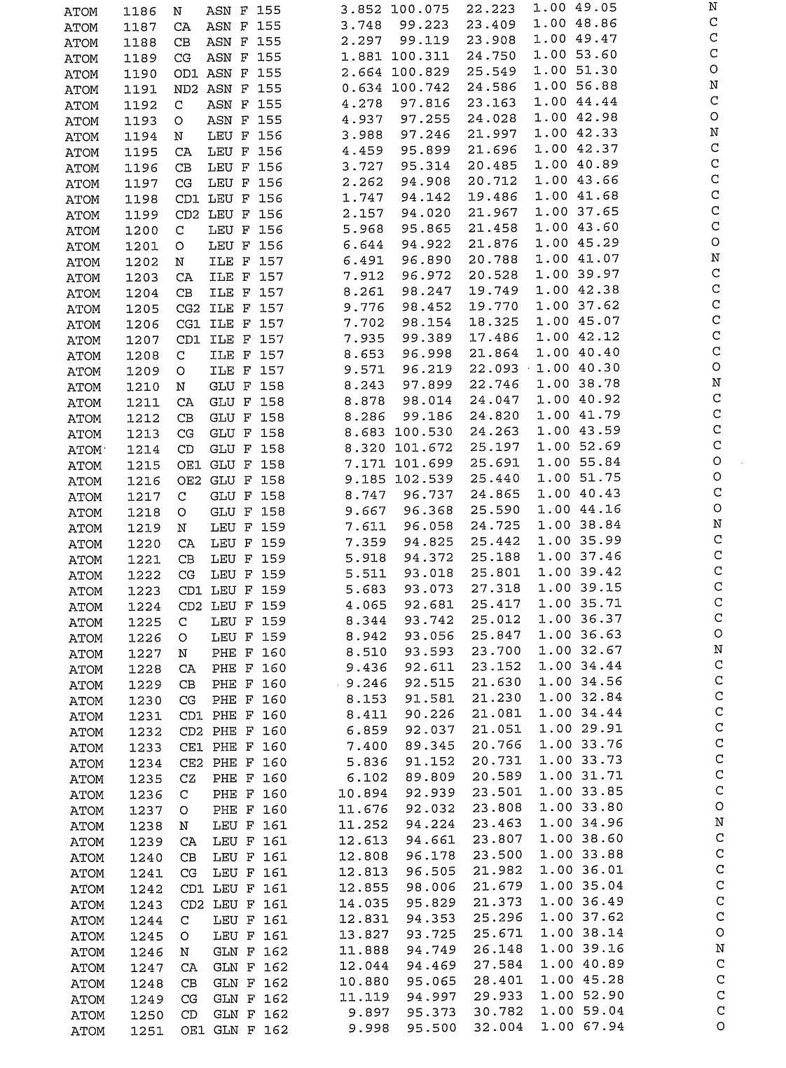

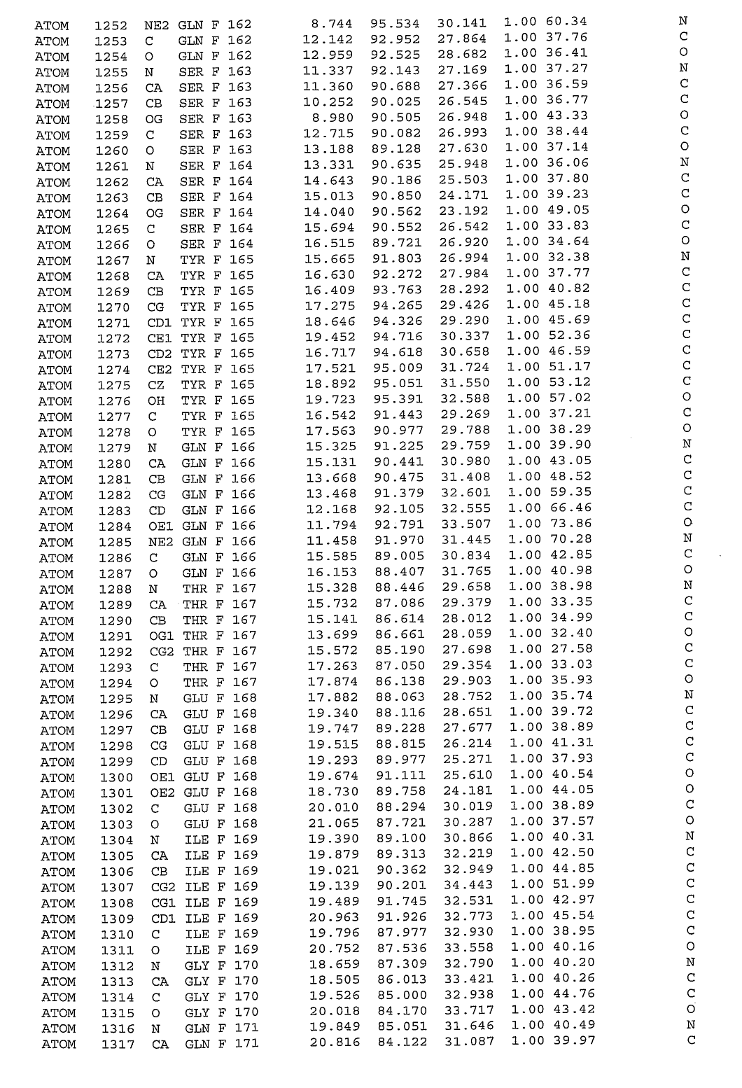

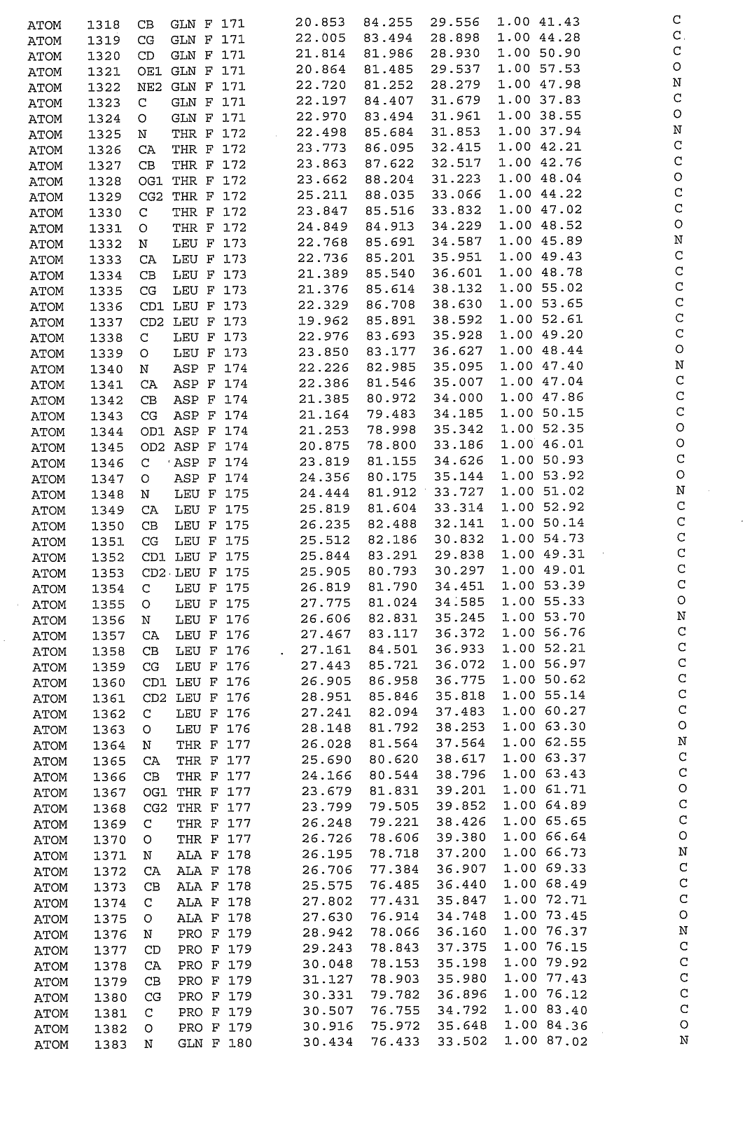

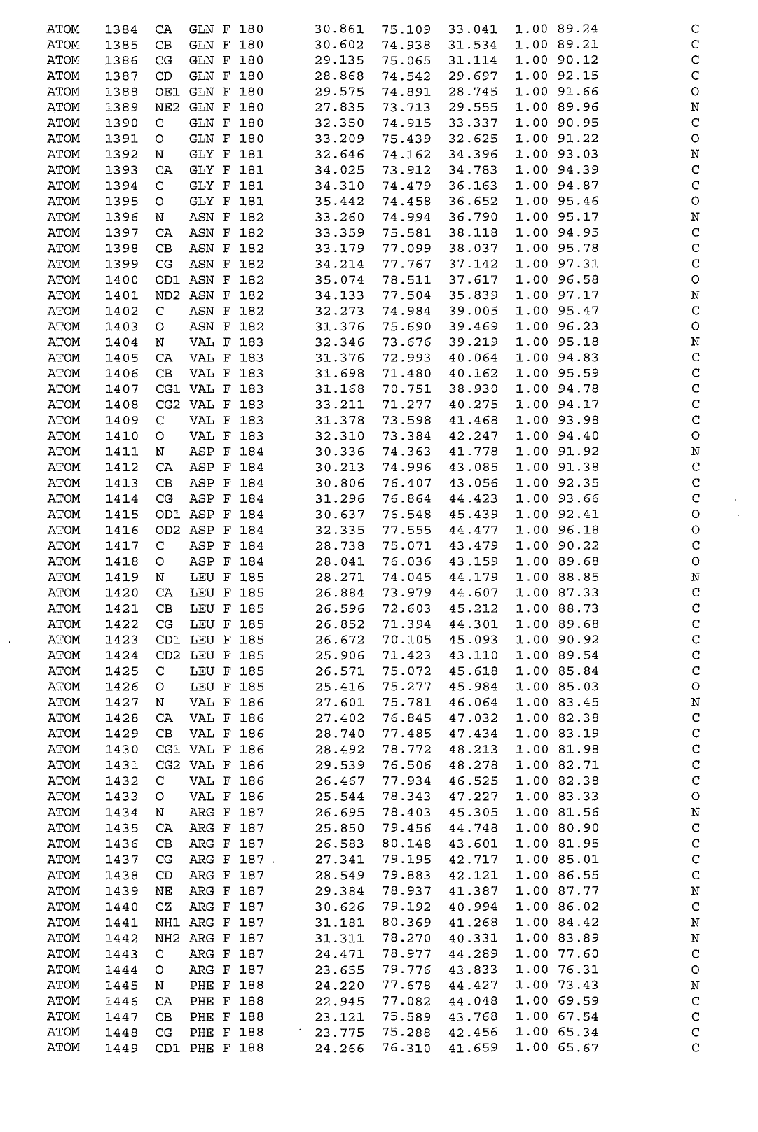

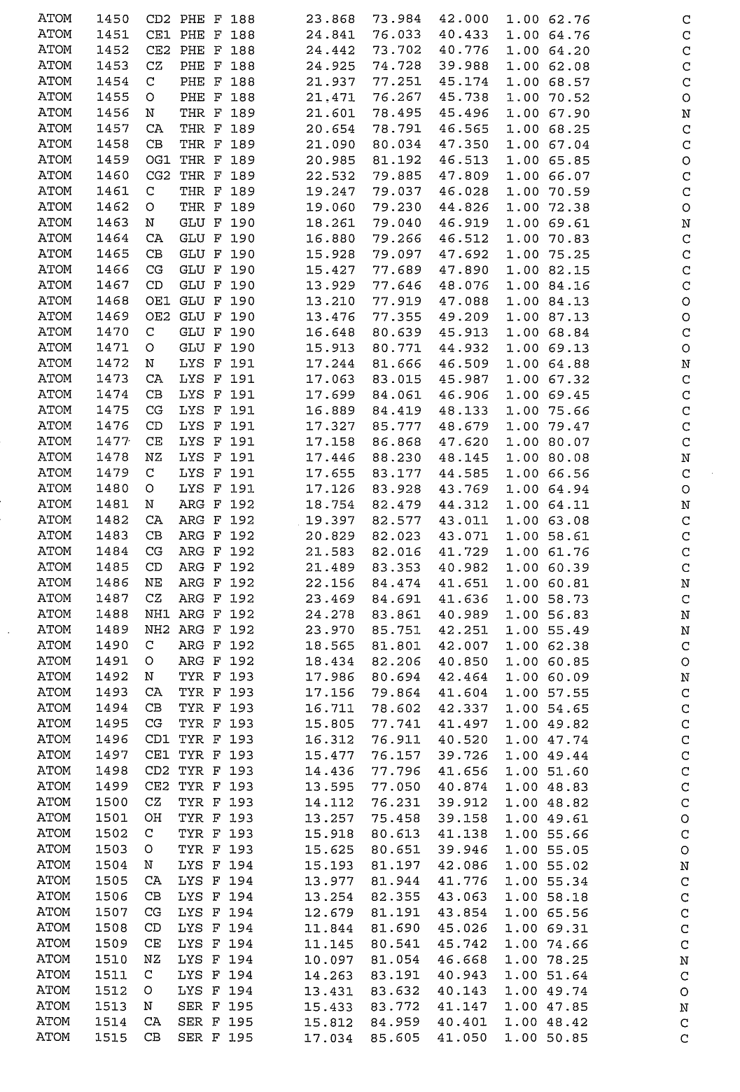

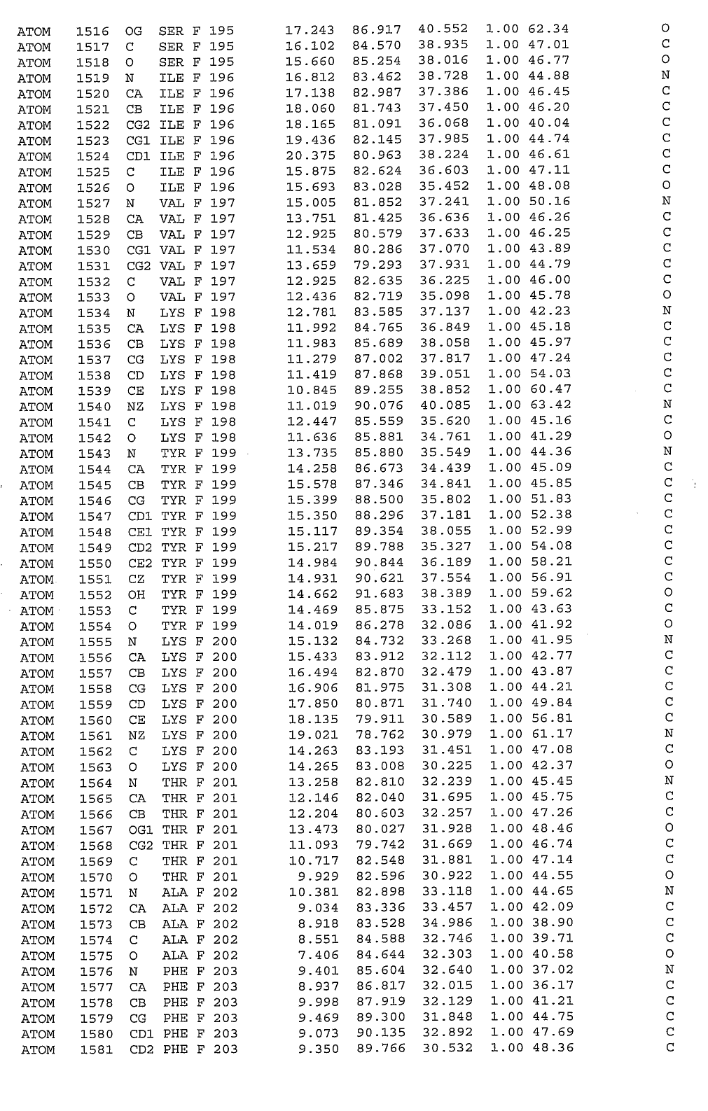

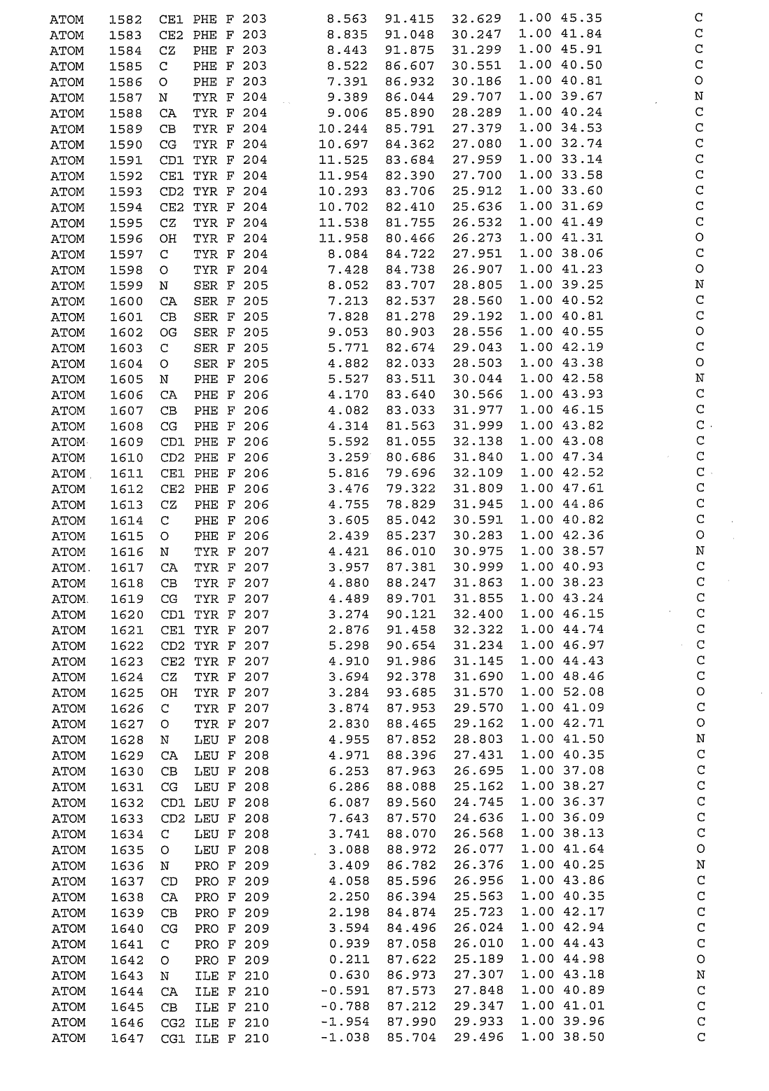

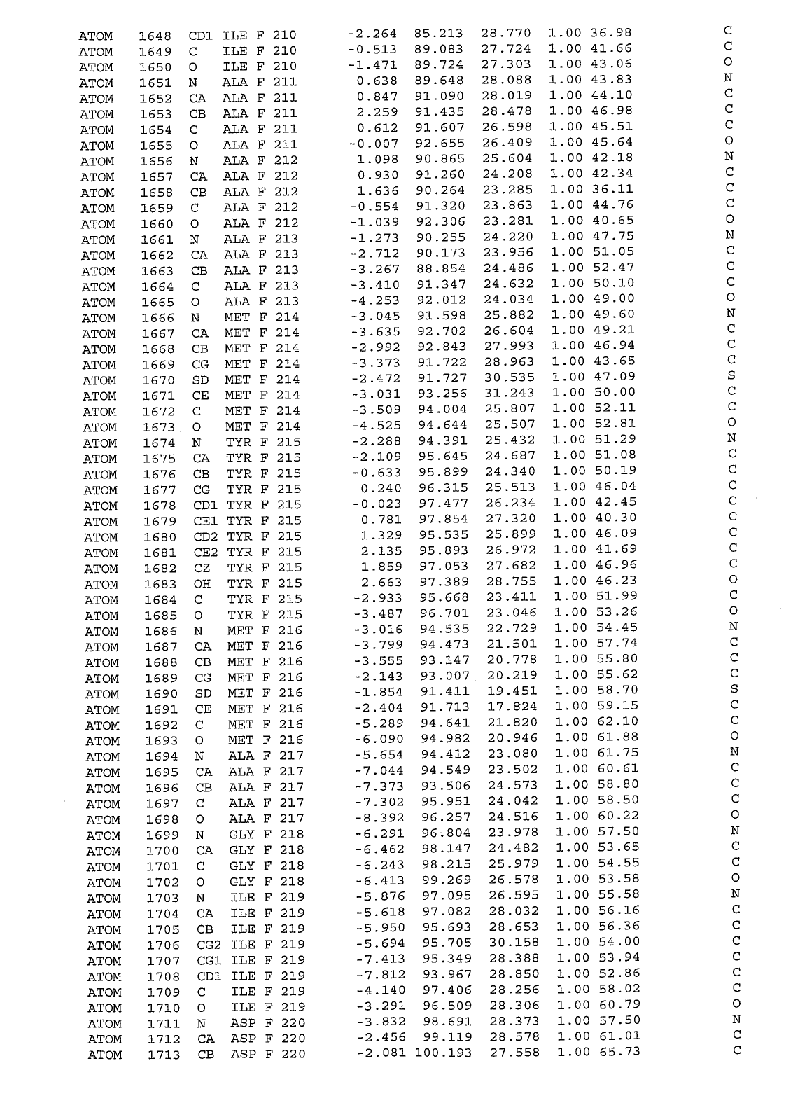

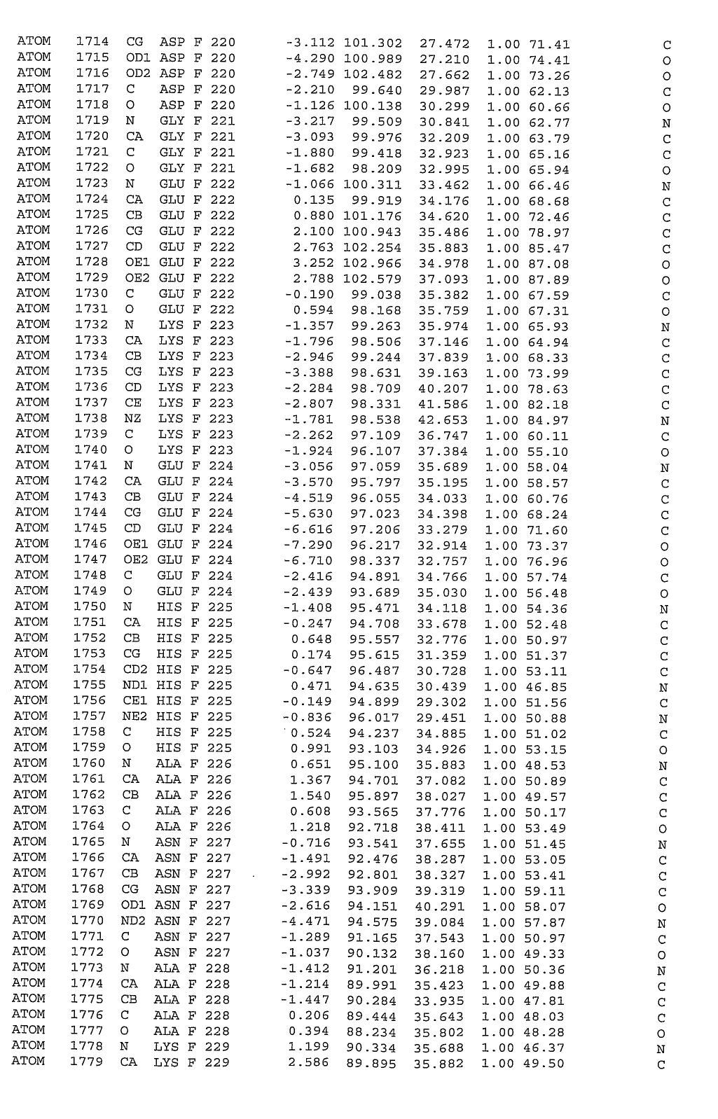

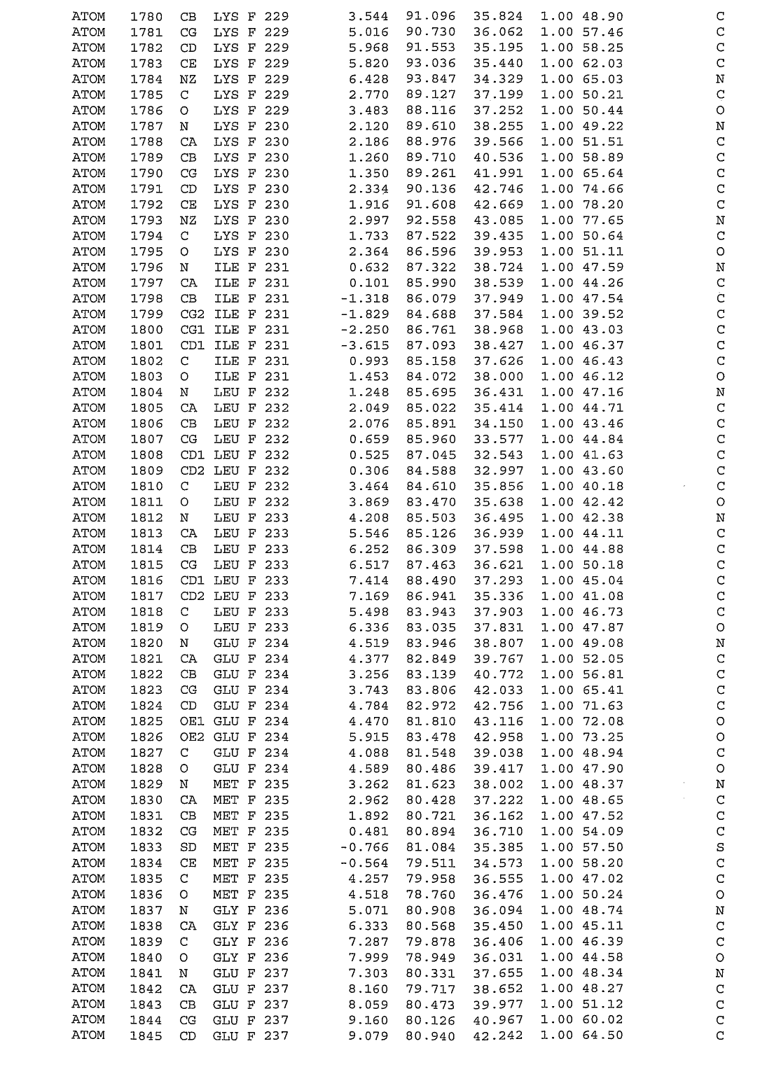

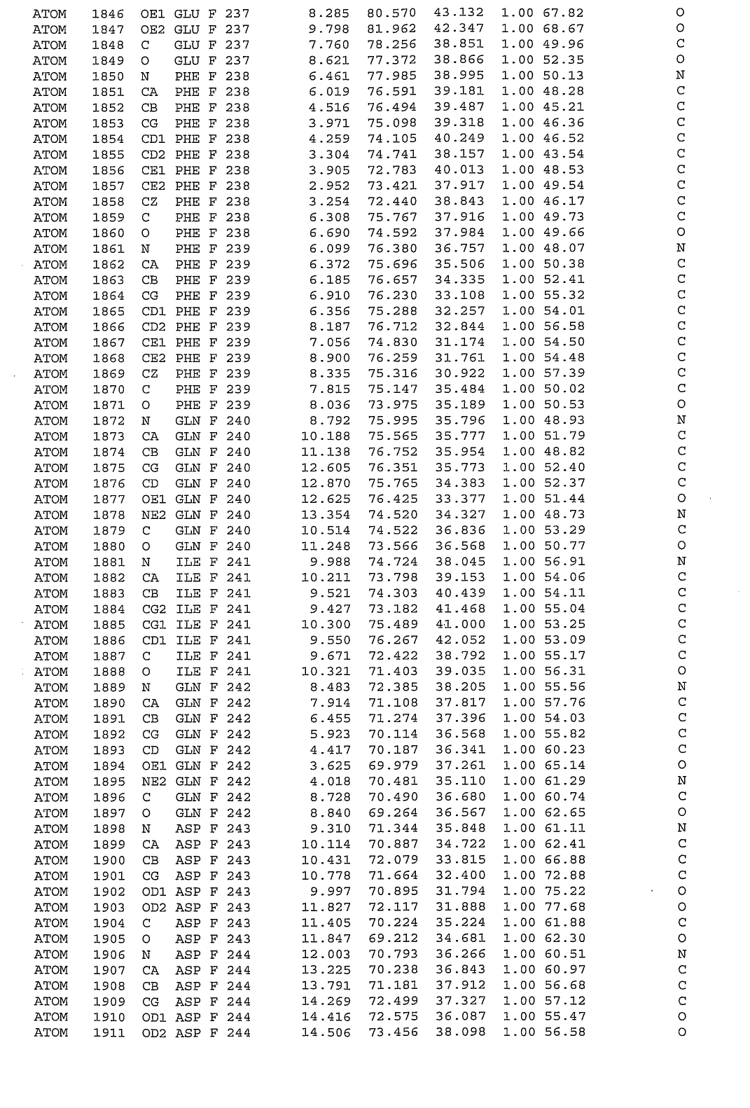

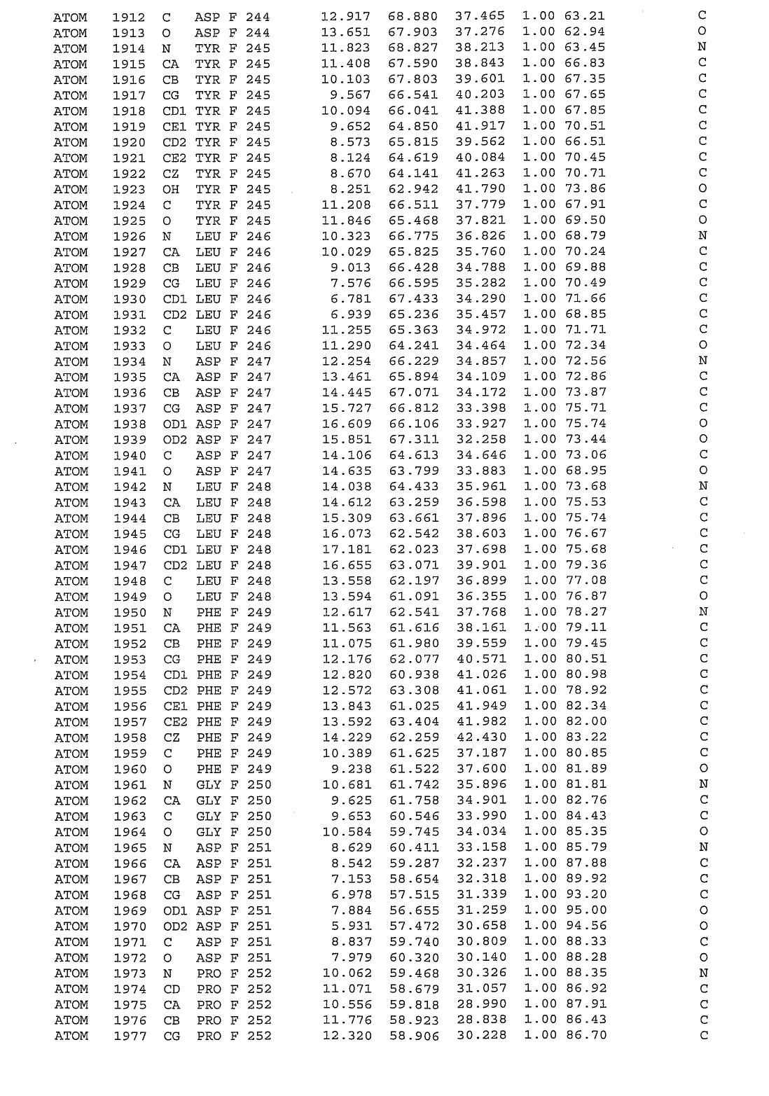

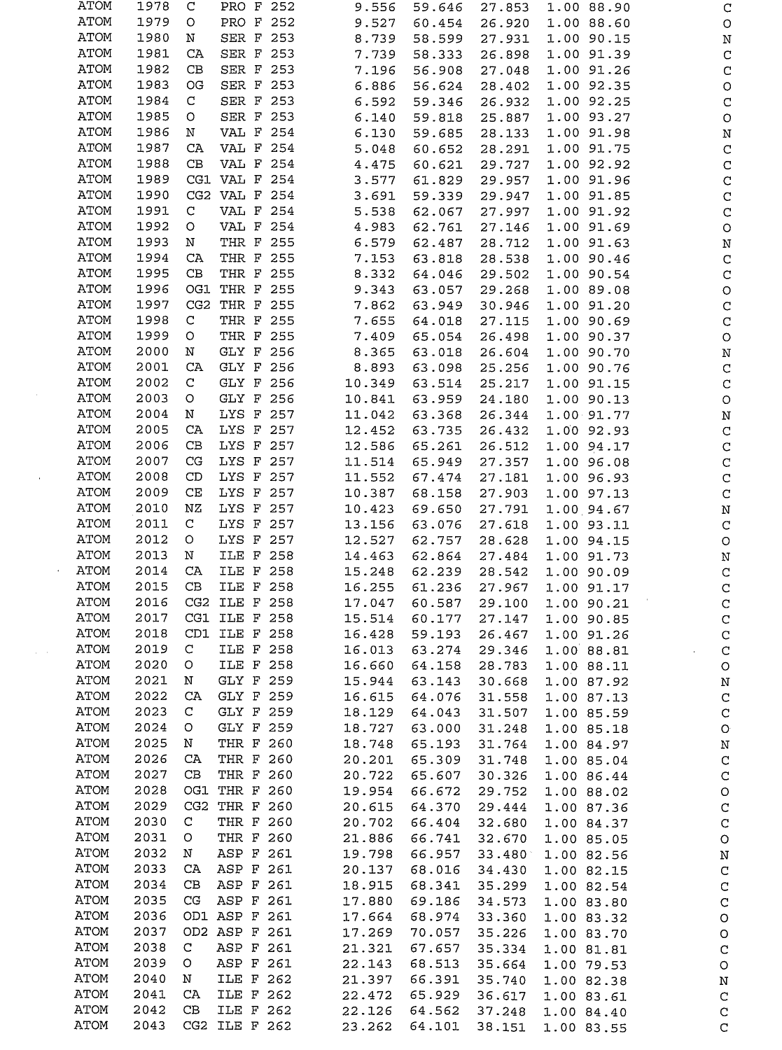

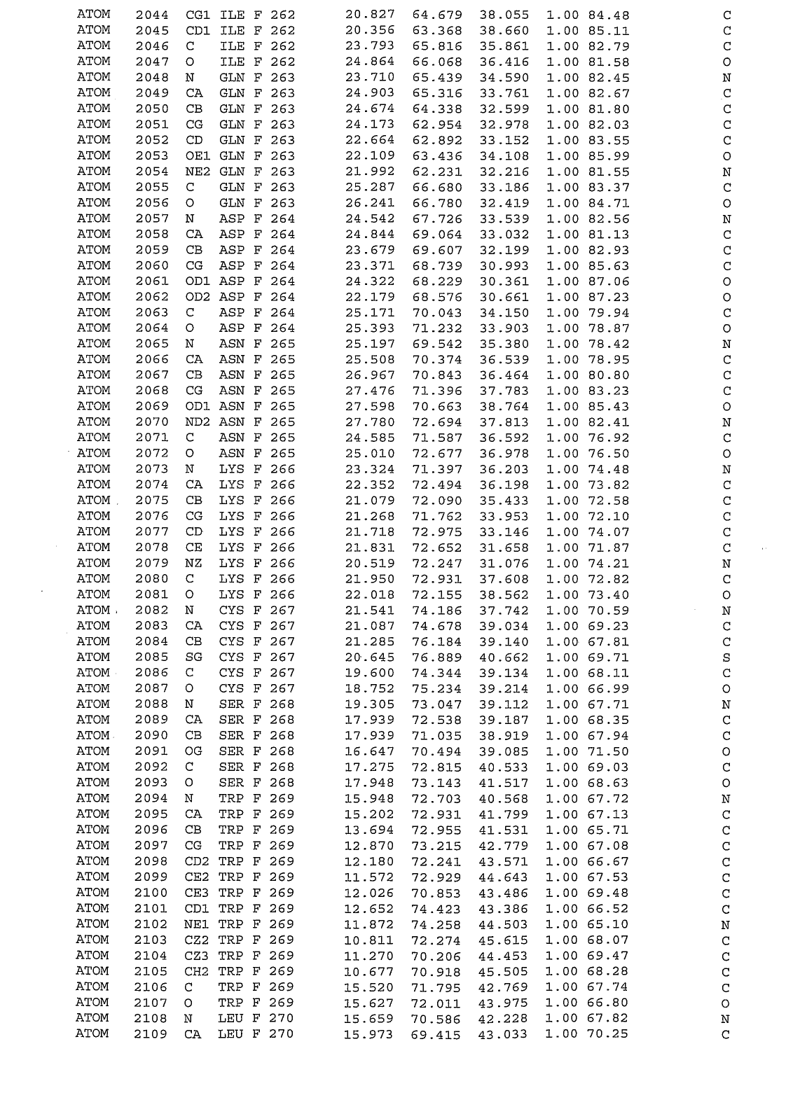

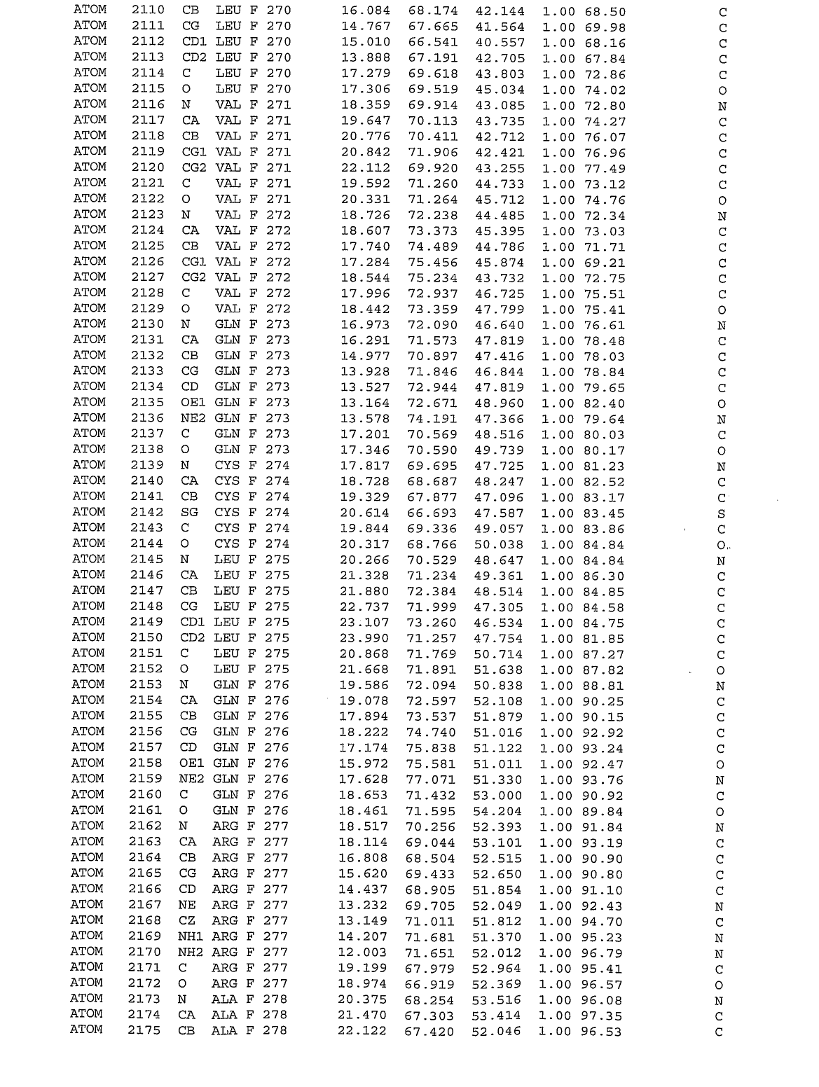

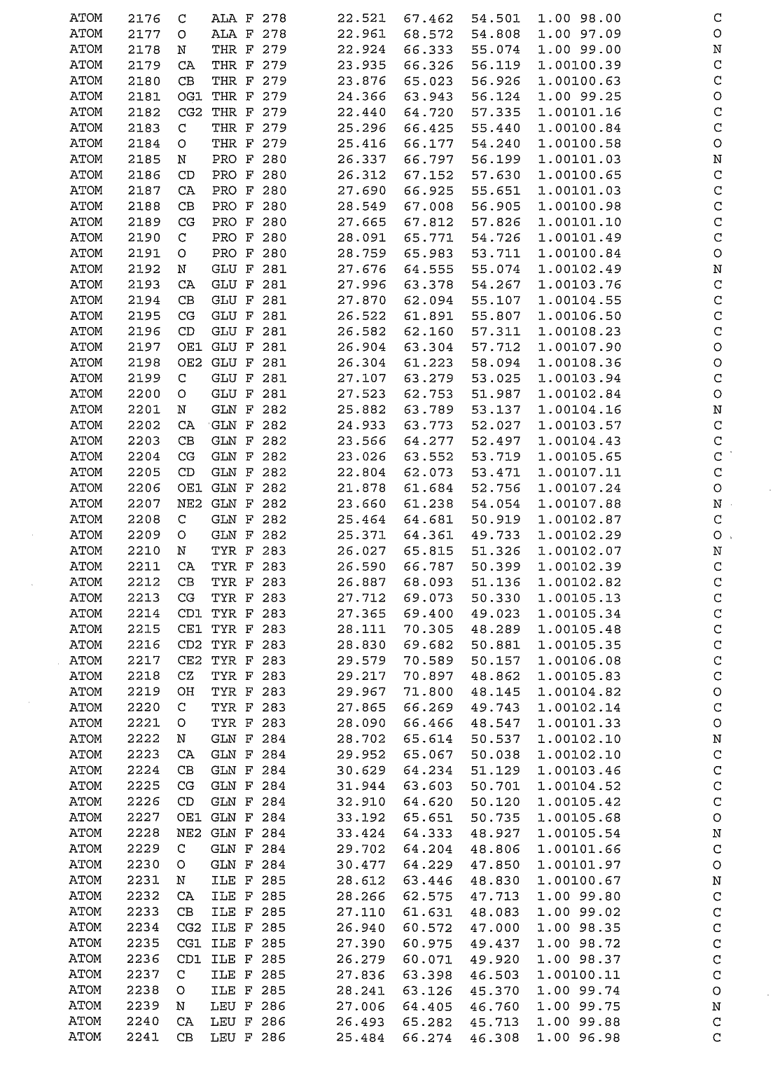

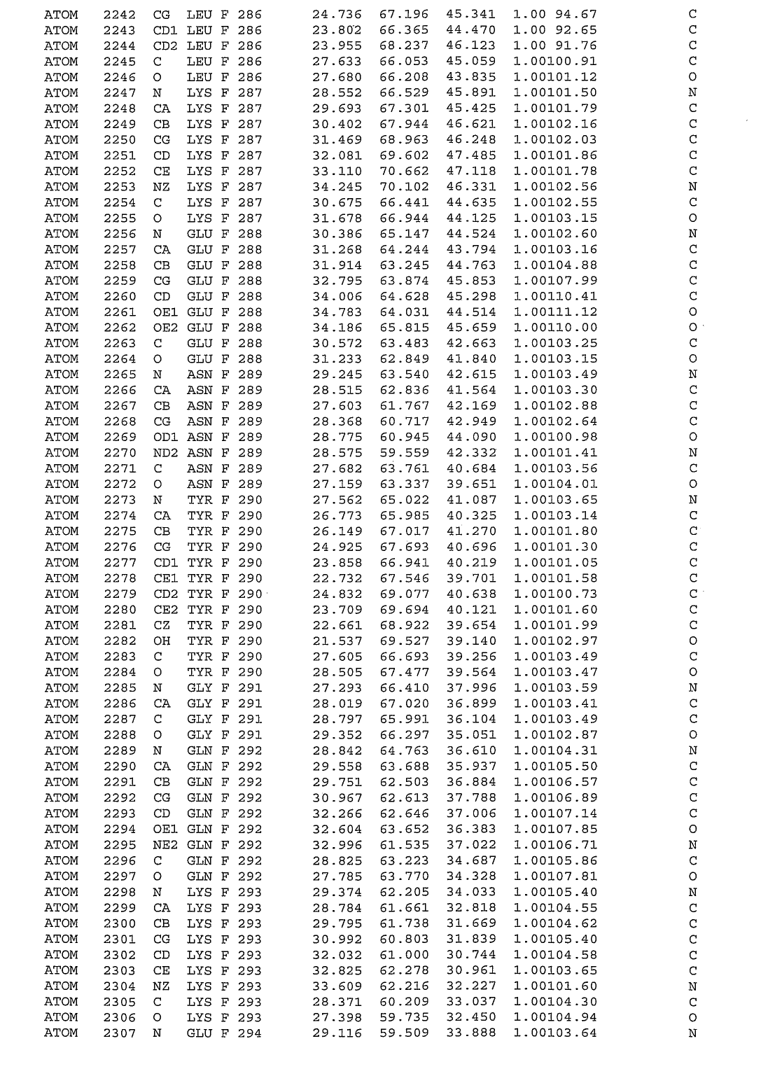

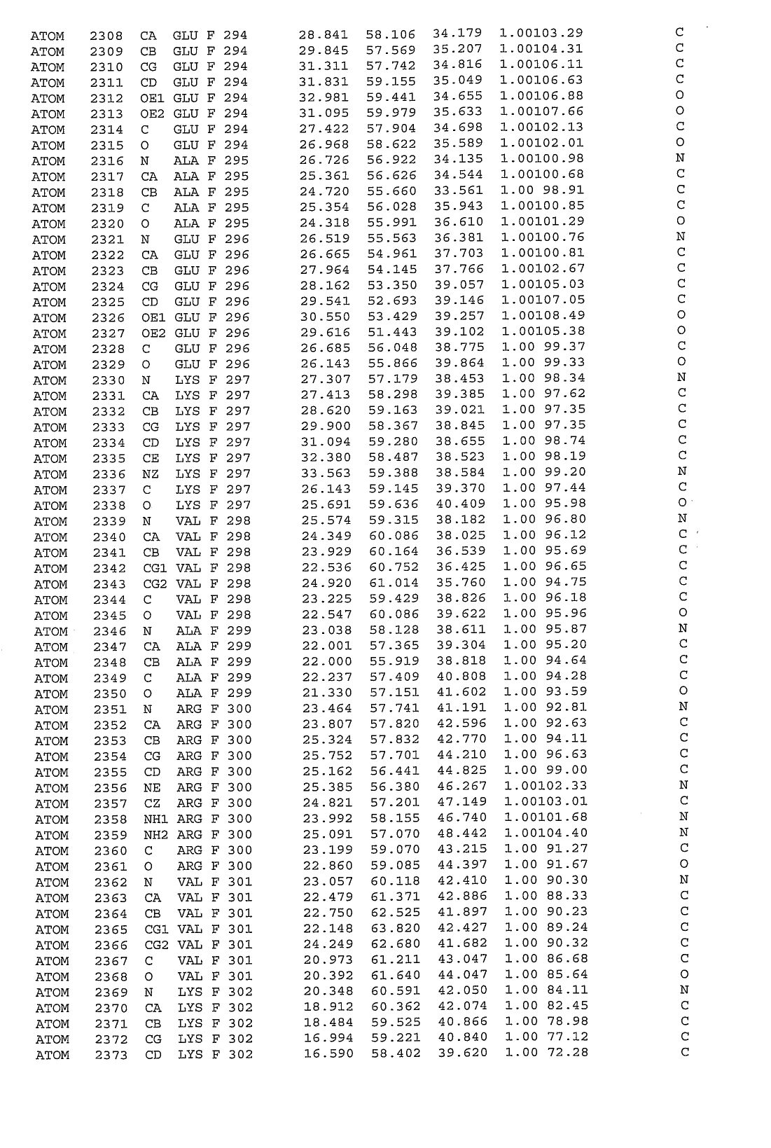

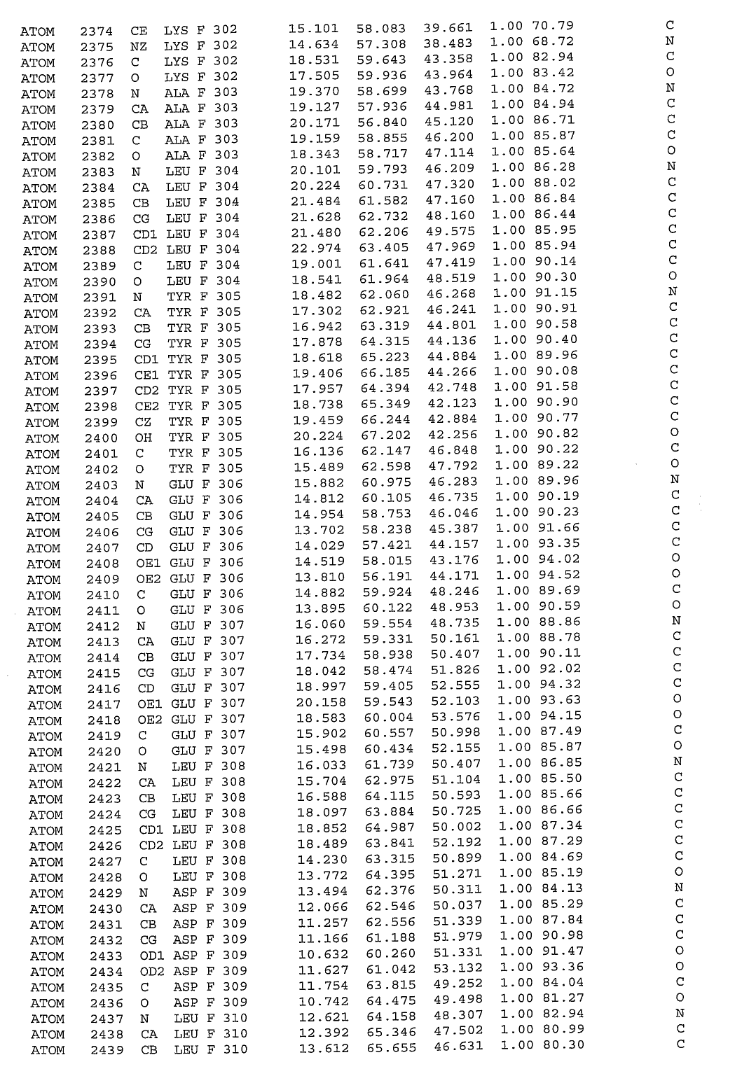

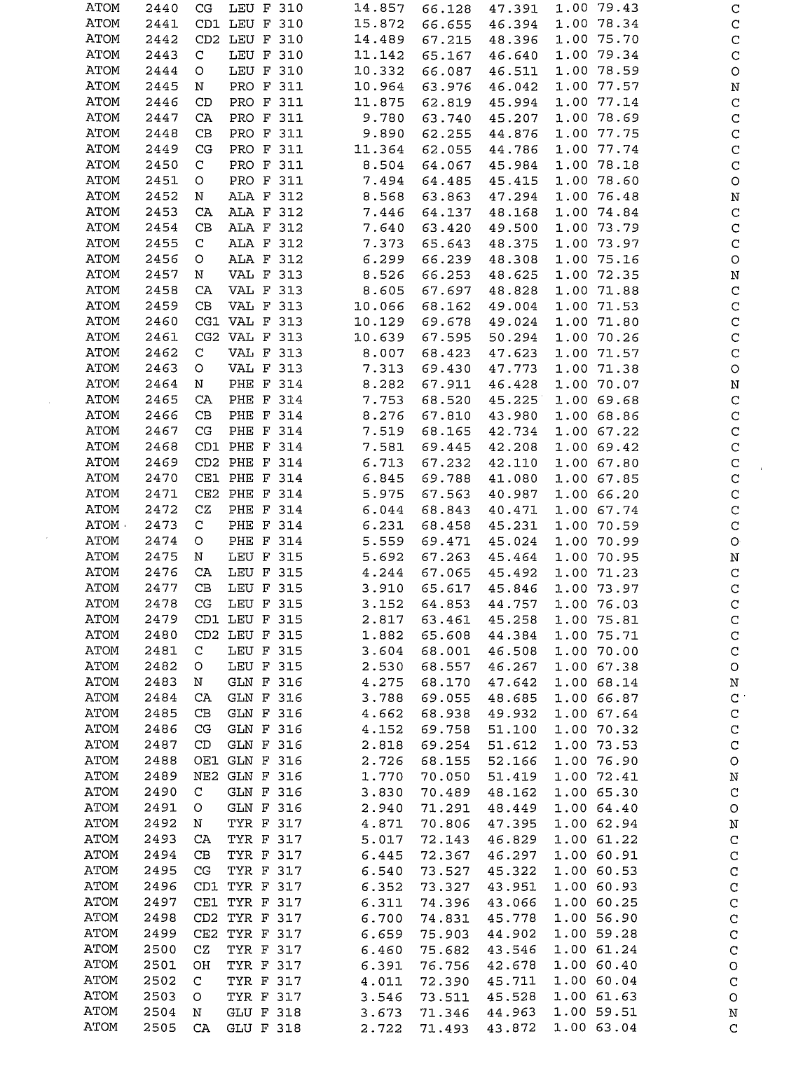

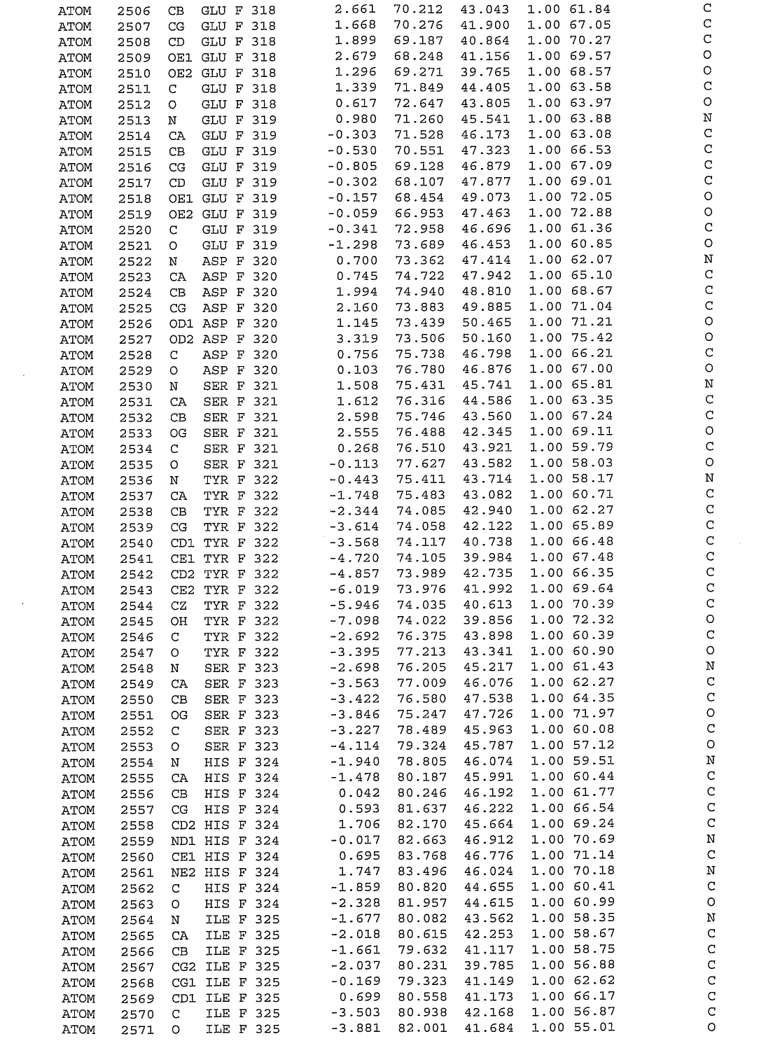

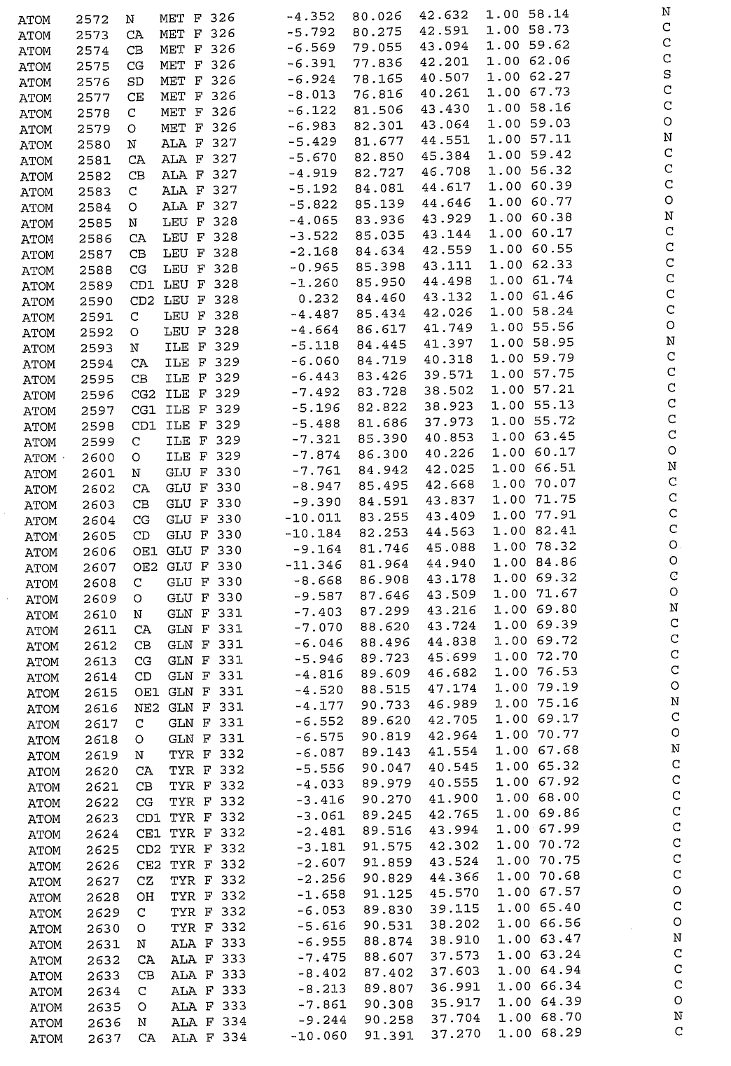

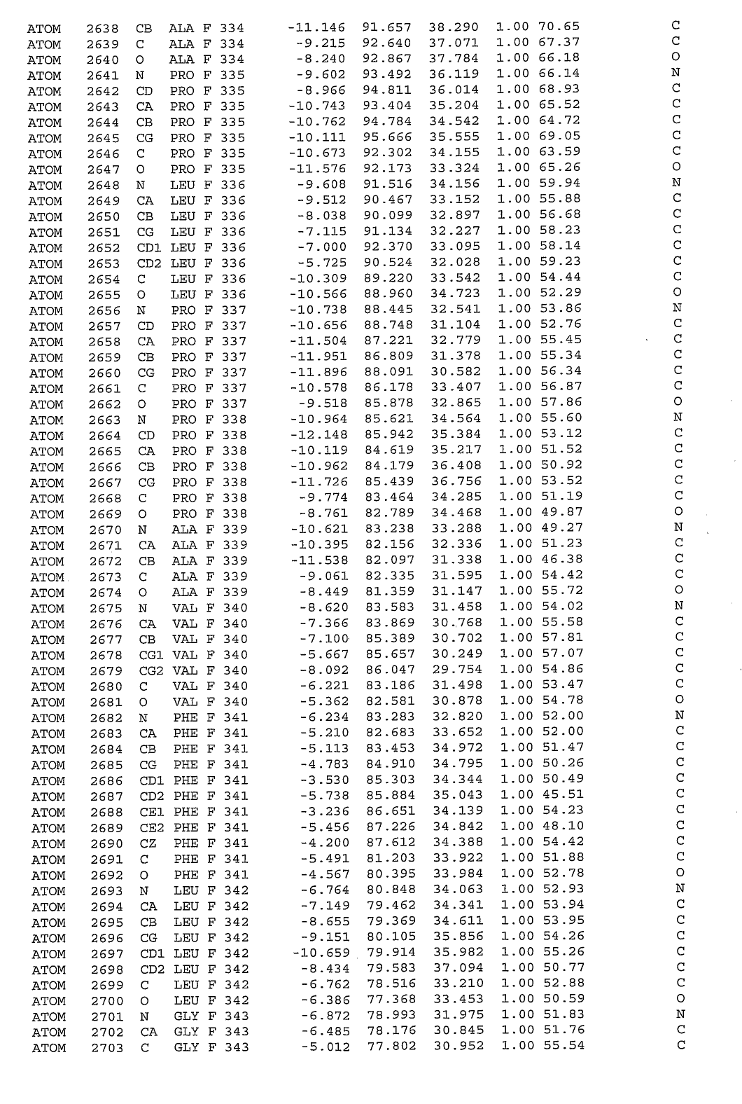

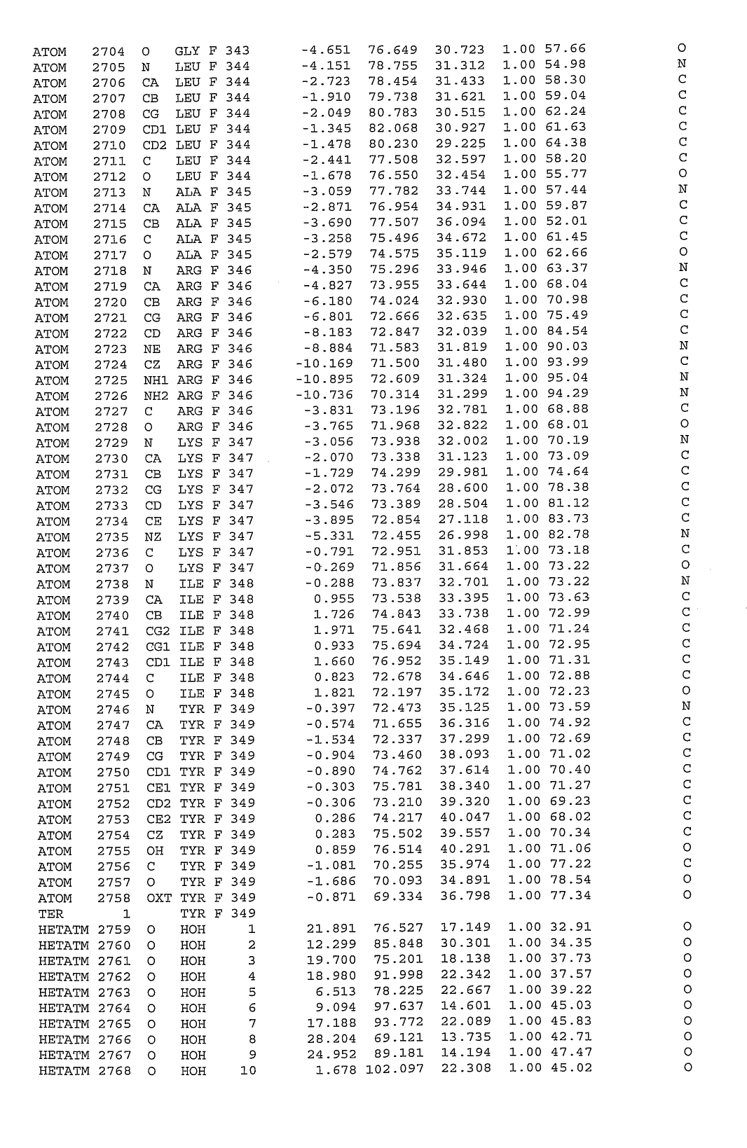

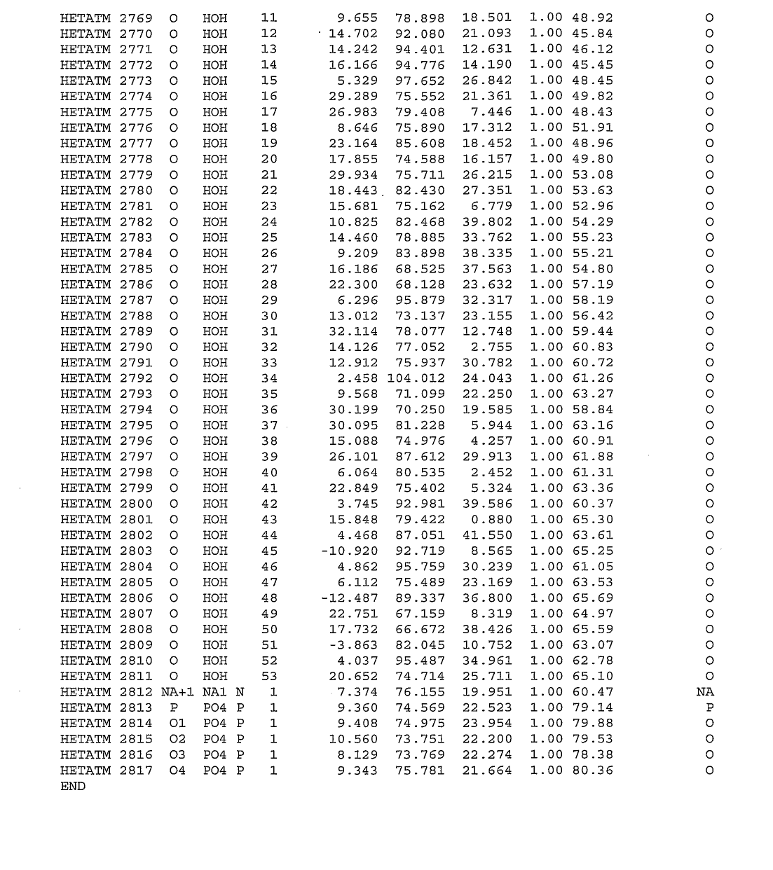

Five complete data sets were collected at the Swiss Light Source for the apo enzyme in the open (2.3 Å) and closed conformation (2.4 Å), as well as for the binary complexes with pamidronate/MnCl₂ (2.6 Å) and zoledronate/MgCl₂ (2.2 Å) and for the ternary complex with IPP and zoledronate/MgCl₂ (2.6 Å) (cf. the Tables of FIGS. 14, 15, 16, 17 and 18).

6b. X-ray Data Collection: FPPS Complex with Ibandronate

X-ray data were collected at 100K using a MARCCD 225 mm detector and synchrotron radiation (Swiss Light Source, beam line PX-II). One single crystal was mounted in a cryo-loop and directly flash-frozen in a cold nitrogen stream. Diffraction data were recorded as 1.00 oscillation images which were processed and scaled with XDS/XSCALE (Kabsch, 1993). The crystal was in space group P4₁2₁2 with one FPPS monomer per asymmetric unit. Crystal data and data collection statistics are shown in Table 1b.

### 7. Structure Determination

The structure of unliganded human FPPS was initially determined by molecular replacement with the program AMoRe (Navaza, Acta Crystallogr. Sect A 50 (1994) 157-163), using data between 15.0 and 3.5 Å resolution and the 2.6 Å structure of avian FPPS (PDB entry 1 FPS) as search model. Human and avian FPPS share 69% (241/345) sequence identity. For both the closed and the open form, a clear molecular replacement solution was found. For the closed form, a correlation coefficient of 69.5% and an R-factor of 0.311 were obtained. For the open form, the correlation coefficient was 51.0% and the R-factor was 0.410. Both structures were then refined with CNX v2002.02 (Brünger et al., Acta Crystallogr. Sect. D; Biol. Crystallogr. 54 (1998) 905-921) using several cycles of torsion angle dynamics and energy minimization, interspersed by model rebuilding steps with the program O (Jones et al., Acta Crystallogr. Sect A, 47 (1991) 110-119). During refinement, the protein_rep.param force field (Engh and Huber, Acta Crystallogr. Sect A, 47 (1991) 392-400) was used; a bulk solvent correction based on the mask method was applied, as well as an initial anisotropic B factor correction. Restrained isotropic atomic B-factors were refined. The refinement target was the maximum-likelihood target using amplitudes. No sigma cut-off was applied on structure factor amplitudes. Cross-validation was used throughout refinement using a test set comprising 10% of the reflections. Water molecules were identified with the CNX script water_pick.inp, and selected based on difference peak height (greater than 3.0σ), hydrogen-bonding and distance criteria. Waters with temperature factors greater than 65 Å² were rejected.

The structures of the binary complexes with pamidronate and zoledronate and of the ternary complex with IPP and zoledronate were determined using an initial rigid-body refinement of the apo structure followed by full refinement using the same procedure as described here above.

Final refinement statistics for all crystallographic models are presented in Table 2.

8. Overall Three-dimensional Structure of hFPPS

Like avian FPPS, human FPPS is a homodimer with two identical active sites. In the crystals, the two subunits are related by a crystallographic dyad. Each subunit is folded as a single domain composed of thirteen α-helices, of which ten form a core helical bundle. Hereafter we adopt the nomenclature first proposed for avian FPPS (Tarshis et al., 1994, Biochemistry; 33:10871-10877) whereby the ten helices of the core bundle are named by the letters A to J while the three short helices inserted between helix H and I are labeled α-1 to α-3. The packing of the ten core helices has been described as a three layer structure, with helices A and B forming the first layer, helices C, D, E and J forming the second layer and helices F, G, H and I forming the third layer (Tarshis et al., above). Helix G exhibits 2 kinks, the first one occurring at the highly conserved Lys200-Thr201 sequence, the second around Pro209.

The two conserved DDXXD motifs are located at the C-terminal end of helices D and H. These two helices, together with helices C, F, G and J, form the walls of the very deep and large FPPS active site. Three prominent loops connecting helices B and C (the “B-C loop”), D and E (the “D-E loop”) and H and I (the “H-I loop”) line the entrance of the enzyme active site. These three loops harbor conserved glycine, lysine and arginine residues: Gly56, Lys57 and Arg60 in the B-C loop/C helix, Arg112, Arg113, and Gly114 in the D-E loop, Gly256, Lys257 and Lys266 in the H-I loop. Other highly conserved residues among prenyl synthetases cluster around the FPPS active site.

The dimer interface consists mainly of helices D and E with additional inter subunit interactions provided by the first two N-terminal α-helices (A and B), which are nearly orthogonal to all other α-helices (FIG. 1).

9. Closed Conformation of hFPPS (“apo closed form”)

Human FPPS crystals prepared according to purification method I were all representative of crystal form I (closed conformation). During the refinement of the structure, it became apparent that the human enzyme was, in comparison to avian FPPS, in a different conformational state, to which reference is made as the closed state (FIG. 1). Furthermore, within the enzyme active site, residual difference electron density was observed corresponding to an endogeneous ligand that was partially interpreted as comprising a phosphate group (FIG. 2). The electron density of this ligand was consistent with a phosphorylated (or sulfated) compound of about 150-200 Da. This finding was confirmed by mass spectrometry of “unliganded” FPPS performed under non-denaturing conditions (Bitsch et al., Anal. Biochem. 373 (2000) 231-241), which showed the presence of a fortuitous ligand with a molecular weight of 200 Da.

Three conserved basic side-chains interacted with the ligand: Lys57 and Arg60 of the B-C loop/C-helix and Arg113 of the D-E loop. It was subsequently found that the same residual electron density was also present in the crystals of the binary complexes with pamidronate or zoledronate, and that this ligand was displaced by IPP in the ternary complex with zoledronate and IPP. Structural overlays indicated that the residual difference electron density was about the size of the diphosphate group of IPP and occupied its binding site.

10. hFPPS in the Open Conformation (“apo open form”)

In order to determine the three-dimensional structure of truly unliganded human FPPS, a second batch of enzyme was prepared and extensively purified using additional chromatographic and dialysis steps. The crystals obtained were in the same tetragonal space group with again one FPPS subunit per asymmetric unit, but showed a 10 Å increase in the length of the c axis (crystal form II) (open conformation). The structure was determined by molecular replacement, again using avian FPPS as search model.

The X-ray analysis confirmed that the above-described endogeneous ligand had been successfully eliminated by the new purification protocol. Nevertheless, a well-defined phosphate ion from the crystallization mother liquor filled the site previously occupied by the putative phosphate group of the ligand. More importantly, the enzyme was found to adopt an open conformation, similar to that originally observed with avian FPPS. When the open and the closed conformation of human FPPS are superimposed using all Cα atoms, an rms deviation of 1.7 Å is obtained. However, the structural differences between the two conformational states are best revealed when the two structures are superimposed using only the first 150 N-terminal Cα atoms.

11. hFPPS Complex with Pamidronate and Mn²⁺
11a. Metal Binding Sites

The enzymatic reaction catalysed by FPPS requires the presence of either Mg²⁺ or Mn²⁺. In order to determine the number and location of the metal sites, human FPPS was crystallized in the presence of MnCl₂ and pamidronate. In an X-ray experiment, Mn²⁺ ions (23 electrons) give a stronger signal than Mg²⁺ (10 electrons), and hence allow the unambiguous identification of the metal binding sites, even at medium resolution. The hFPPS/pamidronate/MnCl₂ data clearly demonstrated the presence of three Mn²⁺ cations within the FPPS active site cavity. The three metal ions are coordinated by the bisphosphonate unit of the inhibitor and three aspartate side-chains from the two conserved DDXXD sequence motifs: Asp103, Asp107 and Asp243. Two Mn²⁺ ions, located only 3.3 Å apart, bind to the first DDXXD motif (helix D), with the carboxylate group of Asp103 acting as a bridging ligand and Oδ2 of Asp107 coordinating both metal ions. The third Mn²⁺ ion binds to the second DDXXD motif (helix H) through Oβ2 of Asp243 and is 4.9 Å and 6.2 Å away, respectively, from the other two metal sites. All carboxylate oxygen atoms coordinate the metal centers with the commonly observed syn geometry, with the exception of Oδ2 of Asp107, which uses both the syn and the anti coordination geometry.

11b. Binding of Pamidronate to hFPPS

Moreover, the X-ray analysis establishes that N-containing bisphosphonate inhibitors, e.g. pamidronate, bind to the allylic substrate site of FPPS, and, contrary to previous models, interact with both DDXXD motifs through the trinuclear metal center. The side-chain amino group of pamidronate does not have well defined electron density, suggesting that this substituent does not make strong interactions with the enzyme active site or adopts more than one orientation in the complex. Nevertheless, the phenol hydroxyl moiety of Tyr204 would be in a suitable position to form a hydrogen-bonded interaction with the pamidronate primary amino group (FIG. 5). In addition, three basic side-chains are involved in direct salt-bridge interactions with the bisphosphonate unit of pamidronate: Lys200, Arg112 and Lys257. Worth of note, Lys257 is part of the mobile H-I loop. Hence, pamidronate binding stabilizes the closed conformation of FPPS by interacting with both DDXXD motifs and Lys257 of the H-I loop. Furthermore, in this complex, the IPP binding site is occupied by the ligand already observed in the closed form of “apo” FPPS. The presence of this endogeneous ligand further stabilizes the closed state of the enzyme.

12. hFPPS complex with zoledronate and Mg²⁺

In order to unravel the molecular basis of the higher inhibitory potency of zoledronate with respect to pamidronate, the FPPS complex with zoledronate was co-crystallized in presence of MgCl₂ and the structure was determined to 2.20 Å resolution. The enzyme was found to adopt the closed conformation, again with the IPP binding site occupied by the above-described ligand. Three Mg²⁺ sites matching the positions of the manganese ions in the pamidronate complex were observed. Moreover, the better resolution of the hFPPS/zoledronate/MgCl₂ data revealed the details of the coordination spheres of the three magnesium ions, which all have six coordinating ligands in an approximately octahedral arrangement. Furthermore, the electron density was well-defined for all zoledronate atoms, including the imidazolium ring. The protonated ring nitrogen is within hydrogen-bonding distance of both the main-chain carbonyl oxygen of Lys200 and the side-chain hydroxyl of Thr201, two conserved amino acid residues located at the first kink of helix G. The hydroxyl substituent on the bisphosphonate carbon atom makes a water-mediated H-bond to Oδ1 of Gln240, as well as a direct polar contact to Oδ2 of Asp243, but the geometry of the latter interaction does not seem to be very favorable for a good hydrogen bond.

In comparison to pamidronate, the higher binding affinity of zoledronate appears to derive from the increased rigidity and bulkiness of the imidazole ring and from the polar interactions mentioned here above. Furthermore, it has been proposed that nitrogen-containing bisphosphonates act as transition state analogs mimicking the putative carbocation intermediate formed during the enzymatic reaction. Hence, the increased potency of zoledronate also derives from the fact that its sp²-hybridized imidazolium ring is a better transition state mimic than the primary ammonium group of pamidronate, and is better positioned than the latter in the enzyme active site.

13. hFPPS Complex with IPP, Zoledronate and Mg²⁺

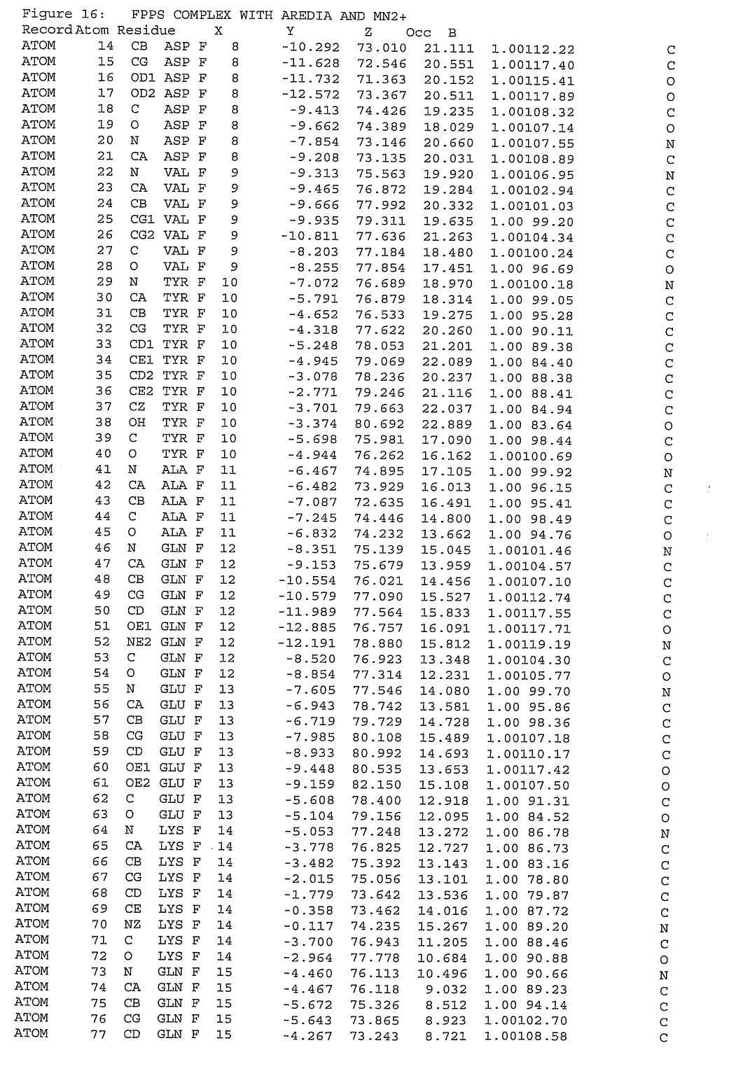

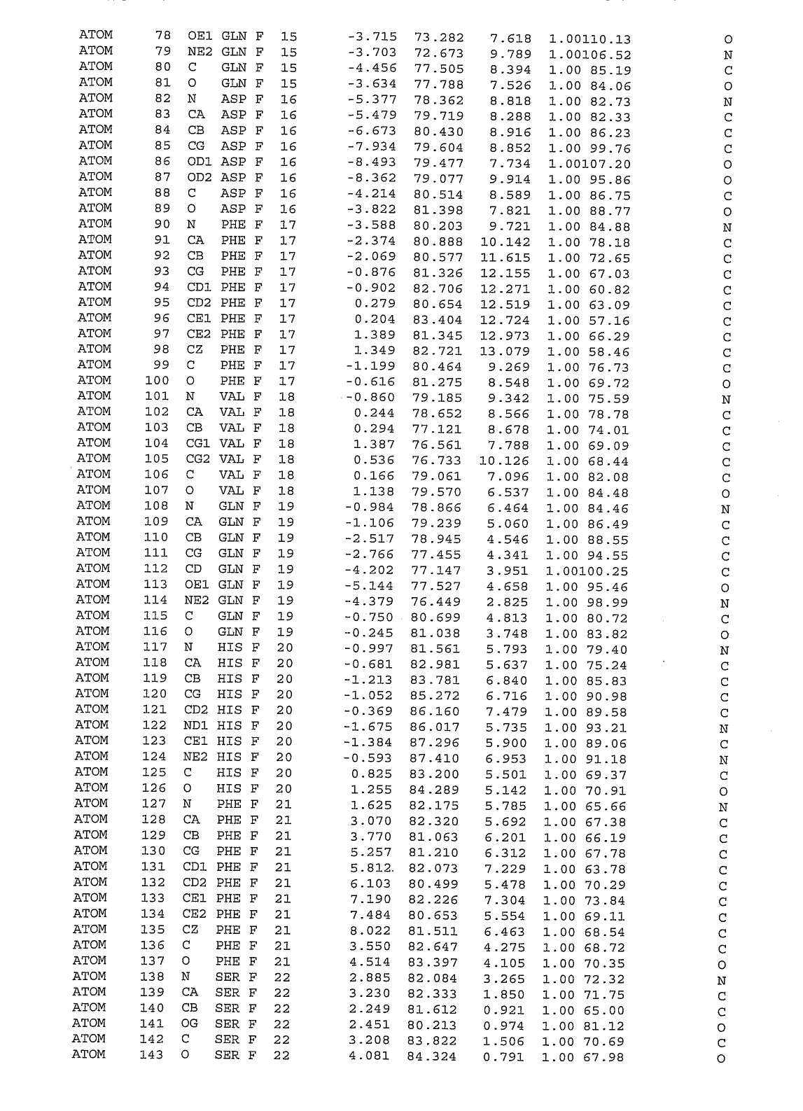

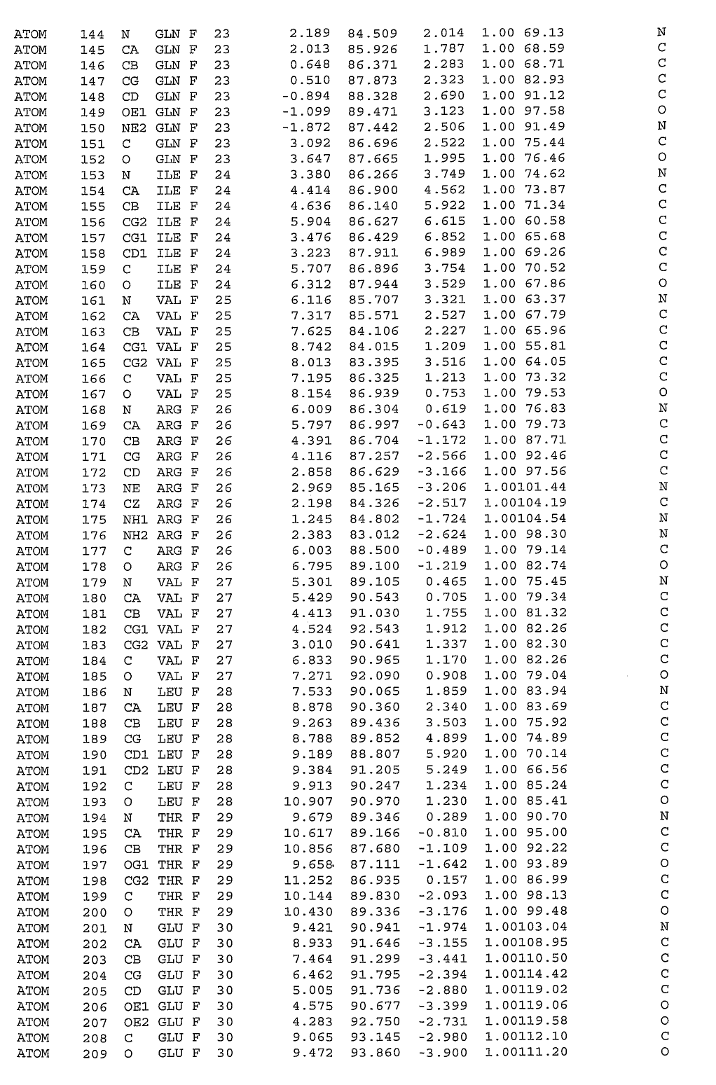

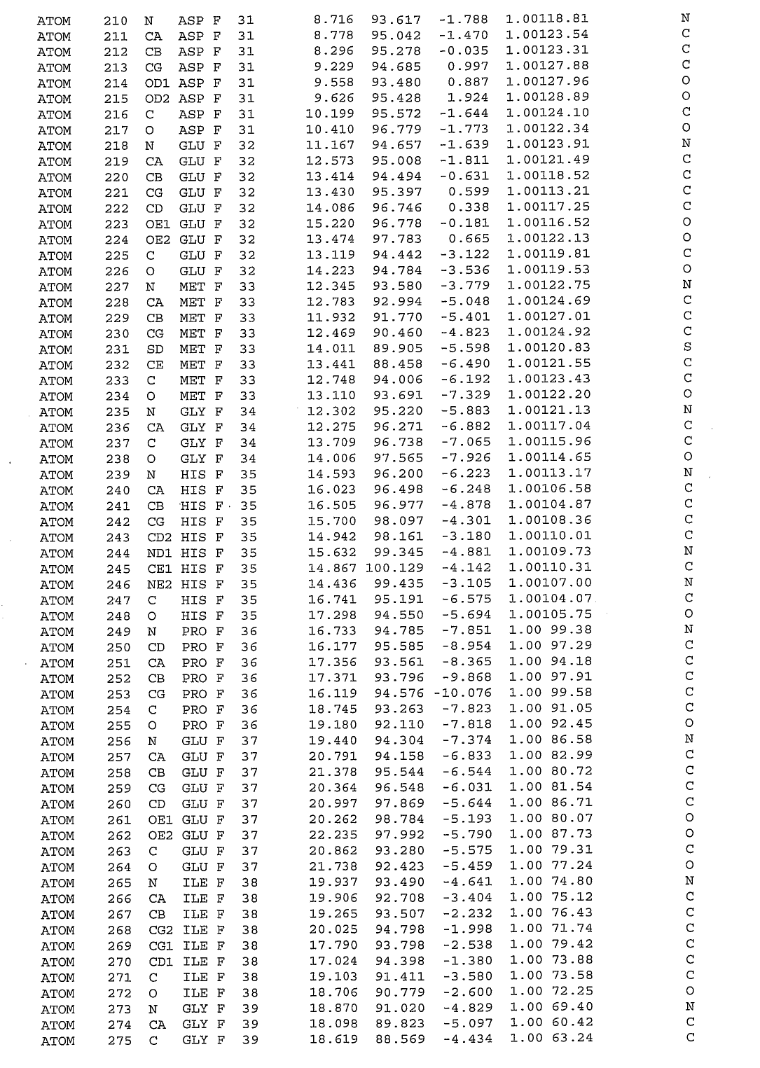

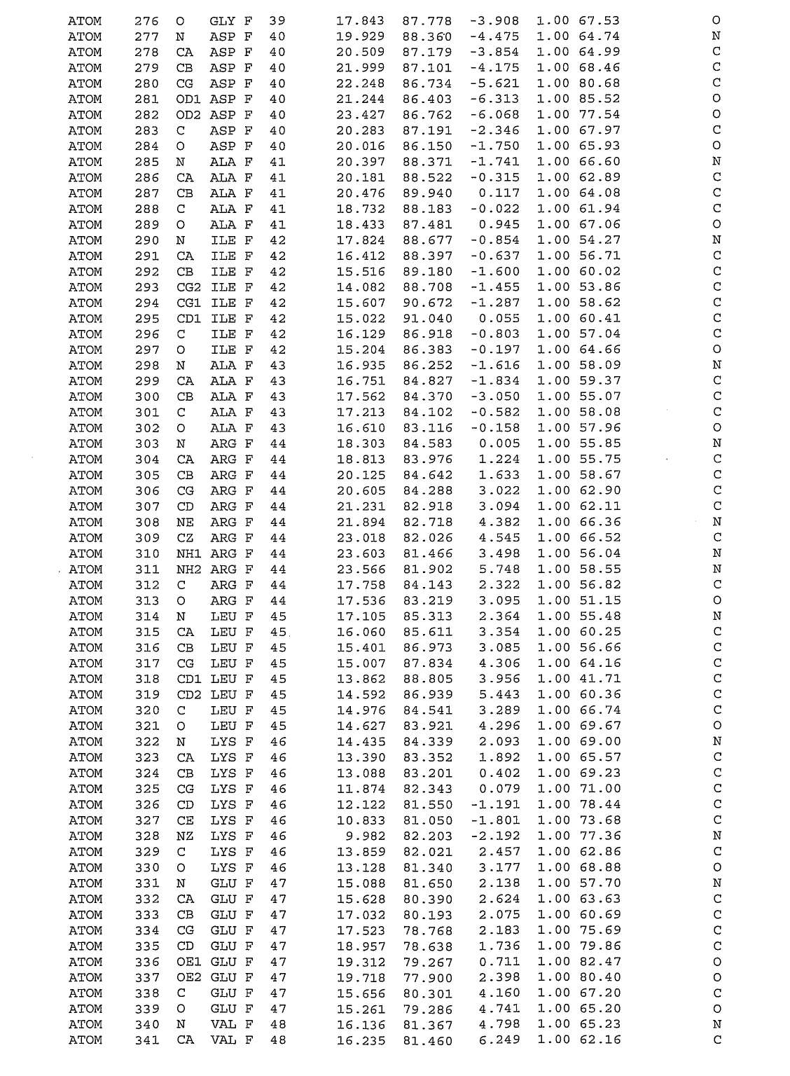

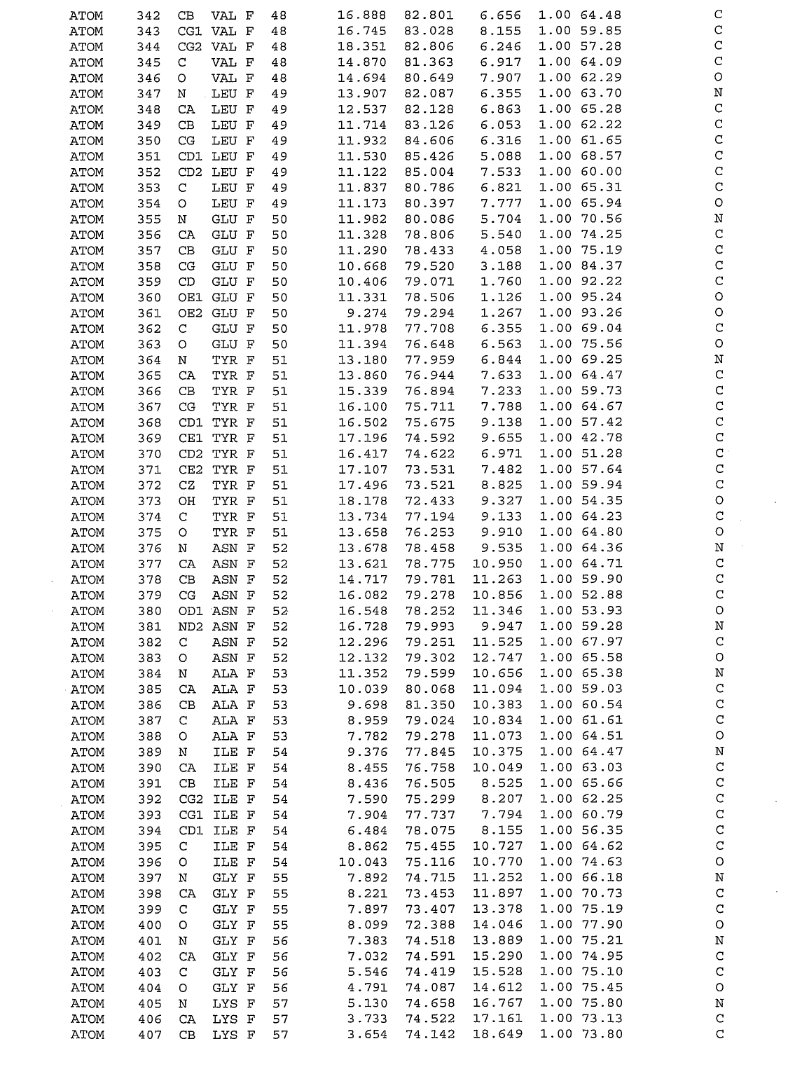

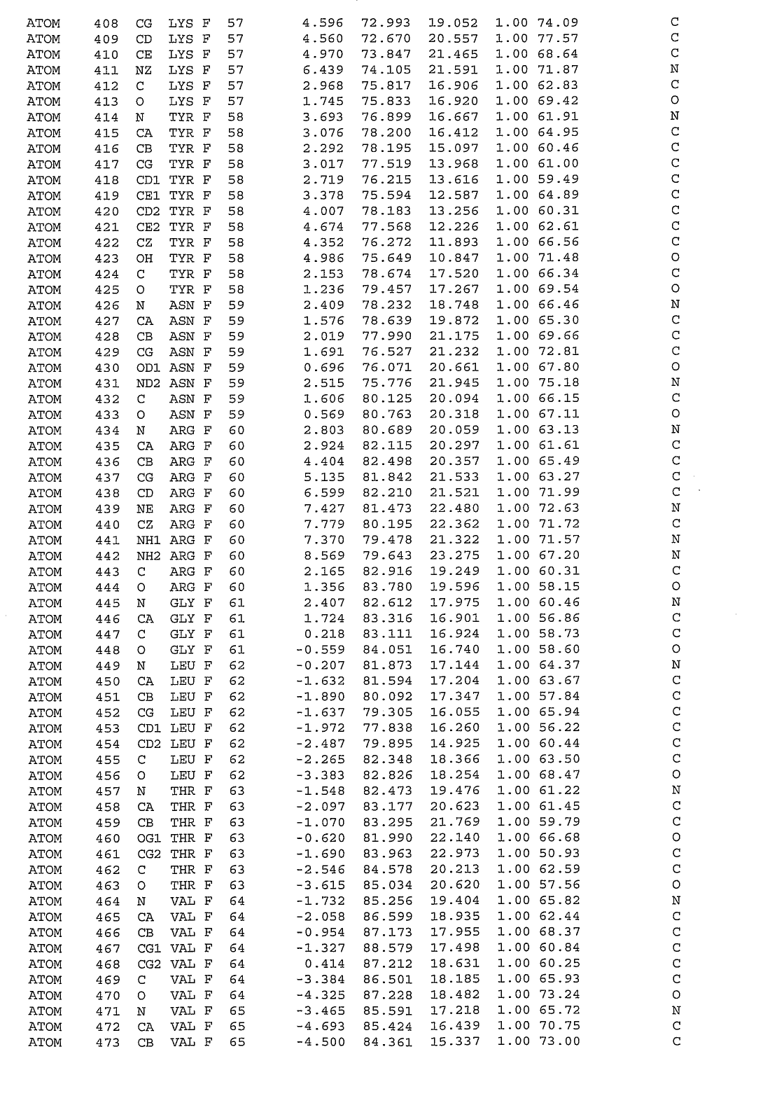

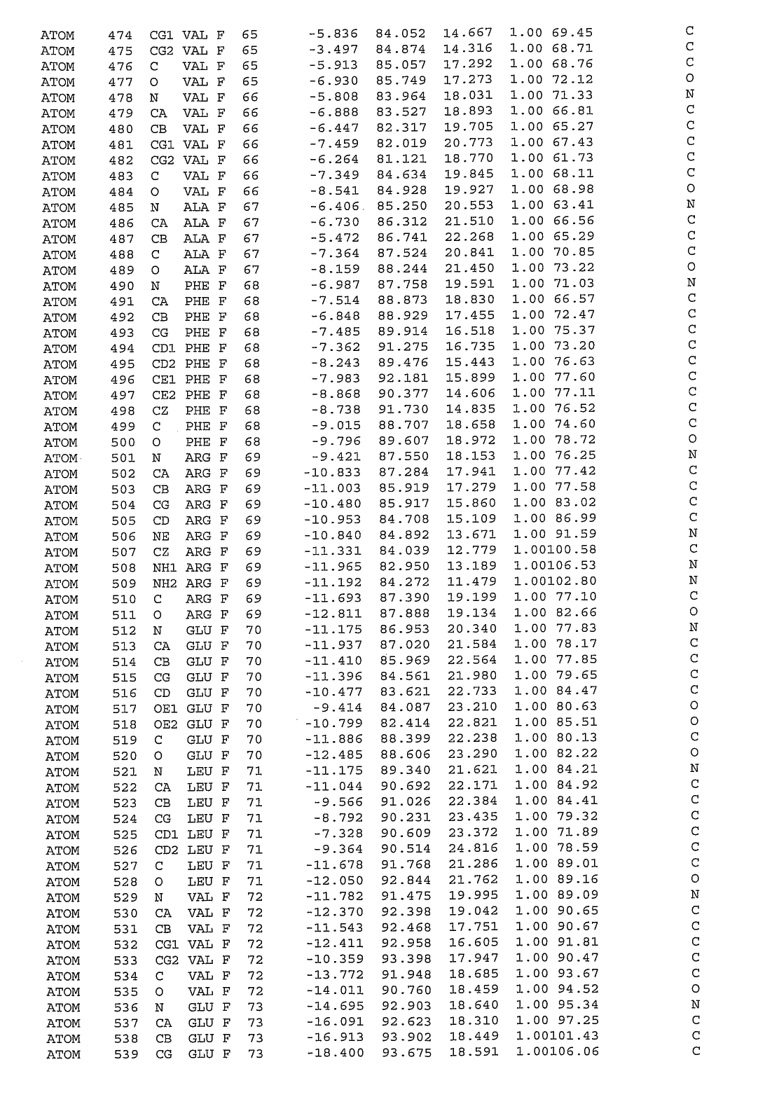

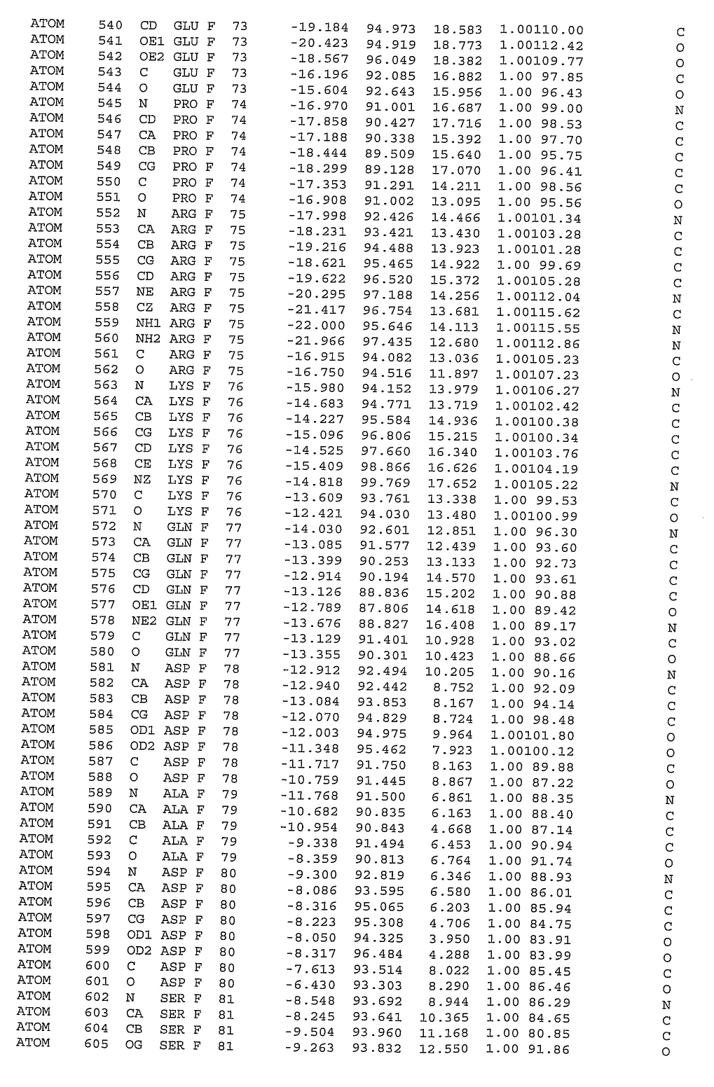

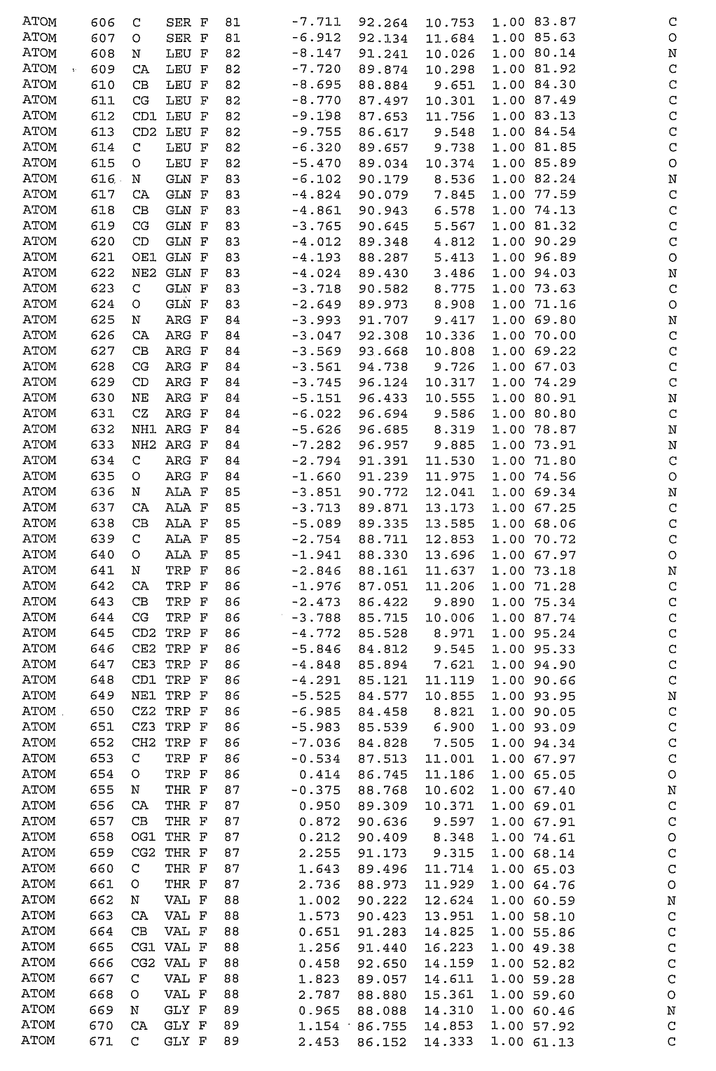

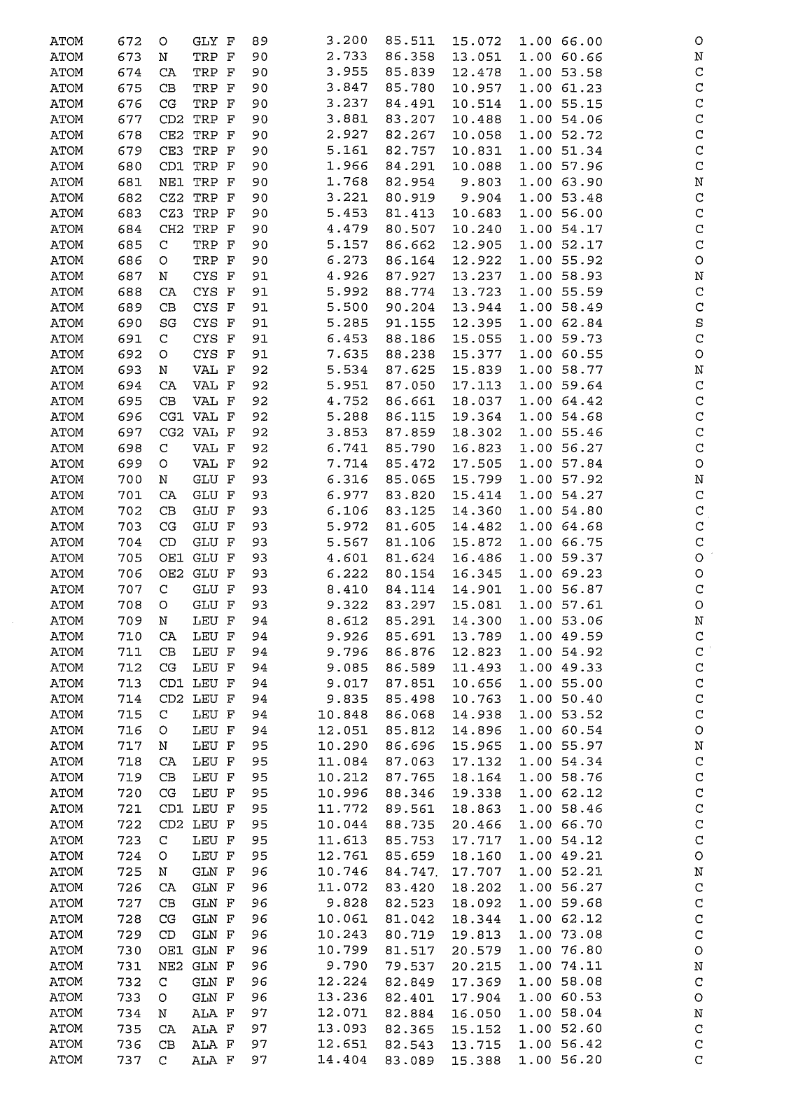

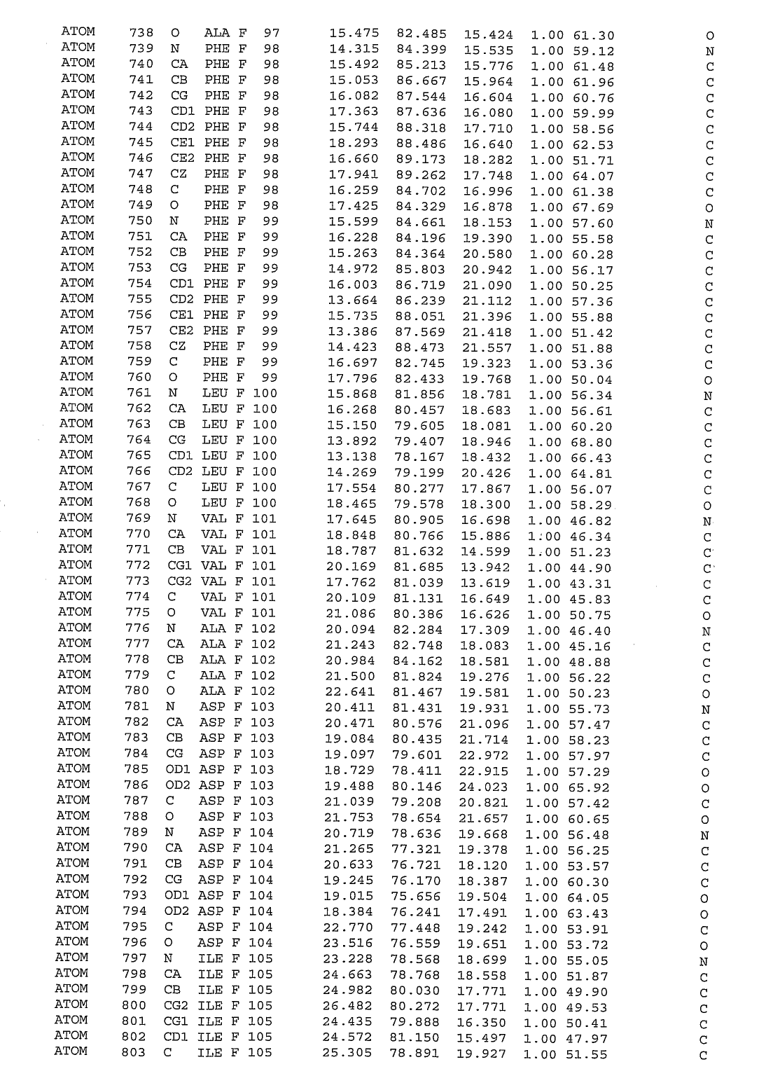

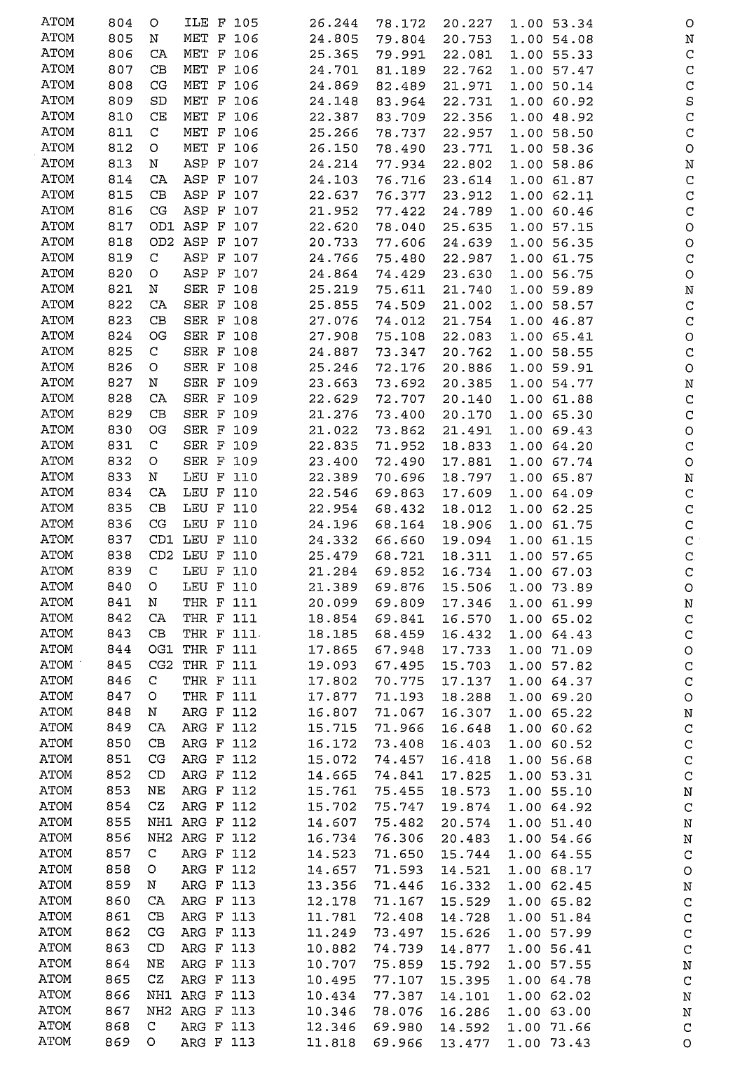

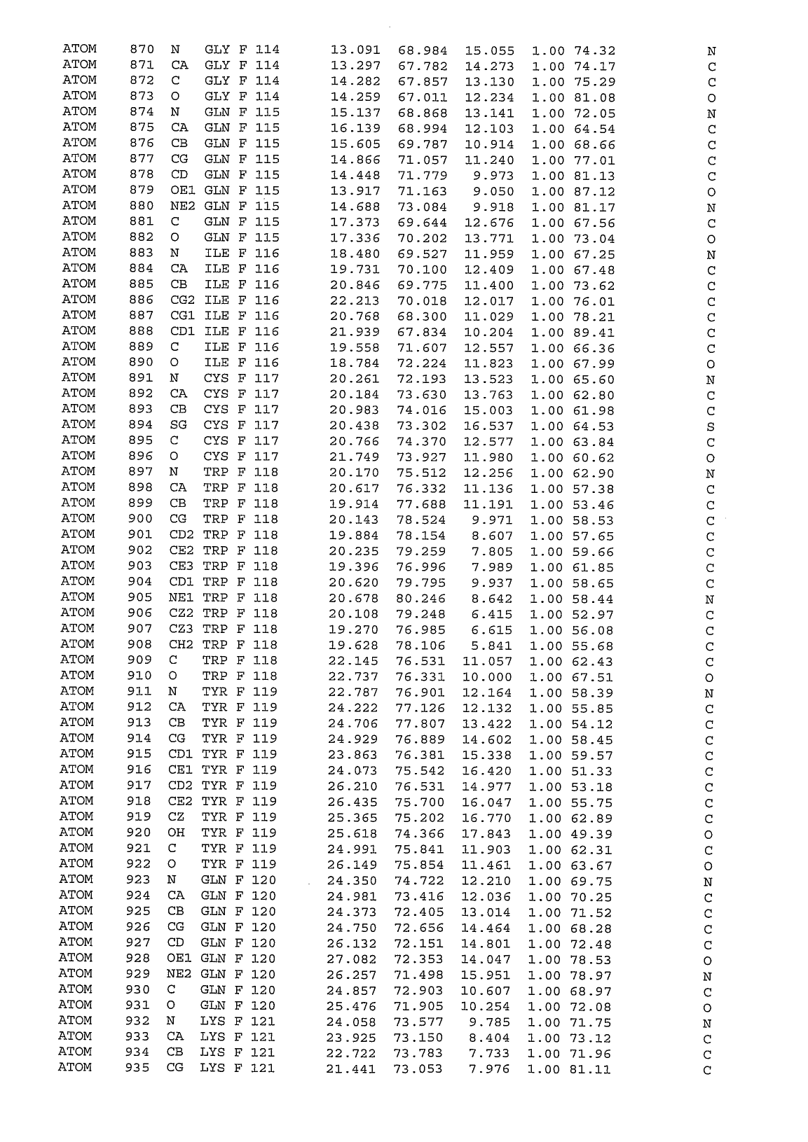

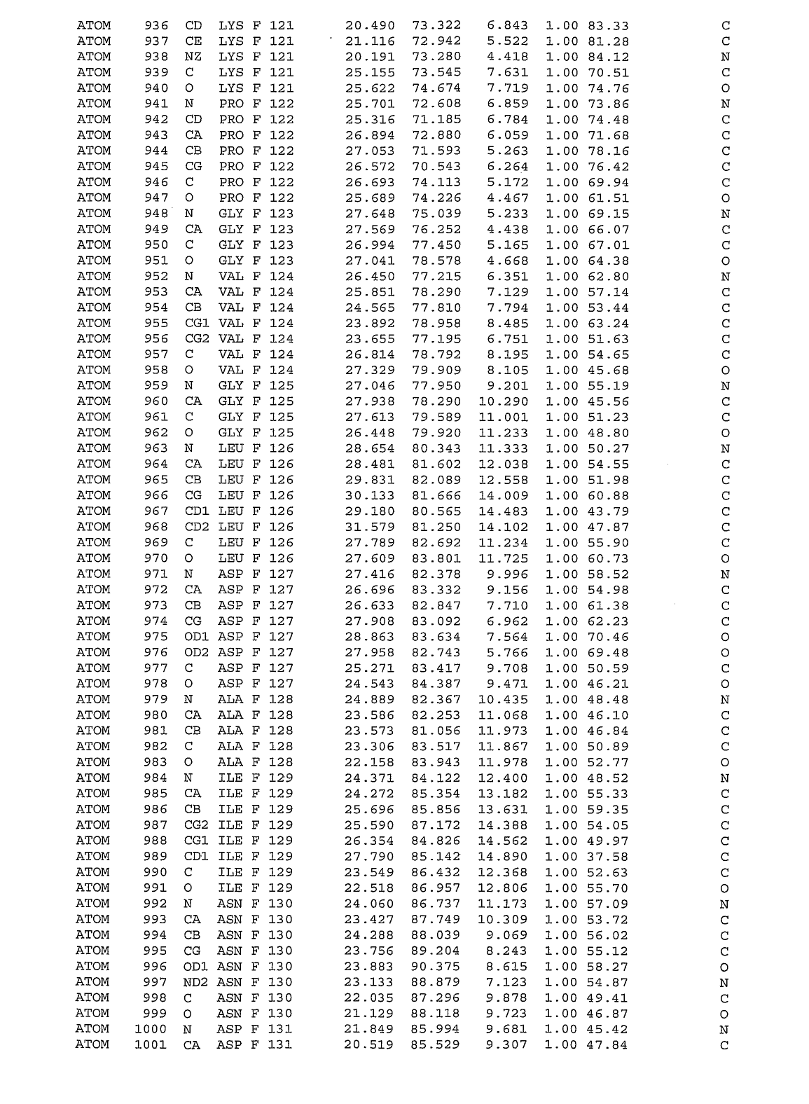

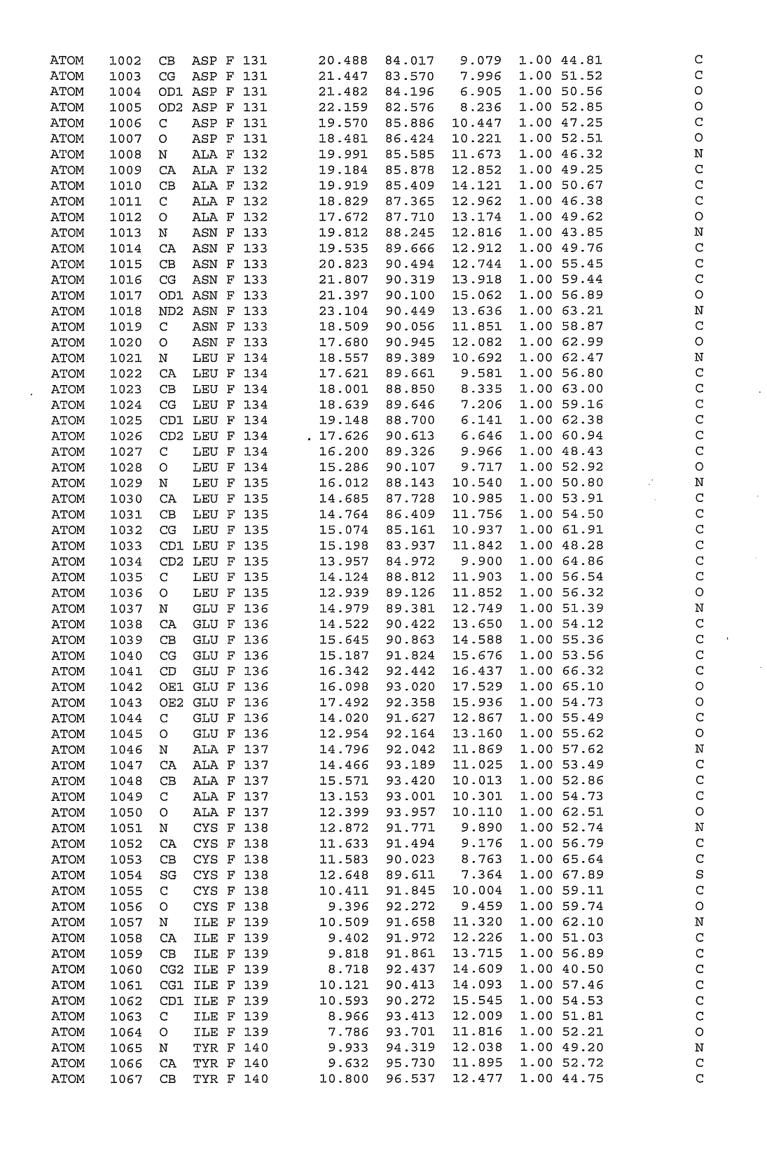

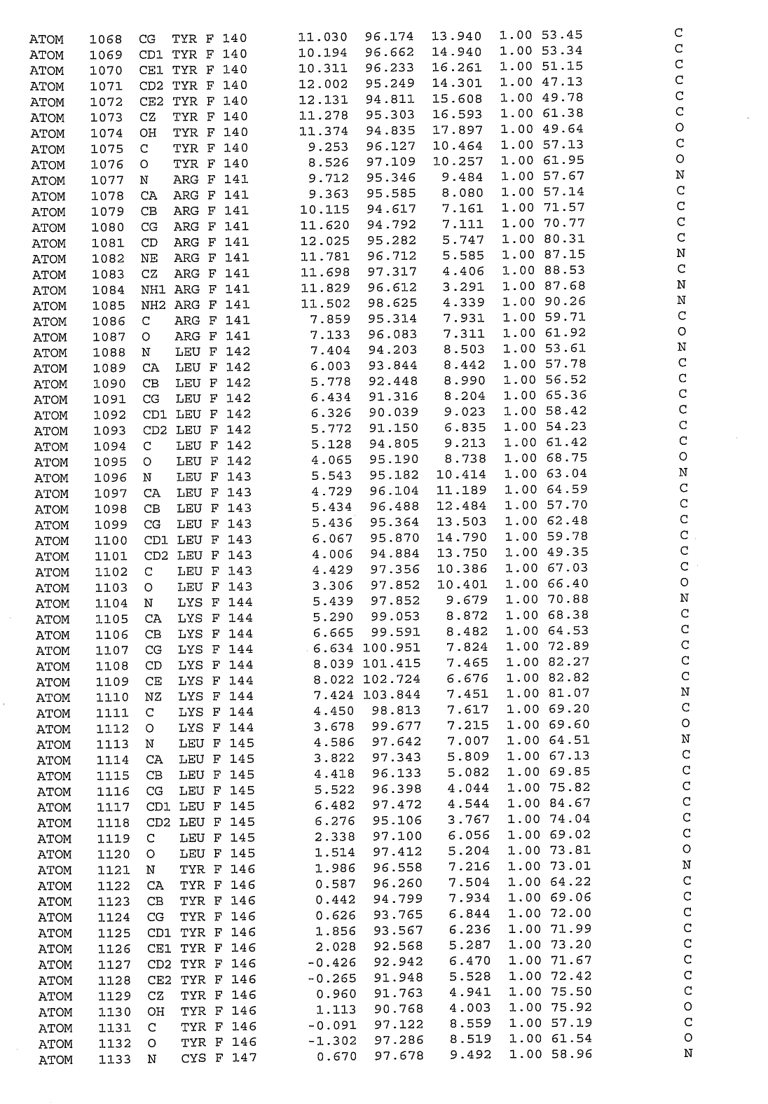

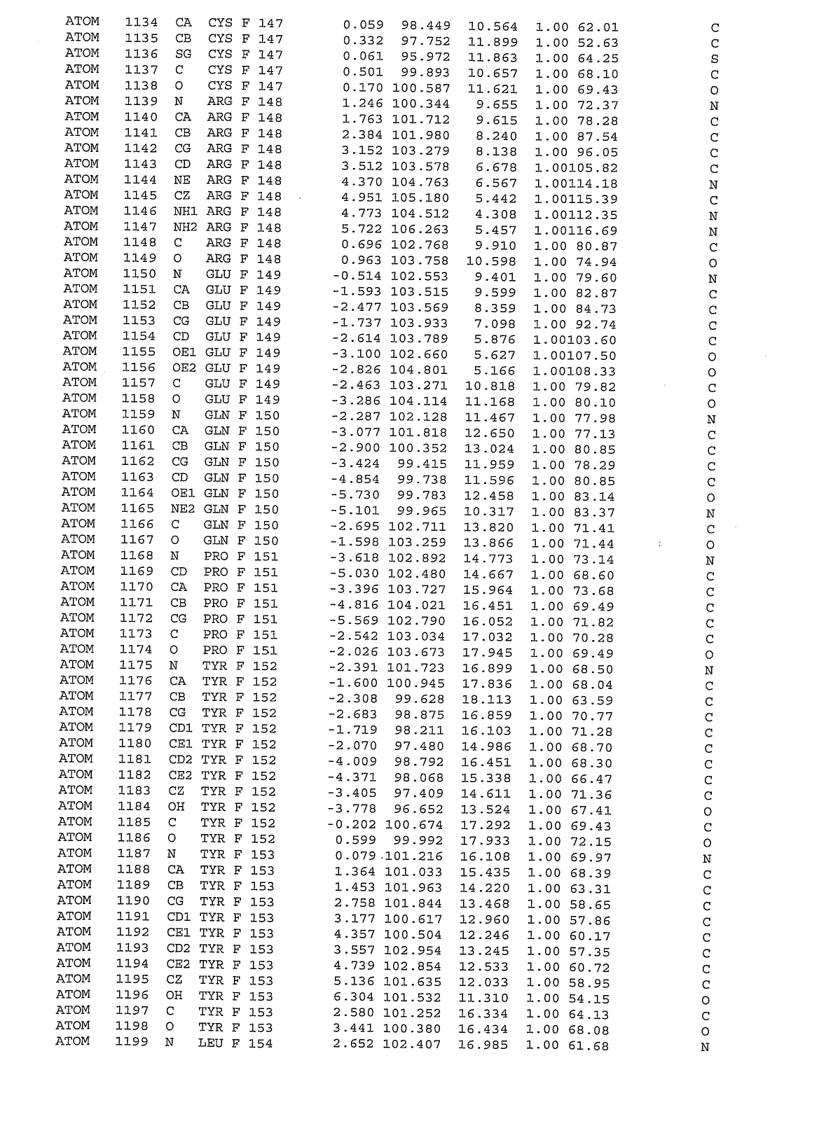

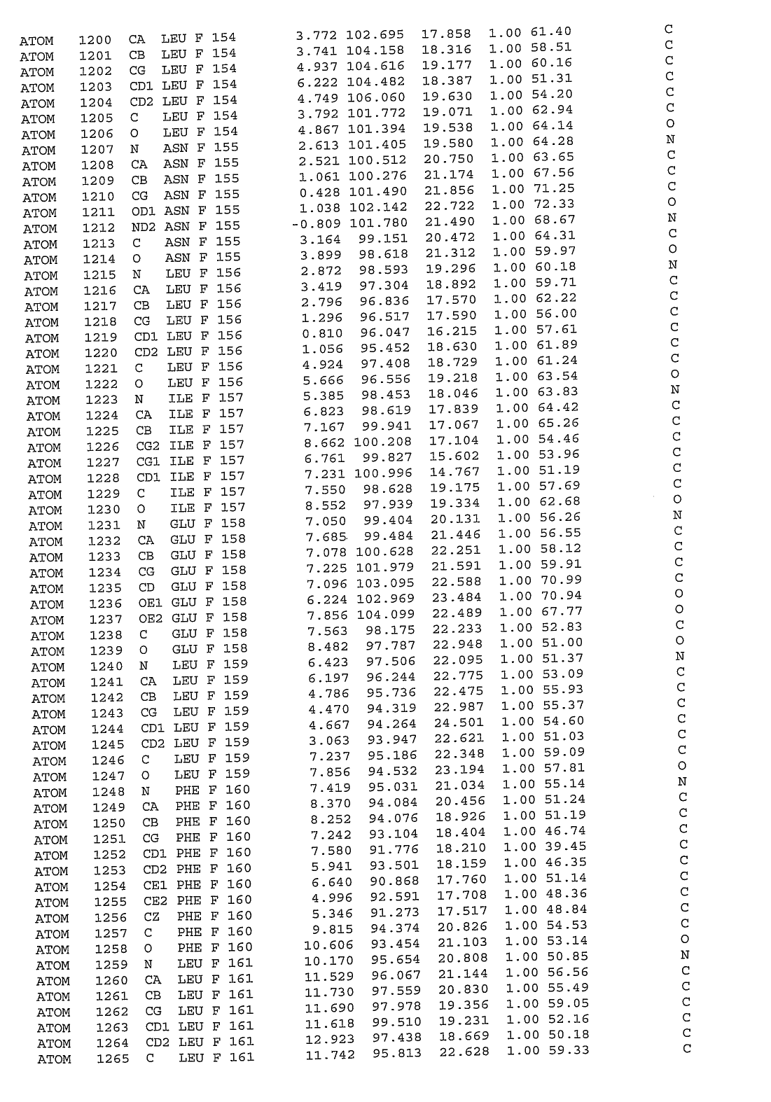

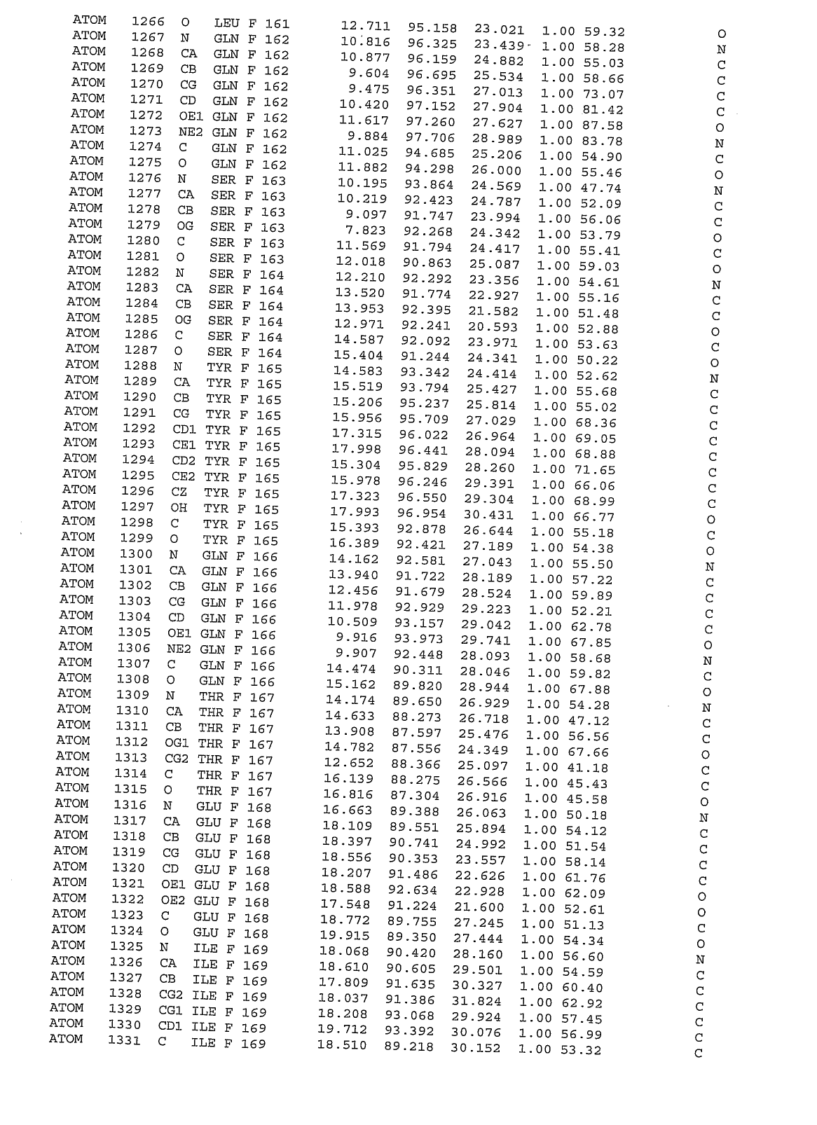

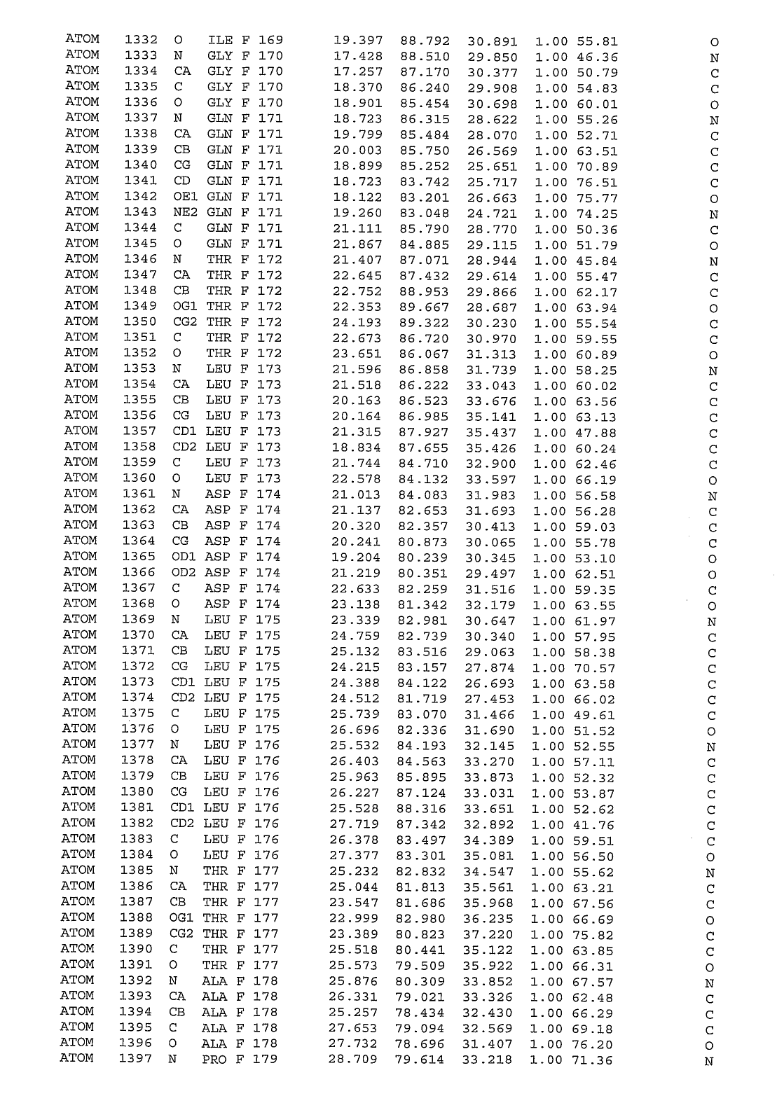

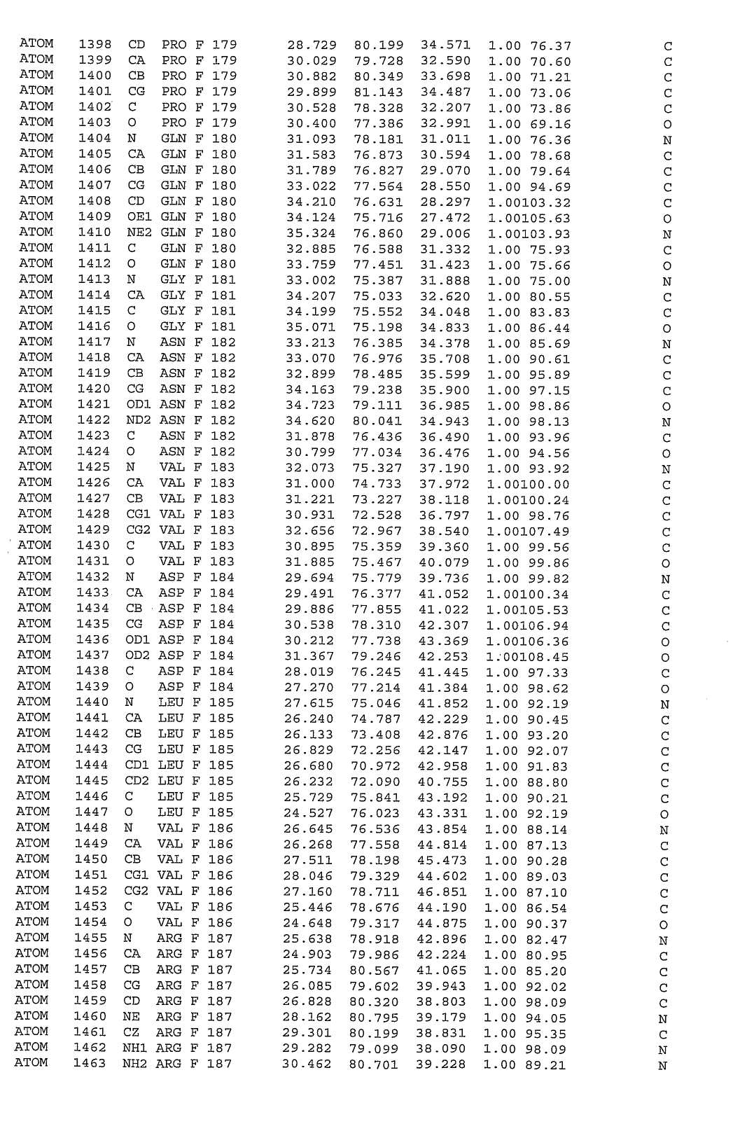

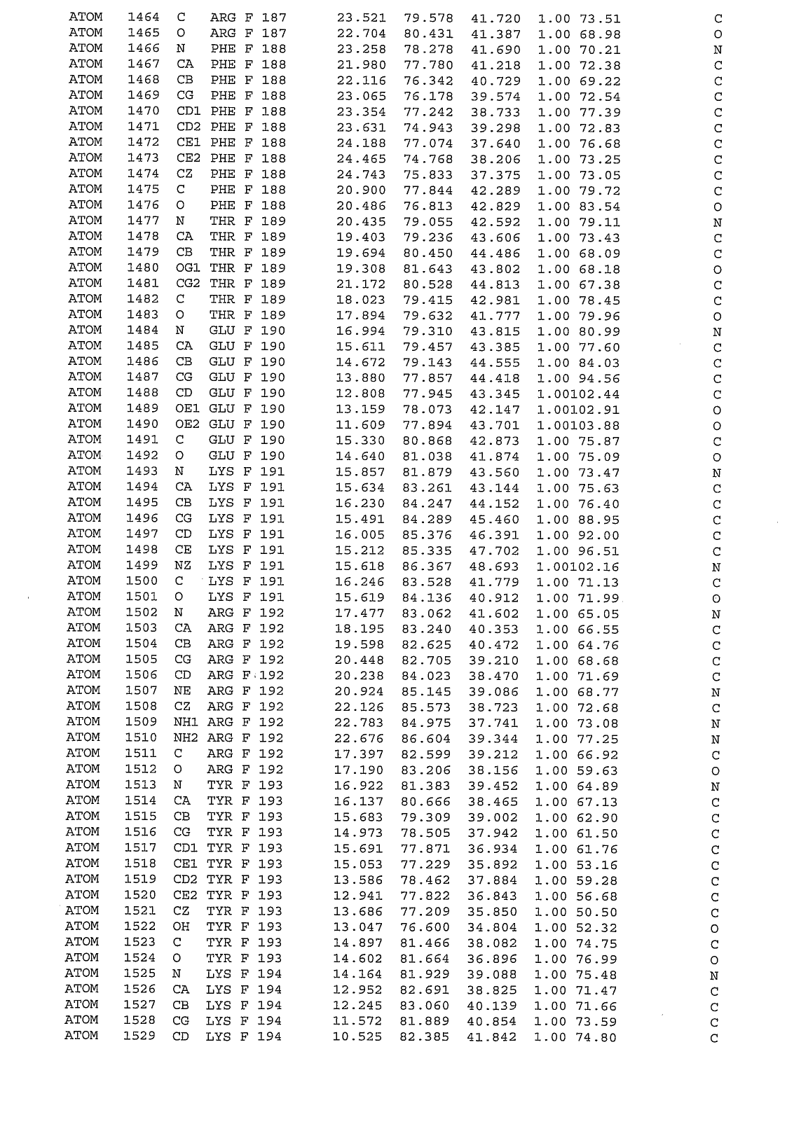

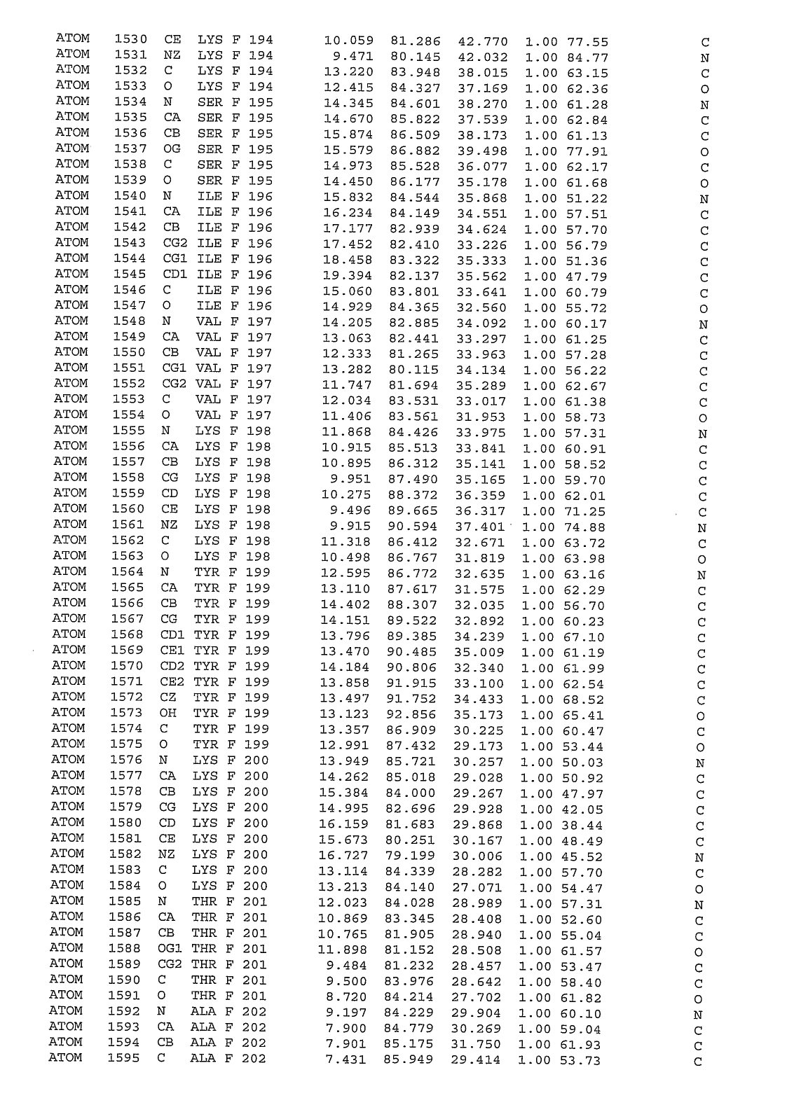

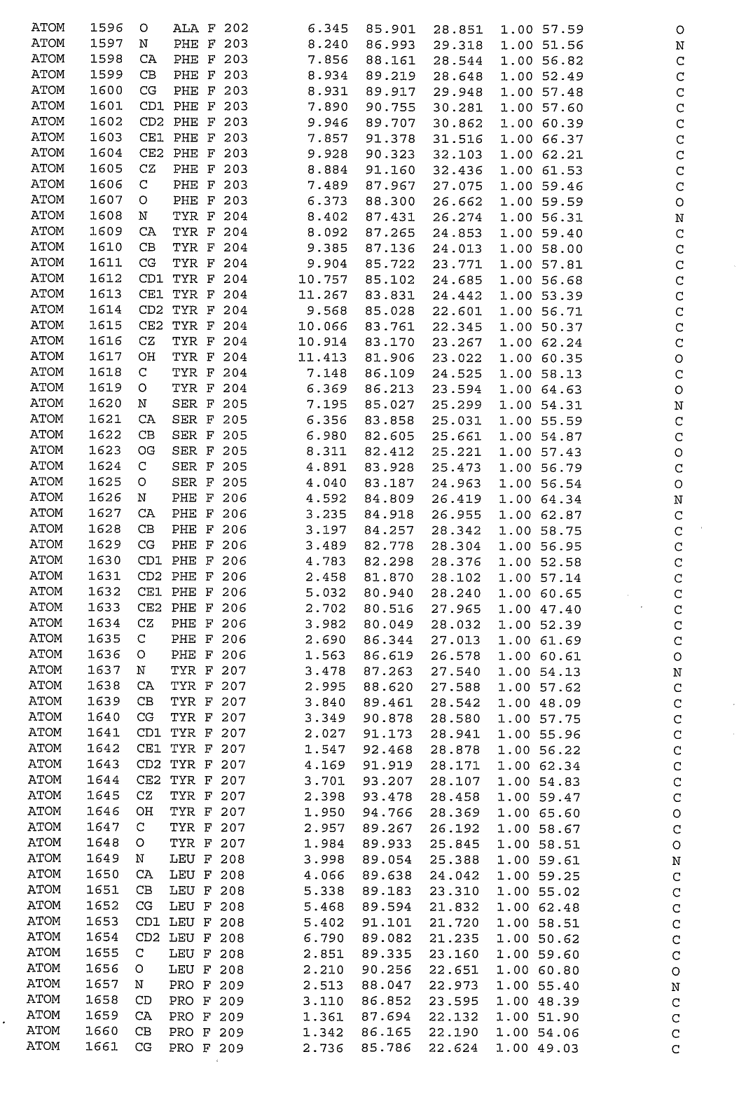

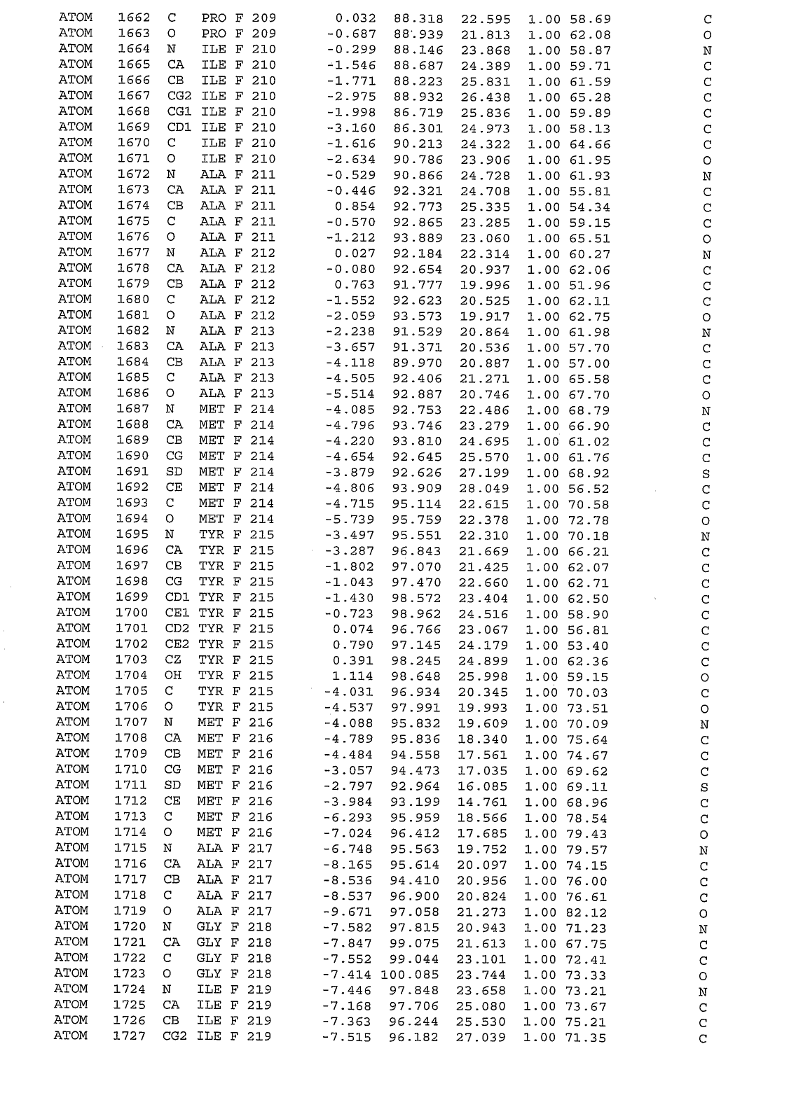

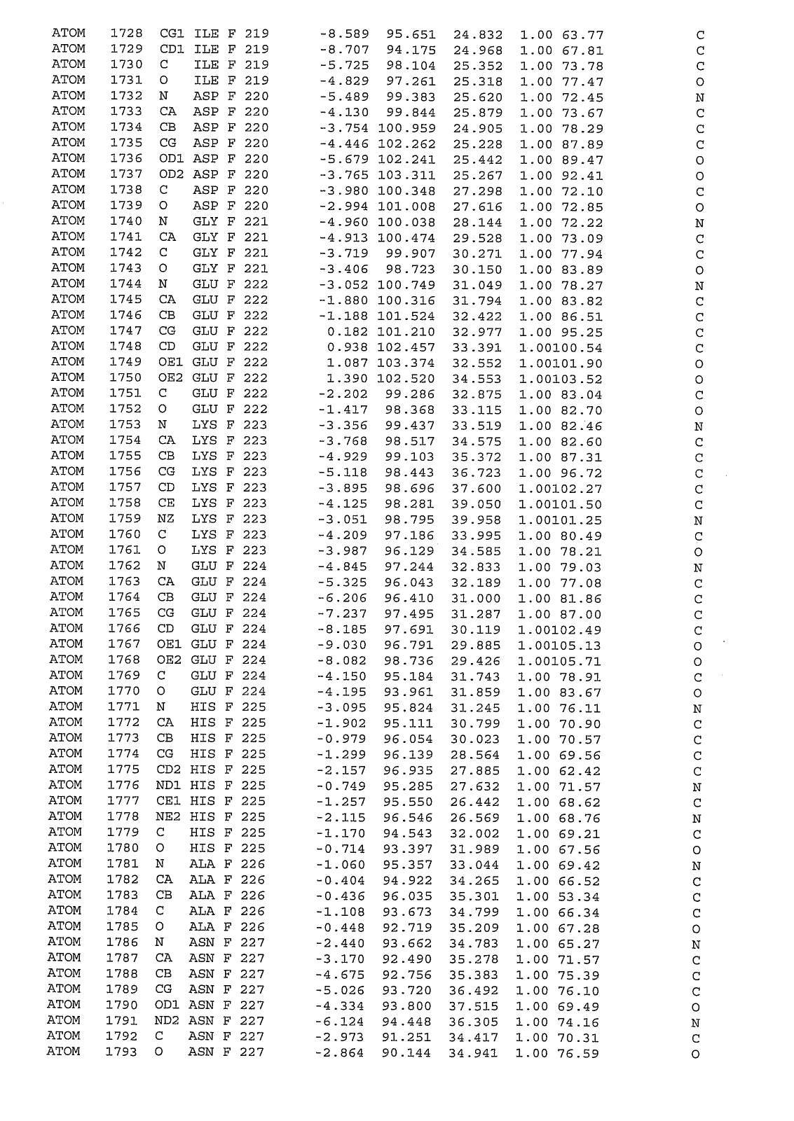

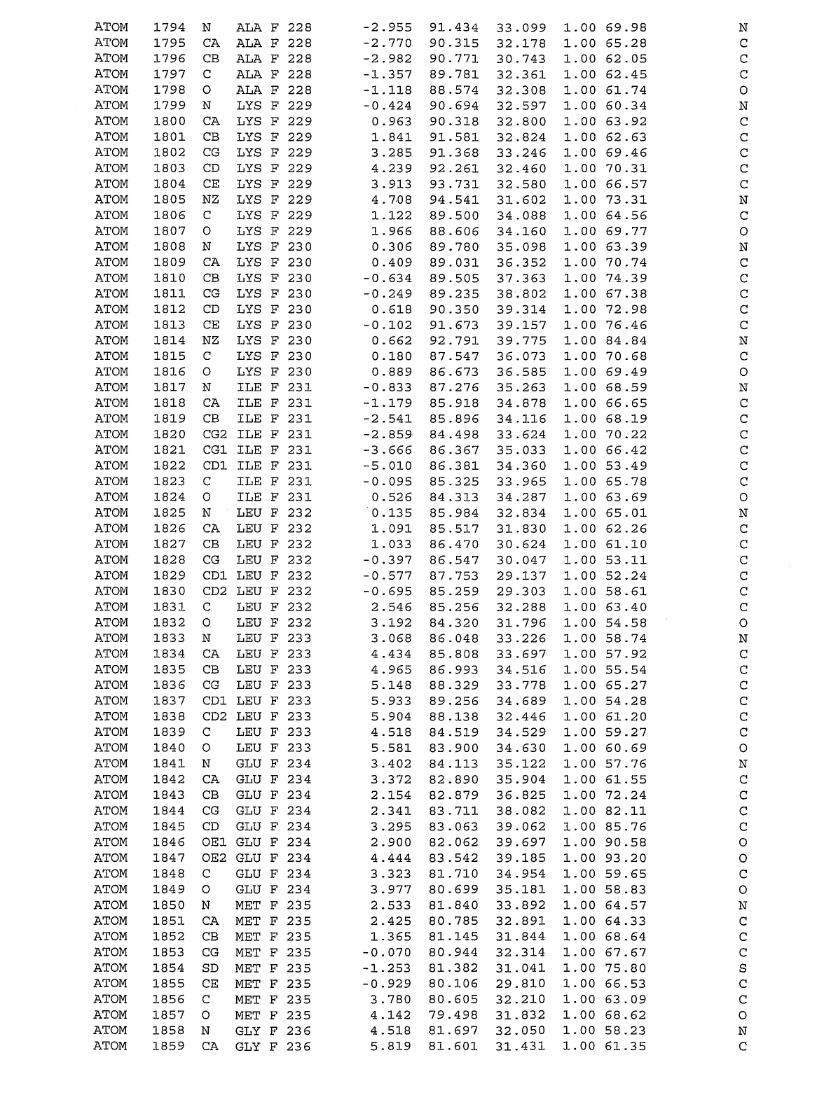

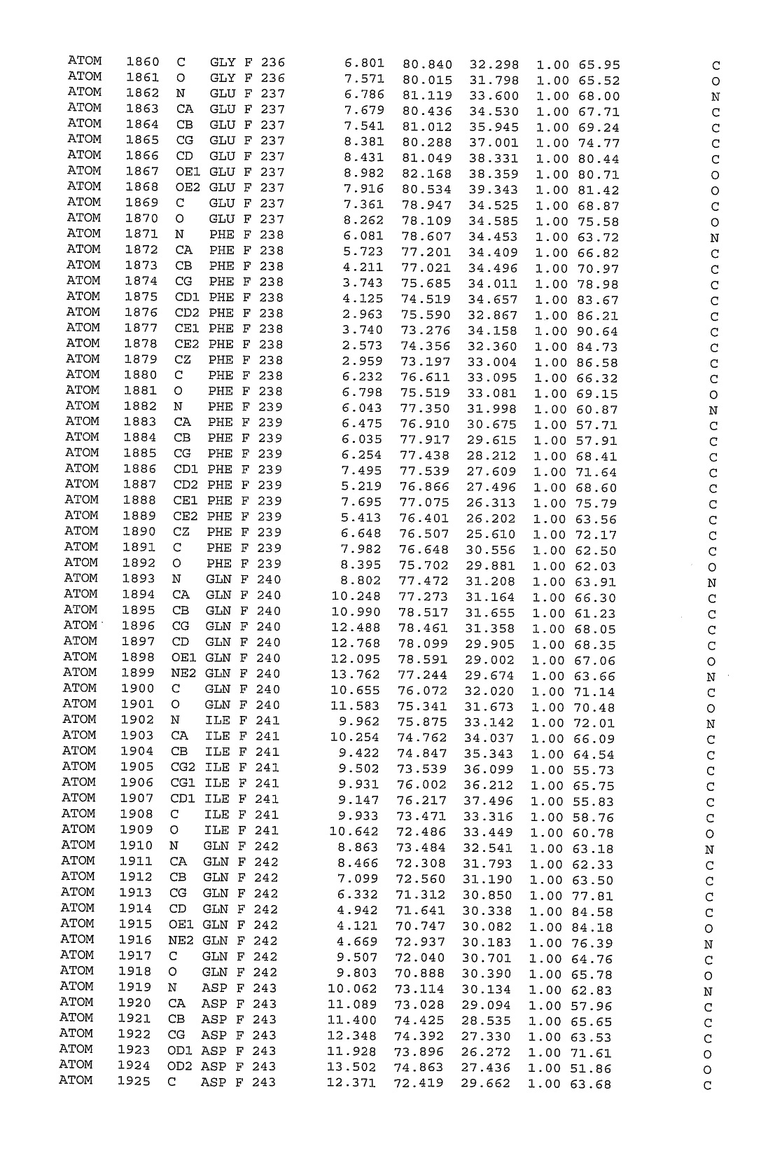

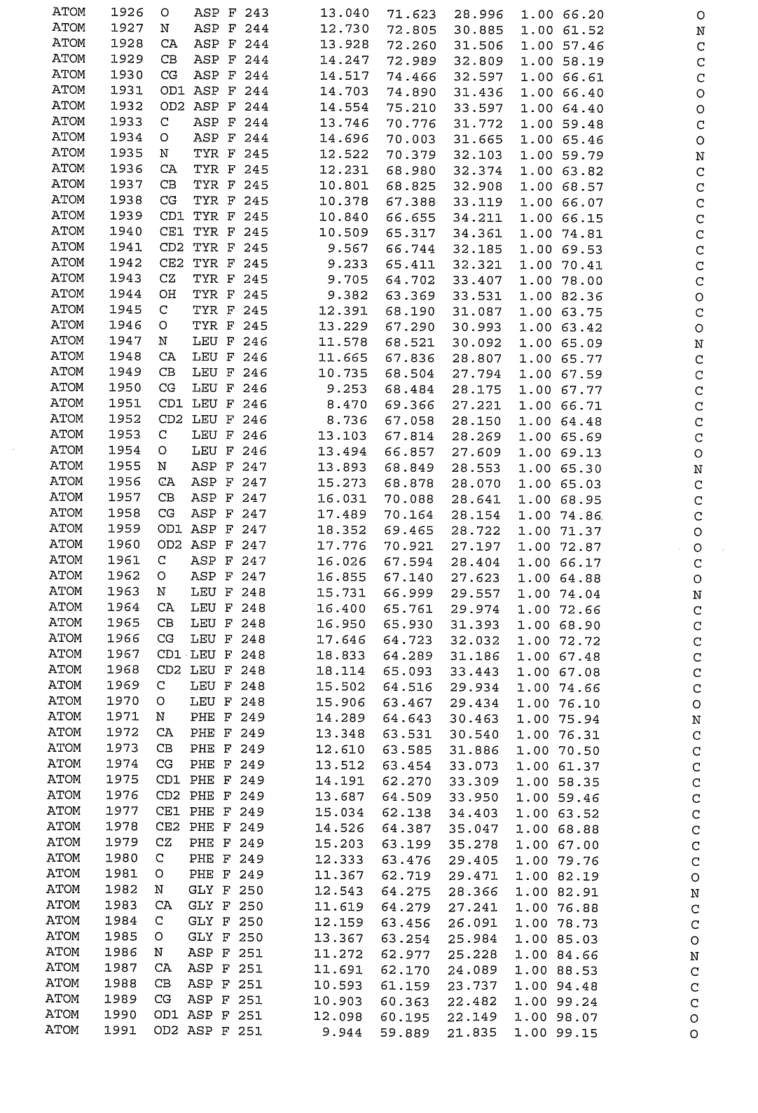

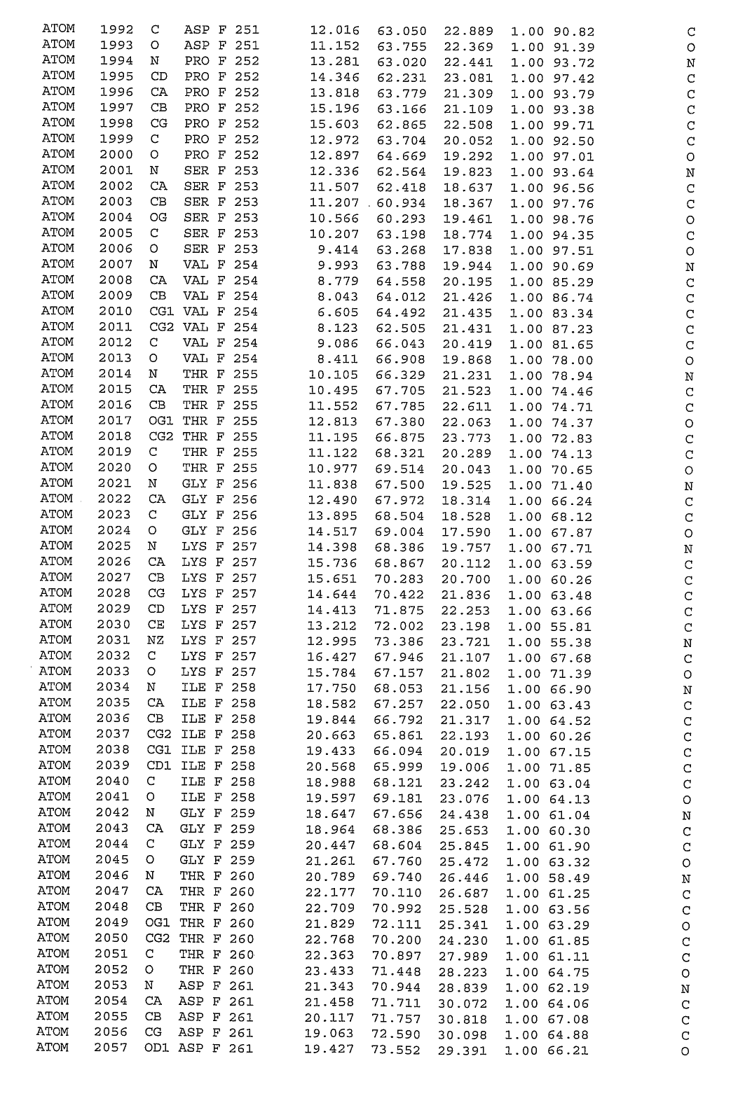

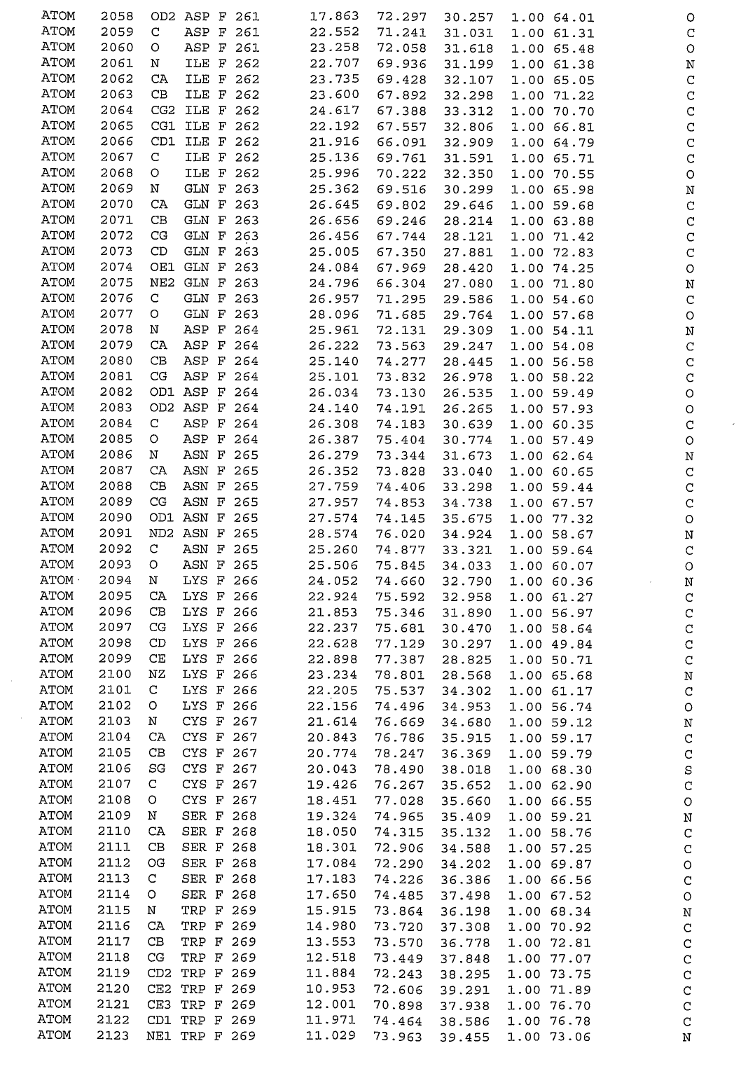

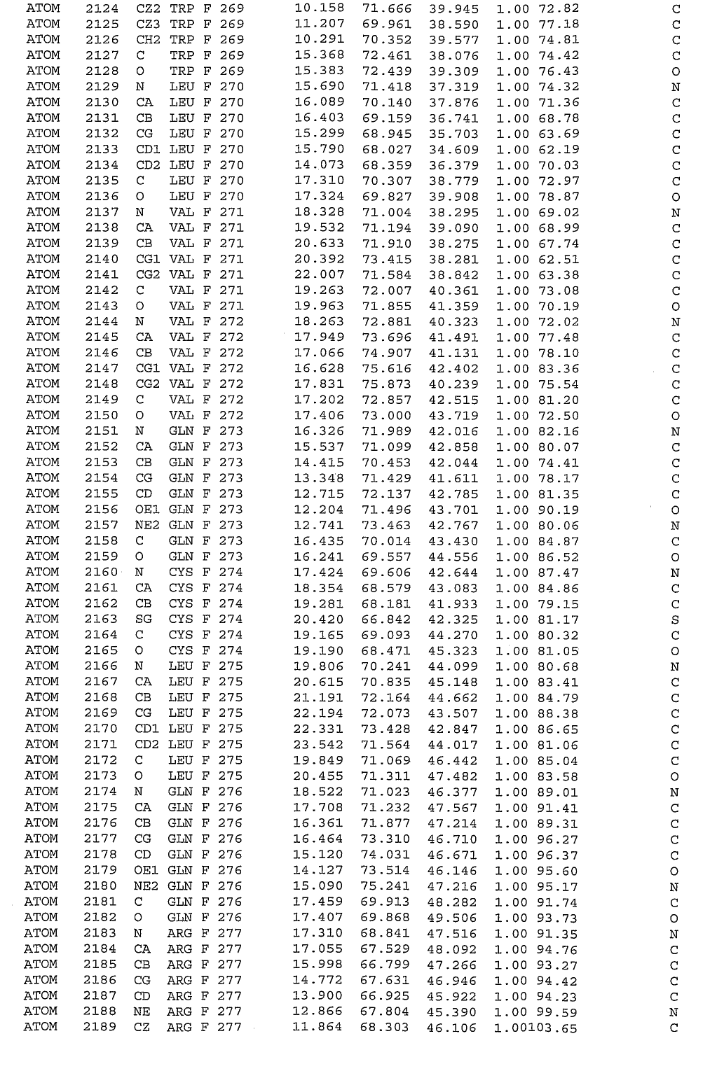

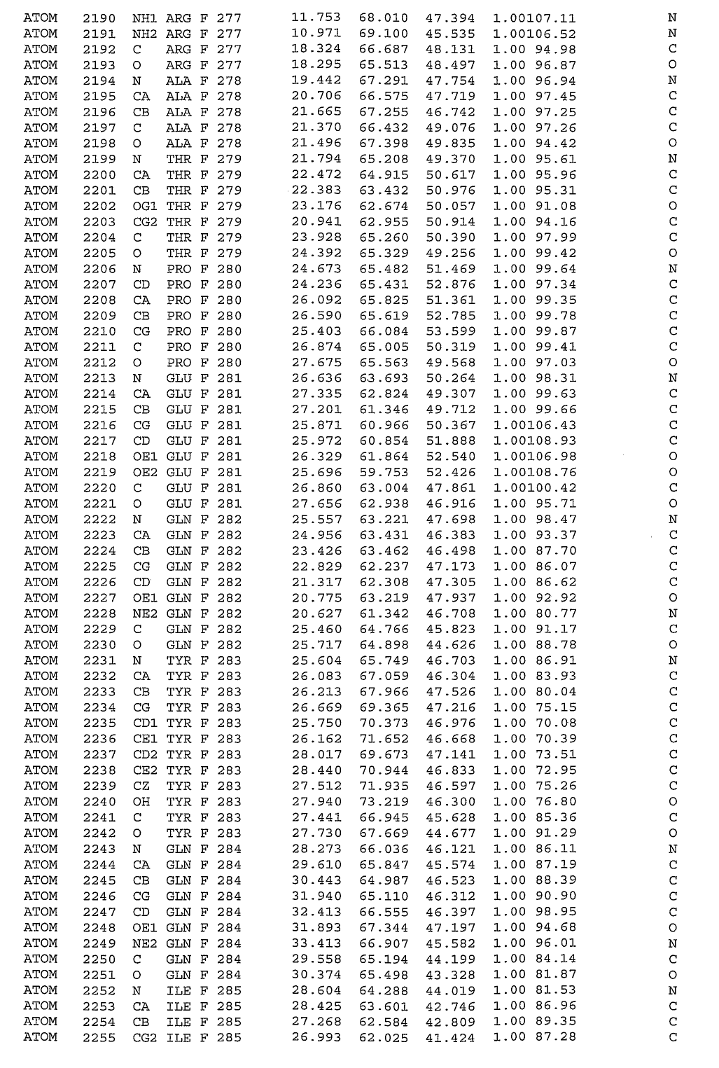

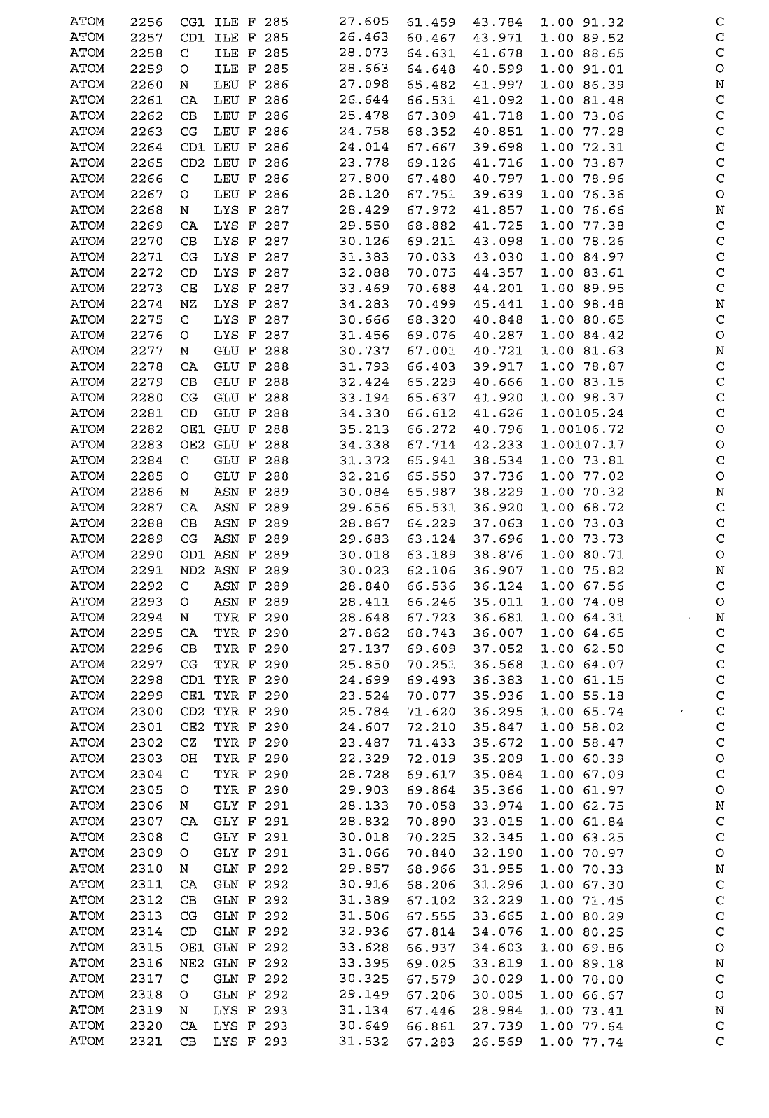

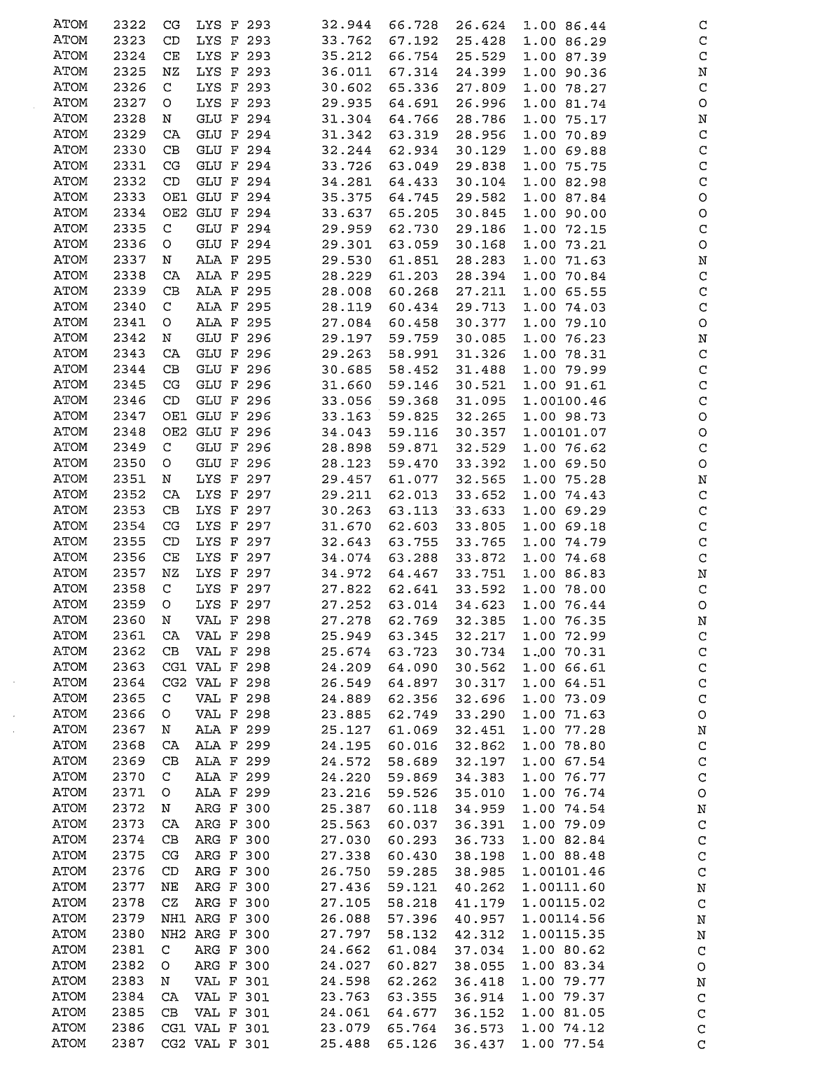

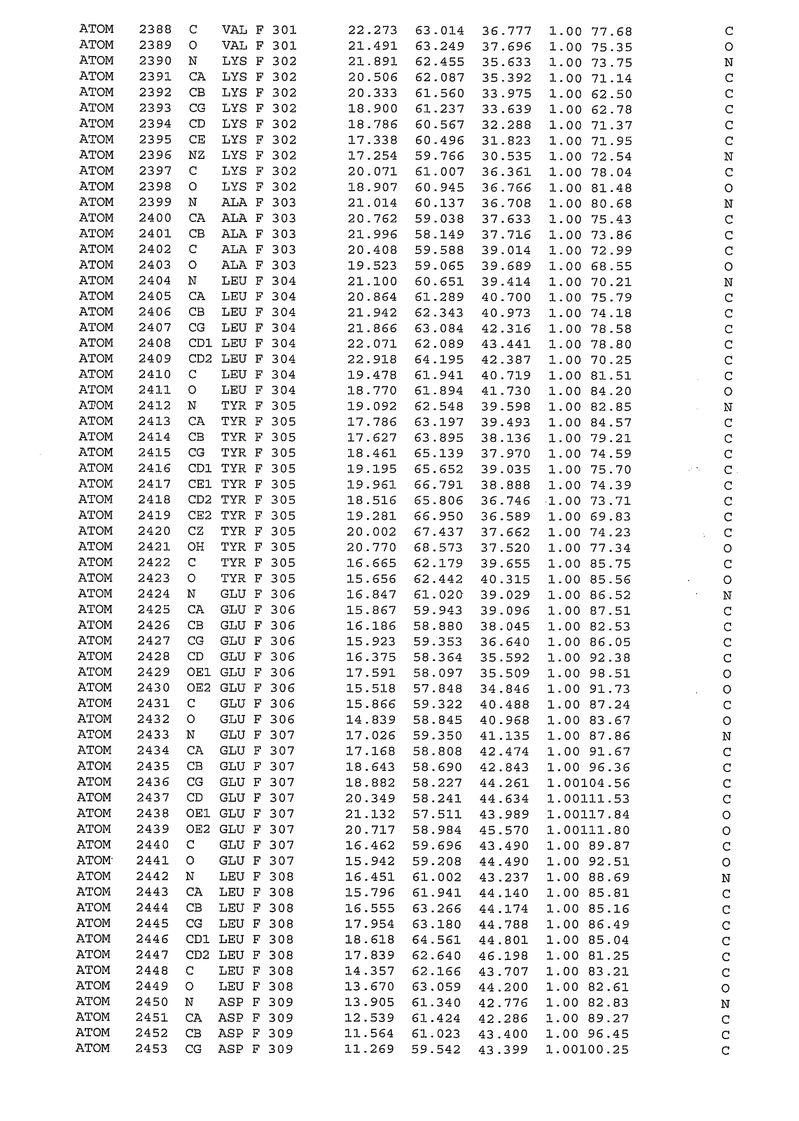

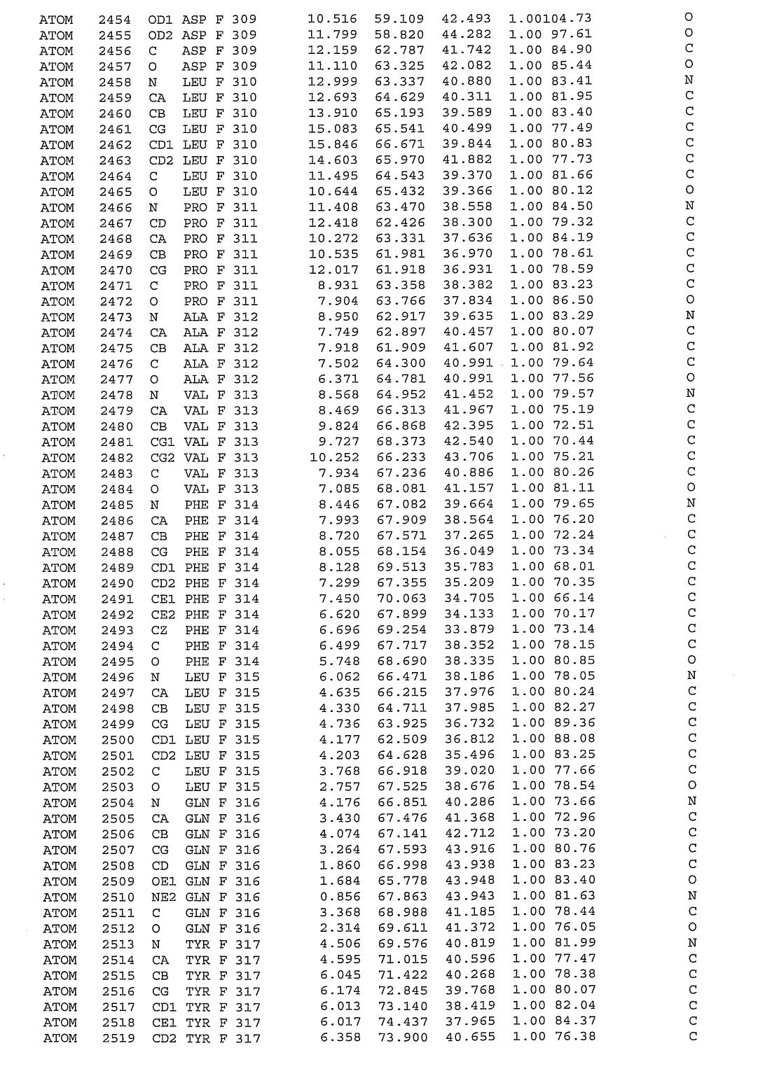

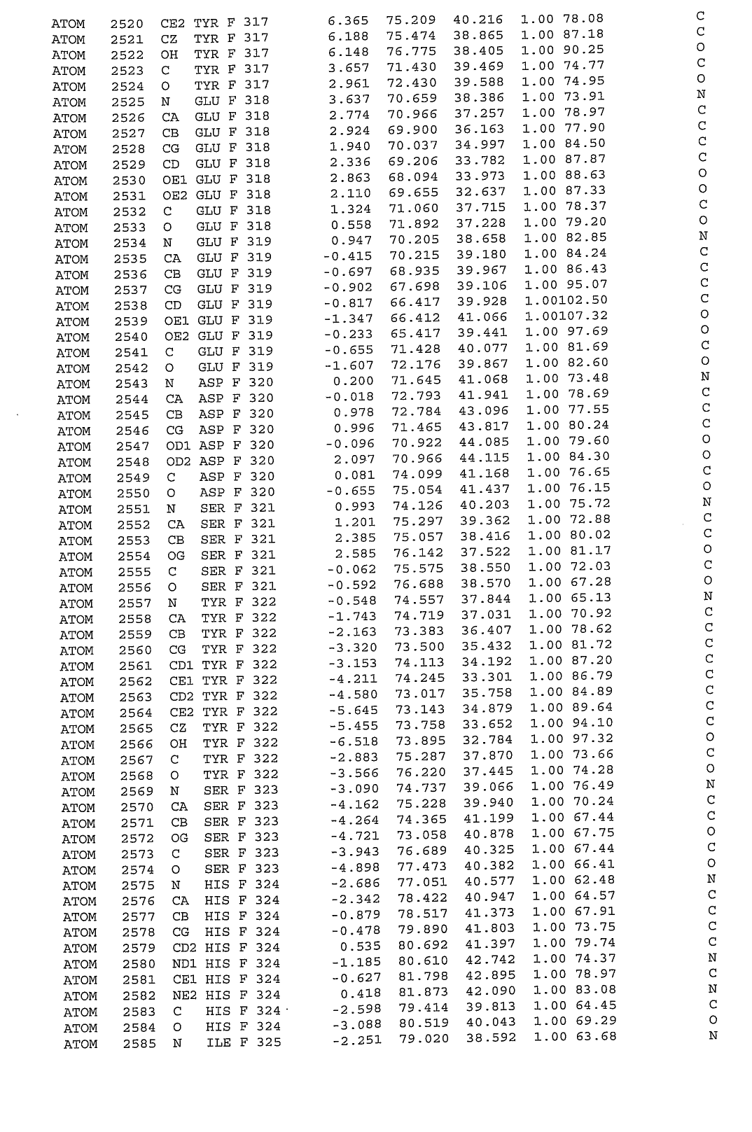

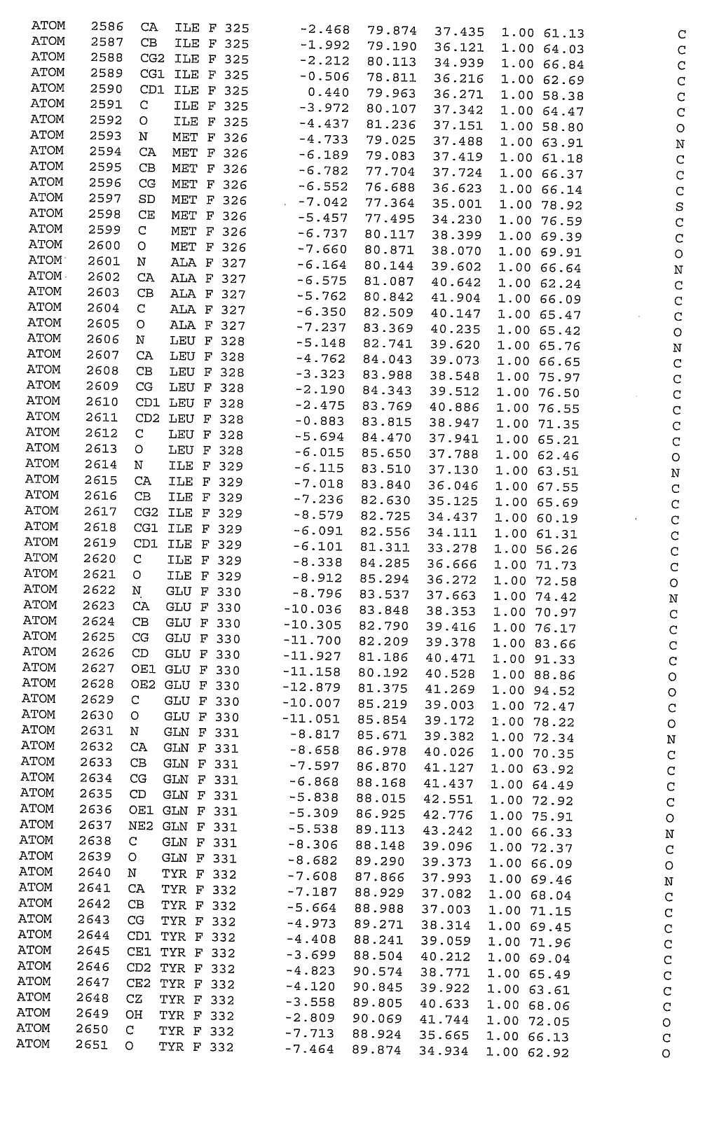

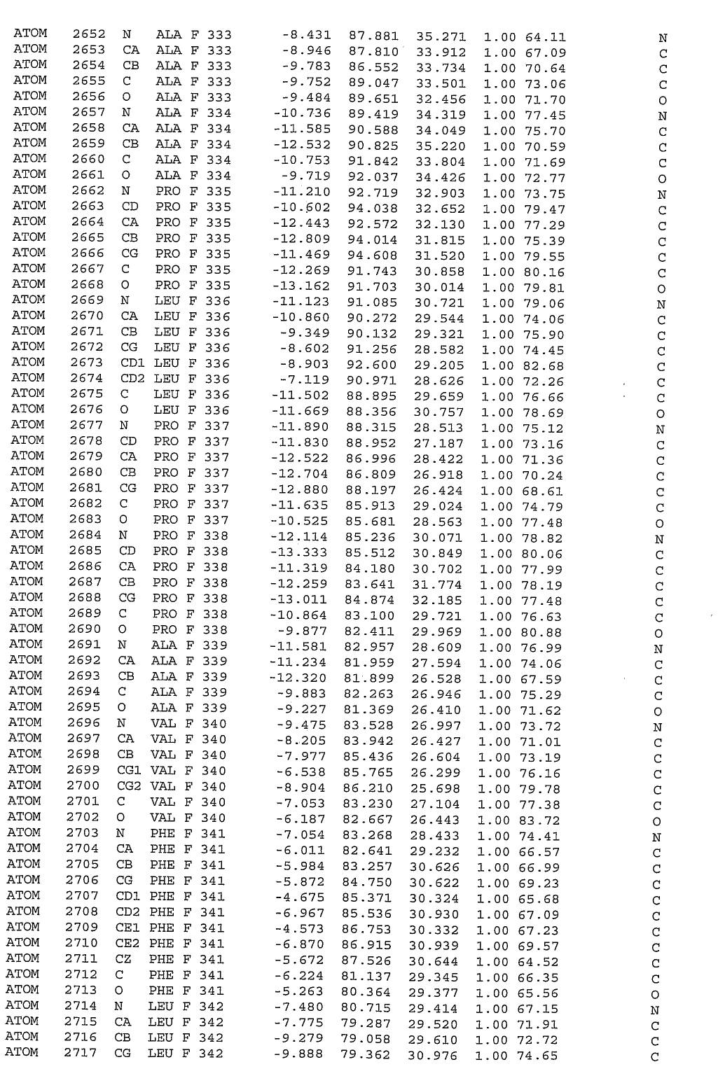

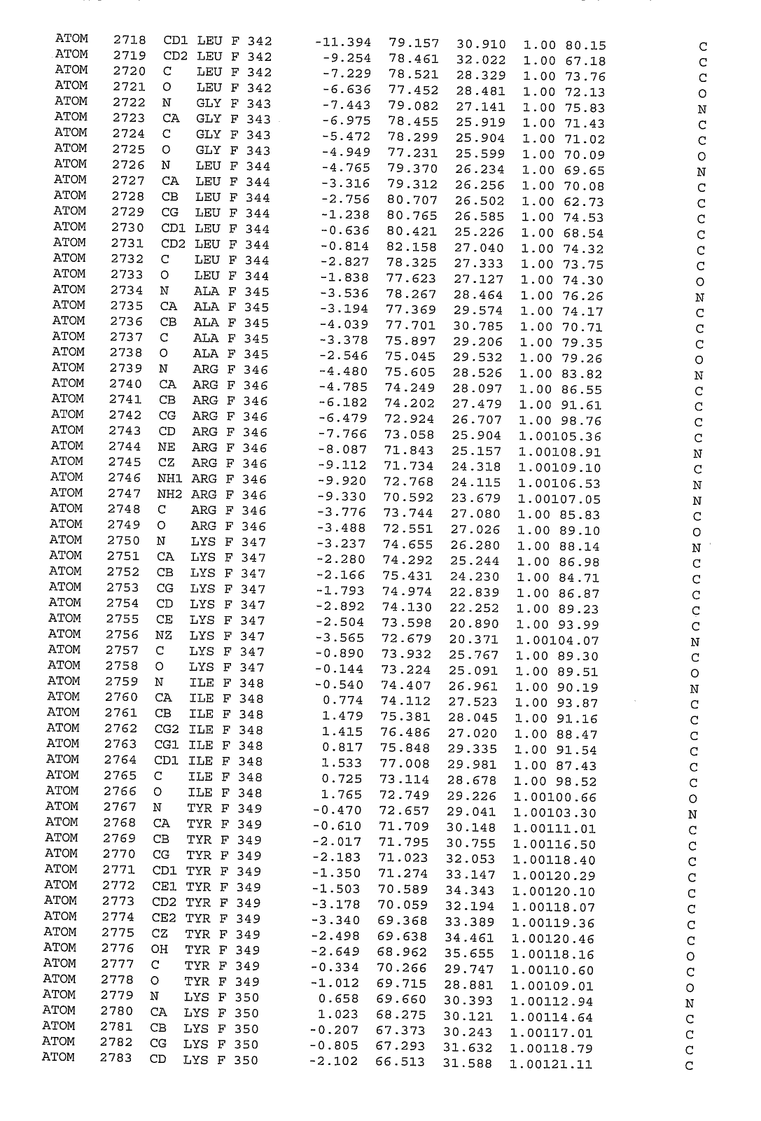

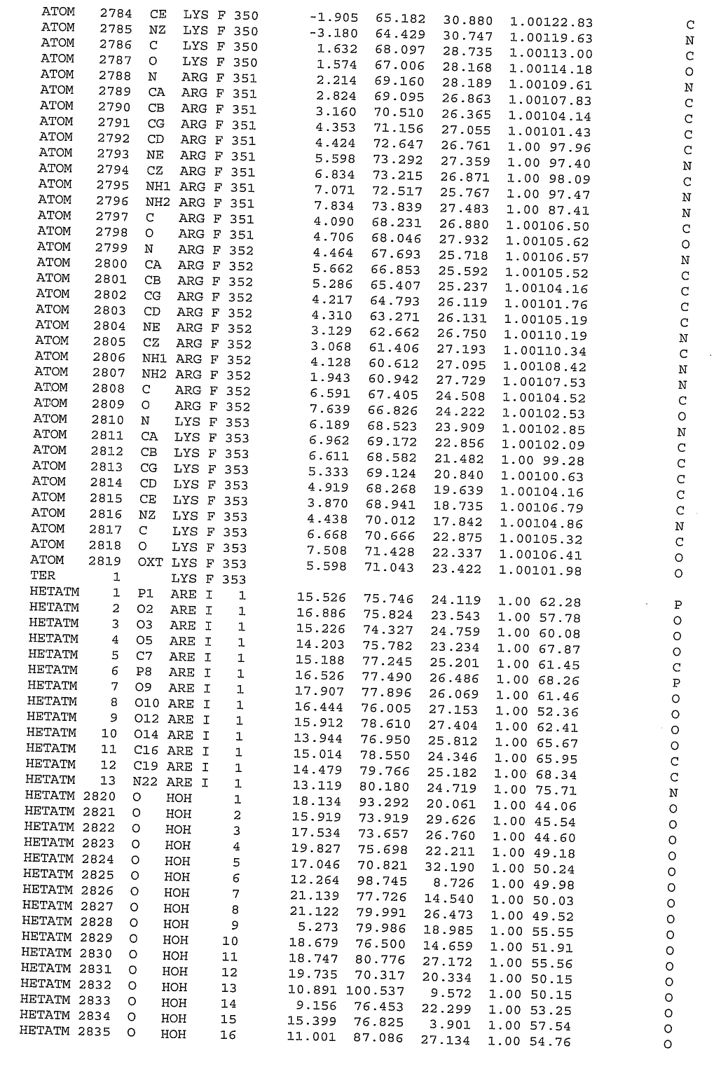

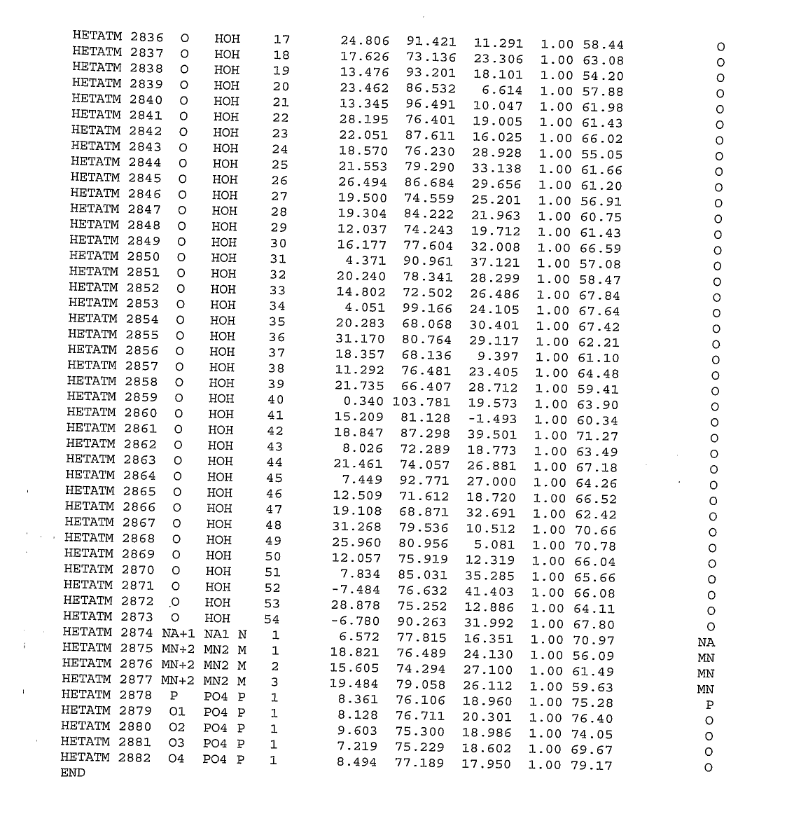

A complete diffraction data set of good quality (R_{merge}=0.072) was collected to 2.6 Å for the ternary complex of hFPPS with Mg²⁺, IPP and zoledronate. Good difference density was observed for both the substrate and the inhibitor (FIG. 15). The data fully confirmed the assignment of the IPP binding site to the pocket previously occupied by the endogeneous ligand, and revealed the details of the IPP binding interactions. Several conserved basic residues (Lys57, Arg60, and Arg113) make direct interactions to the IPP substrate, and three others (Arg112, Lys257, and Arg351) have their positively-charged group within 5.0 Å of the diphosphate unit of IPP. Worth of note, several of these residues (Phe239, Gln240, Lys257 and Arg351) are part of the secondary structure elements (H helix, H-I loop and C-terminal tail) which are affected by the conformational switch of FPPS. Therefore, IPP binding contributes to the stabilization of the closed form of the enzyme. The hydrocarbon moiety of IPP binds between the conserved Phe239 and the imidazole ring of zoledronate (FIG. 16). Binding of the zoledronate/Mg²⁺ trinuclear cluster is unchanged in the IPP ternary complex in comparison to the previous structure with the above-described ligand. Since the substituted nitrogen atom of the imidazolium ring mimics the allylic carbocation of the transition state, the observed binding of IPP is consistent with the established stereochemistry of the FPPS condensation reaction: the si-face of the IPP double bond is poised for the condensation reaction with the C1′ carbon atom of the allylic substrate. The observed distance between the C4 atom of IPP and the substituted nitrogen of the imidazolium ring of zoledronate is 3.8 Å.

### 14. Comparison to Avian FPPS

A comparison of crystallographic data of human and avian FPPS shows that human and avian FPPS share 69% sequence identity. As expected, both enzymes show the same three-dimensional fold. Also, the dimer interface and relative orientation of the subunits is conserved. However, structural comparisons reveal that avian FPPS was observed in the open conformation only. The open form of human FPPS can be superimposed on the published avian structures with an rms deviation of about 0.95 Å for 334 structurally equivalent Cα atoms. The conformation of the H-I loop of avian FPPS differs from that observed in the human enzyme. Also, the carboxy-terminal residues of avian FPPS adopt a different conformation, pointing away from the enzyme active site.

Crystallographic work with avian FPPS in complex with allylic substrates has revealed the presence of only 2 magnesium binding sites and showed that the diphosphate moiety was interacting with only the first DDXXD motif located on helix D (Tarshis, Proc. Natl. Acad. Sci USA, 93 (1996) 15018-15023). Based on these results, it was proposed that isopentenyl diphosphate would bind to the second DDXXD motif located at the C-terminal end of helix H. In sharp contrast, our results show that pamidronate and zoledronate bind to both DDXXD motifs, together with three divalent cations, and that IPP binds in a basic site lined by the B-C, D-E and H-J loops.

The X-ray analyses of human FPPS presented here provide for the first time the three-dimensional structure of this important drug target, both in the open and in the closed conformation, and reveal the binding sites of substrates of FPPS such as IPP as well as of inhibitors of FPPS such as pamidronate and zoledronate, two important marketed drugs of the nitrogen-containing bisphosphonate class. The new data clarify and correct previous notions regarding IPP and bisphosphonate binding, as well as the number and location of the metal centers.

The conformation switch of FPPS involves a rigid-body movement of the last 130 carboxy-terminal residues, and a shift of the H-I loop and of the last three carboxy-terminal residues with a concomitant transition from a dynamic (disordered) conformational state to an ordered, well-defined conformation in the closed form. Such a conformational switch underpins the ordered reaction mechanism, since the IPP binding site is not formed until the first substrate (DMAPP or GPP) has bound. Also, the carbonium generated during the enzymatic reaction is protected from solvent within the closed enzymatic active site. Conserved basic residues involved in substrate binding are found in the mobile loops.

Based on avian FPPS crystal structures, IPP binding was thought to involve the N-term DDXXD motif, which was not consistent with the observation that binding was not Mg dependent. The structures presented here show that the diphosphate group of IPP does not interact with any of the two DDXXD motifs, explaining why IPP binding does not require Mg²⁺. Our data demonstrate that pamidronate and zoledronate act as allylic pyrophosphate analogues, which is fully in agreement with the observation that FPPS inhibition by alendronate, another nitrogen-containing bisphosphonate compound, is competitive with respect to allylic substrates but not IPP. Furthermore, the three-dimensional structure of the FPPS complex with zoledronate explains well the available structure-activity data.

TABLE 1
X-ray data collection statistics

| | Apo | Apo | pamidronate/ | Zoledronate/ | IPP/zoledronate/ |
|---|---|---|---|---|---|
| Data set | Open form | Closed form | Mn²⁺ | Mg²⁺ | Mg²⁺ |
| Synchrotron/Beamline | SLS/XS06A | SLS/XS06A | SLS/XS06A | SLS/XS06A | SLS/XS06A |
| Wavelength | 0.97933 Å | 0.97935 Å | 1.00003 Å | 1.00003 Å | 1.00033 Å |
| Detector type | MARCCD | MARCCD | MARCCD | MARCCD | MARCCD |
| Number of crystals | 1 | 1 | 1 | 1 | 1 |
| Space group | P4₁2₁2 | P4₁2₁2 | P4₁2₁2 | P4₁2₁2 | P4₁2₁2 |
| Unit cell dimensions | a = b = 110.89 Å | a = b = 111.31 Å | a = b = 111.57 Å | a = b = 111.84 Å | a = b = 112.16 Å |
| | c = 77.00 Å | c = 66.88 Å | c = 66.48 Å | c = 66.04 Å | c = 65.72 Å |
| Nb of monomers/a.u. | 1 | 1 | 1 | 1 | 1 |
| Packing coefficient | 2.95 Å³/Da | 2.58 Å³/Da | 2.58 Å³/Da | 2.57 Å³/Da | 2.57 Å³/Da |
| Solvent content | 58% | 52% | 52% | 52% | 52% |
| Resolution range | 100.0-2.30 Å | 100.0-2.40 Å | 100.0-2.60 Å | 100.0-2.20 Å | 100.0-2.60 Å |
| Nb of observations | 311,259 | 175,022 | 181,286 | 311,908 | 101,738 |
| Nb of rejected observations | 17 (0.005%) | 1541 (0.88%) | 1169 (0.64%) | 1792 (0.57%) | 732 (0.72%) |
| Nb of unique reflections | 21,795 | 17,205 | 13,491 | 21,820 | 13,248 |
| Data processing program | XDS | HKL | HKL | HKL | HKL |
| Overall | | | | | |
| Data redundancy | 14.3 | 10.2 | 13.4 | 14.3 | 7.7 |
| Data completeness | 99.5% | 99.9% | 99.9% | 99.8% | 99.9% |
| <I/σ (I)> | 23.3 | 10.0 | 8.0 | 10.8 | 8.8 |
| R_{merge} | 0.072 | 0.060 | 0.070 | 0.055 | 0.072 |
| Highest resolution shell | | | | | |
| Resolution range | 2.37-2.30 Å | 2.49-2.40 Å | 2.69-2.60 Å | 2.28-2.20 Å | 2.69-2.60 Å |
| Completeness for shell | 99.9% | 99.9% | 99.8% | 100.0% | 100.0% |
| R_{merge} for shell | 0.325 | 0.468 | 0.434 | 0.423 | 0.481 |
| Reflections with I ≧ σ(I) 3σ | 62.5% | 45.7% | 49.7% | 57.8% | 37.2% |

TABLE 1b
Data collection statistics: FPPS complex with Ibandronate:

| Synchrotron/Beamline | SLS/PX-II |
|---|---|
| Wavelength | 1.00003 Å |
| Detector type | MAR225 CCD |
| Number of crystals | 1 |
| Space group | P4₁2₁2 |
| Unit cell dimensions | a = b = 111.35 Å, c = 68.89 Å |
| | α = β = γ = 90° |
| Number of monomers/a.u. | 1 |
| Packing coefficient | 2.66 Å³/Da |
| Solvent content | 54% |
| Resolution range | 50.0-1.94 Å |
| Number of observations | 433,776 |
| Number of rejected observations | 192 (0.044%) |
| Number of unique reflections | 32,351 |
| Overall | |
| Data redundancy | 13.4 |
| Data completeness | 99.2% |
| <I/σ (I)> (XDS) | 24.3 |
| R_{merge} | 0.066 |
| Highest resolution shell | |
| Resolution range | 2.00-1.94 Å |
| Completeness for shell | 96.3% |
| <I/σ (I)> for shell (XDS) | 6.72 |
| R_{merge} for shell | 0.422 |
| Reflections with I ≧ 3σ(I) | 45.1% |

TABLE 2
Refinement statistics

| | Apo | Apo | pamidronate/ | Zoledronate/ | IPP/zoledronate/ |
|---|---|---|---|---|---|
| Structure | open form | closed form | Mn²⁺ | Mg²⁺ | Mg²⁺ |
| Data used in refinement | | | | | |
| resolution range | 41.7-2.30 Å | 57.3-2.40 Å | 57.1-2.60 Å | 56.9-2.20 Å | 56.7-2.61 Å |
| intensity cutoff (σ(F)) | 0.0 | 0.0 | 0.0 | 0.0 | 0.0 |
| number of reflections | 21,795 | 16,965 | 13,389 | 21,775 | 13,212 |
| completeness (incl. free set) | 99.4% | 99.9% | 99.9% | 99.7% | 99.8% |
| Fit to data used in refinement | | | | | |
| overall Rcryst | 0.230 | 0.241 | 0.198 | 0.216 | 0.219 |
| overall Rfree | 0.260 | 0.295 | 0.262 | 0.263 | 0.272 |
| Fit in the highest resolution bin | | | | | |
| resolution range | 2.44-2.30 Å | 2.55-2.40 Å | 2.76-2.60 Å | 2.34-2.20 Å | 2.76-2.61 Å |
| bin completeness (incl. free set) | 99.9% | 100.0% | 99.9% | 100.0% | 92.0% |
| bin Rcryst | 0.288 | 0.483 | 0.332 | 0.273 | 0.446 |
| bin Rfree | 0.348 | 0.486 | 0.381 | 0.333 | 0.496 |
| Number of non-hydrogen atoms | | | | | |
| protein atoms | 2,758 | 2,806 | 2,806 | 2,775 | 2,806 |
| inhibitor atoms | — | — | 13 | 16 | 16 |
| substrate atoms | — | — | — | — | 14 |
| waters | 53 | 81 | 54 | 134 | 60 |
| metal ions | 1Na⁺ | 1Na⁺ | 3Mn²⁺, 1Na | 3Mg²⁺, 1Na | 3Mg²⁺, 1Na⁺ |
| phosphate atoms | 5 | 5 | 5 | 5 | — |
| Overall B value from Wilson plot | 49.4 Å² | 49.9 Å² | 67.9 Å² | 39.2 Å² | 62.5 Å² |
| Overall mean B value | 64.1 Å² | 63.0 Å² | 73.1 Å² | 56.6 Å² | 66.0 Å² |
| mean B value for protein | 64.3 Å² | 63.2 Å² | 73.4 Å² | 56.0 Å² | 66.5 Å² |
| mean B value for inhibitor | — | — | 63.8 Å² | 36.6 Å² | 53.6 Å² |
| mean B value for substrate | — | — | — | — | 44.2 Å² |
| mean B value for Mg^{2+/}Mn²⁺ | — | — | 59.1 Å² | 42.3 Å² | 46.3 Å² |
| mean B value for waters | 54.4 Å² | 56.7 Å² | 59.5 Å² | 51.7 Å² | 51.9 Å² |
| CV-estimated coordinate error | | | | | |
| from Luzzati plot | 0.38 Å | 0.49 Å | 0.43 Å | 0.36 Å | 0.48 Å |
| from σ_{A} | 0.36 Å | 0.73 Å | 0.55 Å | 0.32 Å | 0.73 Å |
| Rms deviations from ideal values | | | | | |
| bond lengths | 0.007 Å | 0.007 Å | 0.007 Å | 0.007 Å | 0.007 Å |
| bond angles | 1.1° | 1.1° | 1.2° | 1.2° | 1.3° |
| dihedral angles | 19.2° | 19.3° | 19.0° | 18.8° | 19.4° |
| improper angles | 0.74° | 0.78° | 0.76° | 0.79° | 0.77° |
| Ramachandran plot | | | | | |
| Residues in disallowed region | 0 | 0 | 0 | 0 | 0 |
| PROCHECK G-factor | 0.40 | 0.39 | 0.39 | 0.44 | 0.37 |

TABLE 2b
Refinement statistics of the FPPS complex with Ibandronate

| Data used in refinement | | Overall B value from Wilson plot | 25.9 Å² |
|---|---|---|---|
| resolution range | 40.36-1.94 Å | Overall mean B value | 39.2 Å² |
| intensity cutoff (Sigma(F)) | 0.0 | mean B value for protein (chain F) | 38.8 Å² |
| number of reflections | 32,351 | mean B value for ligand (chain L) | 31.4 Å² |
| completeness (working + test set) | 99.0% | mean B value for Zn²⁺ ions | 28.6 Å² |
| Fit to data used in refinement | | mean B value for phosphate ion | 39.2 Å² |
| overall Rcryst | 0.205 | mean B value for waters | 45.1 Å² |
| overall Rfree | 0.241 | Cross-validated estimated coordinate | |
| Fit in the highest resolution bin | | error (low res. cutoff: 5.0 Å) | |
| resolution range | 2.06-1.94 Å | from Luzzati plot | 0.28 Å |
| bin completeness (working + test set) | 98.0% | from σ_{A} | 0.45 Å |
| bin Rcryst | 0.363 | Rms deviations from ideal values | |
| bin Rfree | 0.387 | bond lengths | 0.007 Å |
| Number of non-hydrogen atoms | | bond angles | 1.0° |
| protein atoms | 2,766 | dihedral angles | 19.9° |
| ligand atoms (2 alternate confor.) | 2 × 19 | improper angles | 0.72° |
| Zn²⁺ ions | 3 | Residues in disallowed region of | 0 |
| phosphate ion | 1 | Ramachandran plot | |
| waters | 207 | | |

### 15. NMR Spectroscopy

The X-ray crystallographic results described above were complemented and corroborated by NMR spectroscopy. FPPS represents a challenge for NMR analysis due to its high molecular weight of 80 kDa. In fact, ¹⁵N,¹H-HSQC or TROSY spectra with ¹⁵N-labeled (non-deuterated) FPPS are essentially non-interpretable due to extremely broad lines. Upon deuteration, however, ¹⁵N,¹H-TROSY spectra are of reasonable quality and allow analysis of individual resonances.

A ¹⁵N,¹H-TROSY spectrum can be regarded as a fingerprint spectrum of a protein. The chemical shifts that result in the characteristic peak pattern are influenced by the protein conformation as well as by ligand binding. If a ligand binds without causing significant conformational changes in the protein, only a few resonances change chemical shifts, namely those in direct vicinity of the ligand. However, if ligand binding gives rise to major conformational changes, many protein resonances experience chemical shift changes, and the fingerprint TROSY spectrum appears significantly different.

Mapping of the binding site of a ligand is usually performed by recording NMR spectra with and without ligand, and identifying those resonances that are affected by ligand binding. This requires assignment of the protein resonances prior to the analysis. While resonance assignment is straightforward for small proteins, it is a challenge for FPPS. According to the invention selected resonances were assigned in an indirect manner, on the basis of their perturbations by known ligands with known binding sites, and by taking advantage of the paramagnetic relaxation enhancement caused by replacement of (diamagnetic) Mg²⁺ with (paramagnetic) Mn²⁺. This procedure allows to identify probes for the respective binding sites.

Upon addition of pamidronate and Mg²⁺, the ¹⁵N,¹H-TROSY spectrum changes significantly. In fact, almost all of the non-overlapping resonances experience chemical shift changes (FIG. 10). This strongly suggests major conformational changes taking place within FPPS upon binding of pamidronate. This corresponds to the transition between the open conformation of apo-FPPS and the closed conformation of pamidronate-bound FPPS that was observed by X-ray crystallography. Addition of Mg²⁺ alone without pamidronate causes only very small chemical shift changes in FPPS, indicating that binding of Mg²⁺ to FPPS in the absence of a bisphosphonate is weak and does not lead to significant conformational changes.

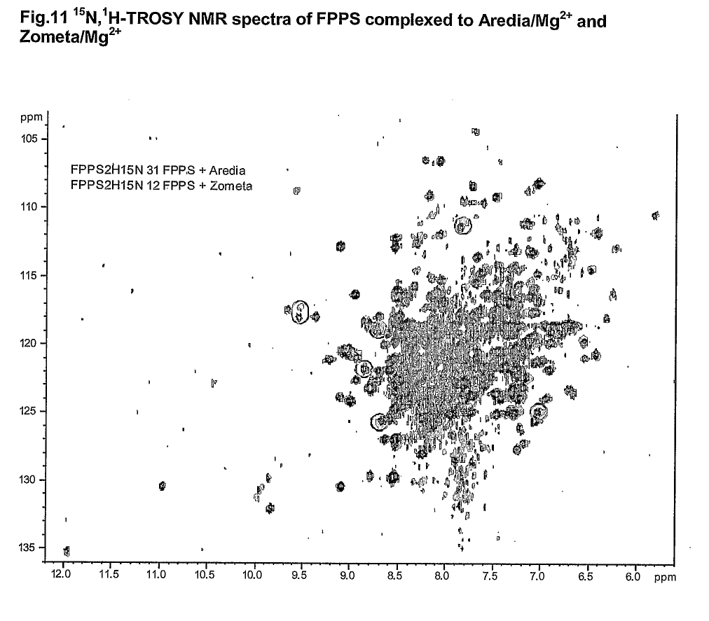

Zoledronate binds more tightly to FPPS than pamidronate does, and it should therefore displace pamidronate from the bisphosphonate binding pocket. An equimolar concentration of zoledronate was added to the sample of FPPS in complex with pamidronate/Mg²⁺. Additional, but far less, chemical shift changes were observed (FIG. 11). These chemical shift changes are due to displacement of pamidronate by zoledronate. Upon exchange of ligand, the FPPS conformation is not significantly altered since it is already in the closed state. The few chemical shift changes are thus directly attributed to local perturbations caused by the different bisphosphonate side chain: resonances which experience chemical shift changes upon pamidronate displacement by zoledronate are to be located in close vicinity to the zoledronate side chain. These shifting resonances, which are circled in FIG. 11, are thus indicators for the zoledronate side chain binding site.

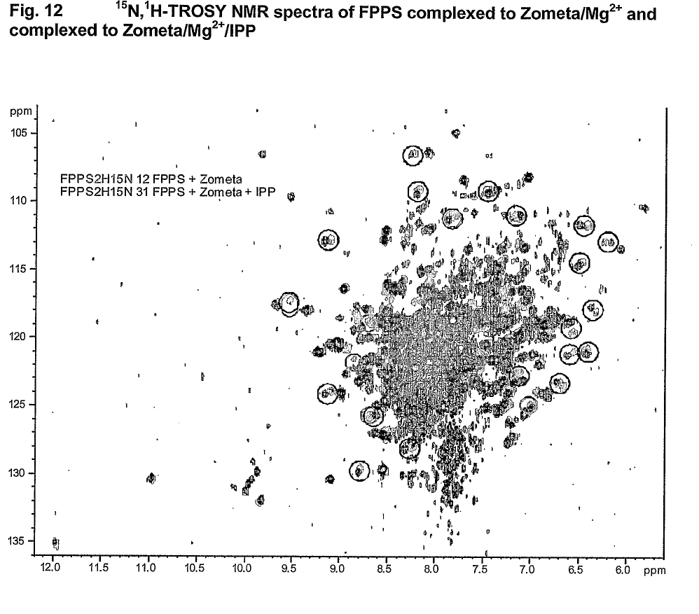

IPP is the FPPS substrate which binds outside the zoledronate binding site, and binds to FPPS even in the presence of zoledronate. Resonances near the IPP binding site were mapped by adding IPP to the FPPS/zoledronate/Mg²⁺ sample. Again, only some resonances changed chemical shift, consistent with the lack of major conformational changes in FPPS. Again, these resonances belong to residues near the IPP binding site, and can be used as indicators to probe binding in the IPP binding site (FIG. 12).

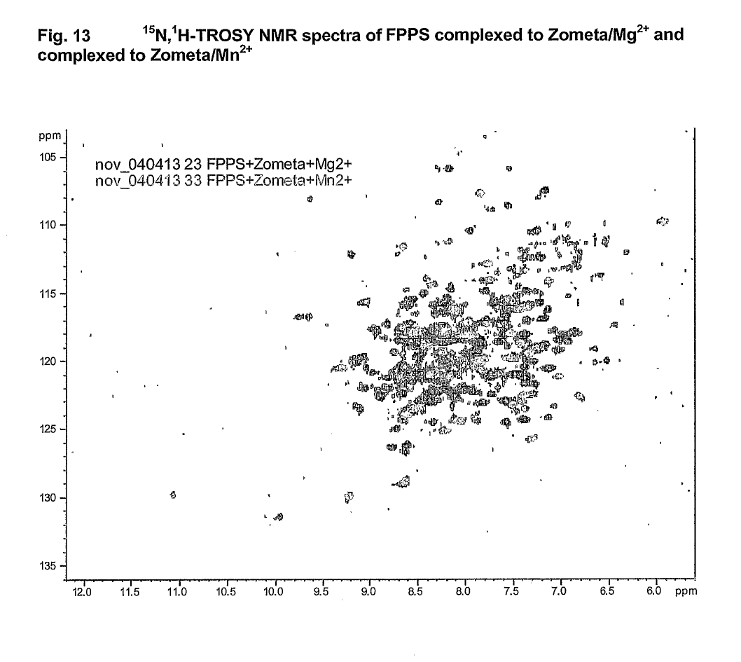

The distance of individual resonances from the Mg²⁺ binding site can be probed by replacing the (diamagnetic) Mg²⁺ by the (paramagnetic) Mn²⁺. Paramagnetic metals, like spin labels, exert distance-dependent relaxation enhancement effects to neighboring nuclei. Any residues unaffected by Mn²⁺ are therefore at least 2 nm away from the paramagnetic center, whereas residues that are strongly affected by Mn²⁺ are within 1.0-1.5 nm distance to the metal ions. FIG. 13 shows the ¹⁵N,¹H-TROSY NMR spectra of FPPS/zoledronate when ligated by Mg²⁺ or Mn²⁺.

The NMR studies confirmed that major conformational rearrangements occur after bisphosphonate binding to FPPS, corresponding to the change from “open” to “closed” conformation observed by X-ray crystallography.

Furthermore, resonances were identified which are characteristic of the zoledronate side chain binding site and of the IPP binding site. This is useful information for the discovery of non-bisphosphonate FPPS inhibitors by fragment-based ligand design, since it allows binding site mapping for fragments even when the X-ray structure cannot be solved.

We claim:

1. Crystalline human farnesyl diphosphate synthase (FPPS) and mutants thereof.

2. A crystalline human FPPS according to claim 1 in a closed conformation.

3. A crystalline human FPPS according to claim 2, comprising unit cell dimensions of a=b=112 Å±20 Å, c=66 Å±20 Å or, more preferably, a=b=112 Å±10 Å and c=66 Å±10 Å.

4. A crystalline human FPPS according to claim 1 in an open conformation.

5. A crystalline human FPPS according to claim 4, comprising unit cell dimensions of a=b=111 Å±20 Å, c=77 Å±20 Å or, more preferably, a=b=111 Å±10 Å and c=77 Å±10 Å.

6. Crystalline human FPPS according to claim 1 in complex with a ligand.

7. Crystalline human FPPS according to claim 6, wherein the ligand is selected from isopentenyl diphosphate (IPP) and/or from zoledronate or pamidronate.

8. Crystalline human FPPS according to claim 1. having one of the following three-dimensional structure:
a) a three-dimensional structure of the closed conformation defined by all or a selected portion of the structural coordinates shown in FIG. 14,
b) a three-dimensional structure of the open conformation defined by all or a selected portion of the structural coordinates shown in FIG. 15,
c) a three-dimensional structure defined by all or a selected portion of the structural coordinates shown in FIG. 16, 17 or 18, or
d) a structure similar to a), b) or c).

9. A method for producing a crystalline human FPPS preparation comprising the steps of:
(i) expressing recombinant human FPPS in E. coli, wherein said recombinant human FPPS corresponds to amino acids fragment 6 to 353,
(ii) purifying expressed human FPPS,
(iii) crystallizing the purified human FPPS.

10. A crystalline human FPPS, obtainable according to the method of claim 9.

11. Use of a crystalline human FPPS according to claim 1, for the generation of crystal structure data of human FPPS.

12. Use of a crystalline human FPPS complexed with a ligand according to claim 6, for the generation of crystal structure data of human FPPS ligand complexes.

13. Use according to claim 11 for determining the respective binding sites.

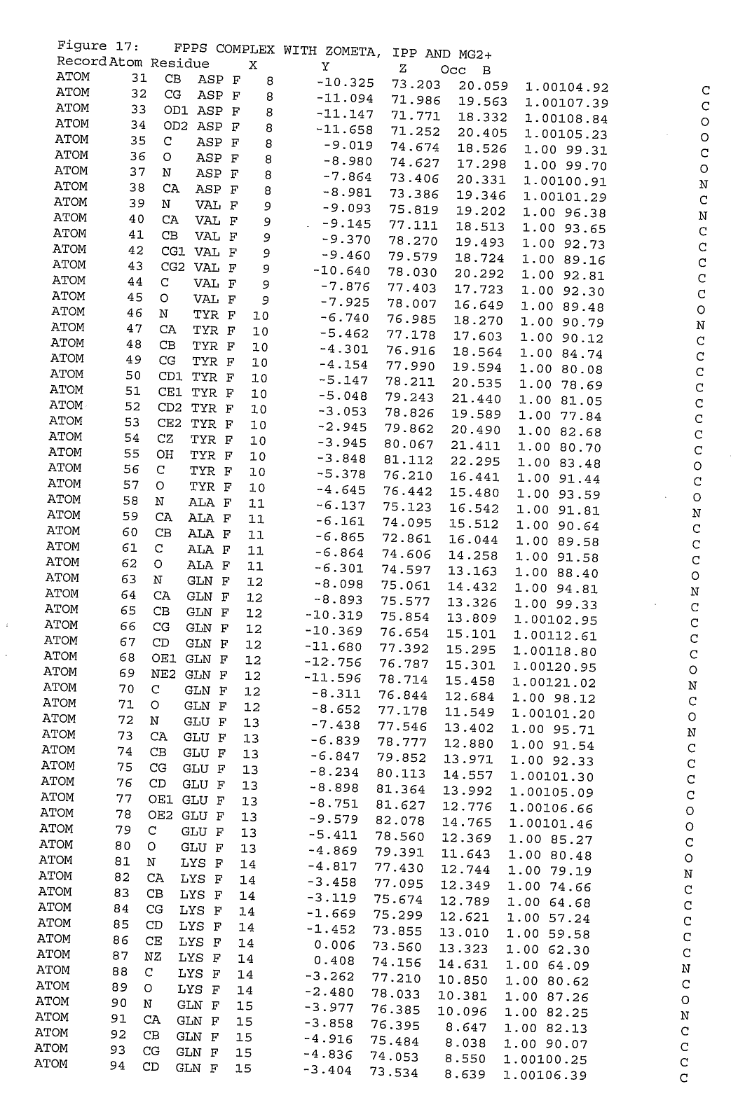

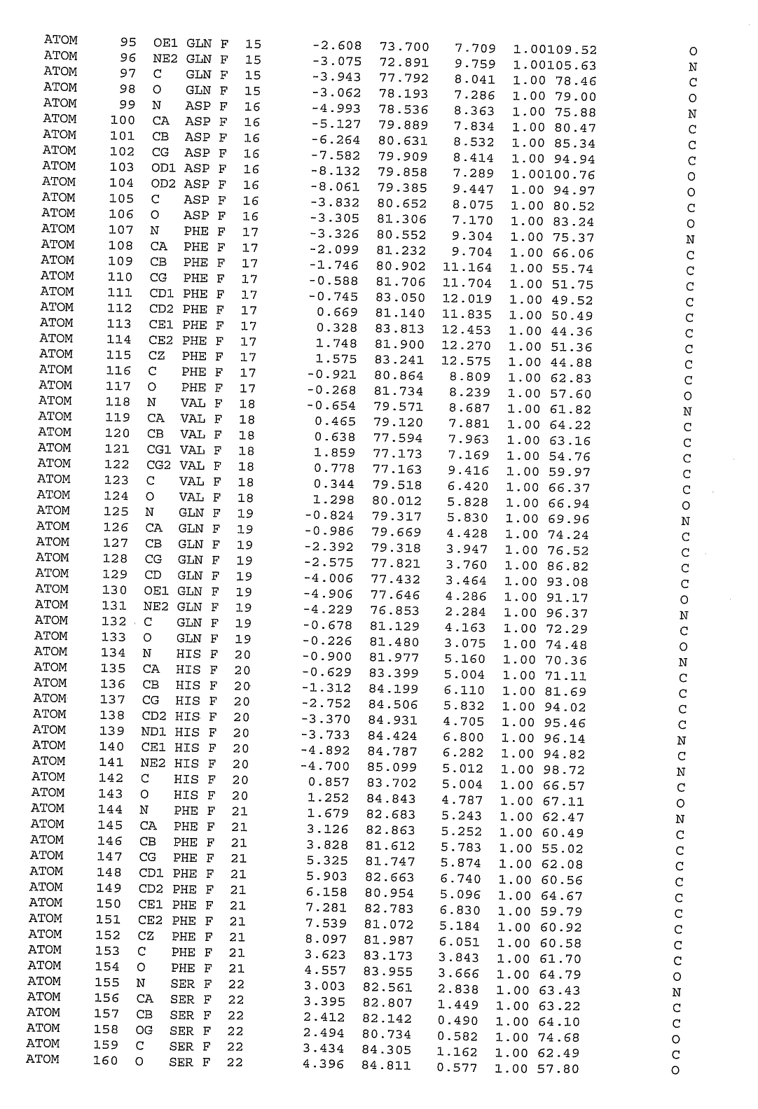

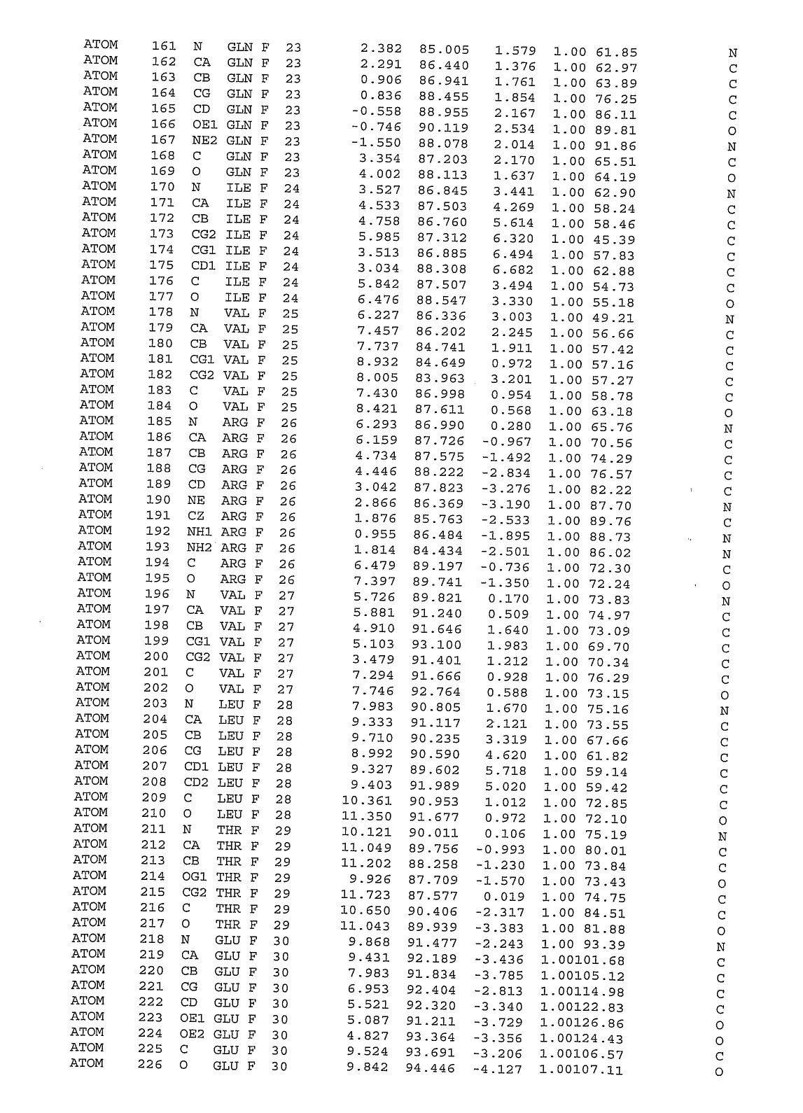

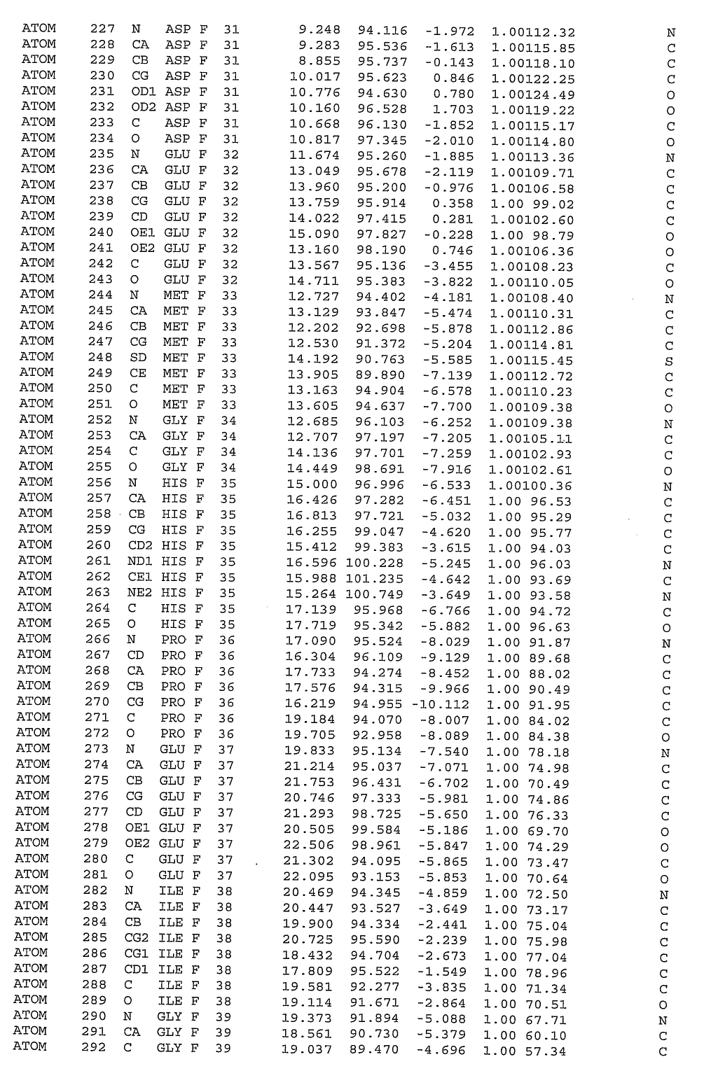

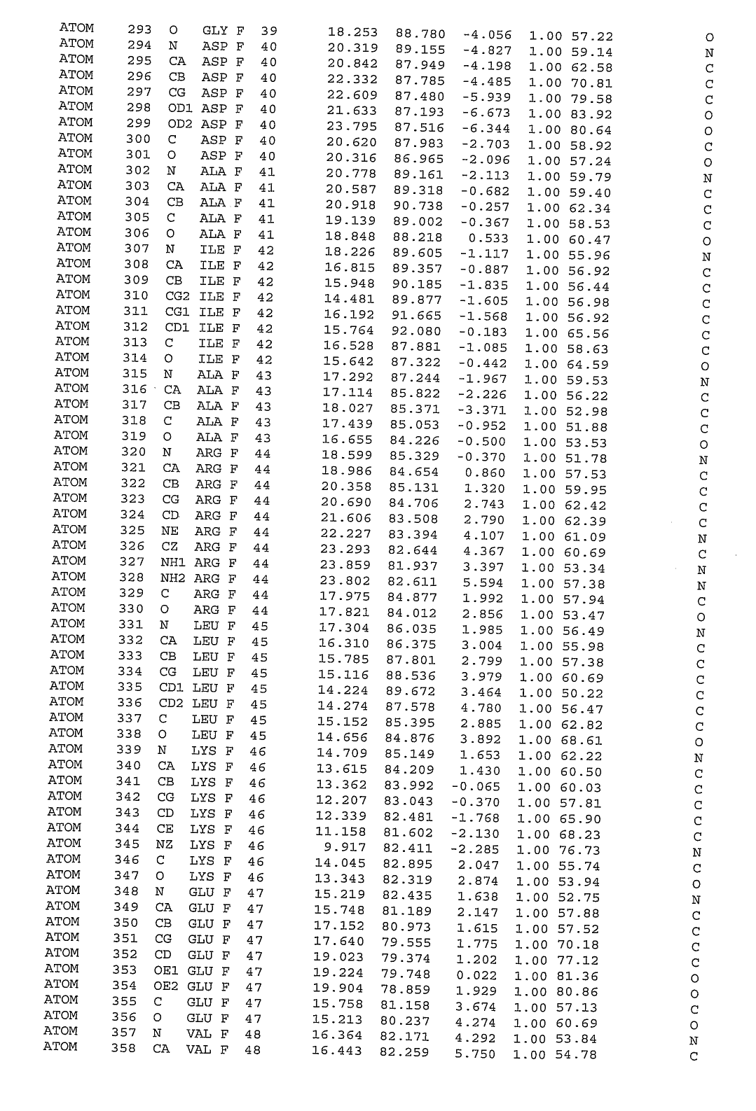

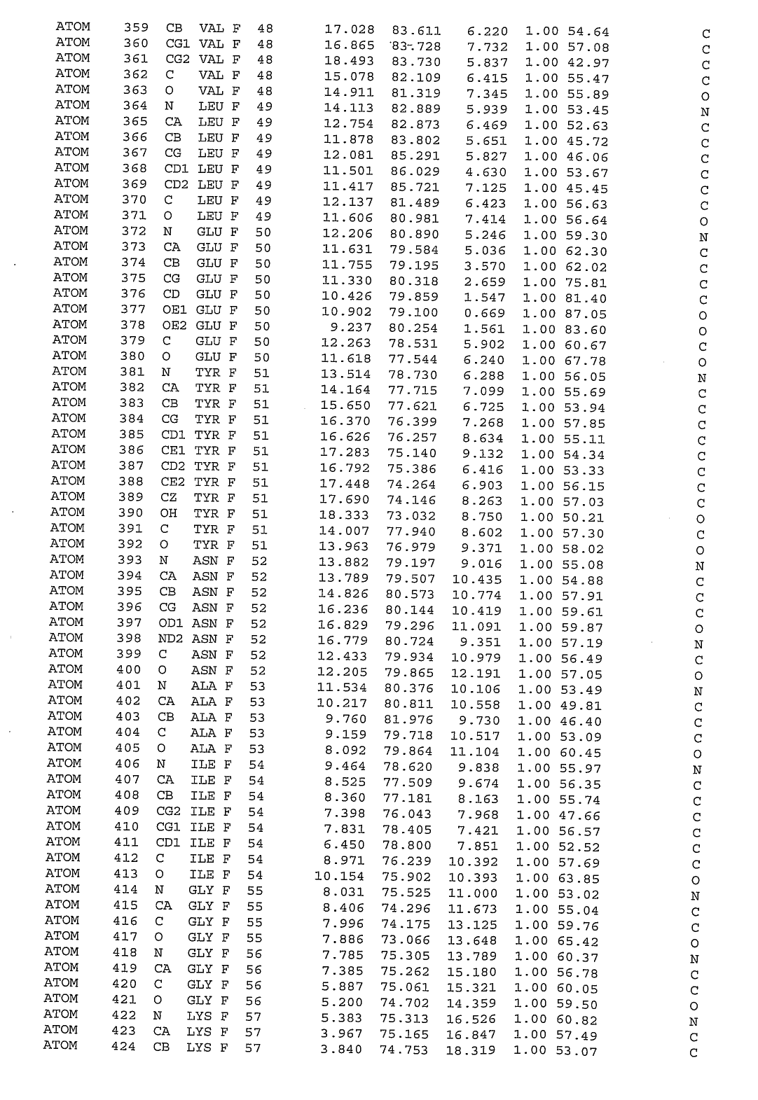

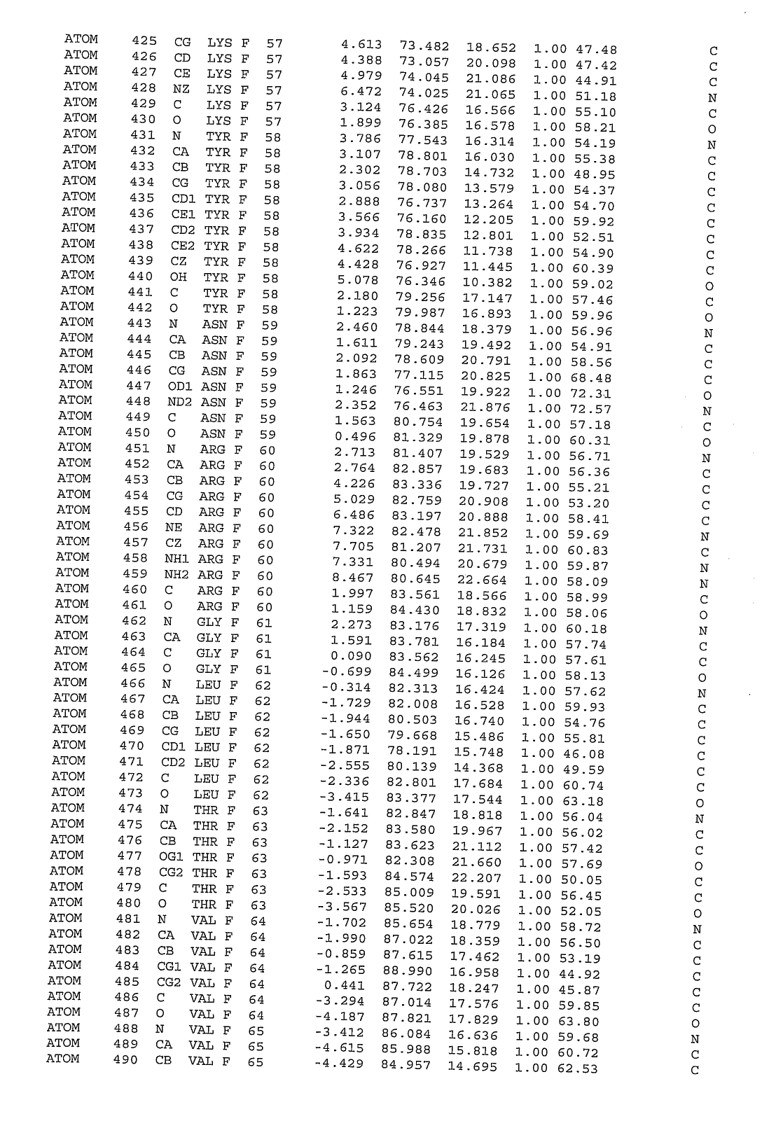

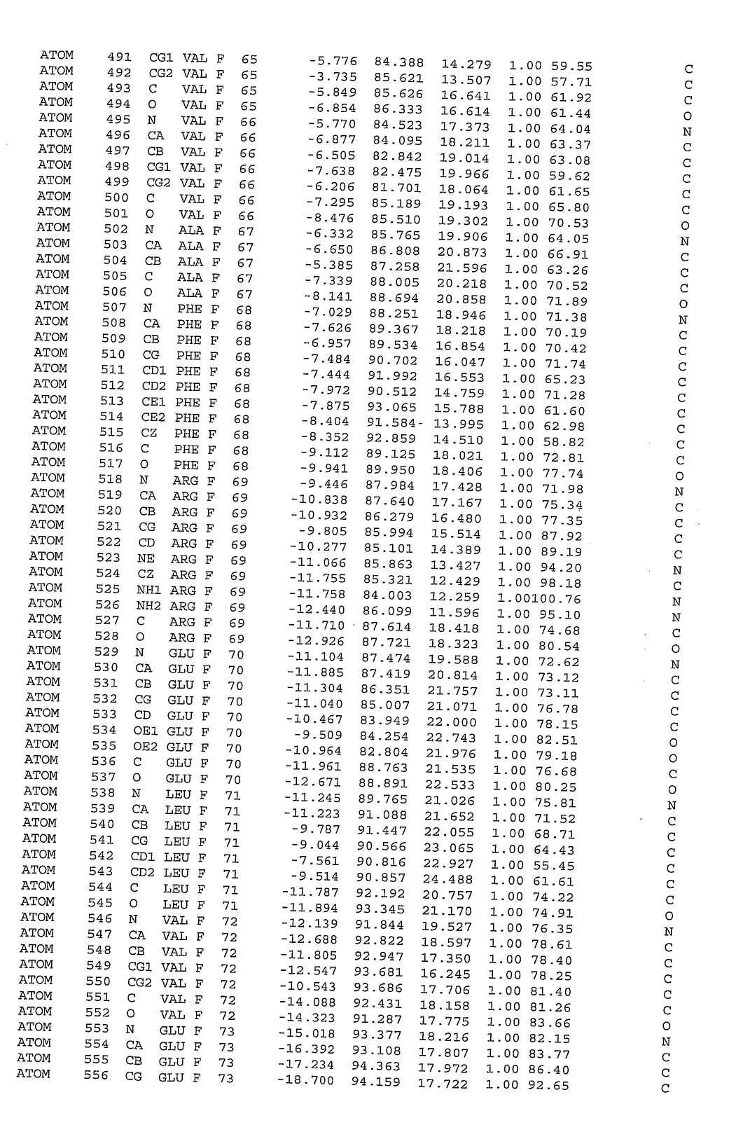

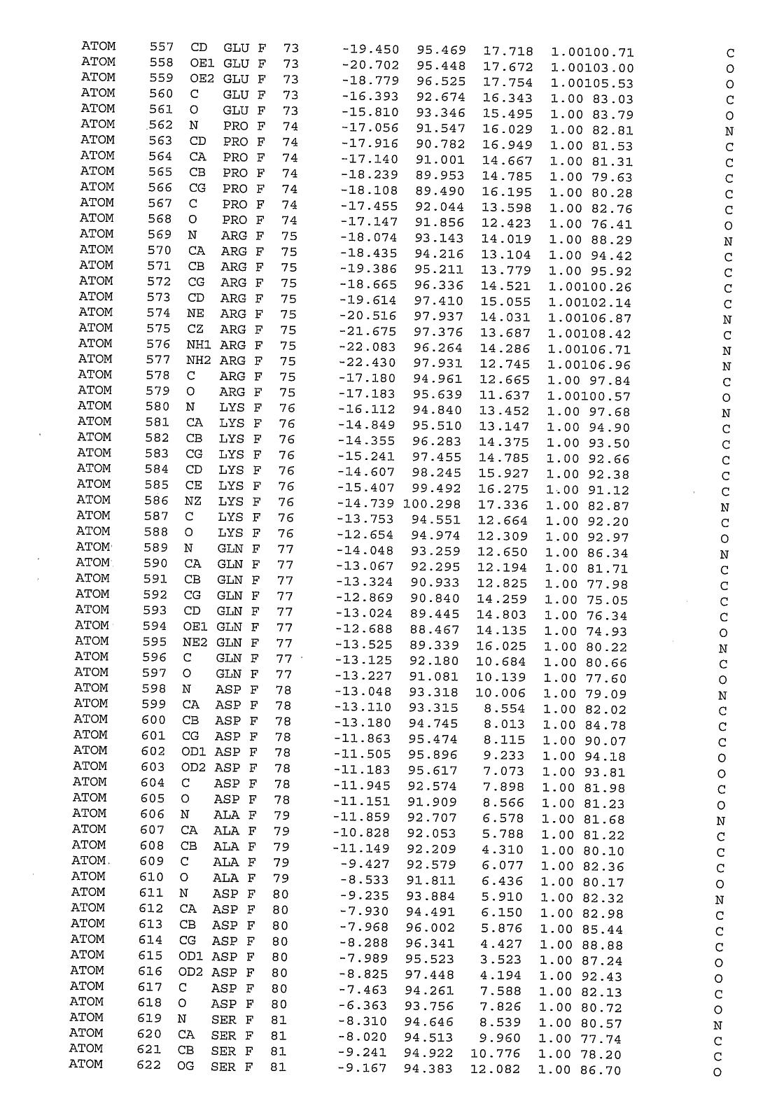

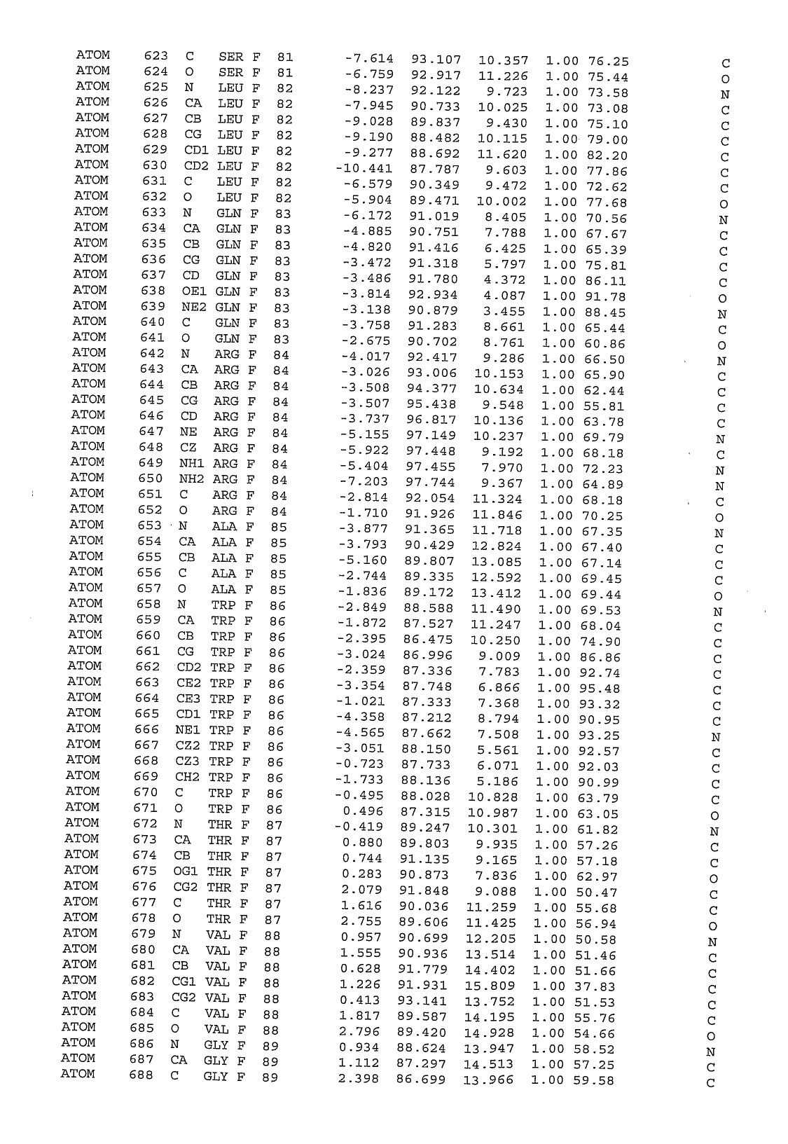

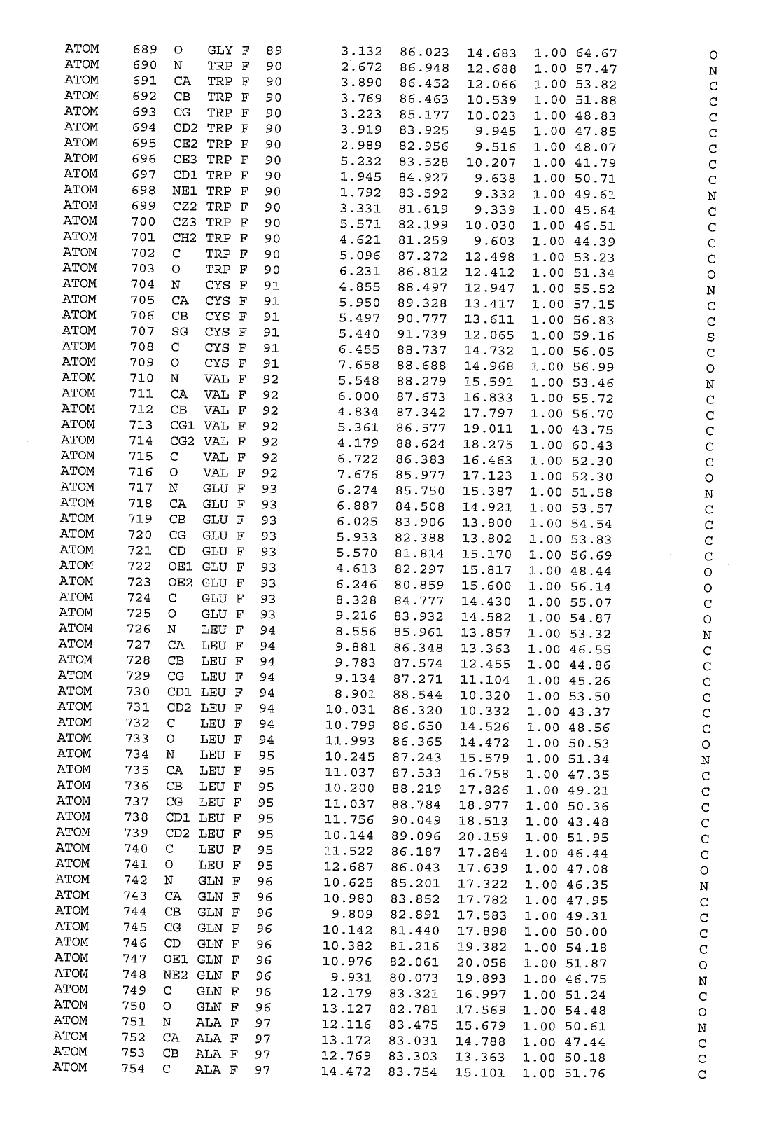

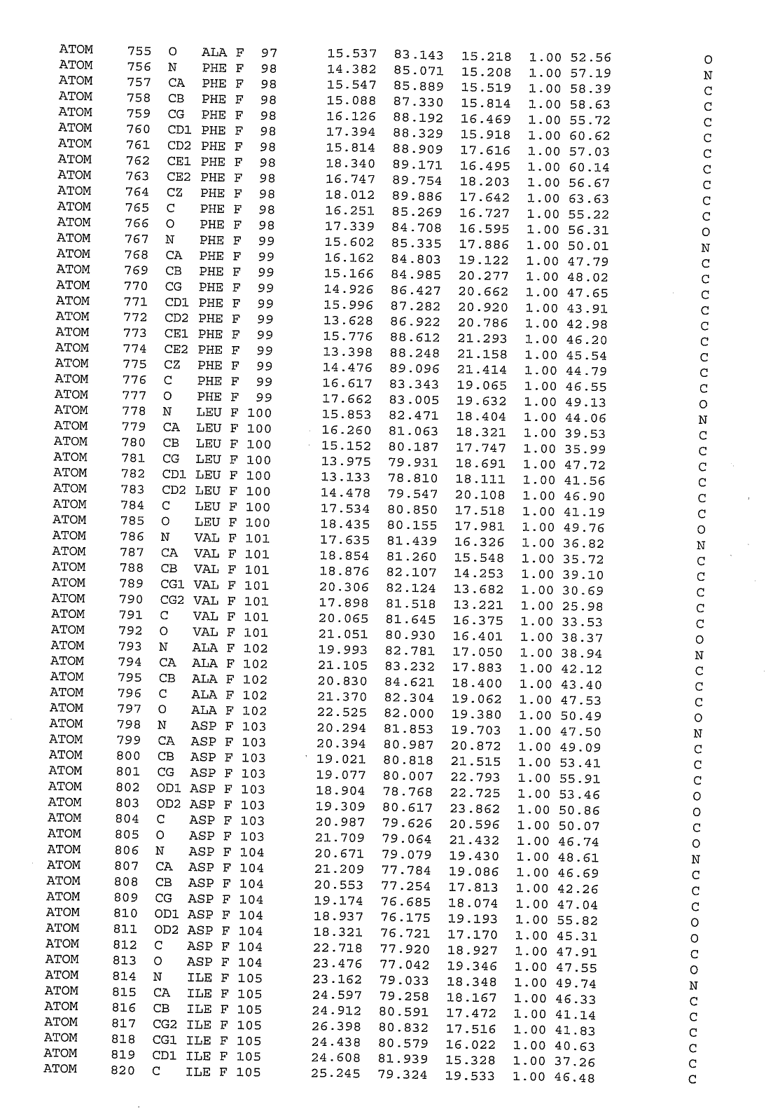

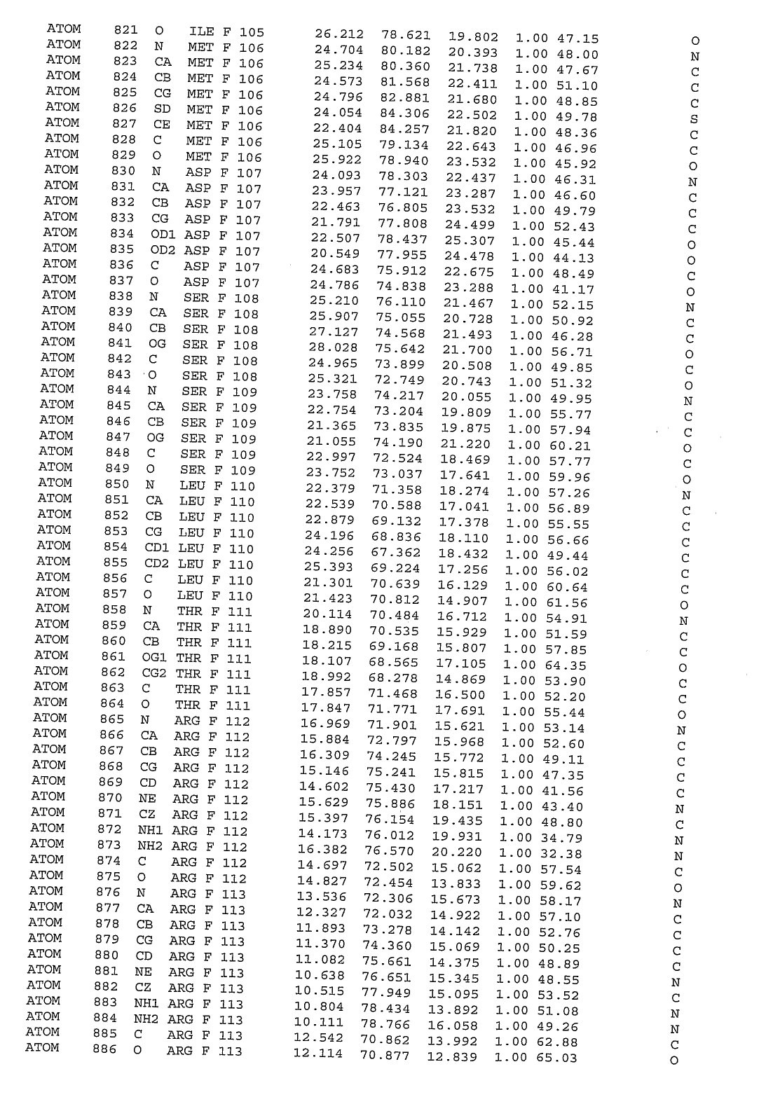

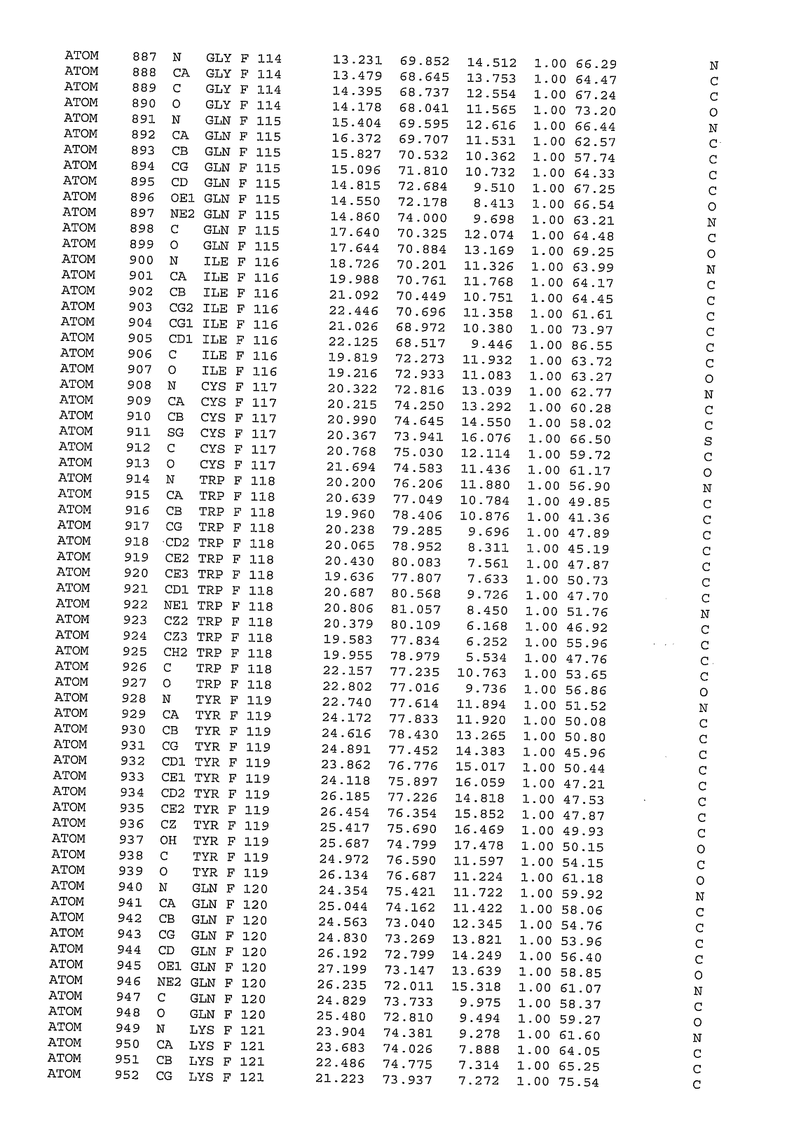

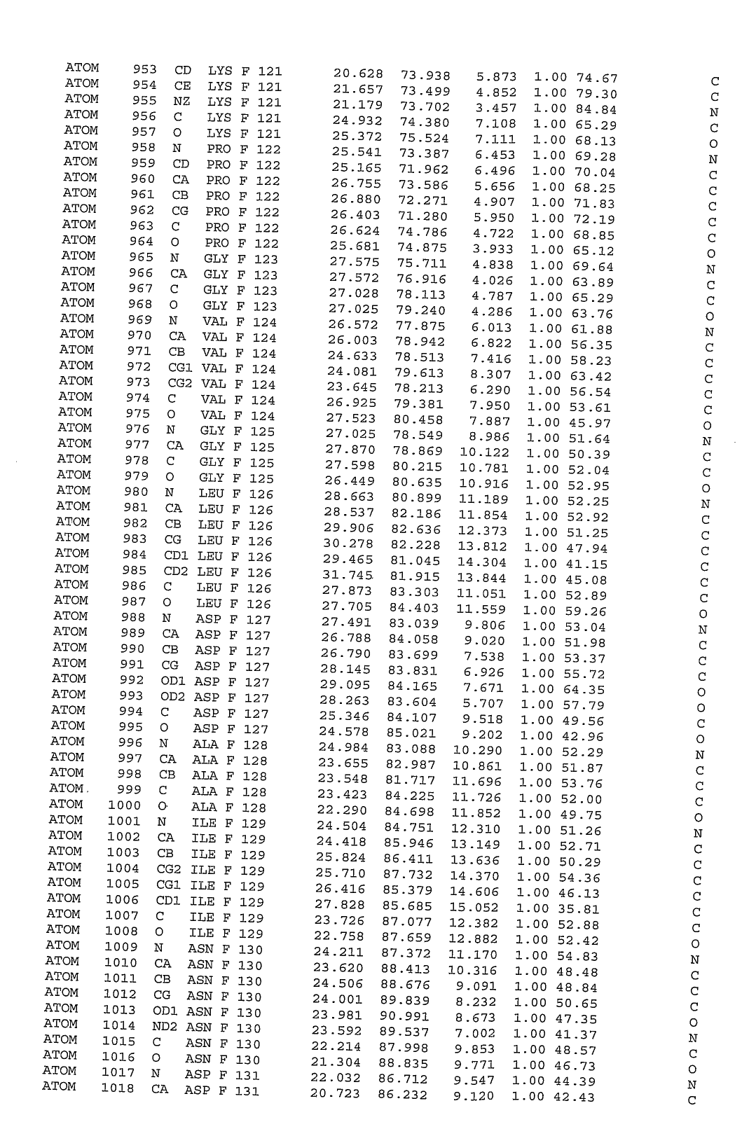

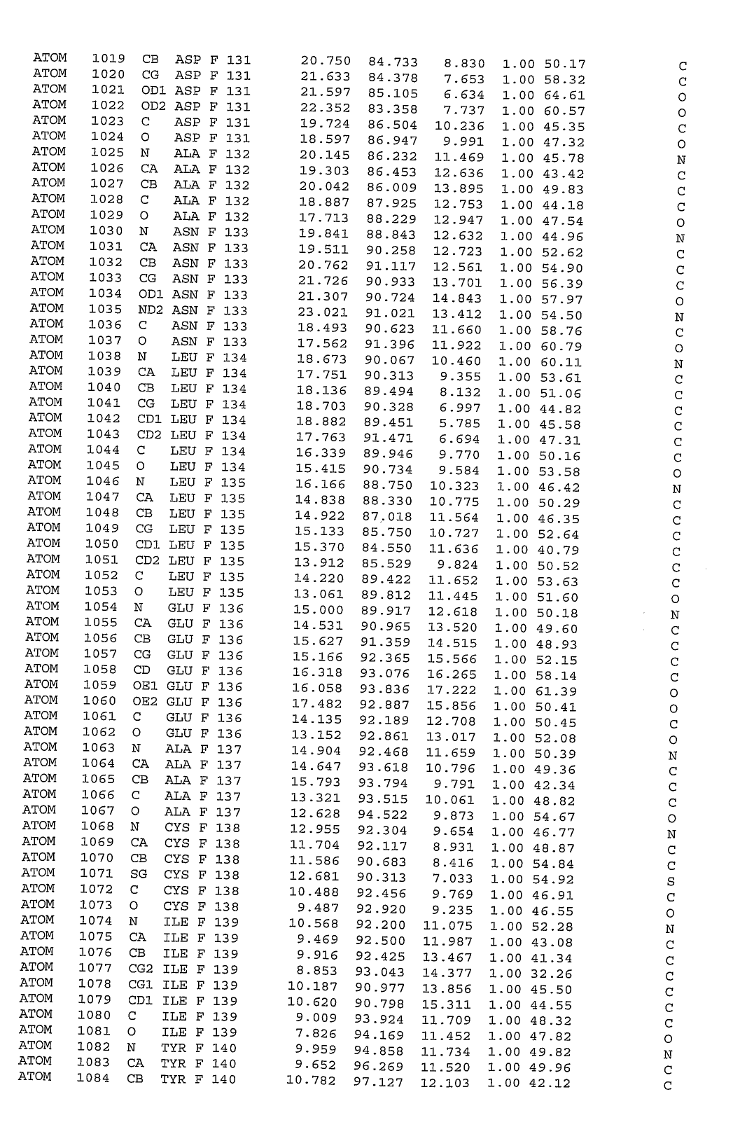

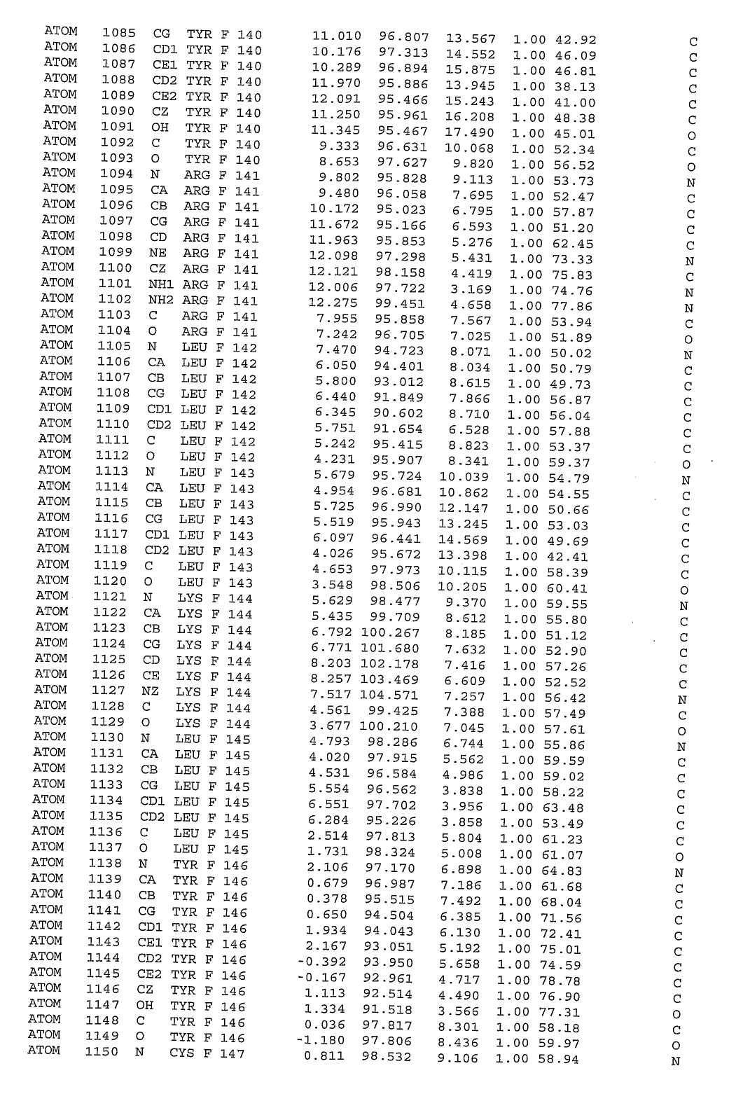

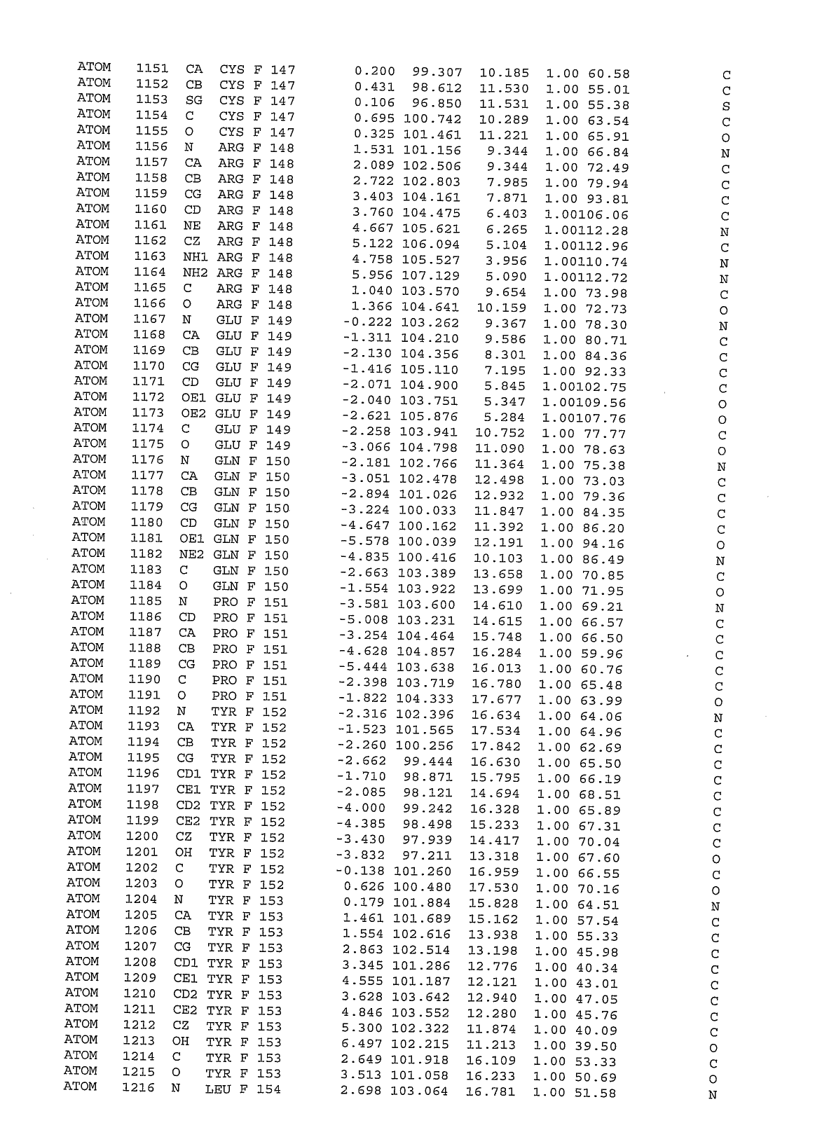

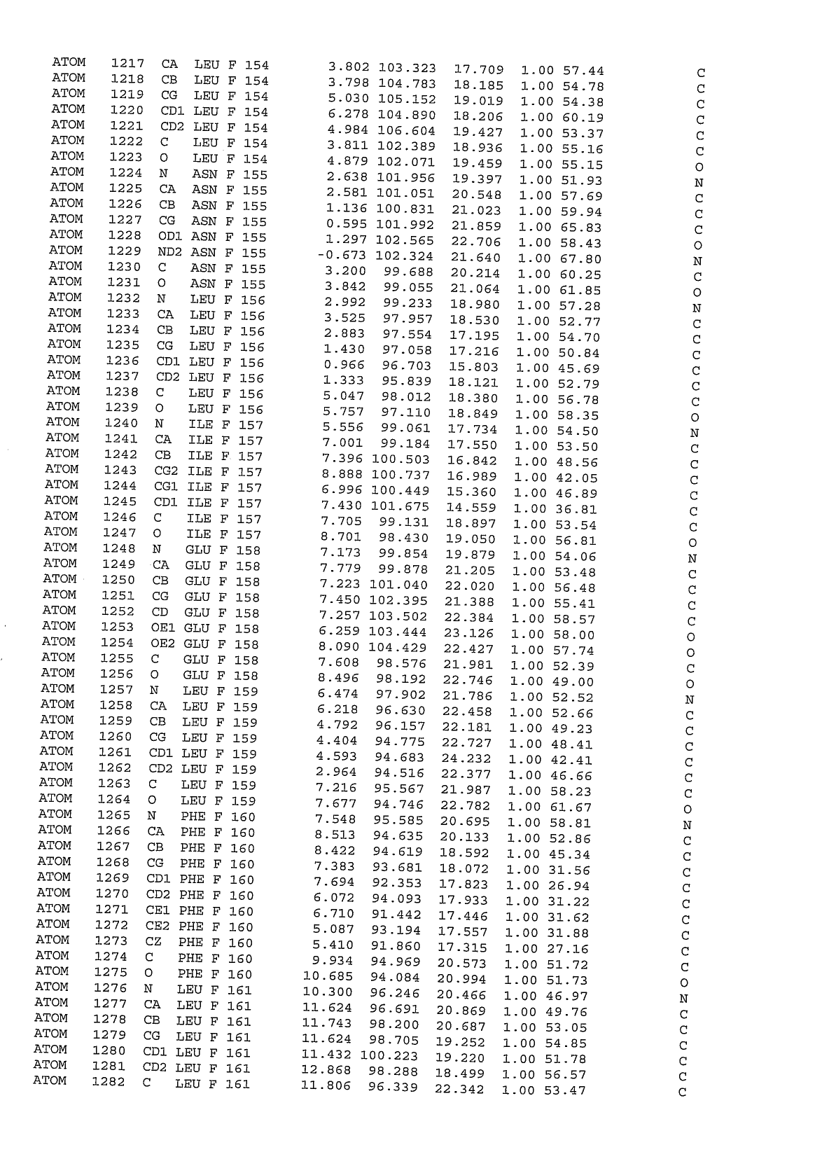

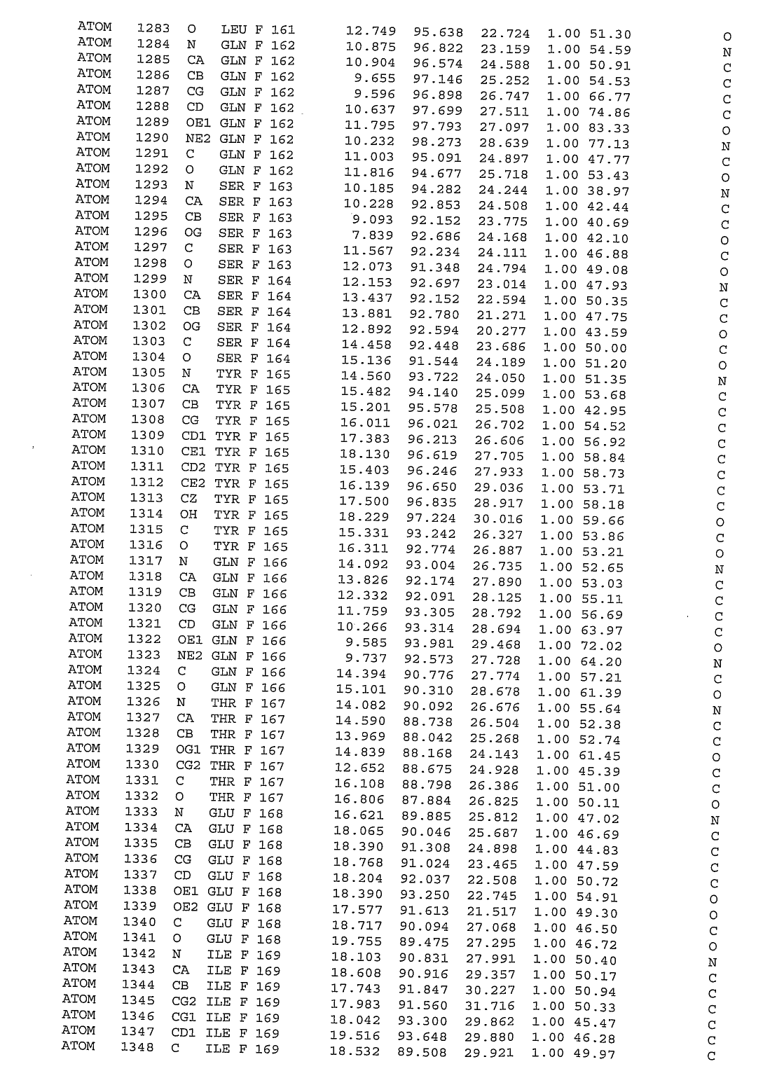

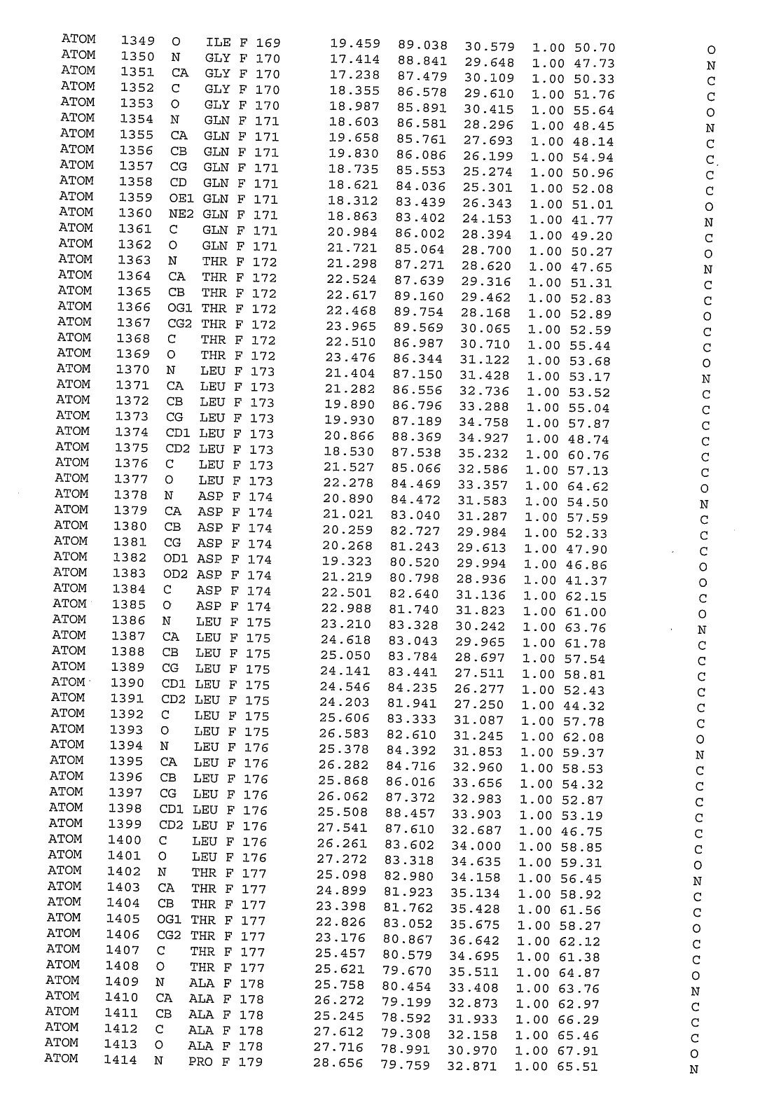

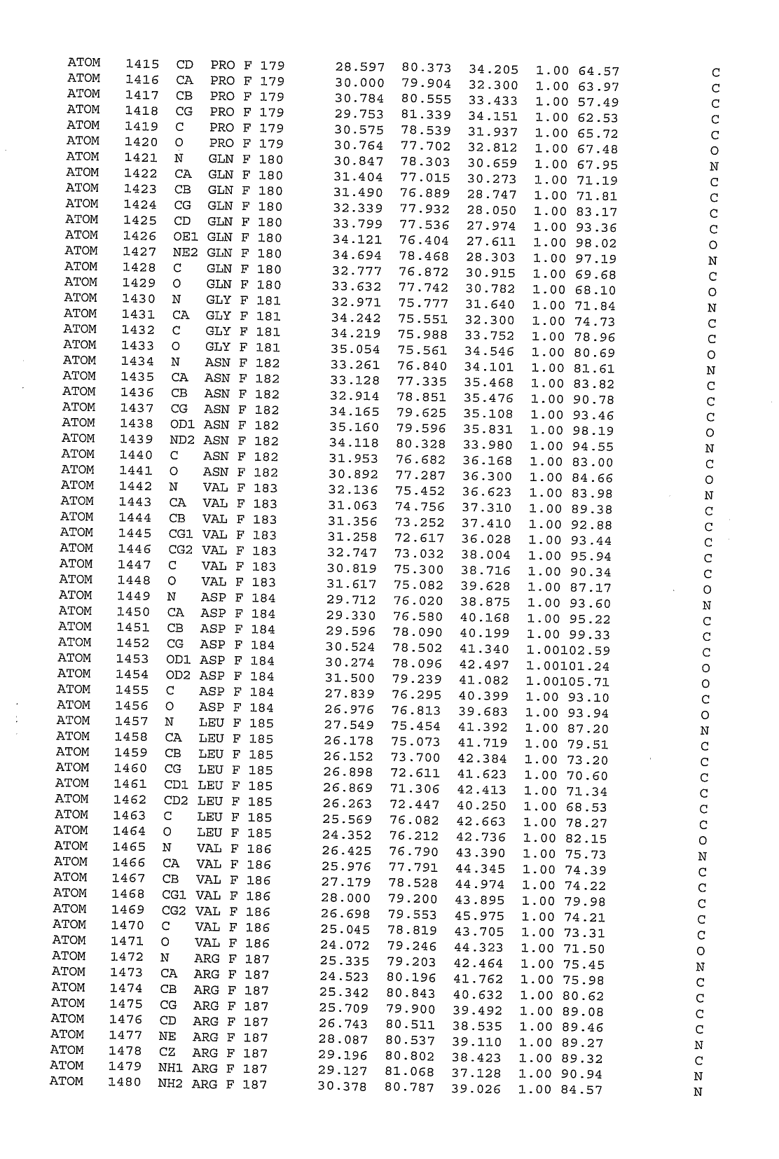

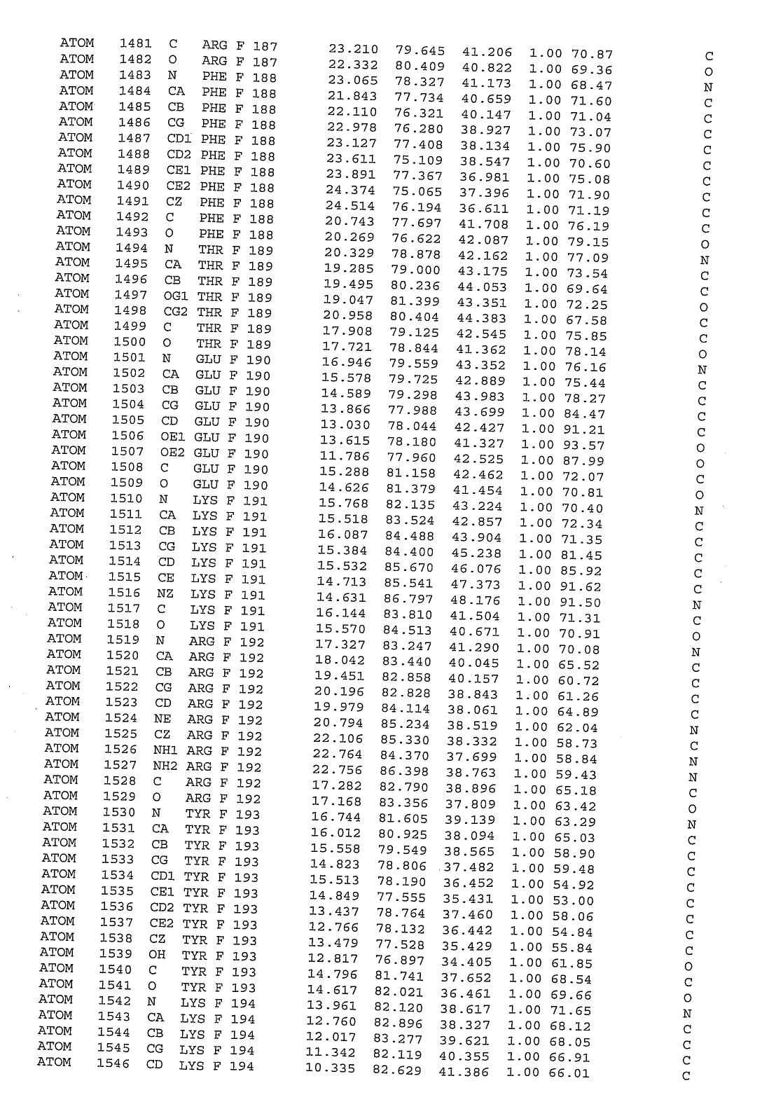

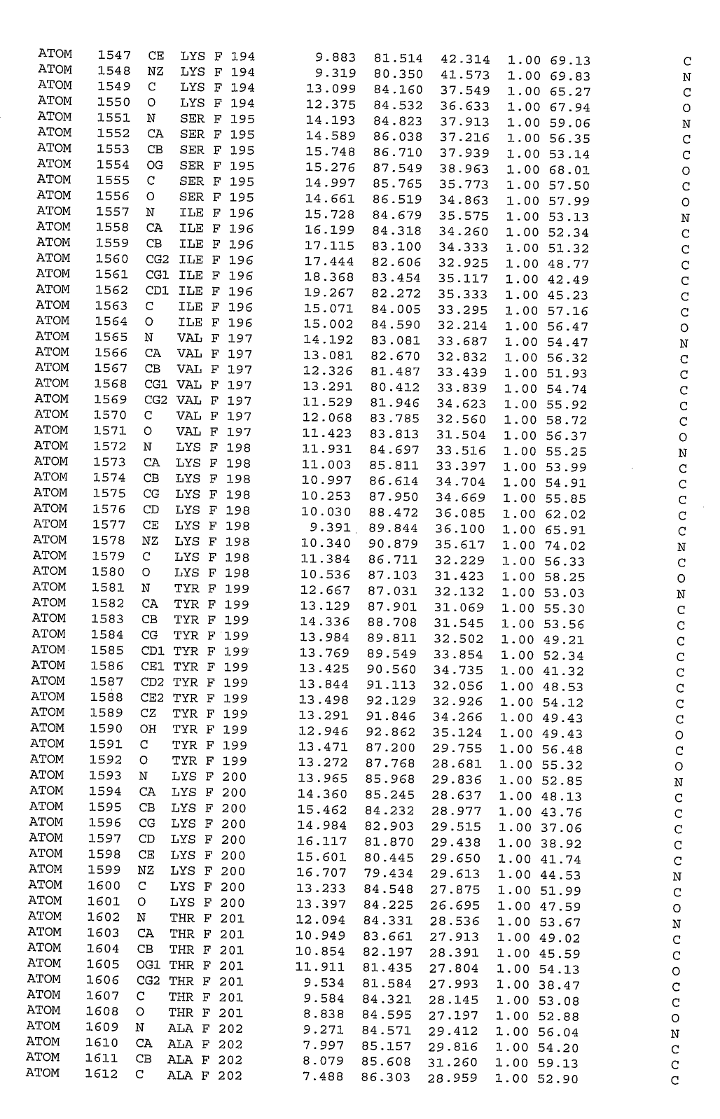

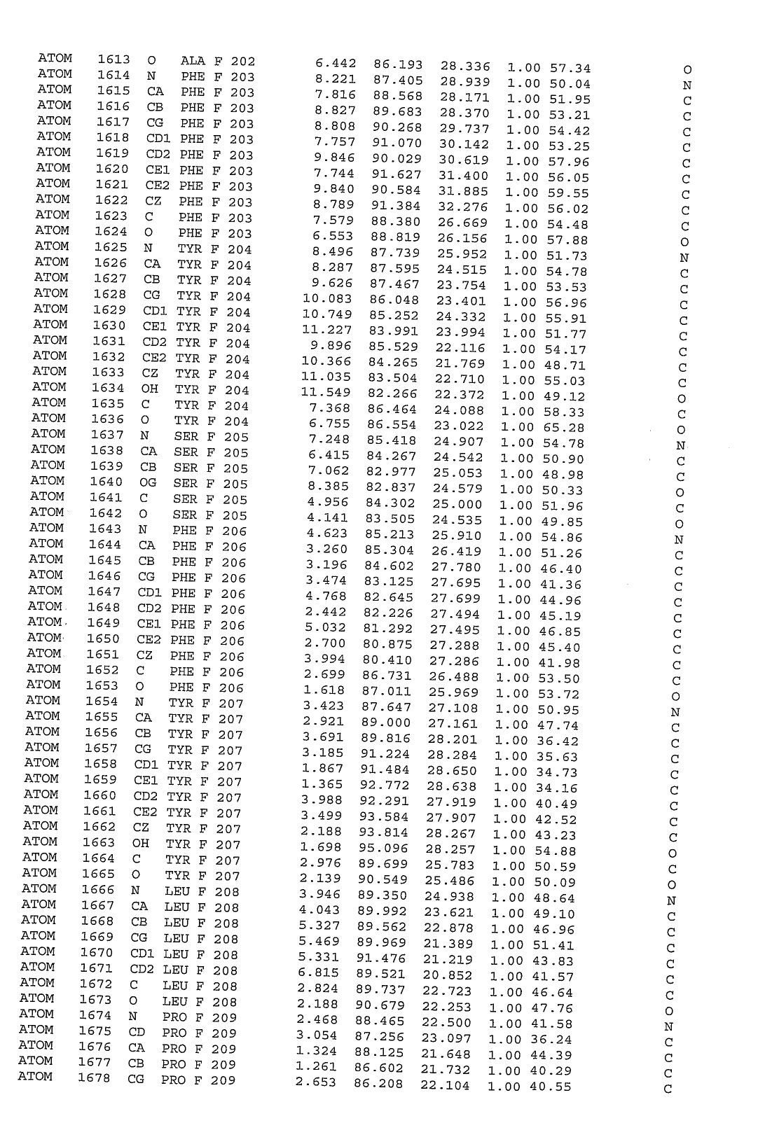

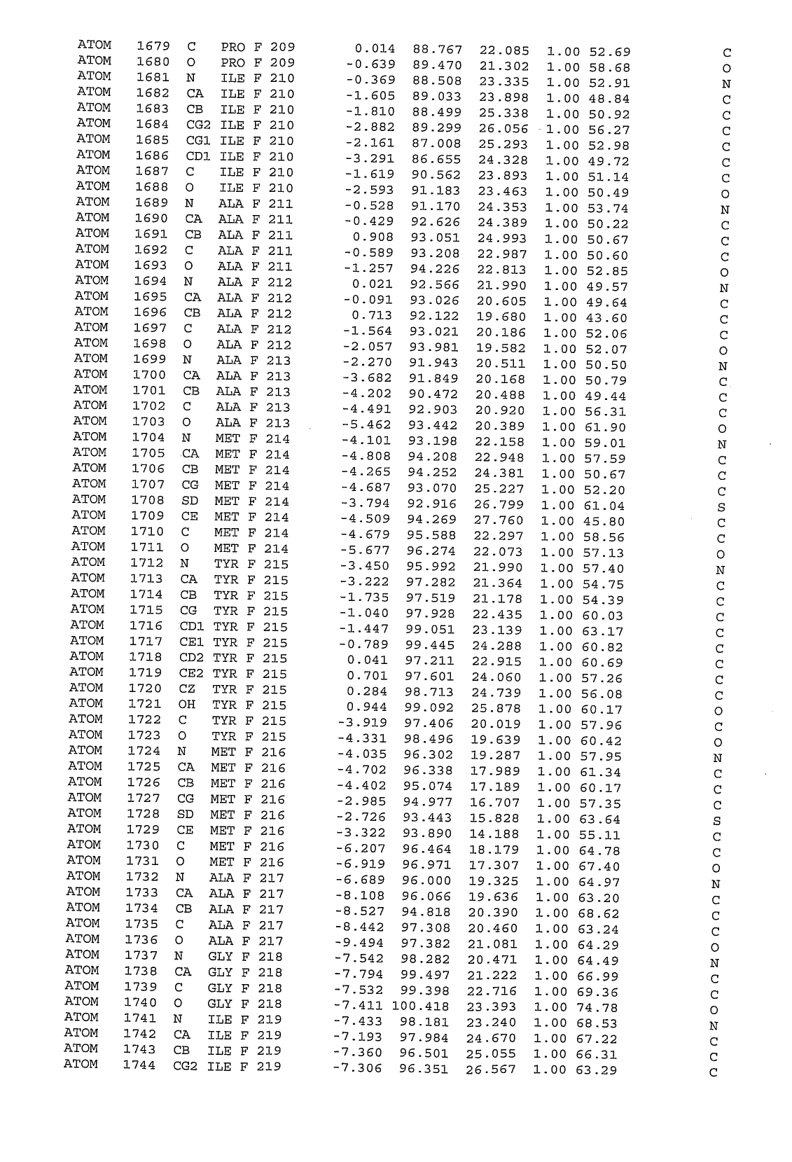

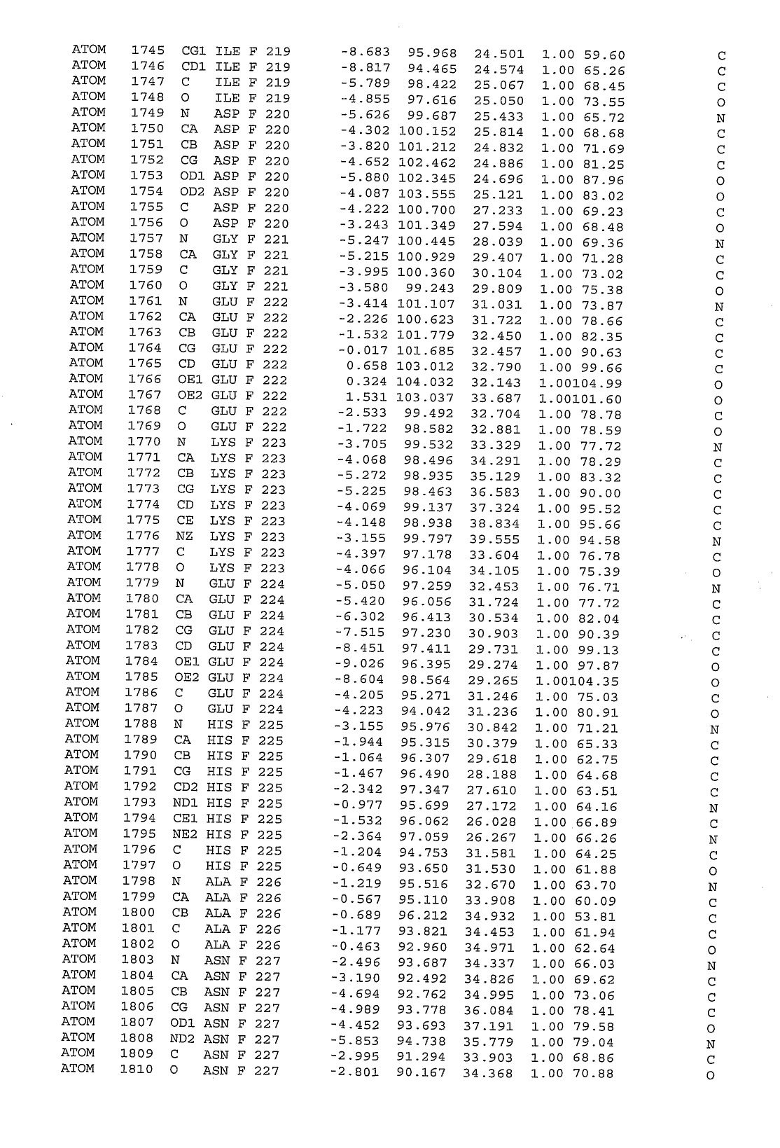

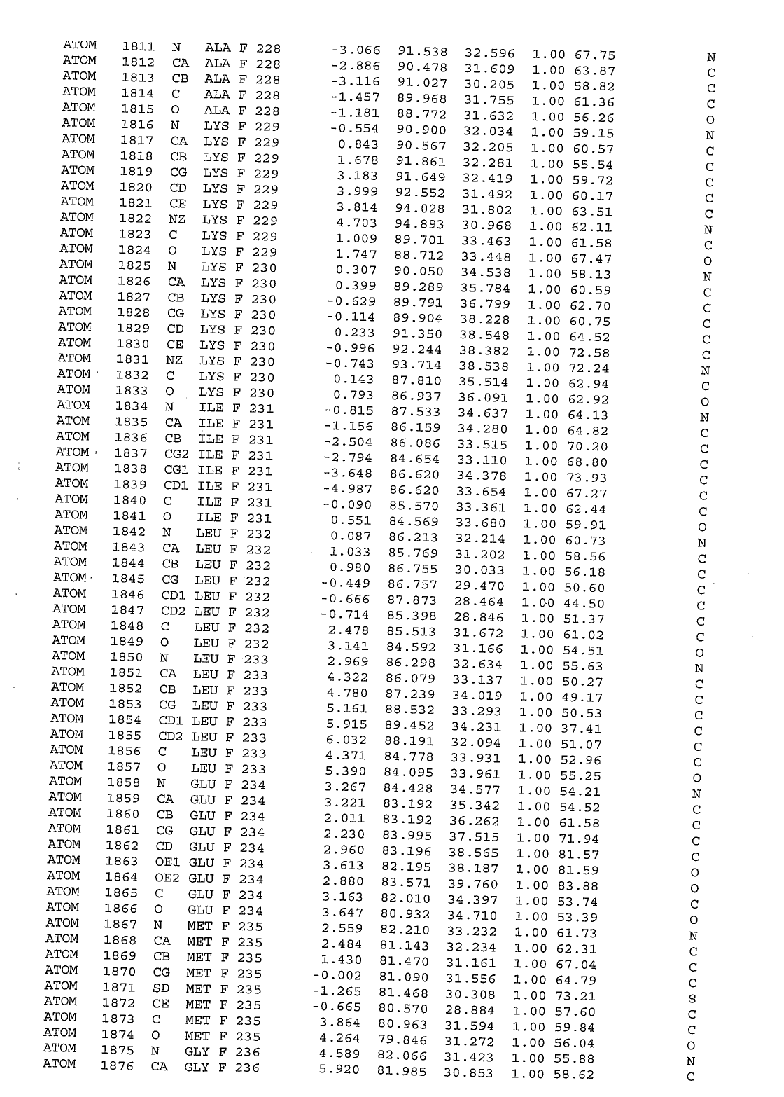

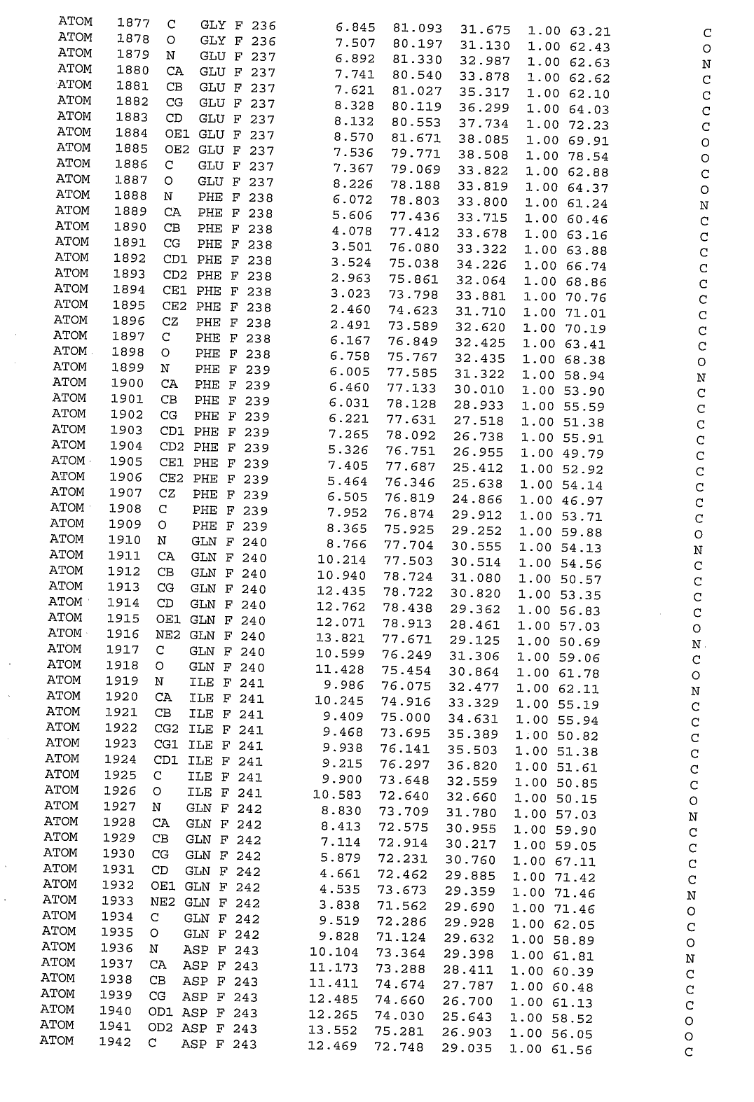

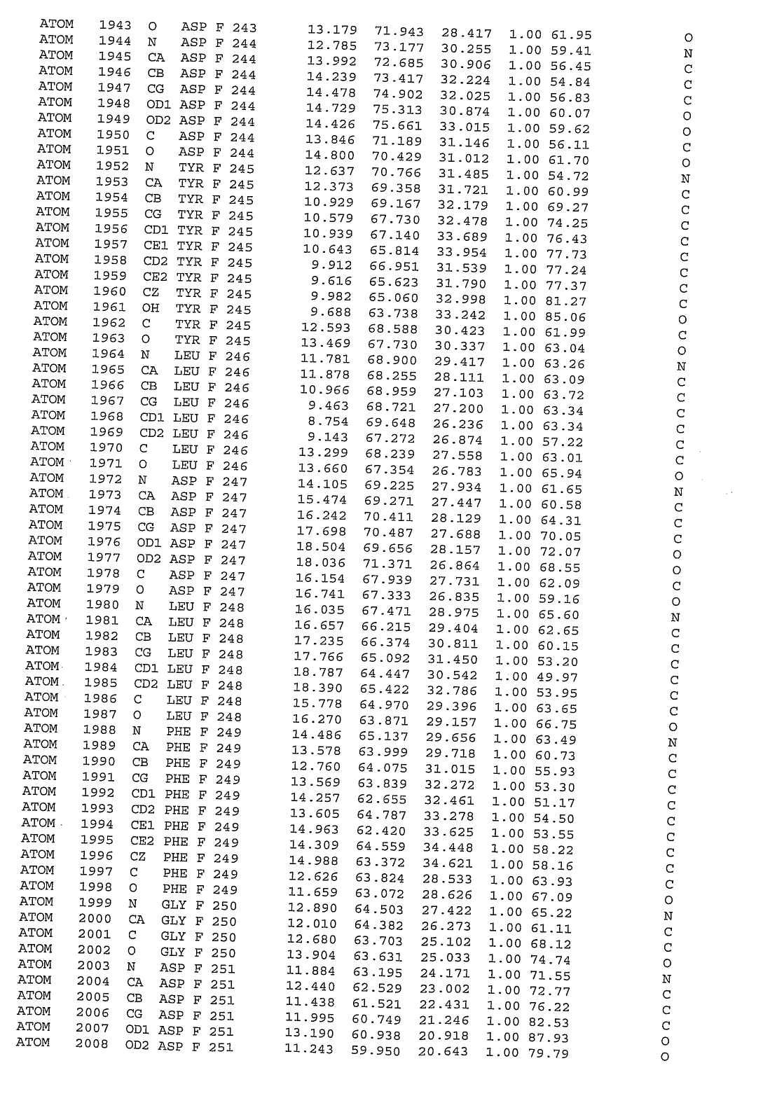

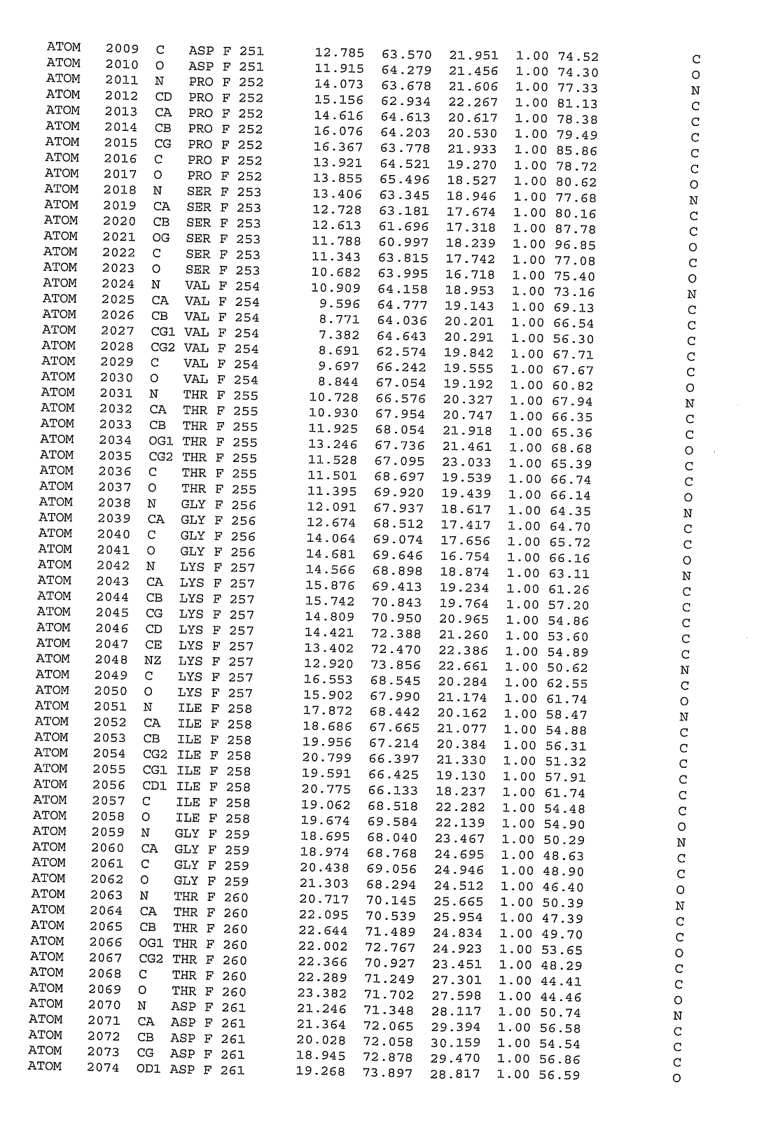

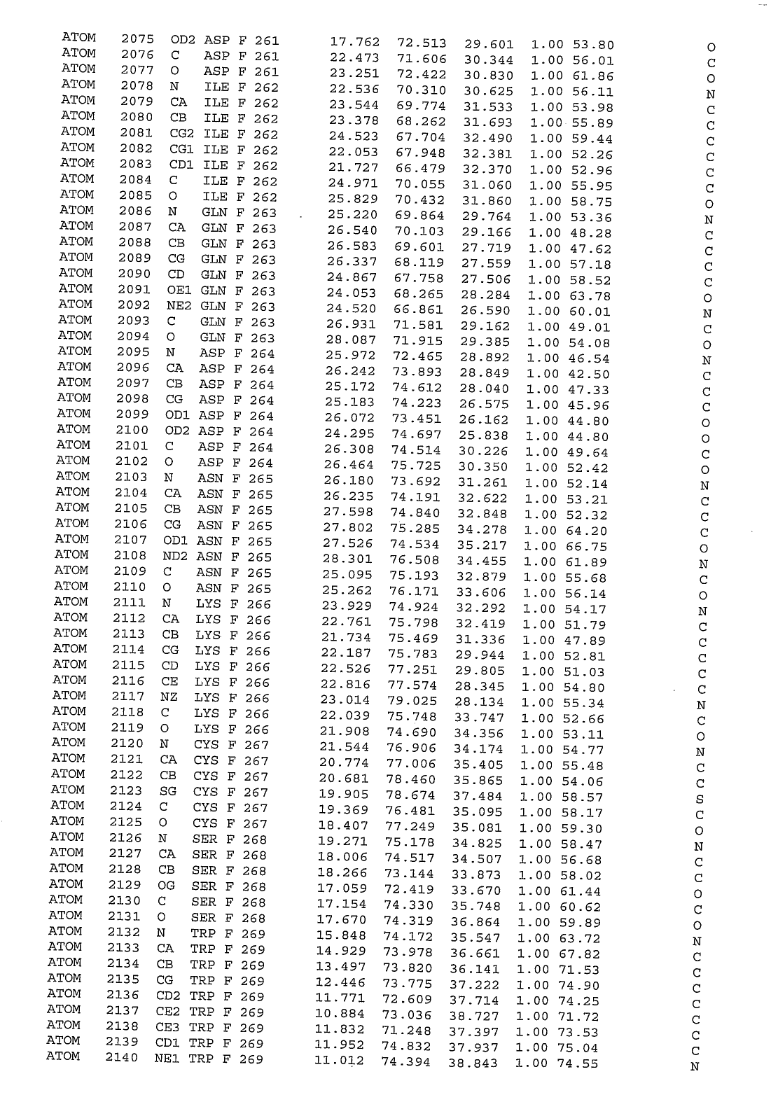

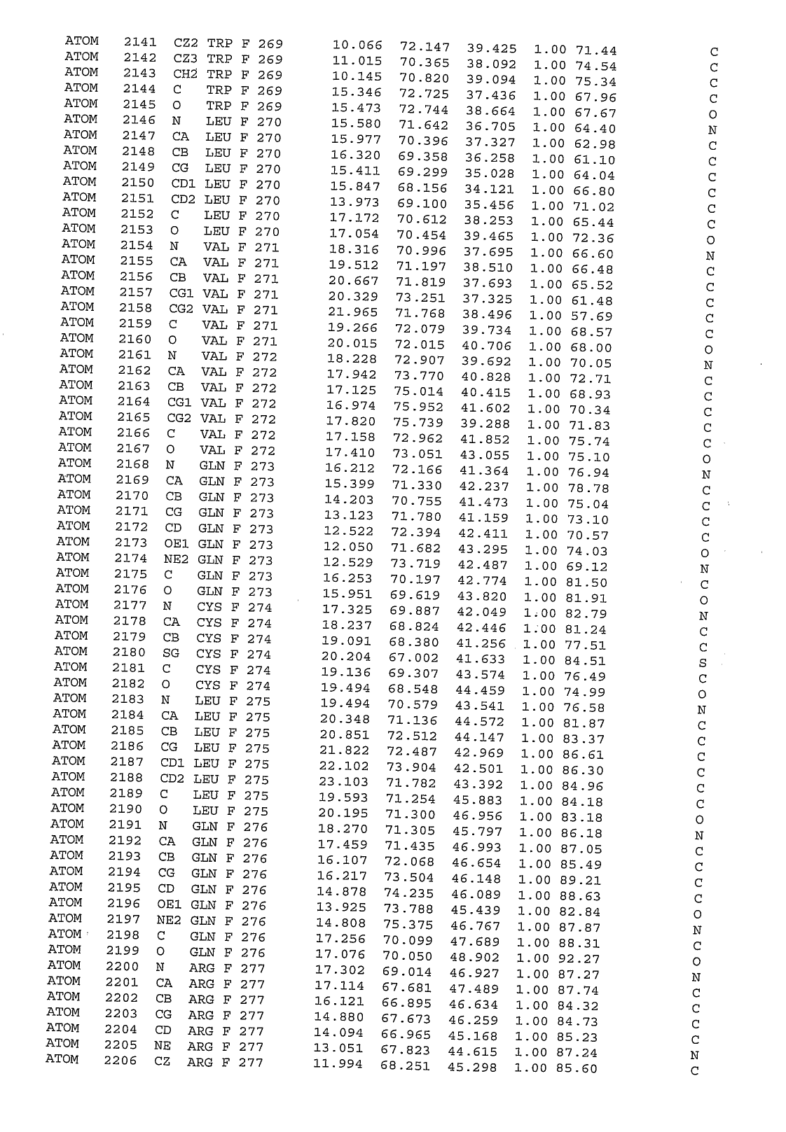

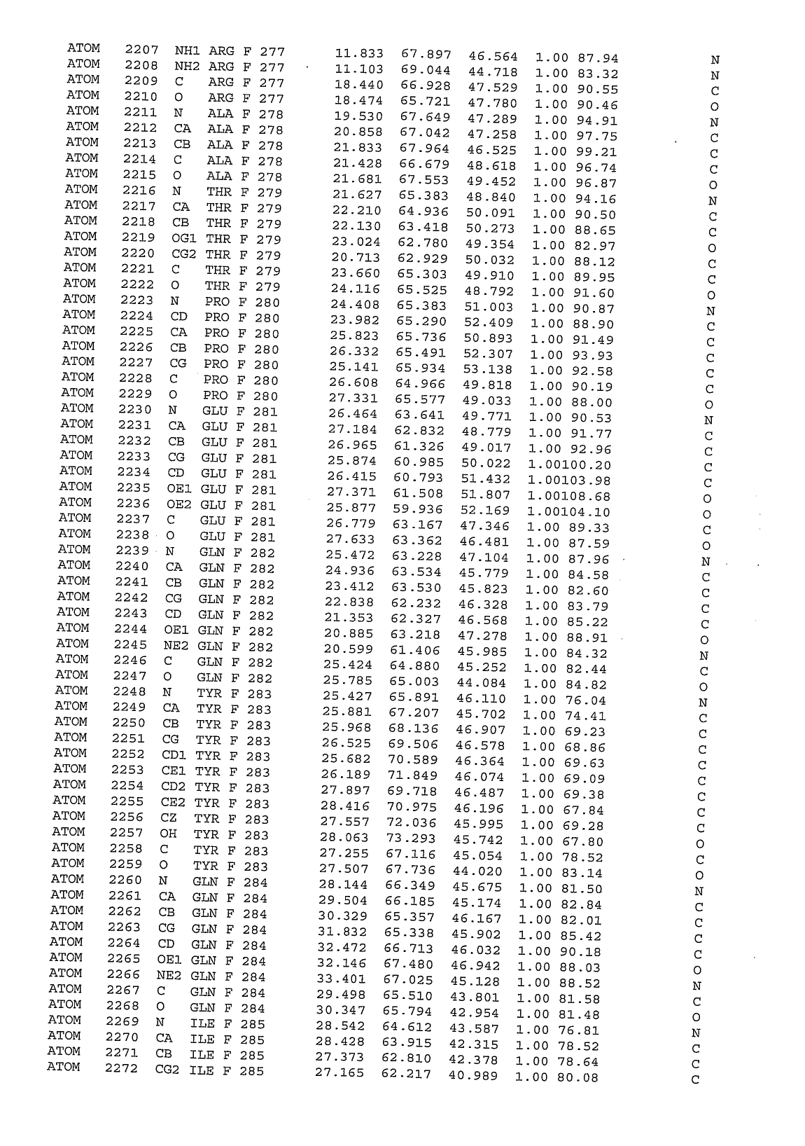

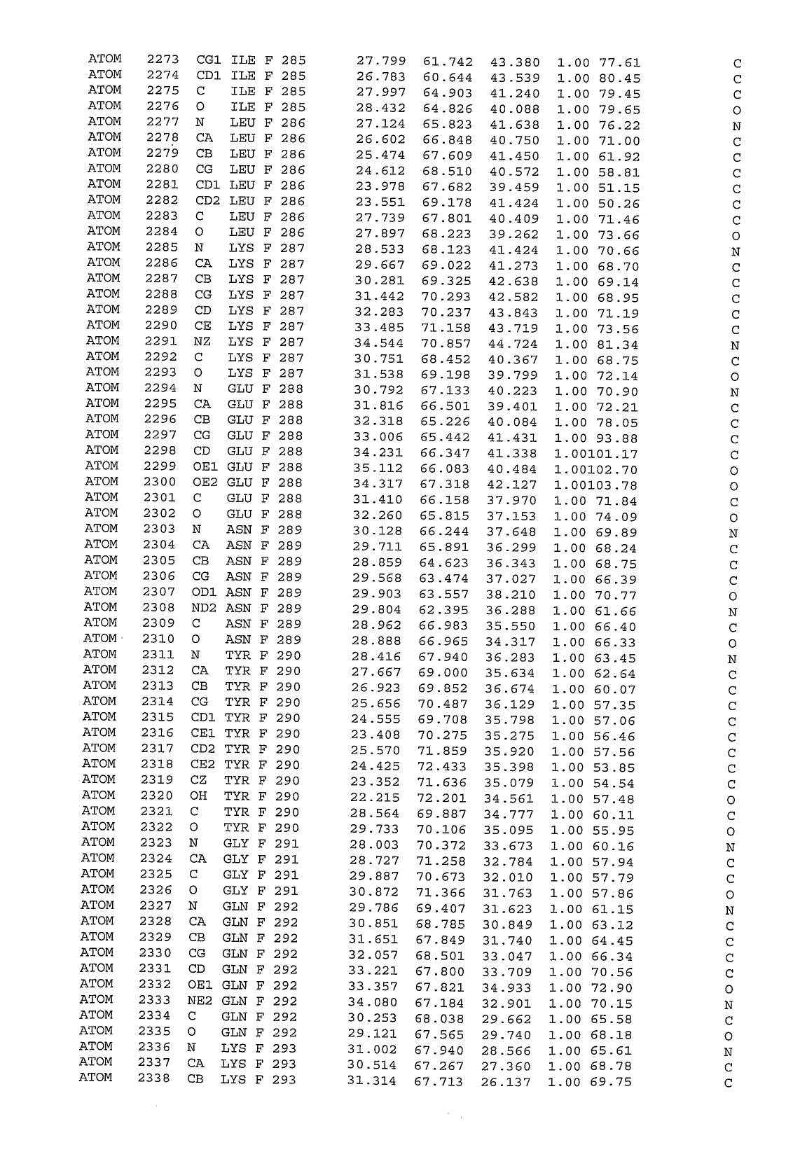

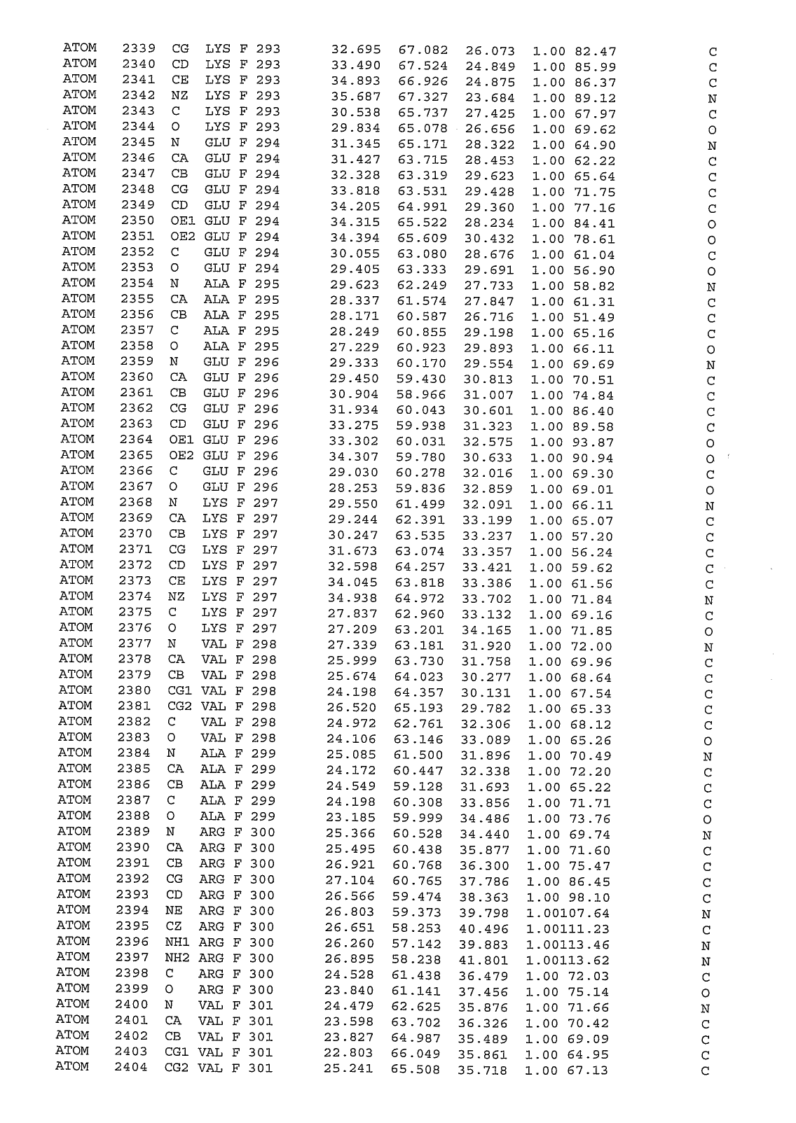

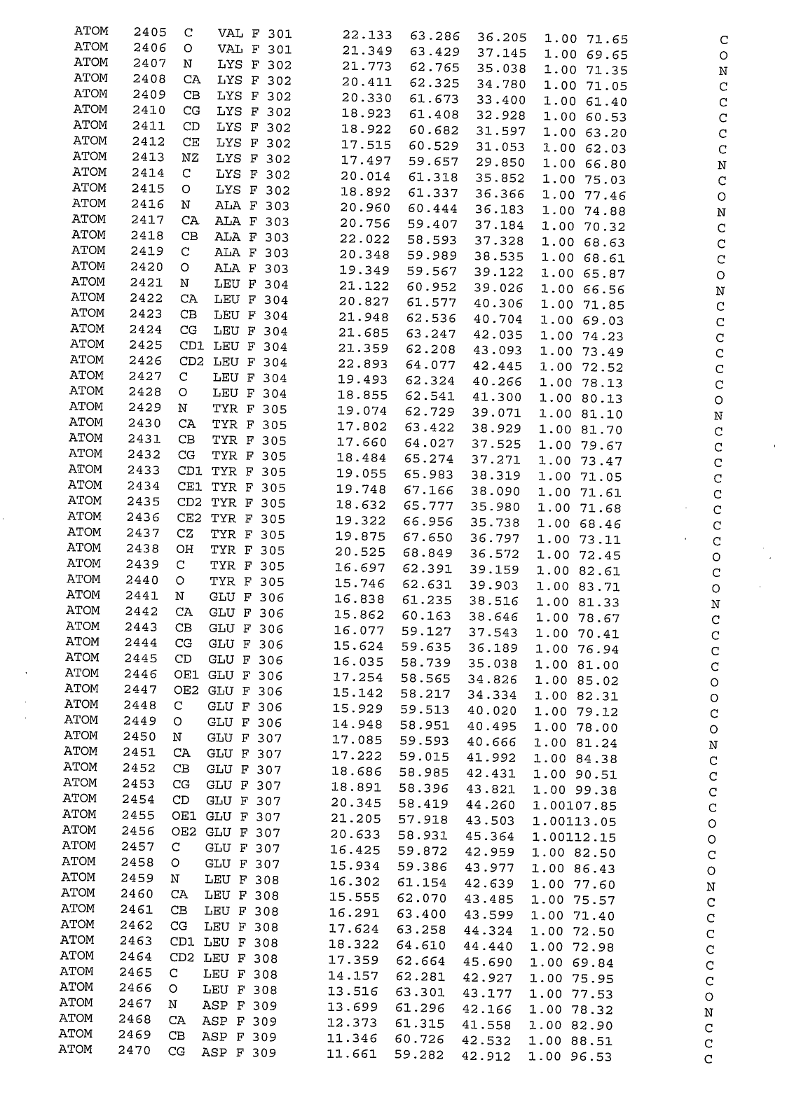

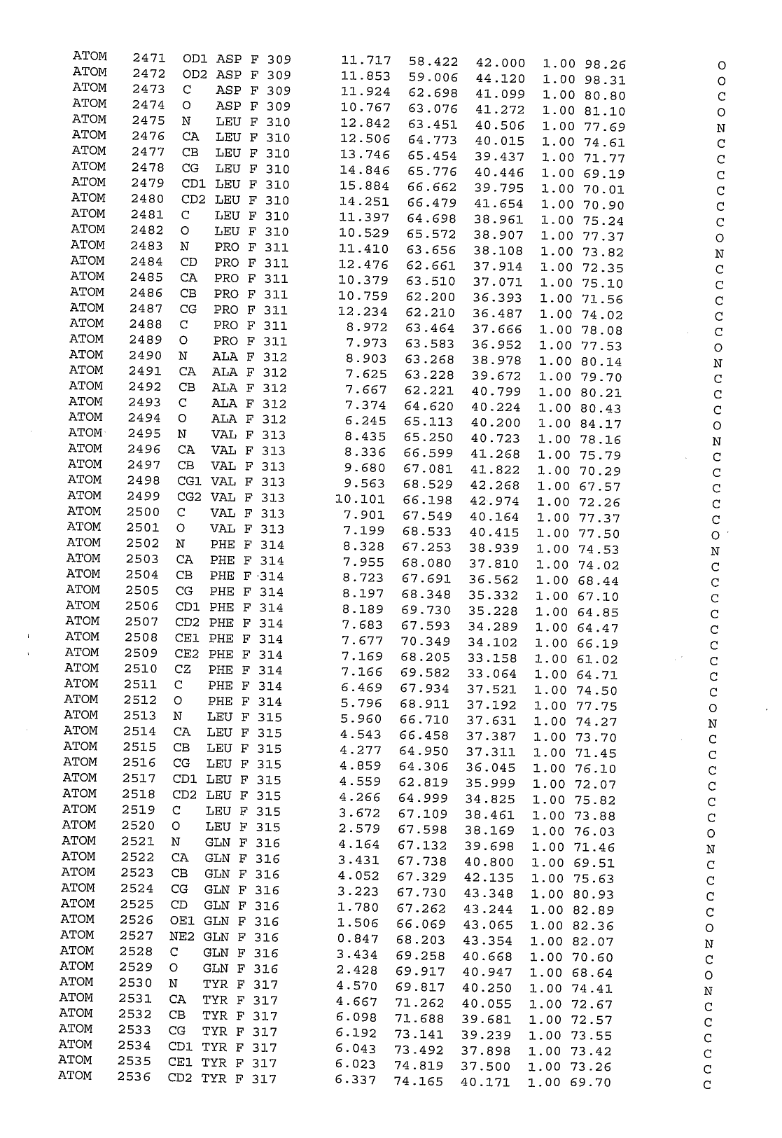

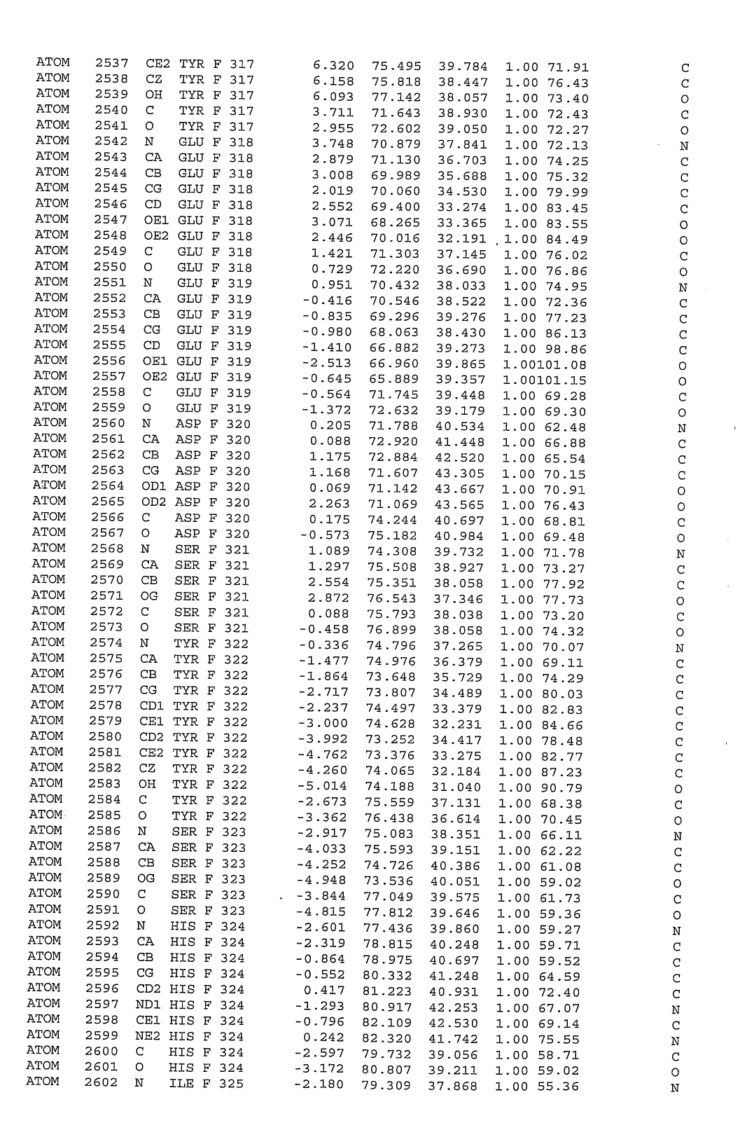

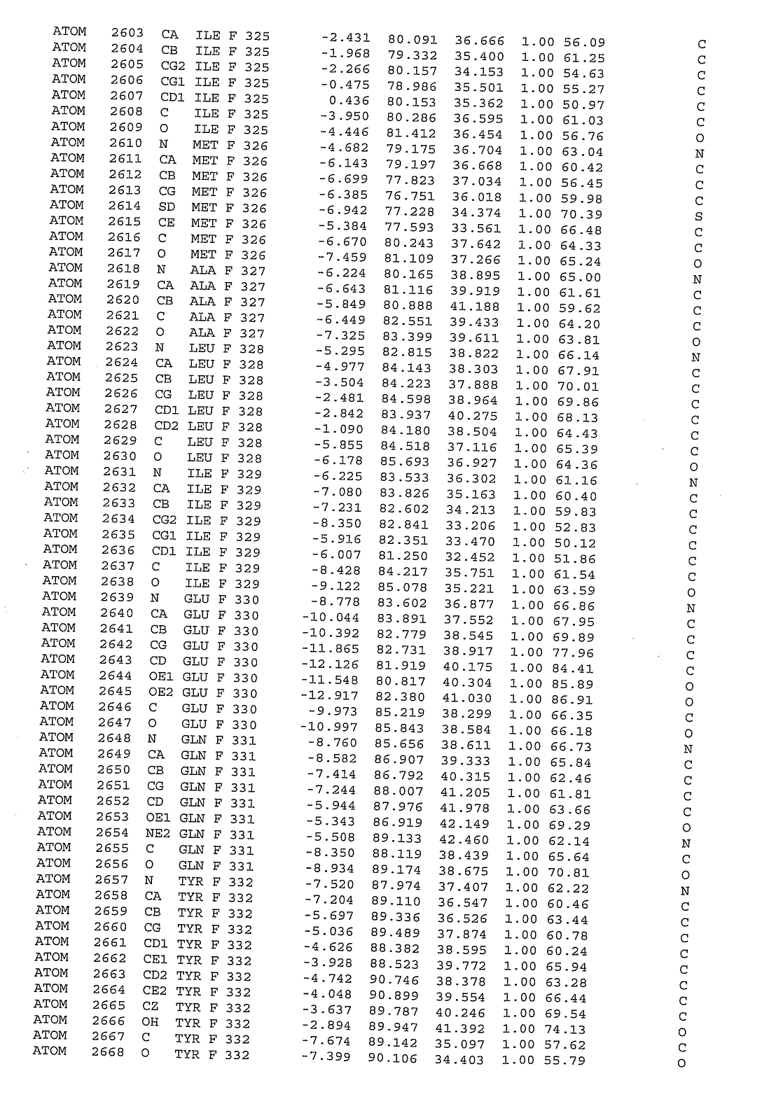

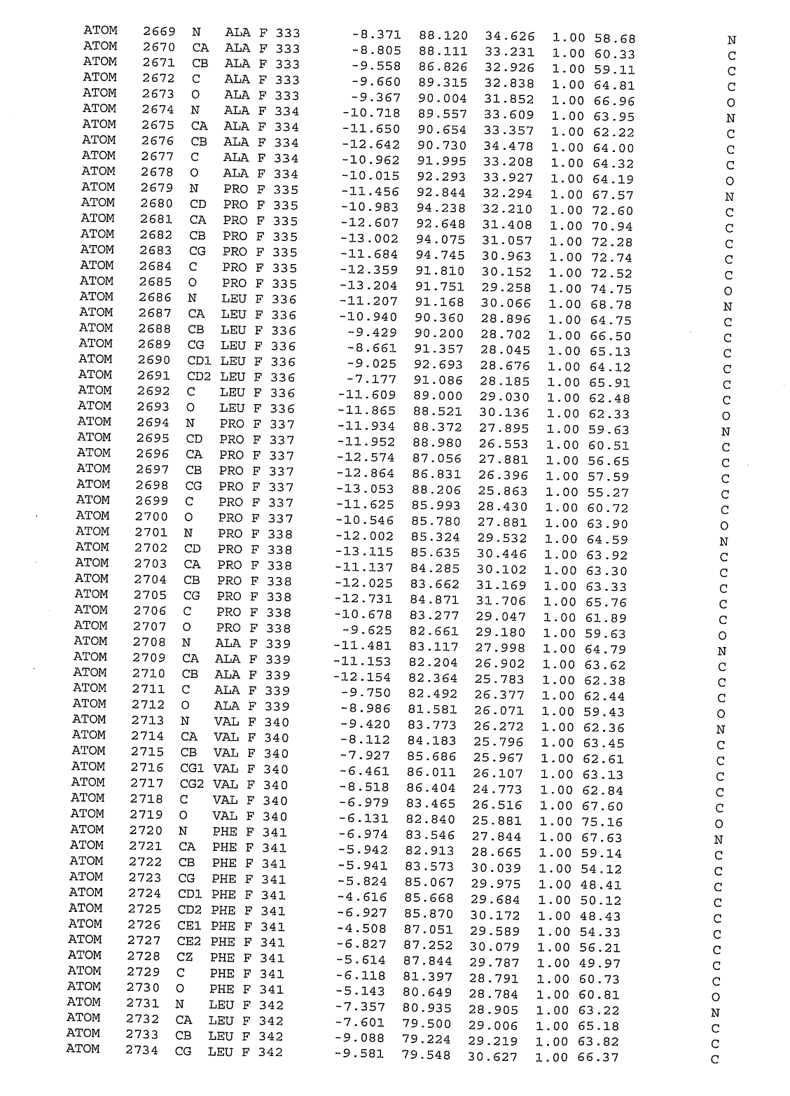

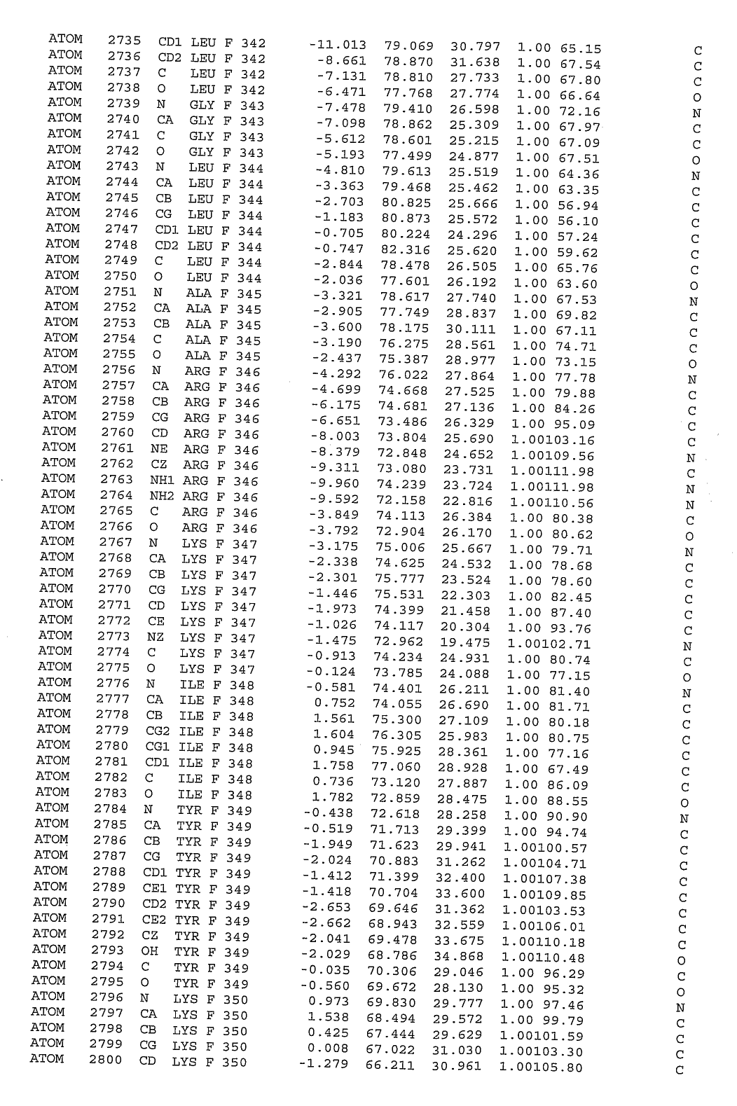

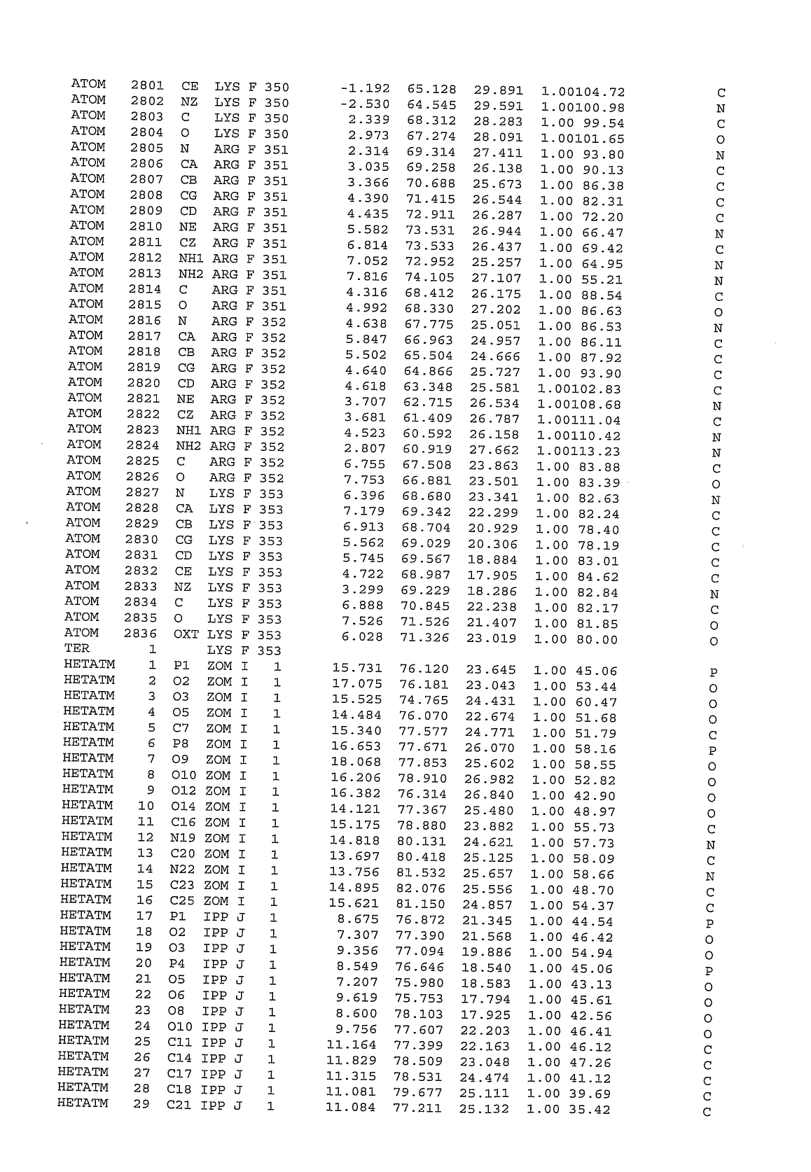

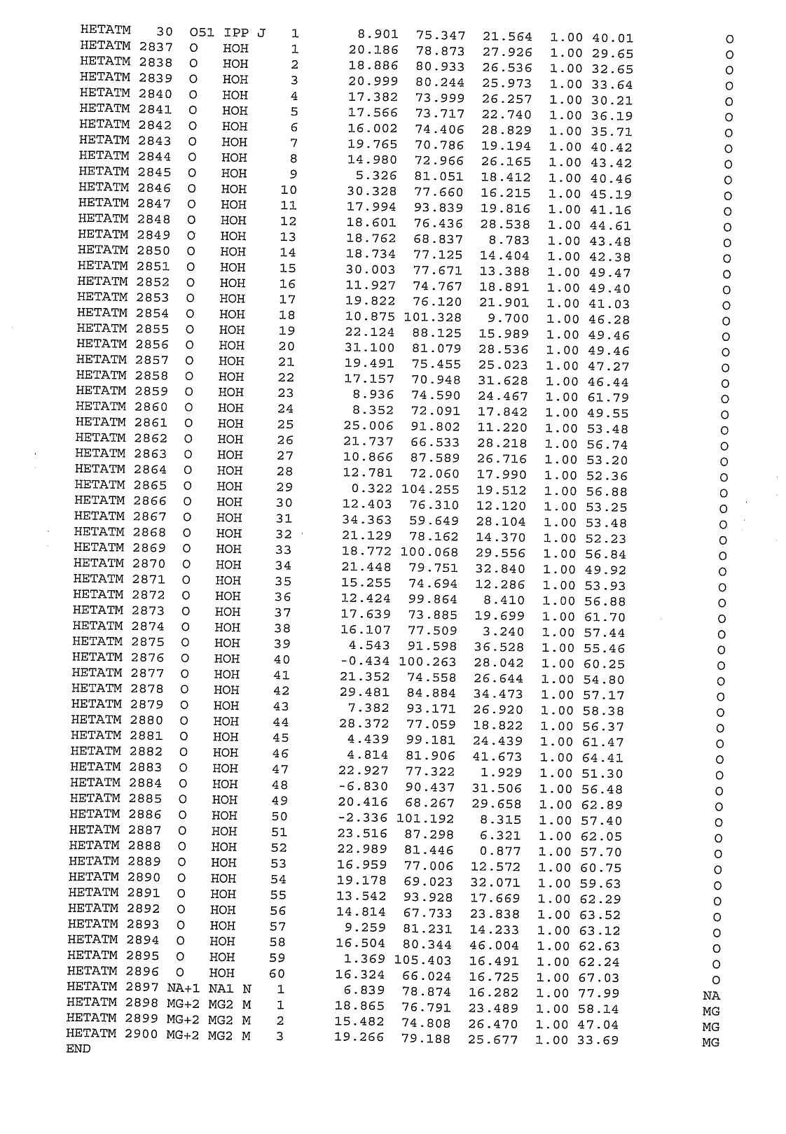

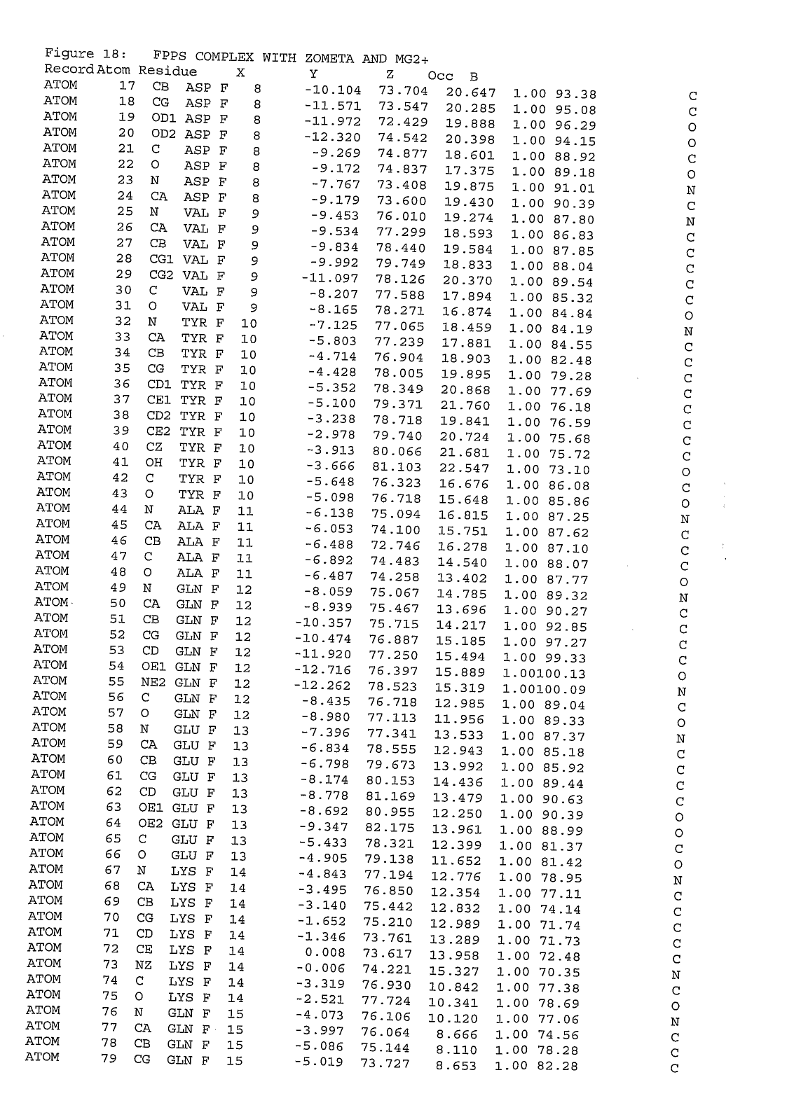

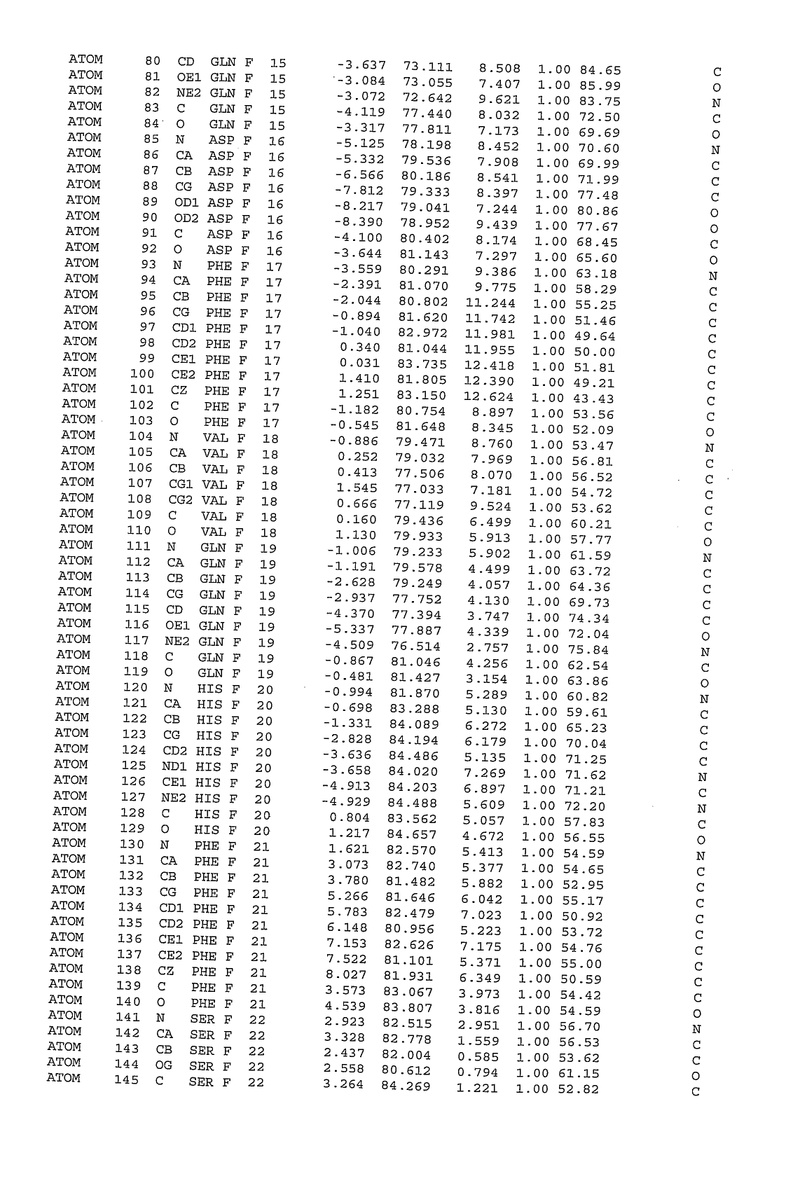

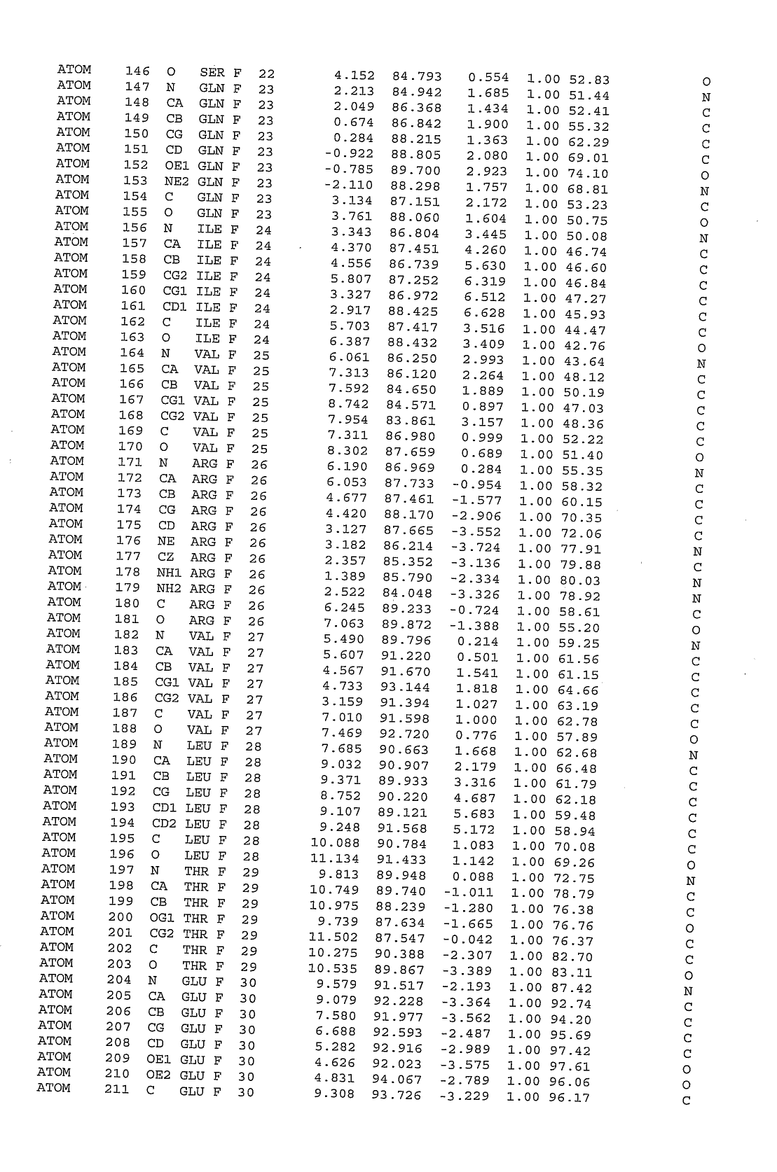

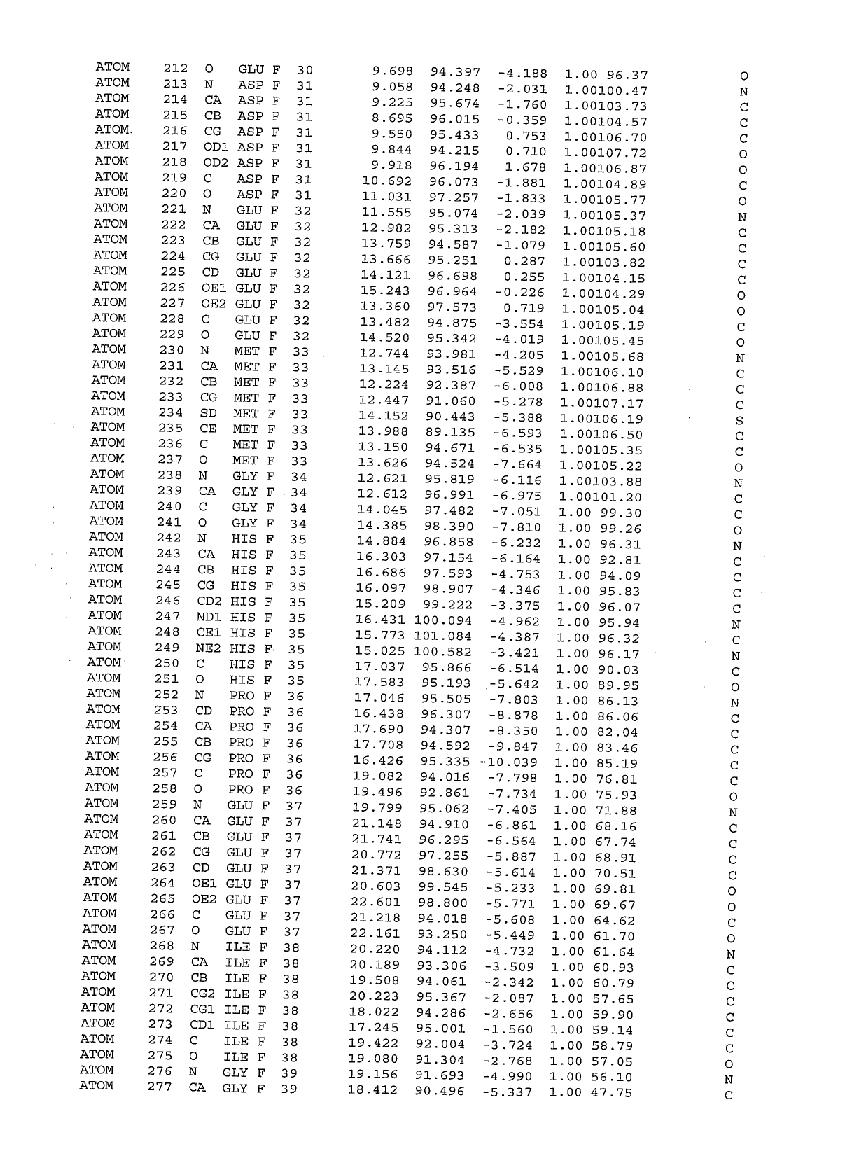

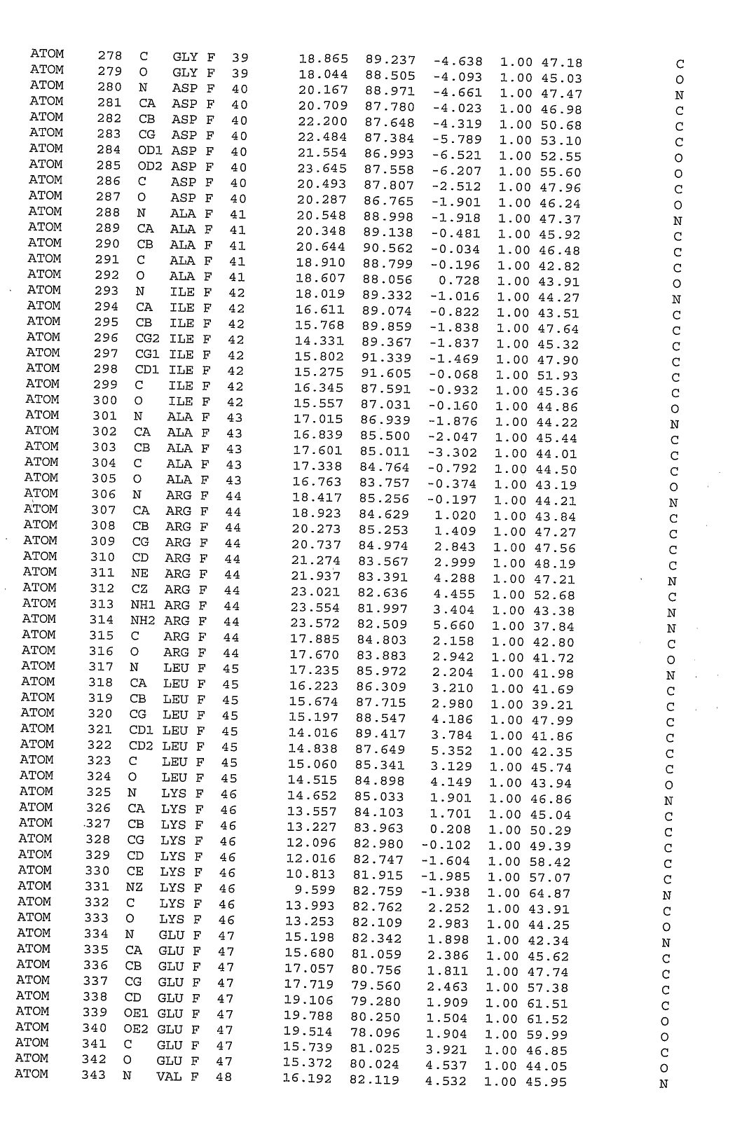

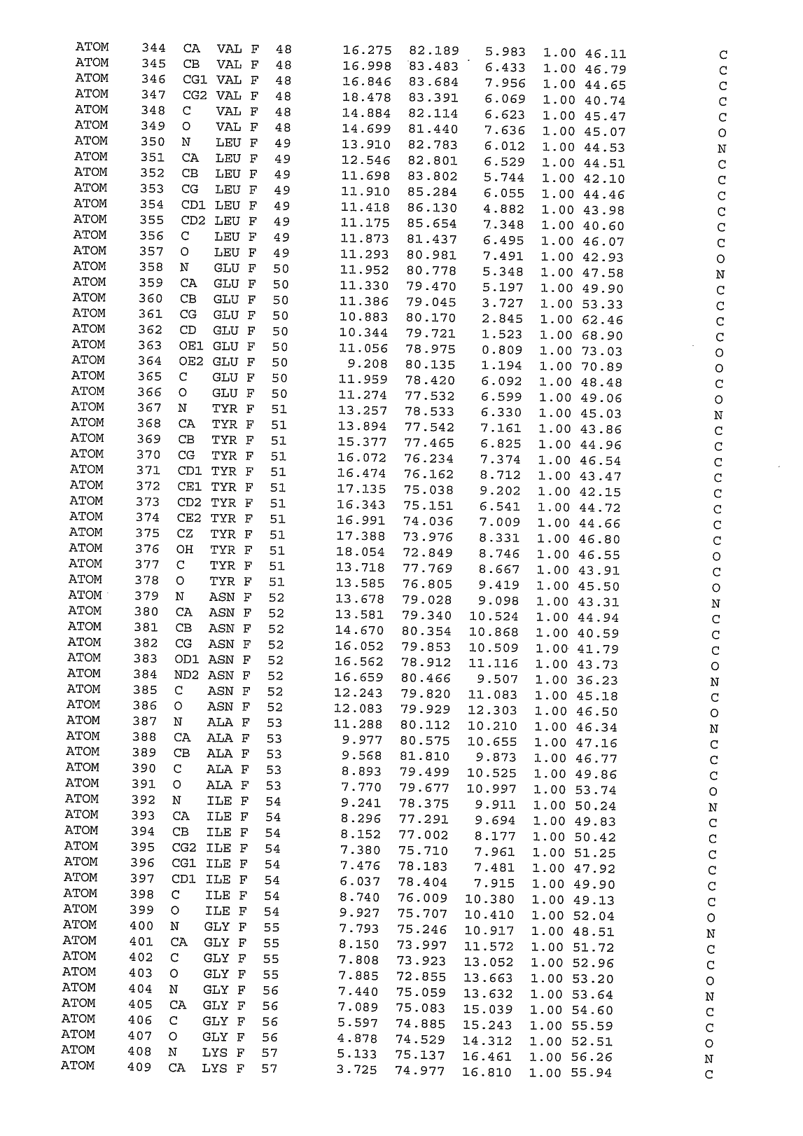

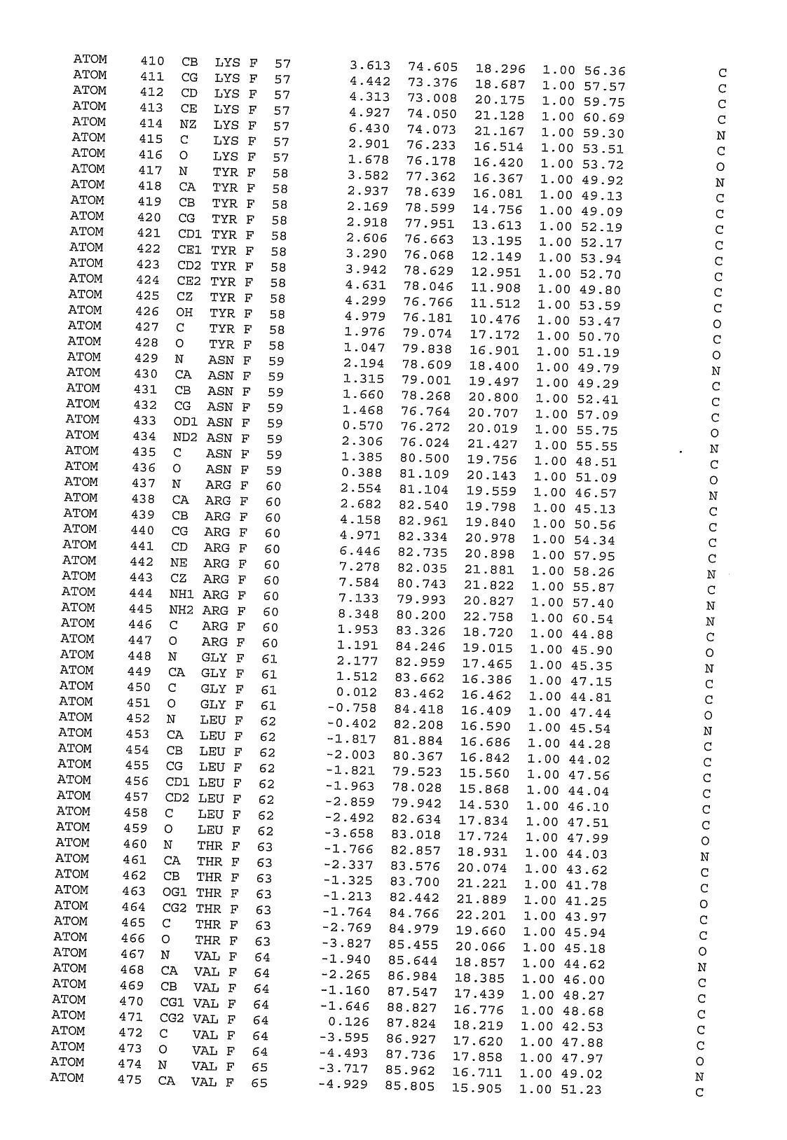

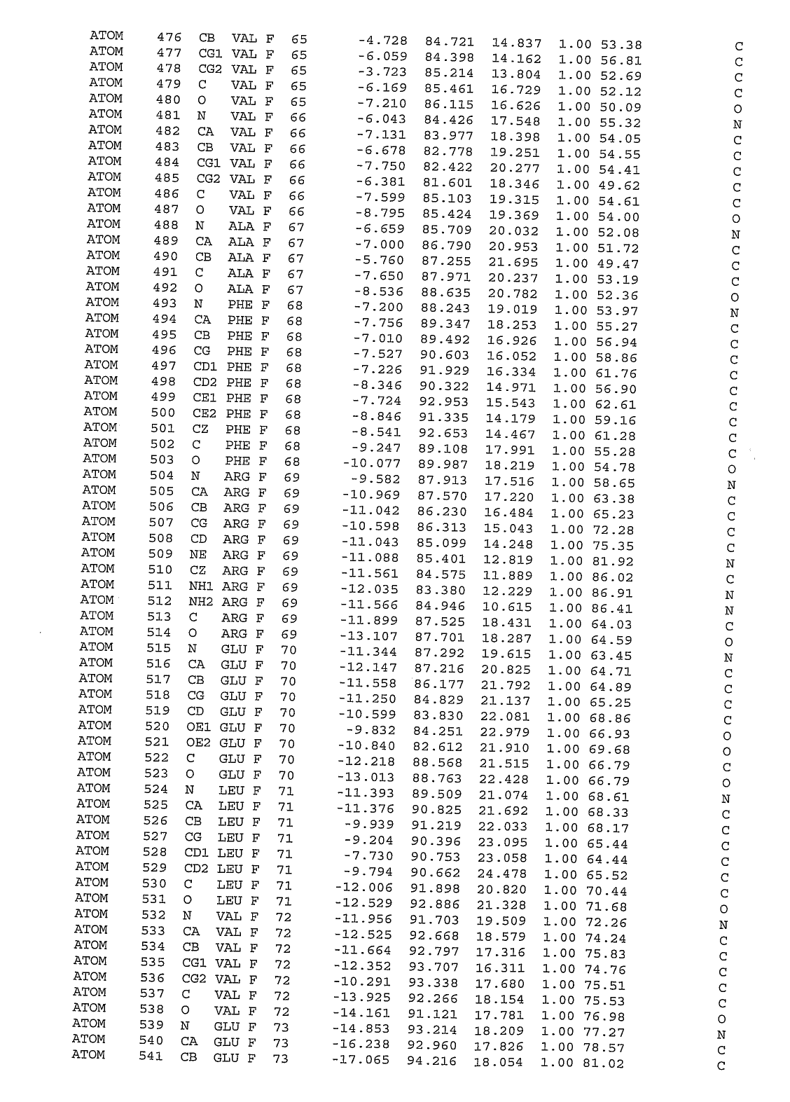

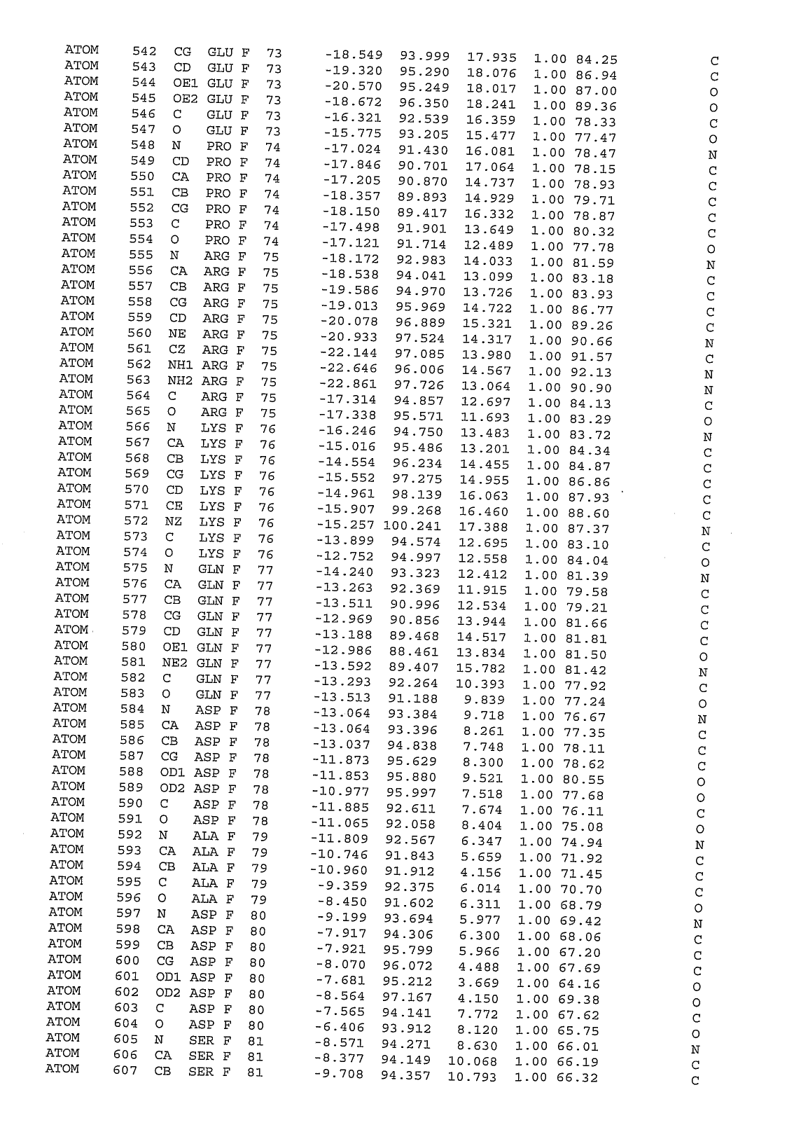

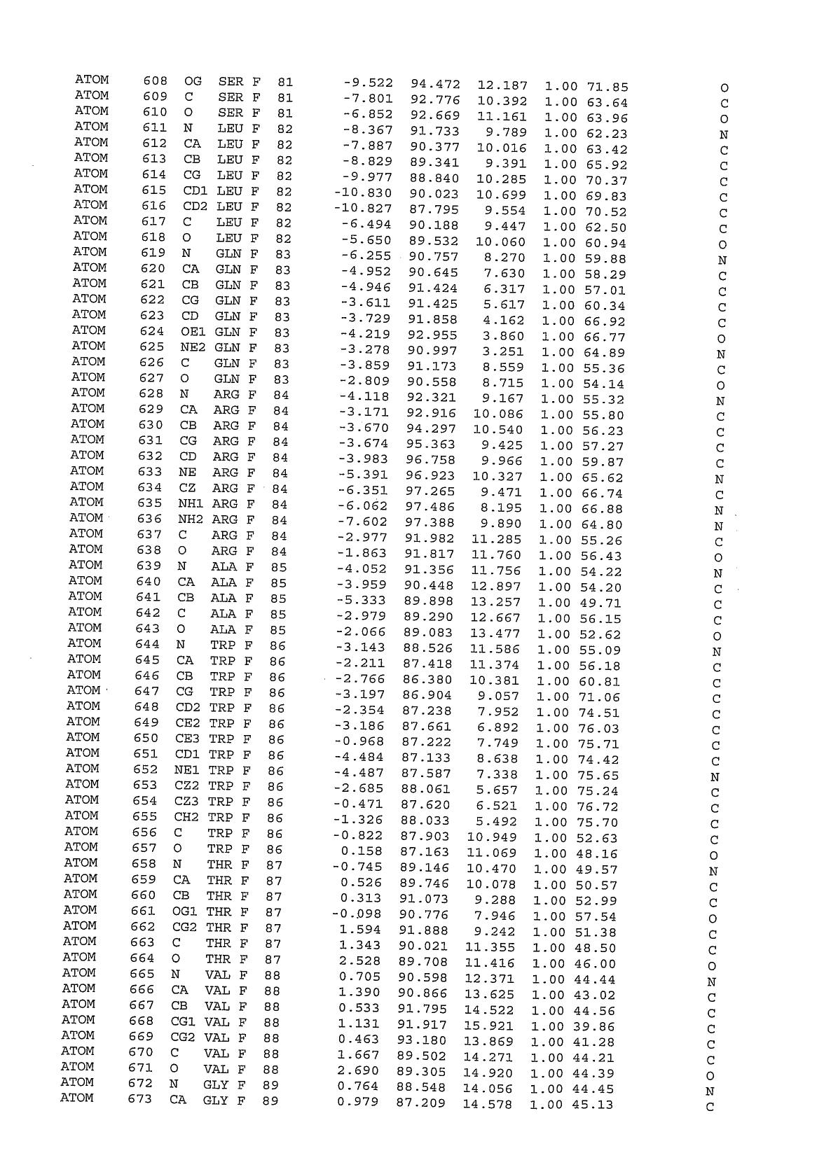

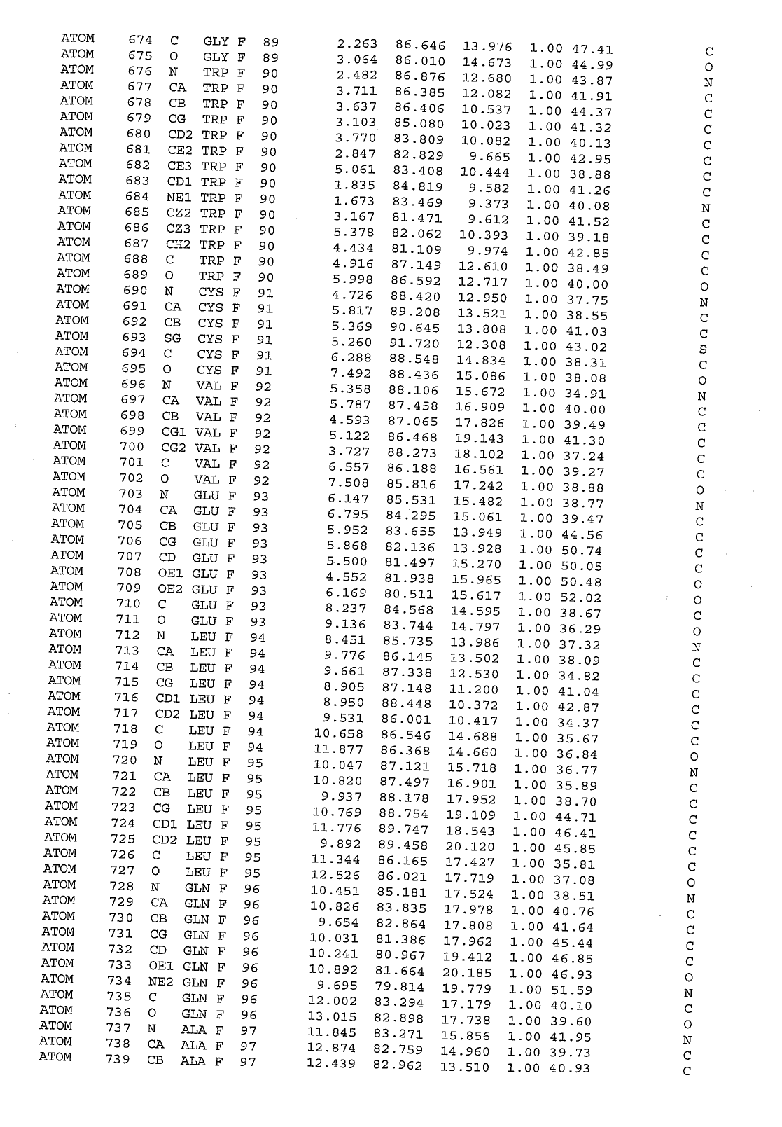

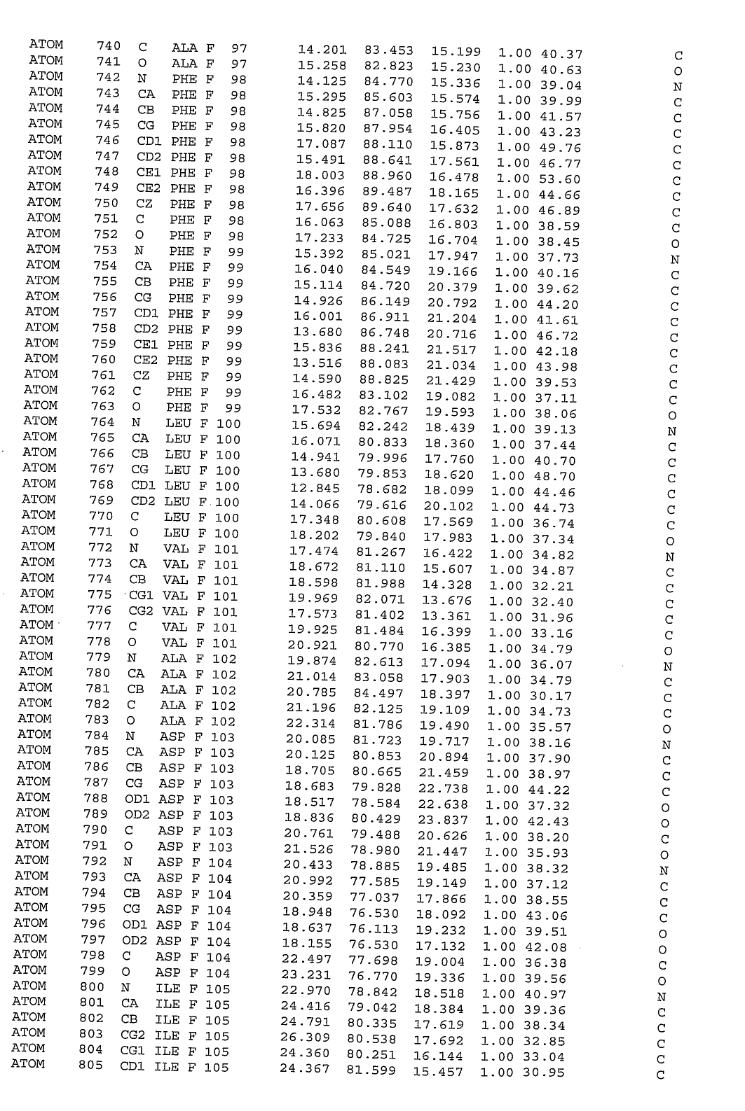

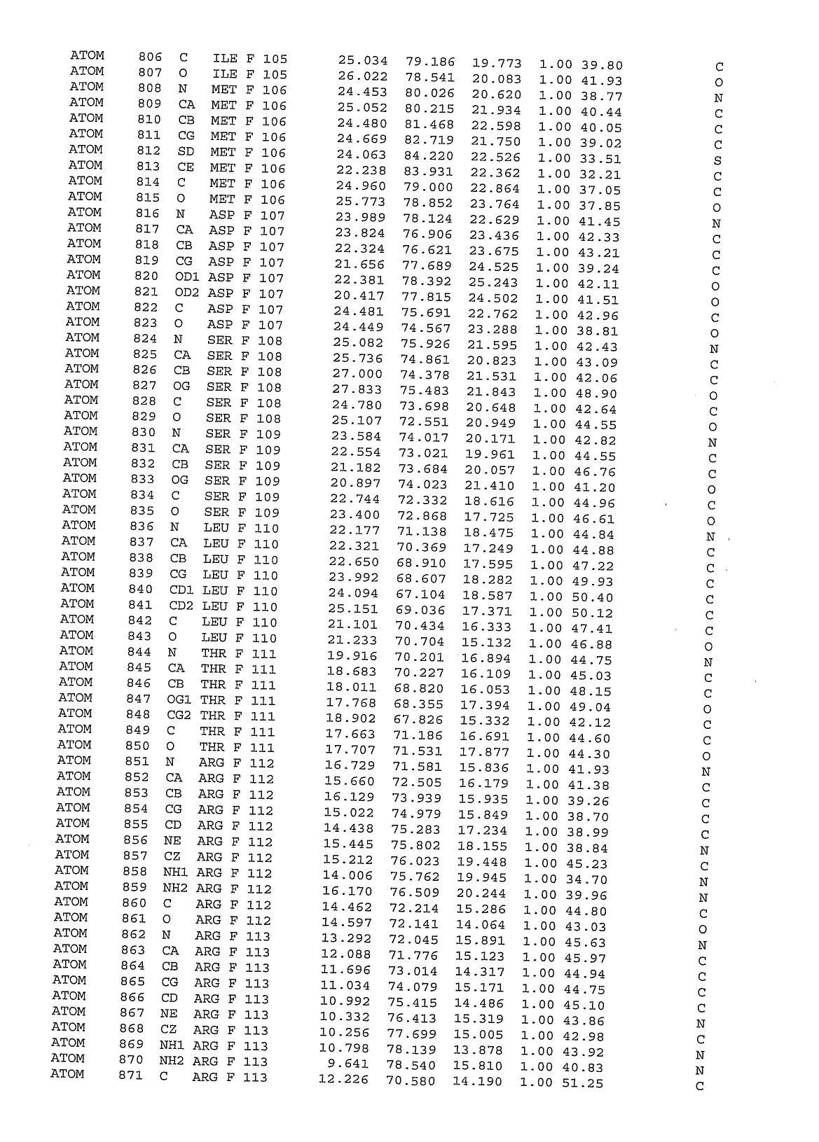

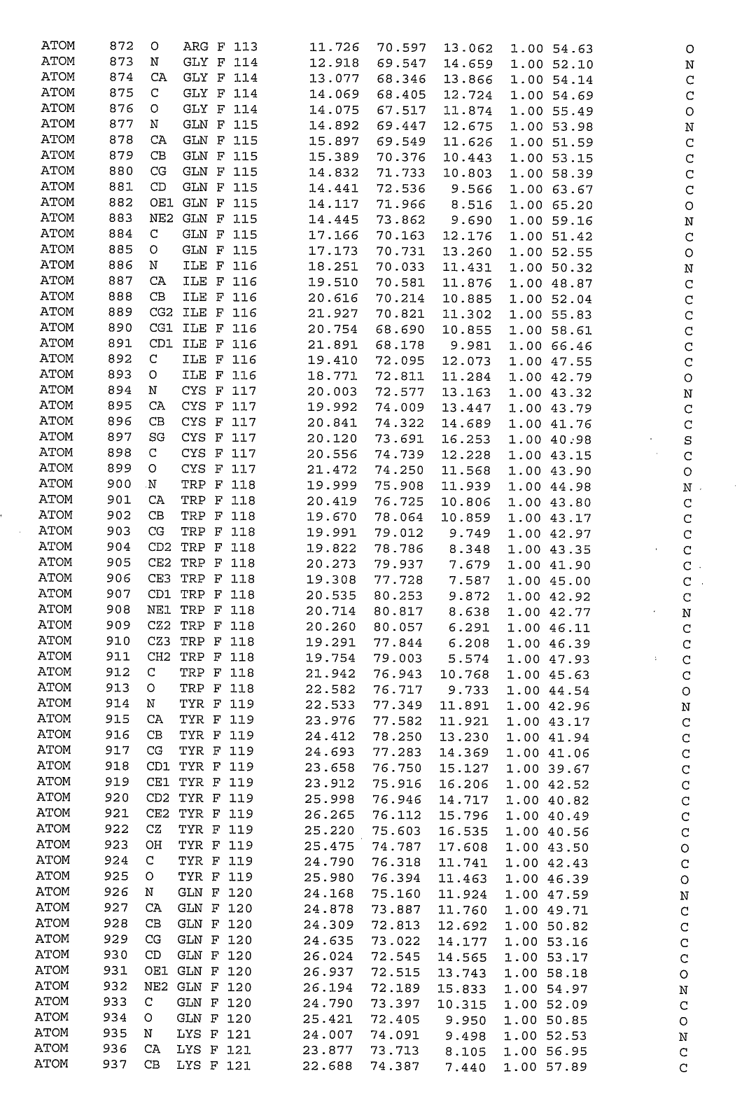

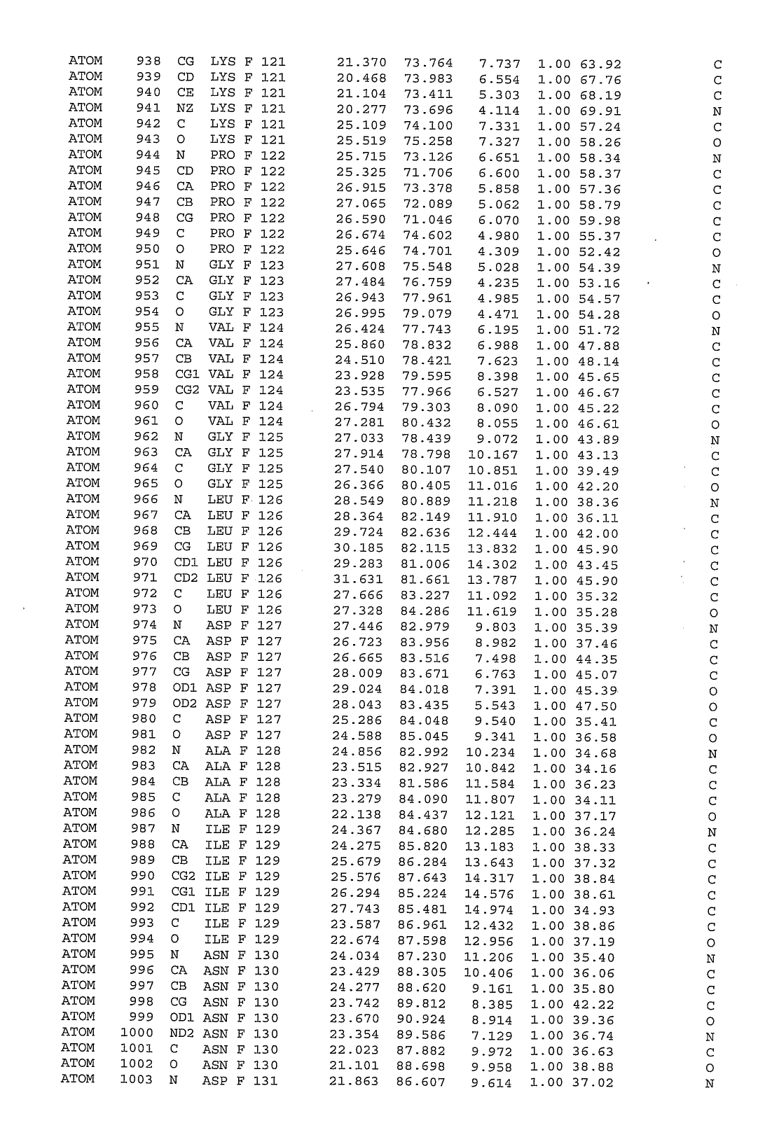

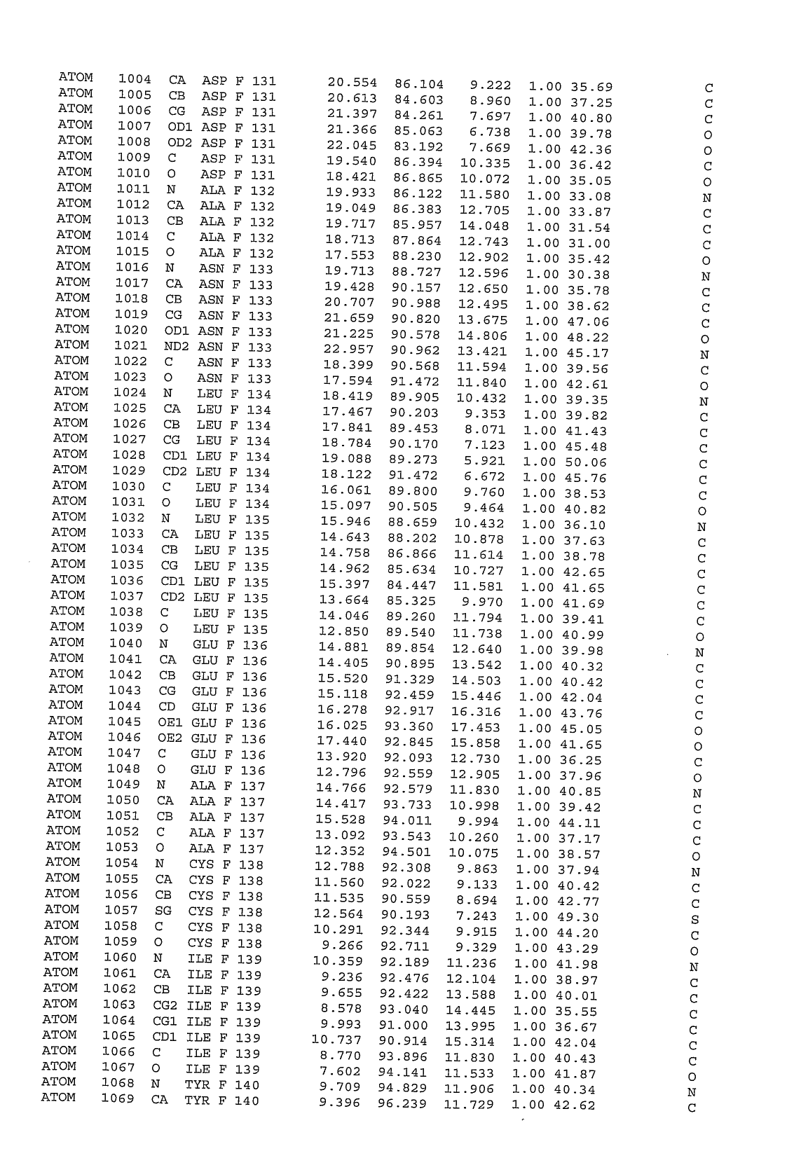

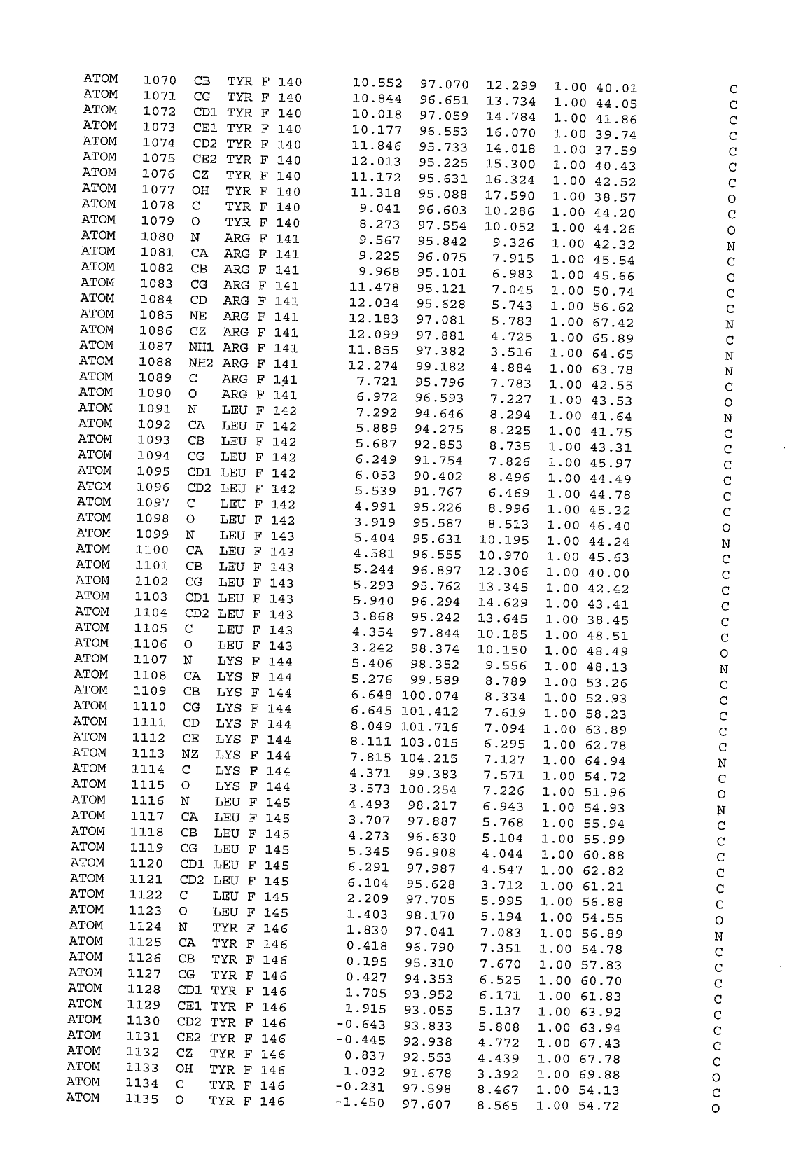

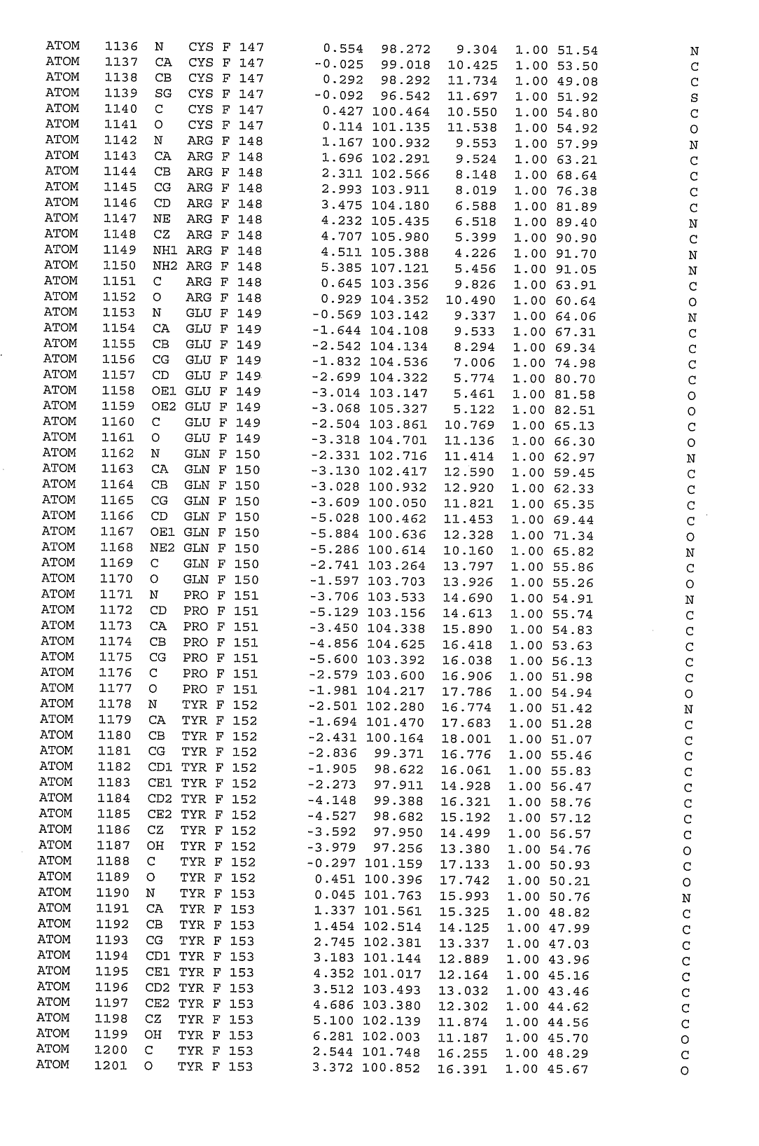

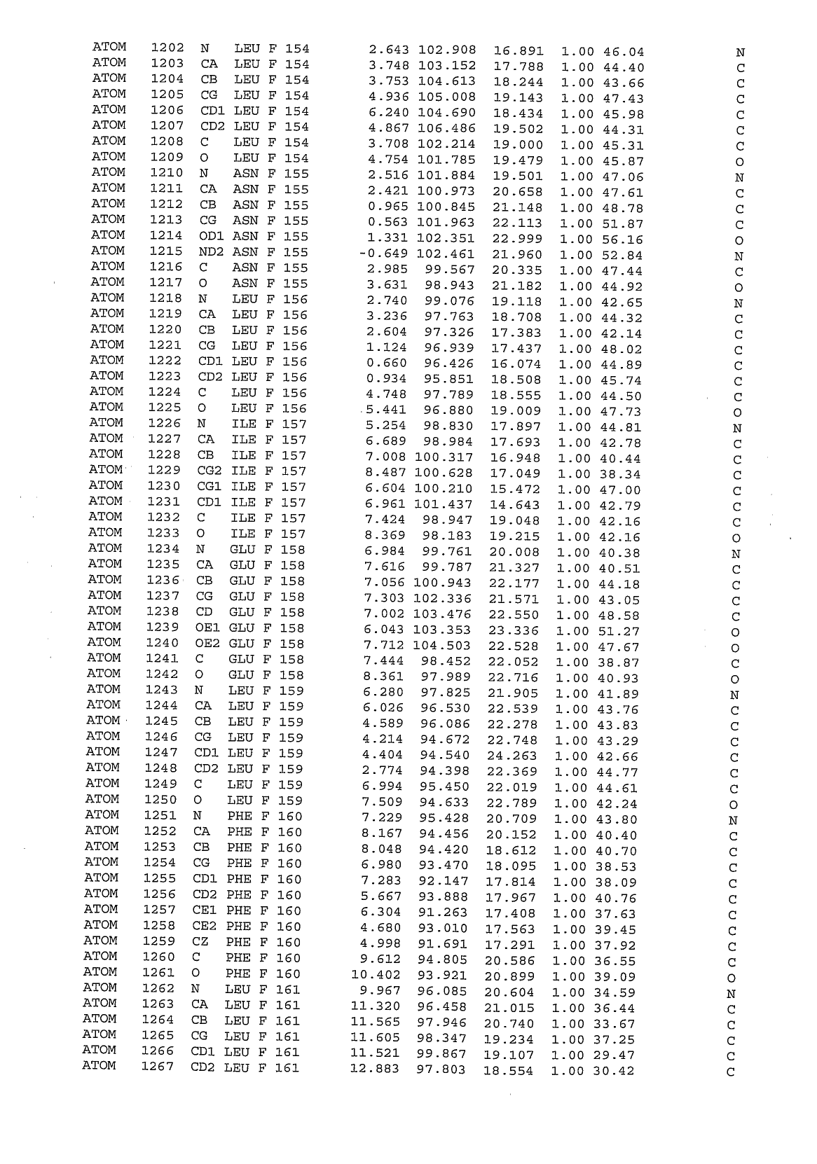

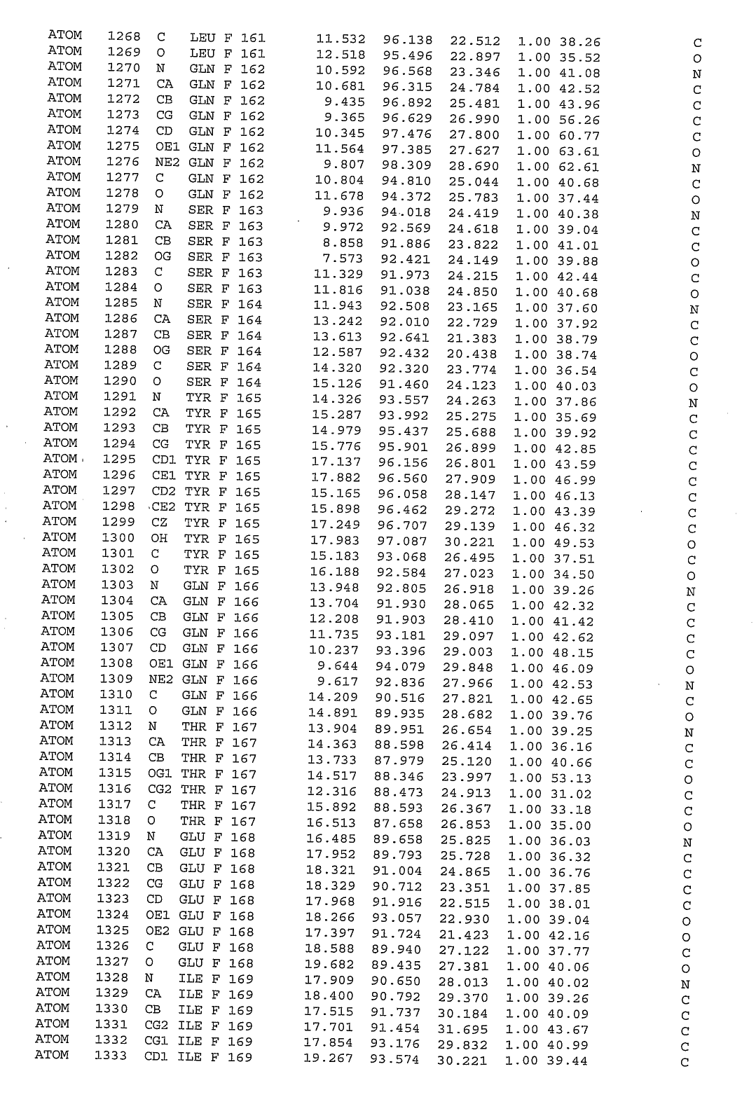

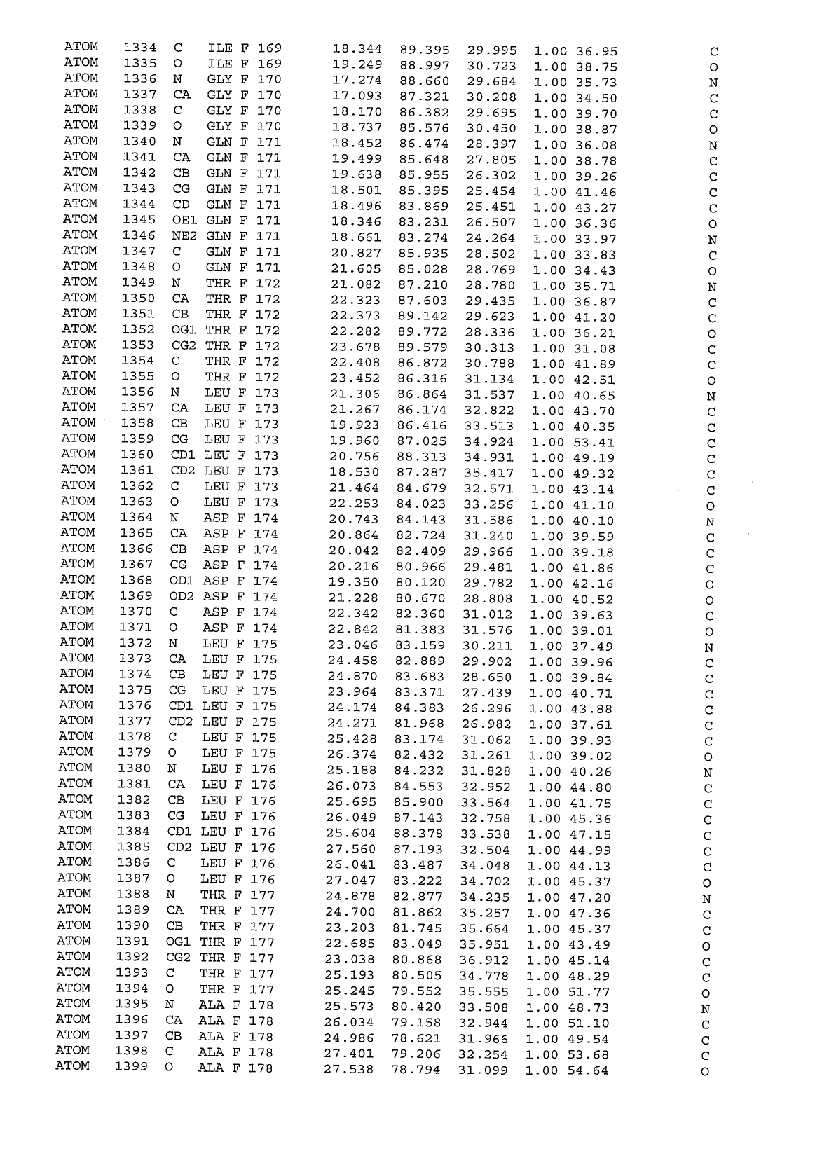

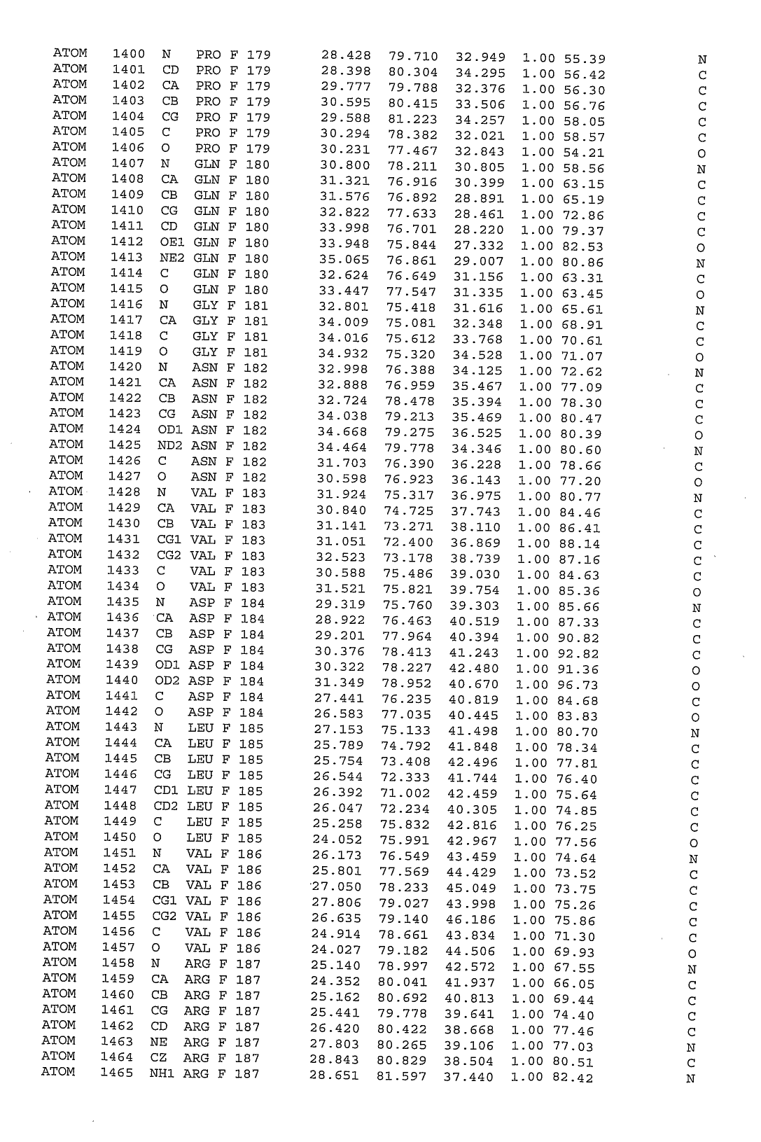

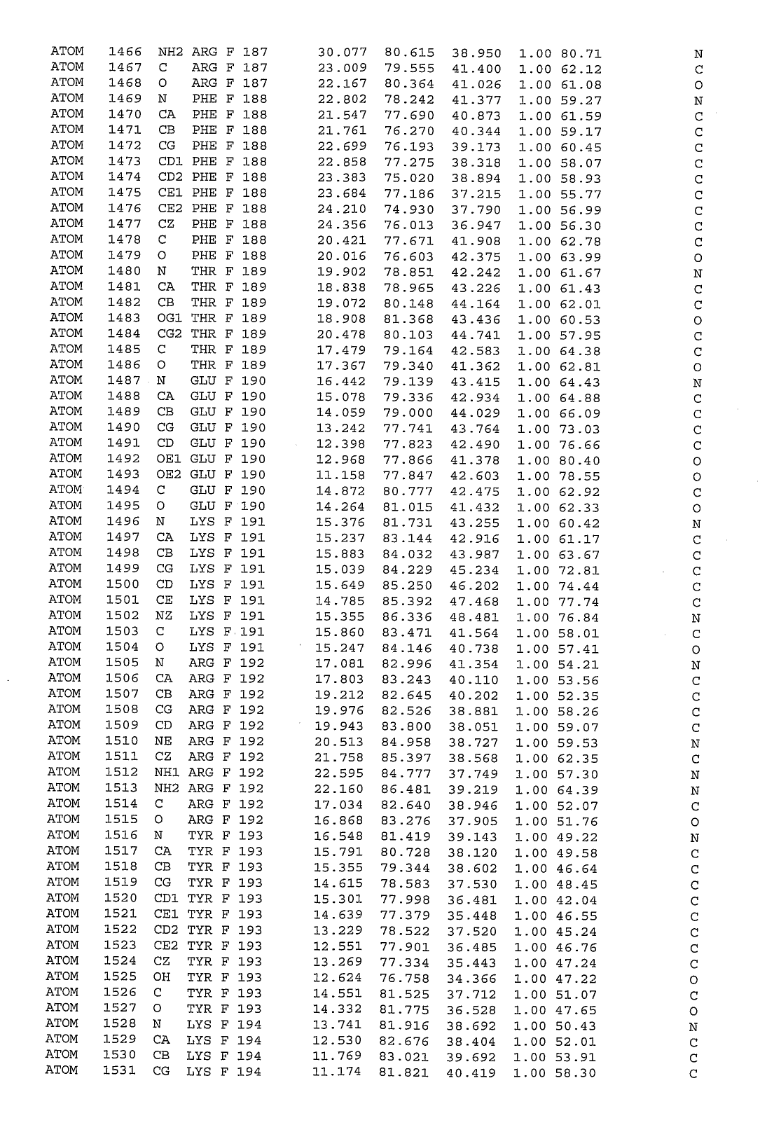

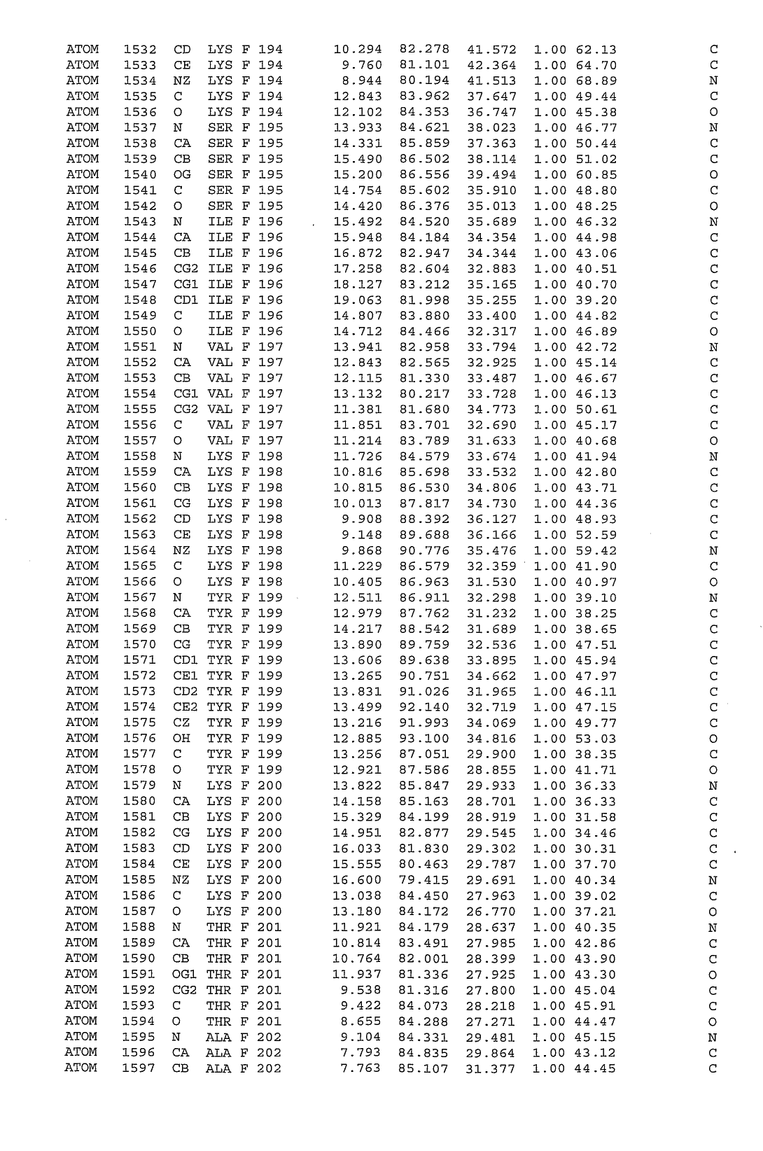

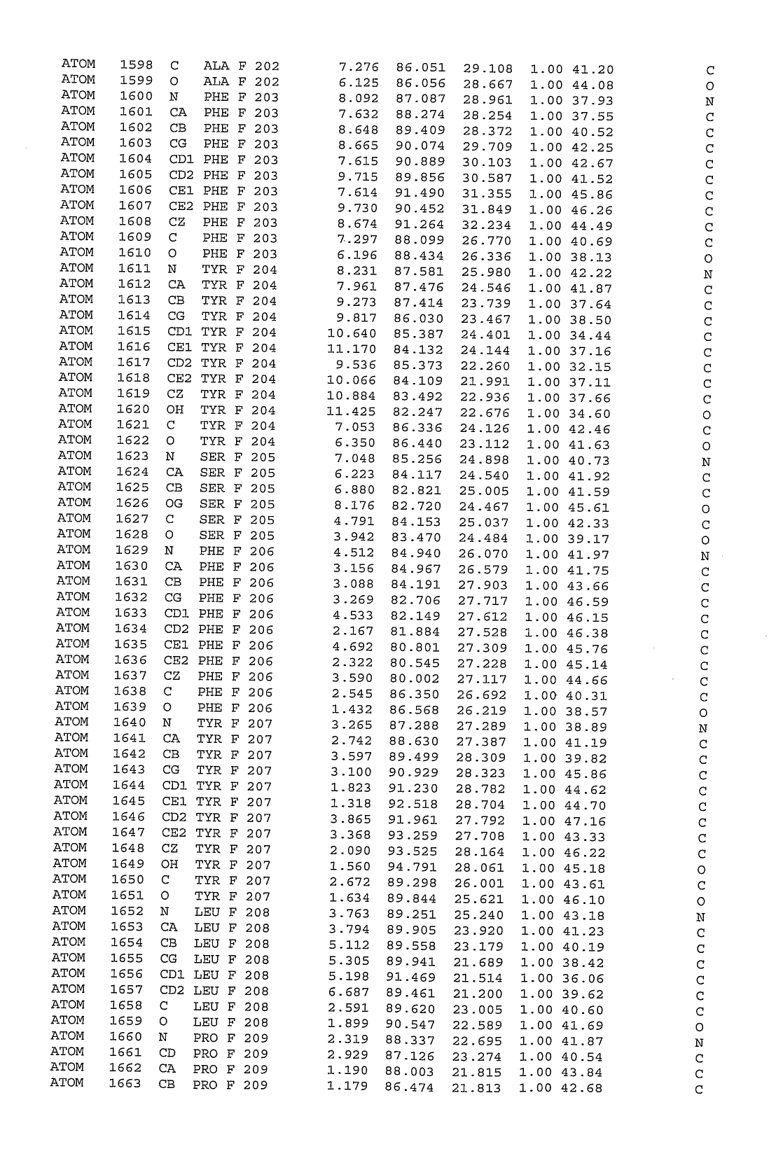

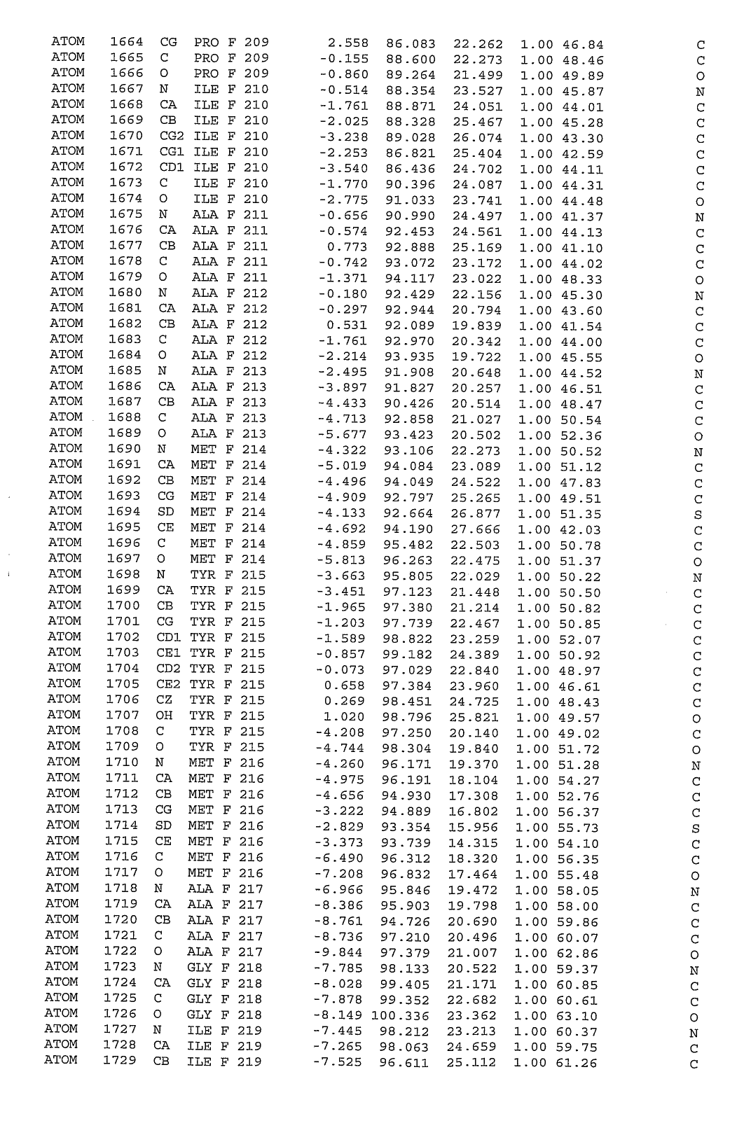

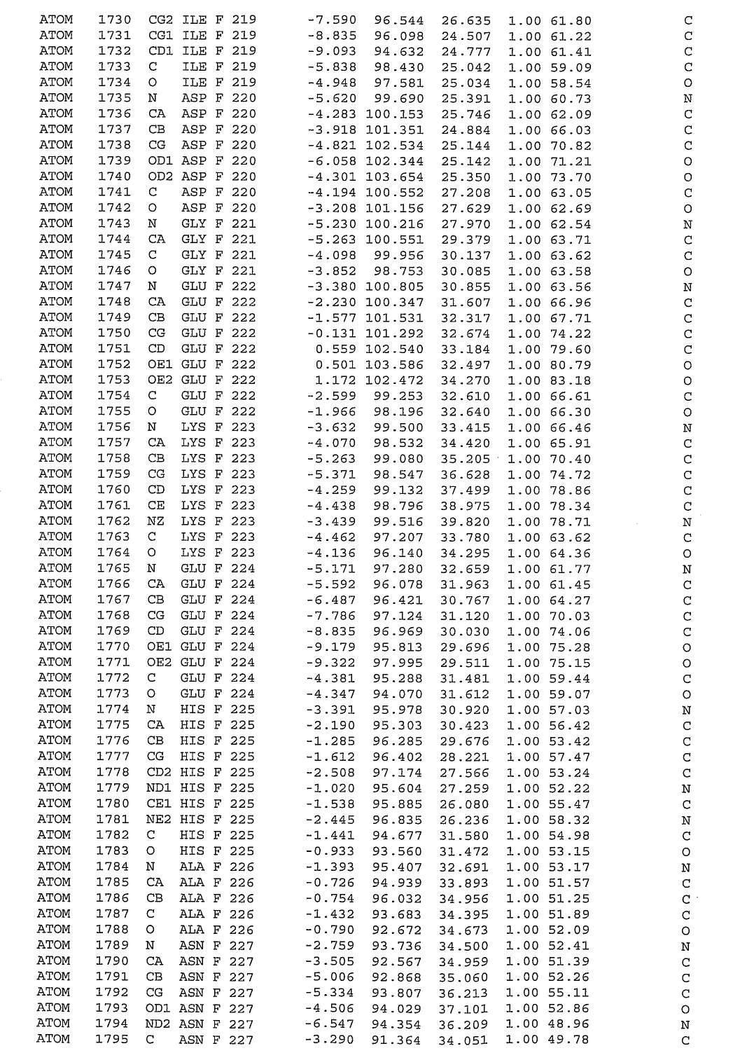

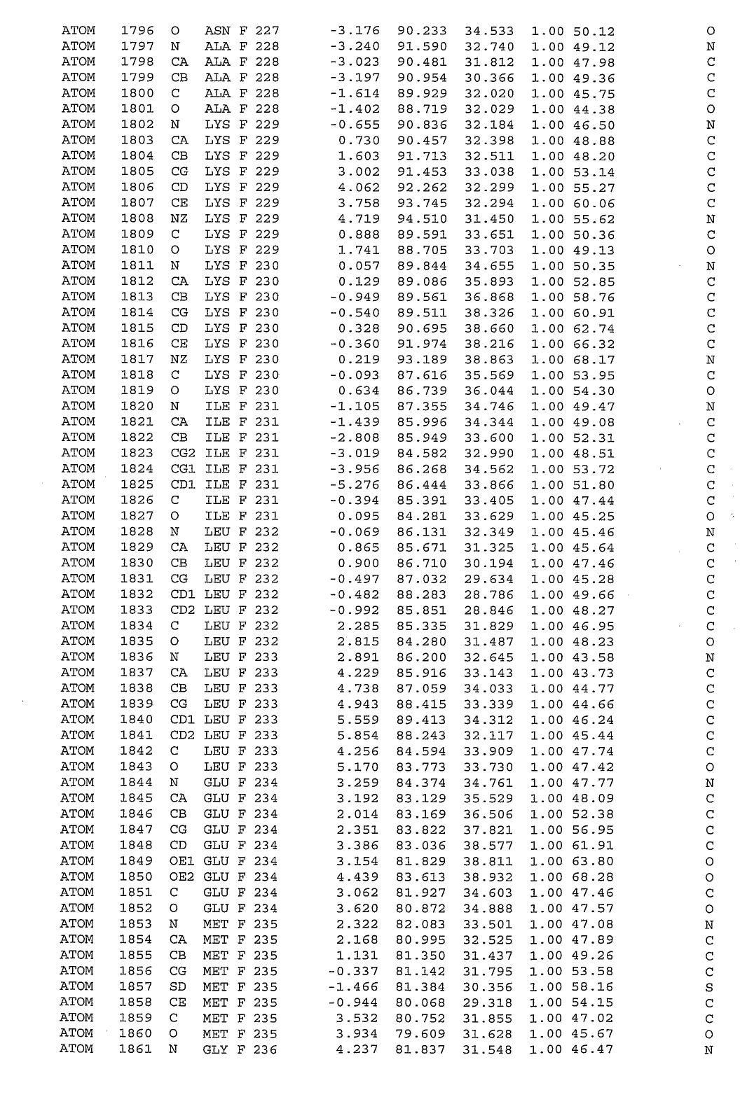

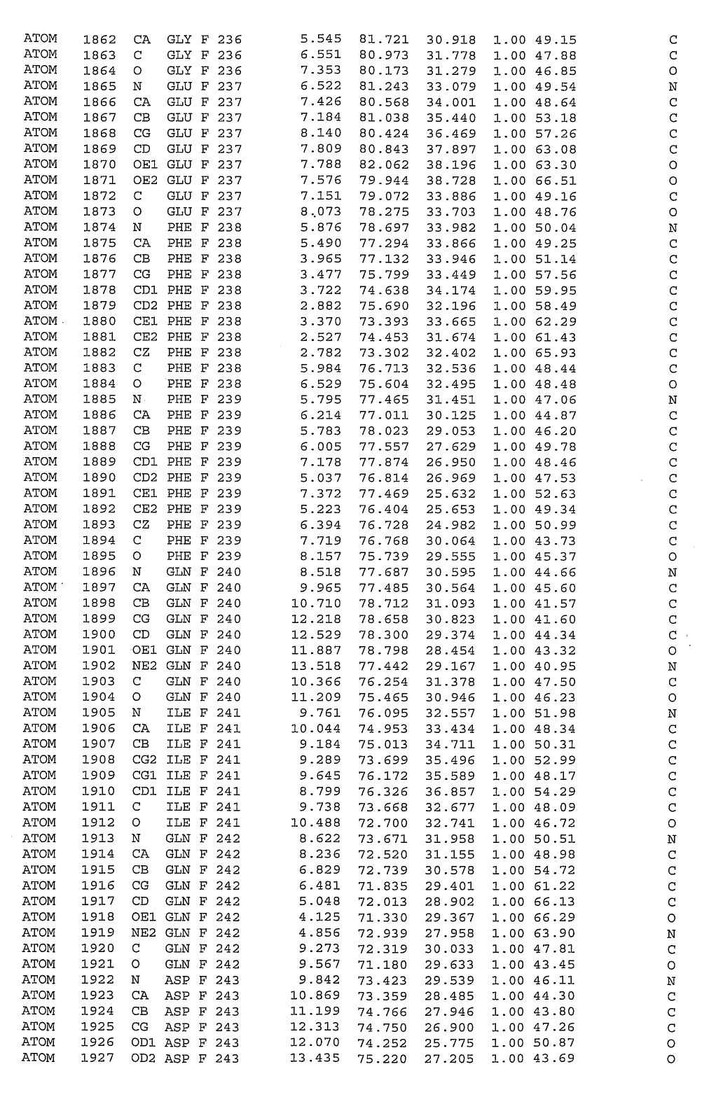

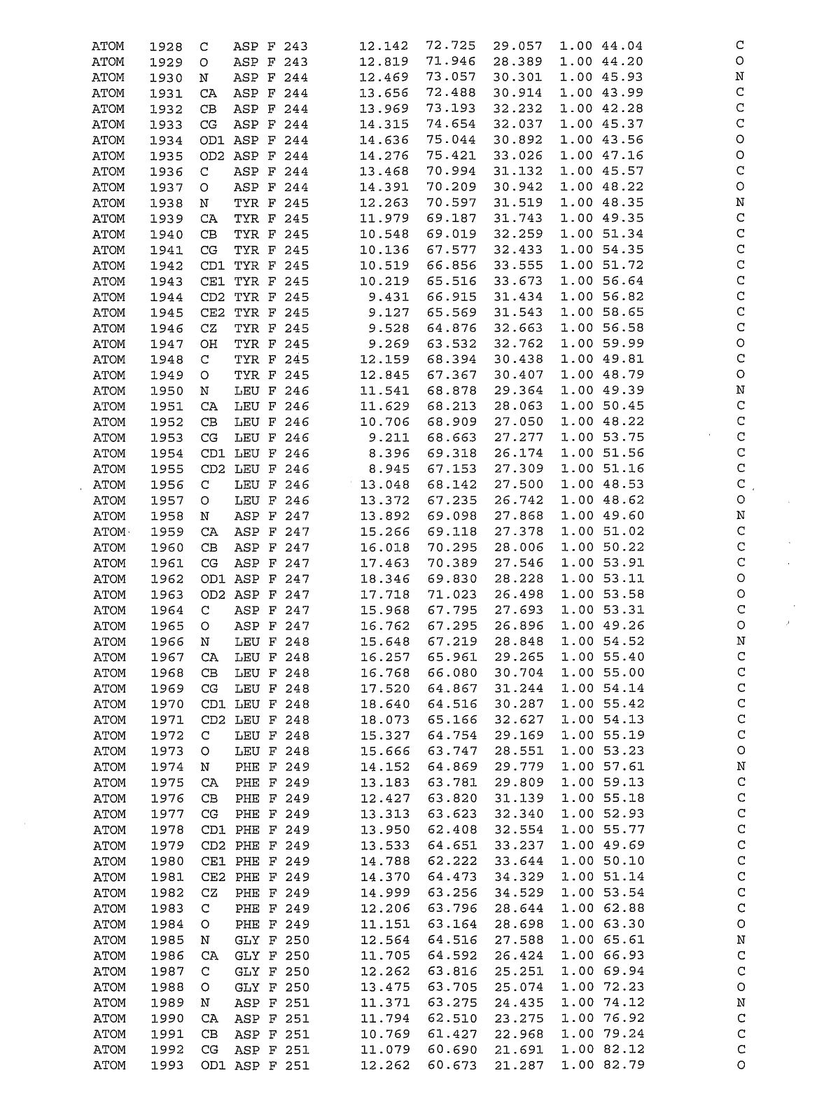

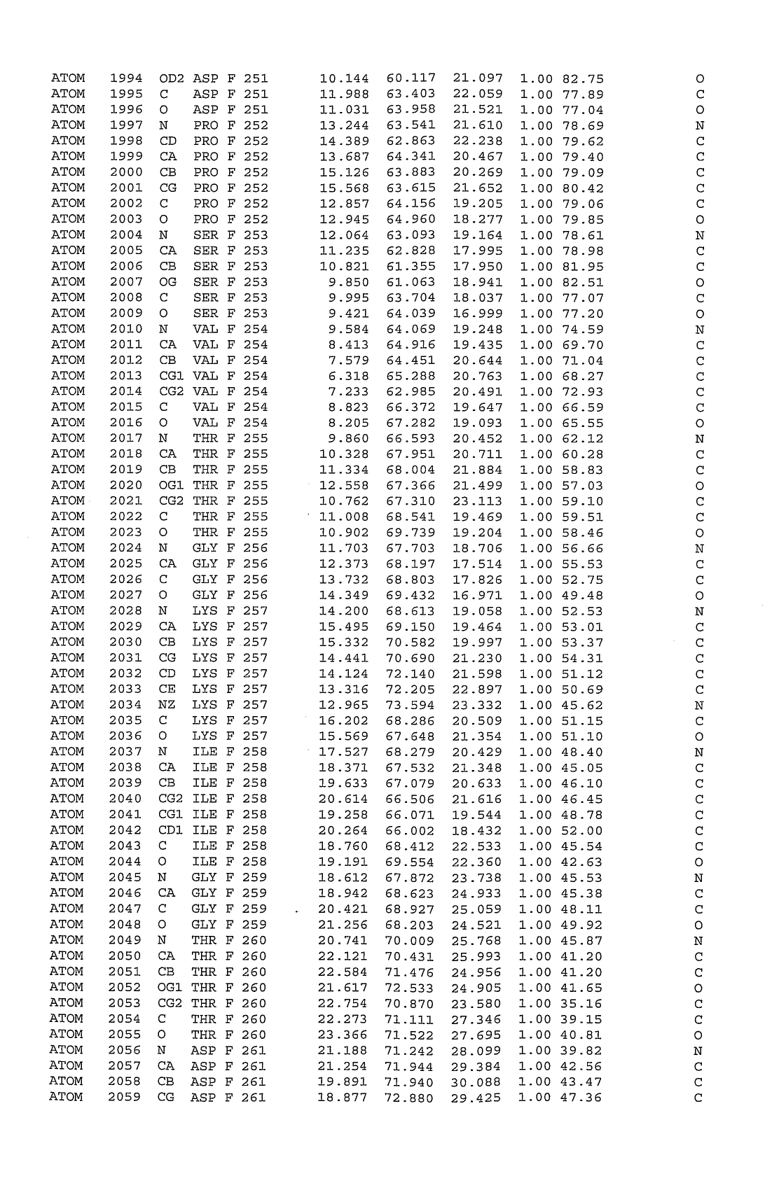

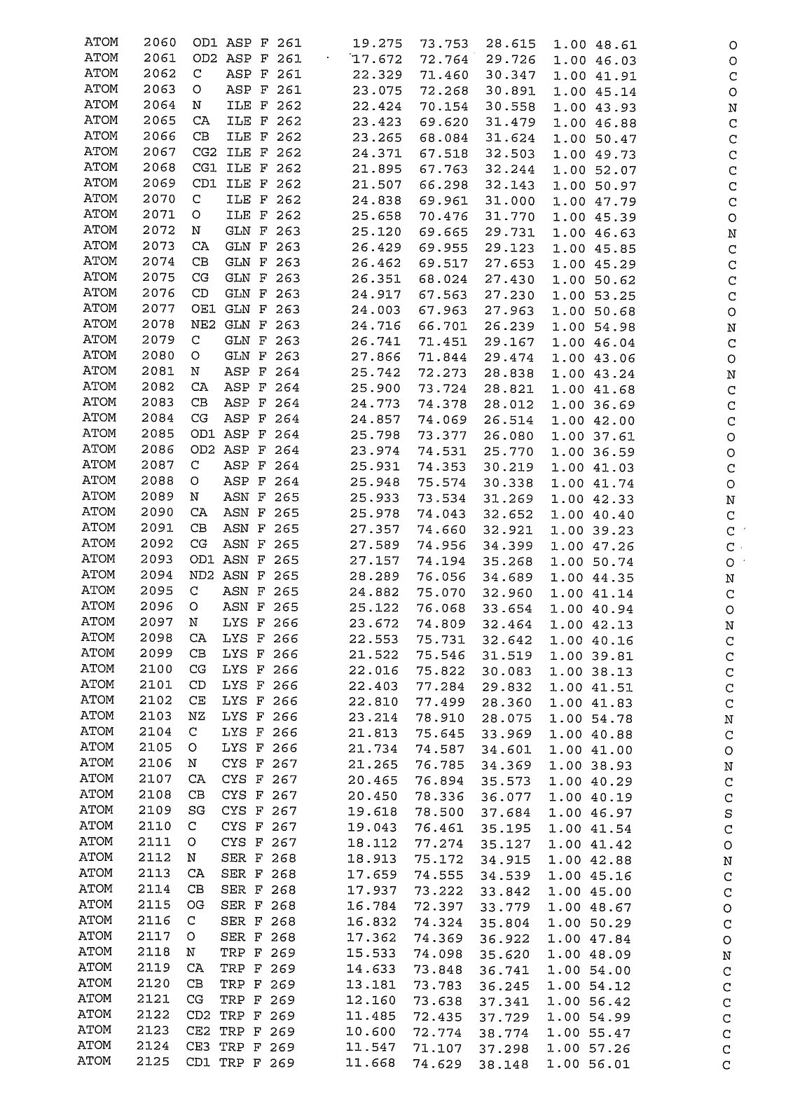

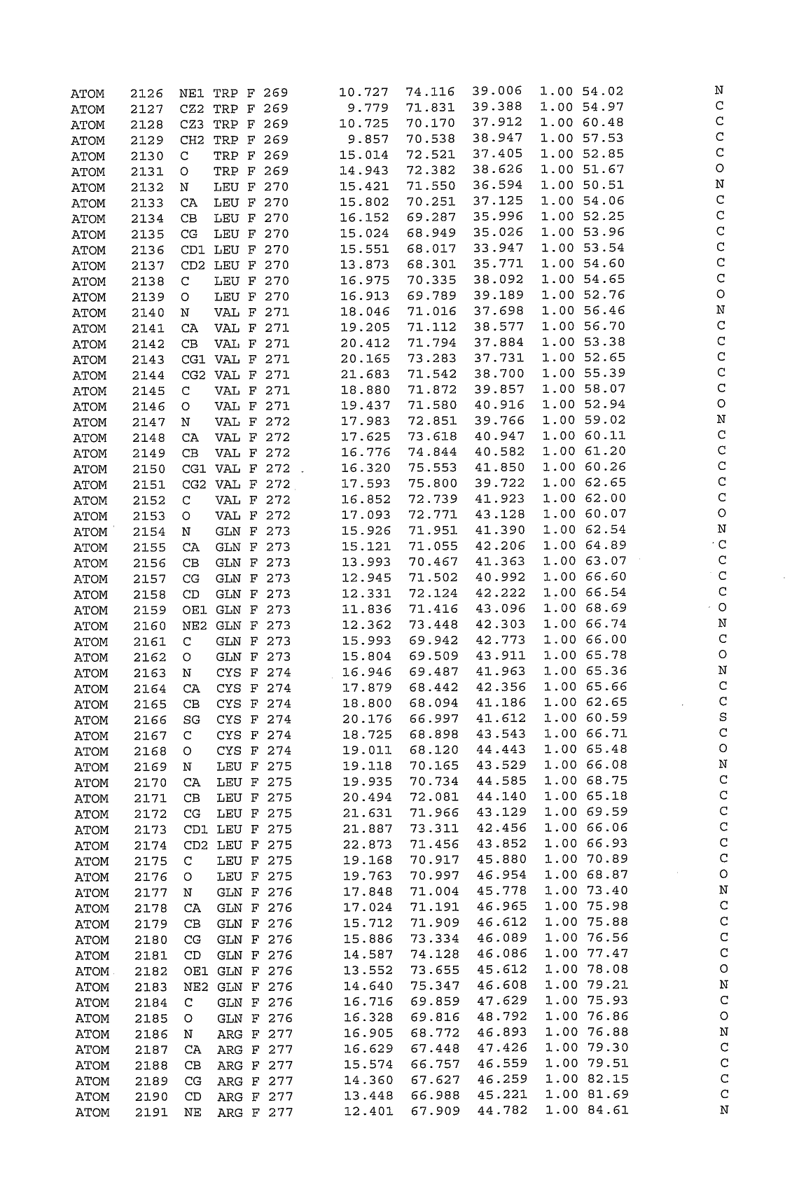

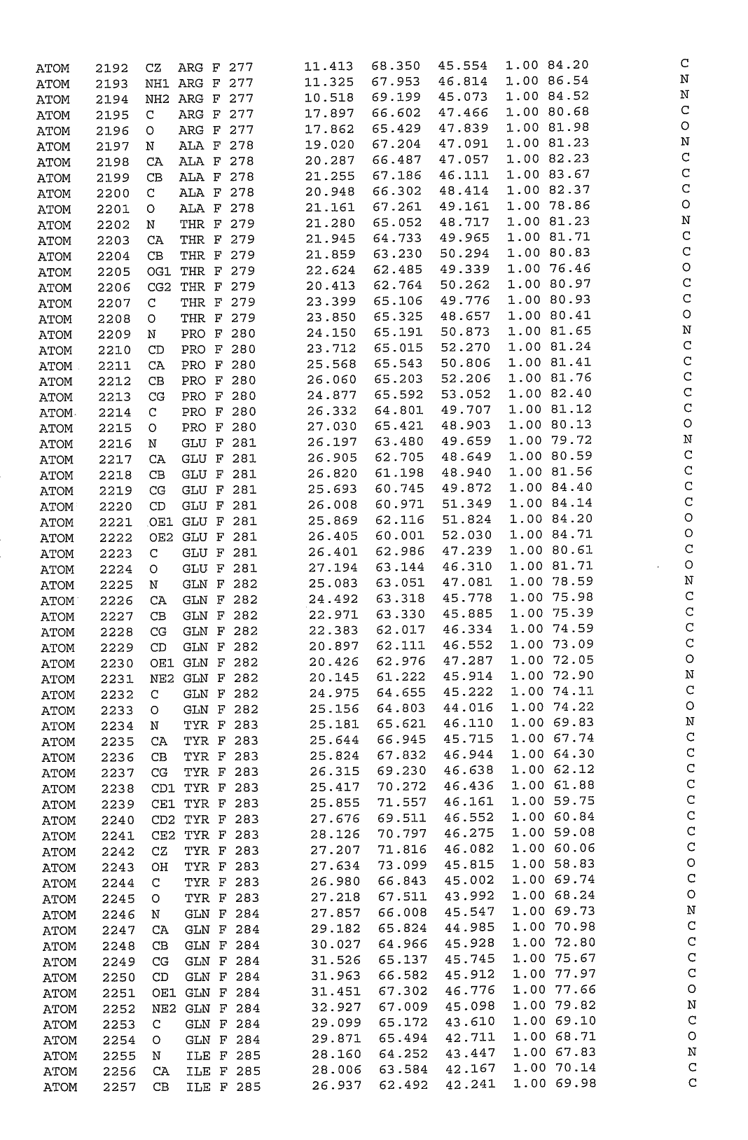

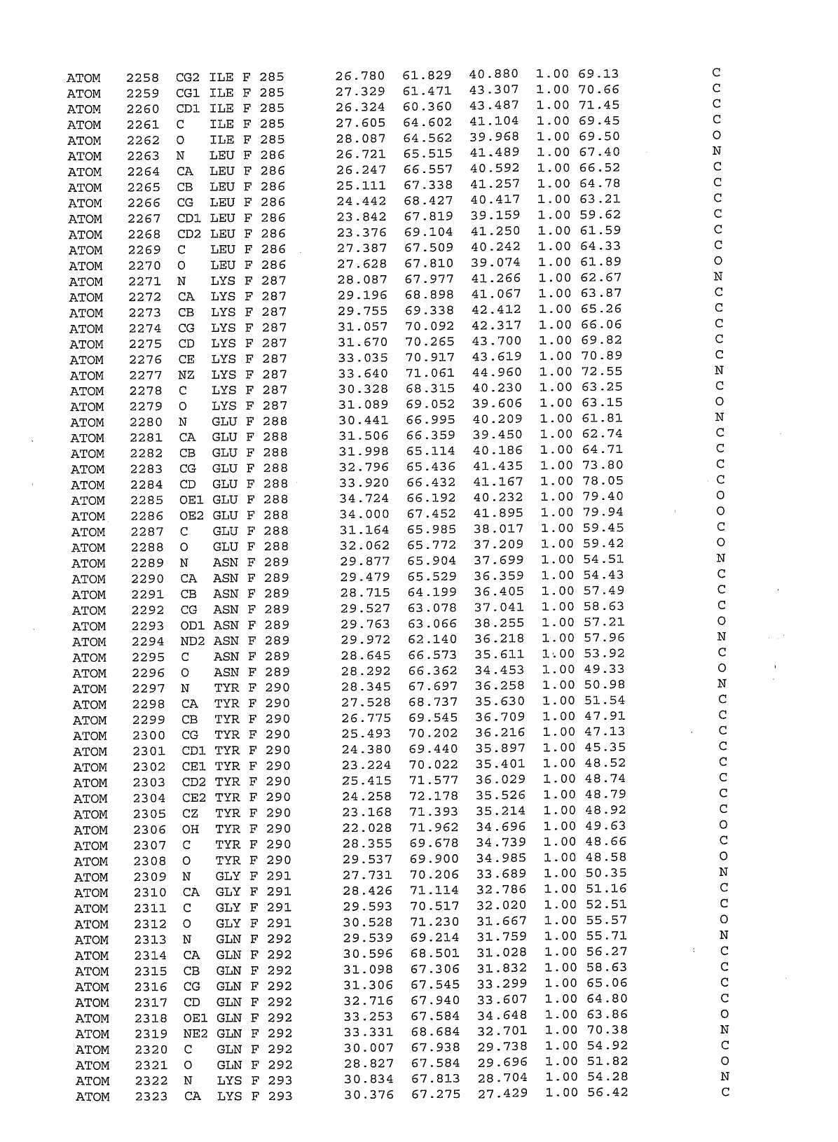

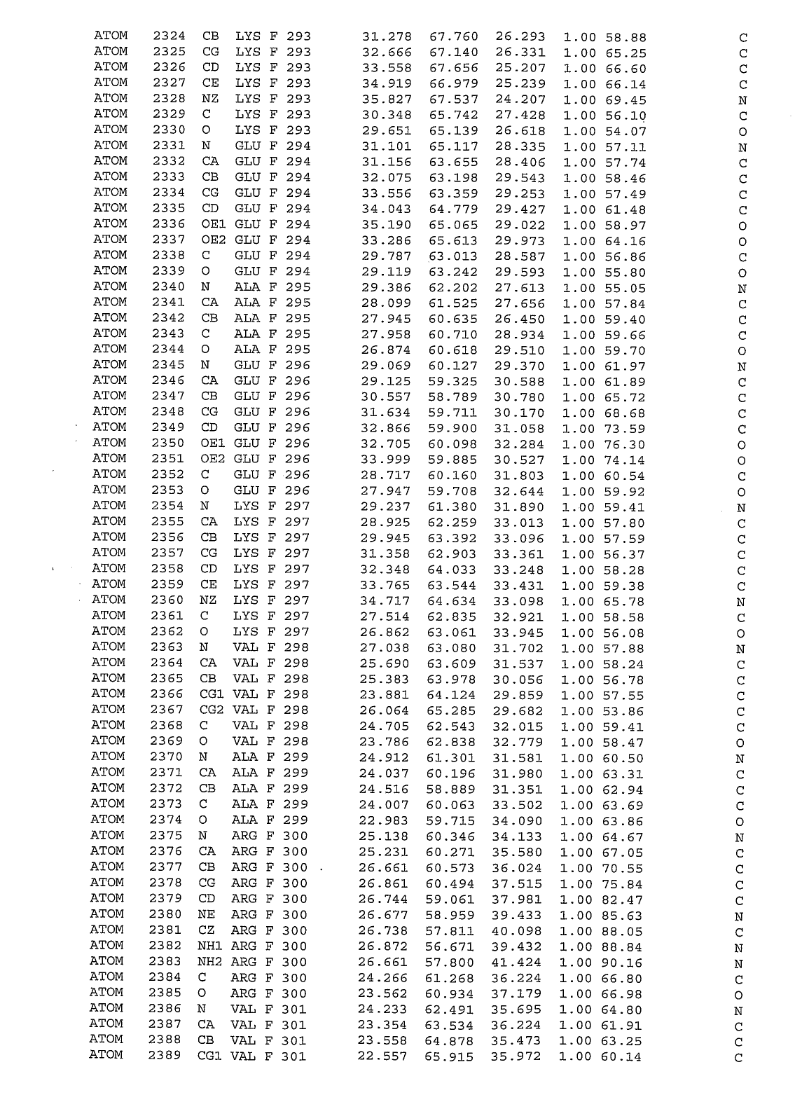

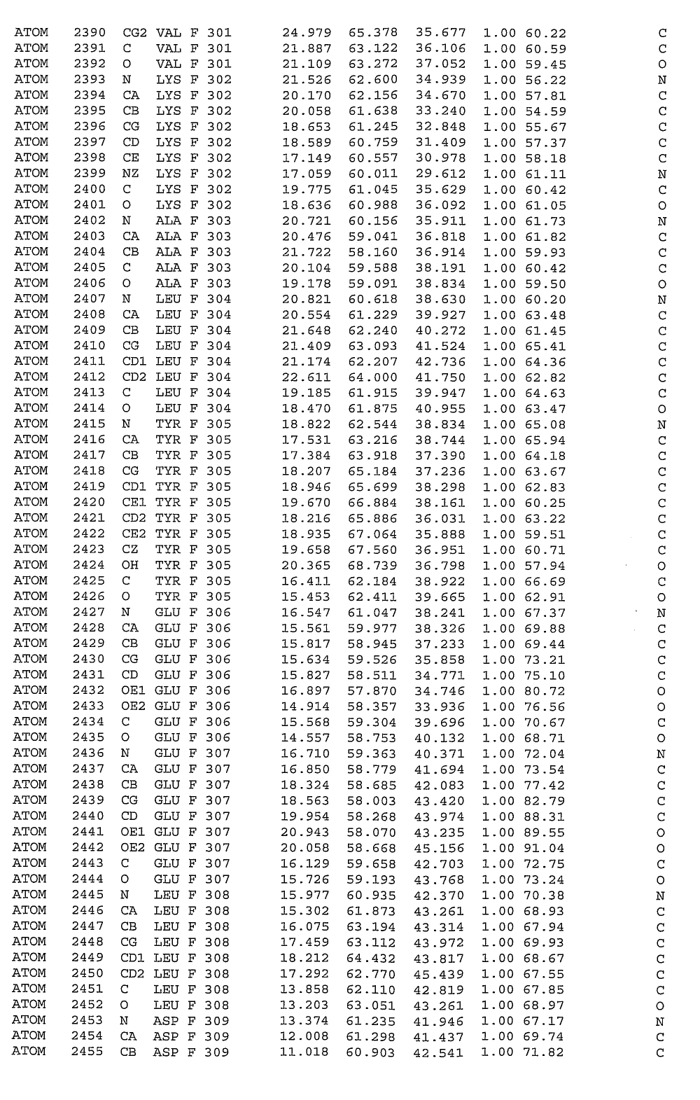

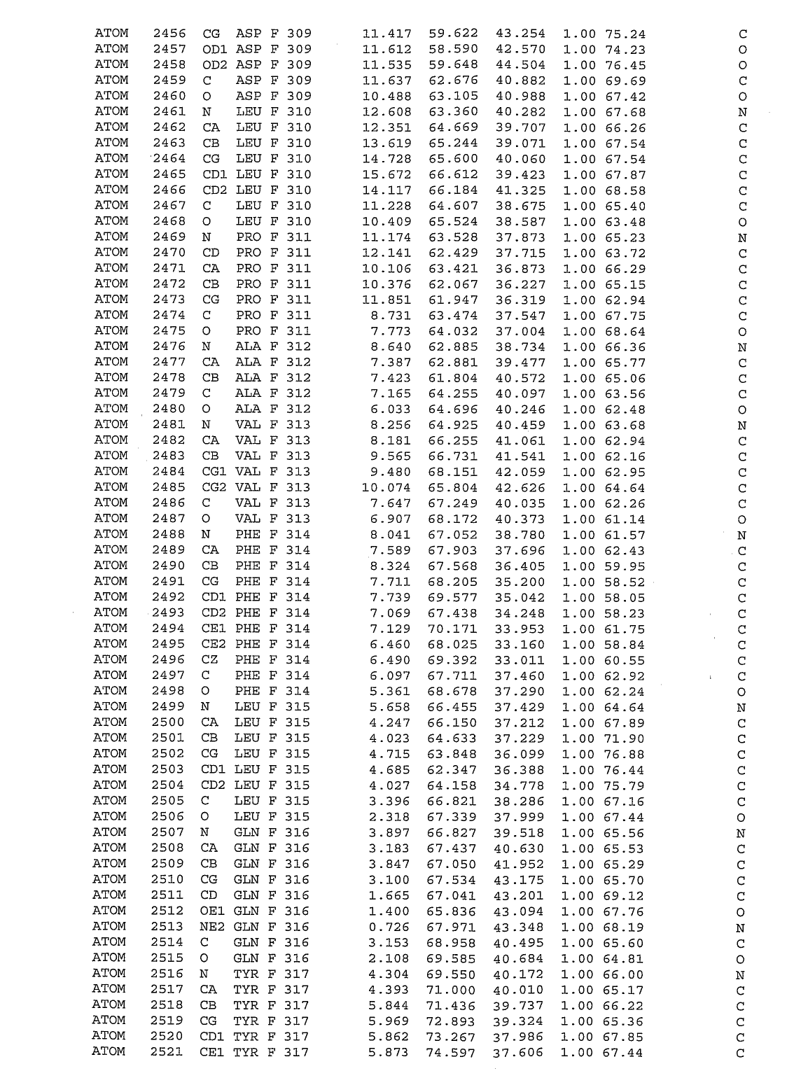

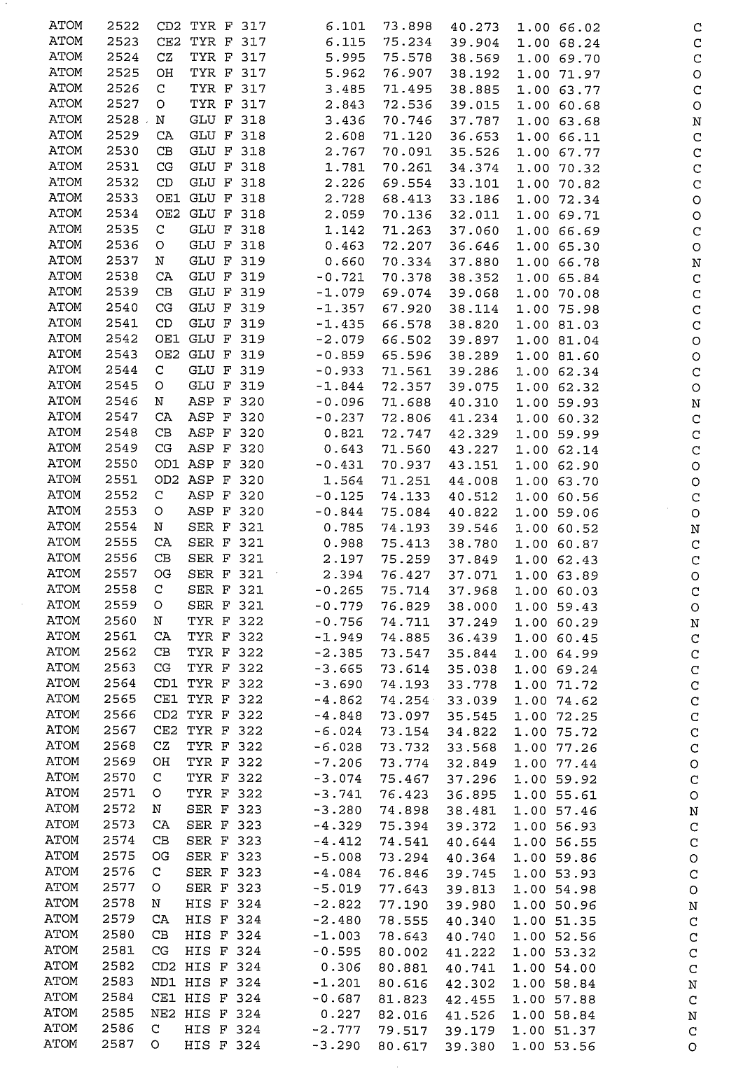

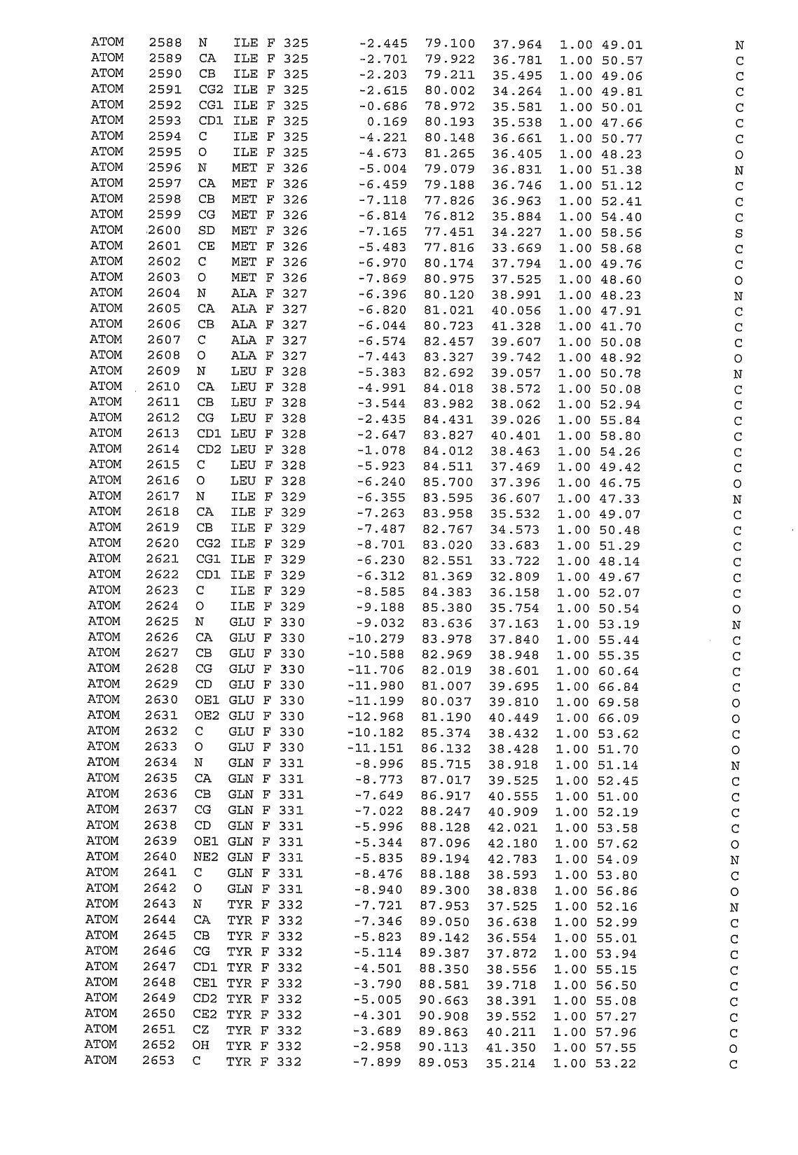

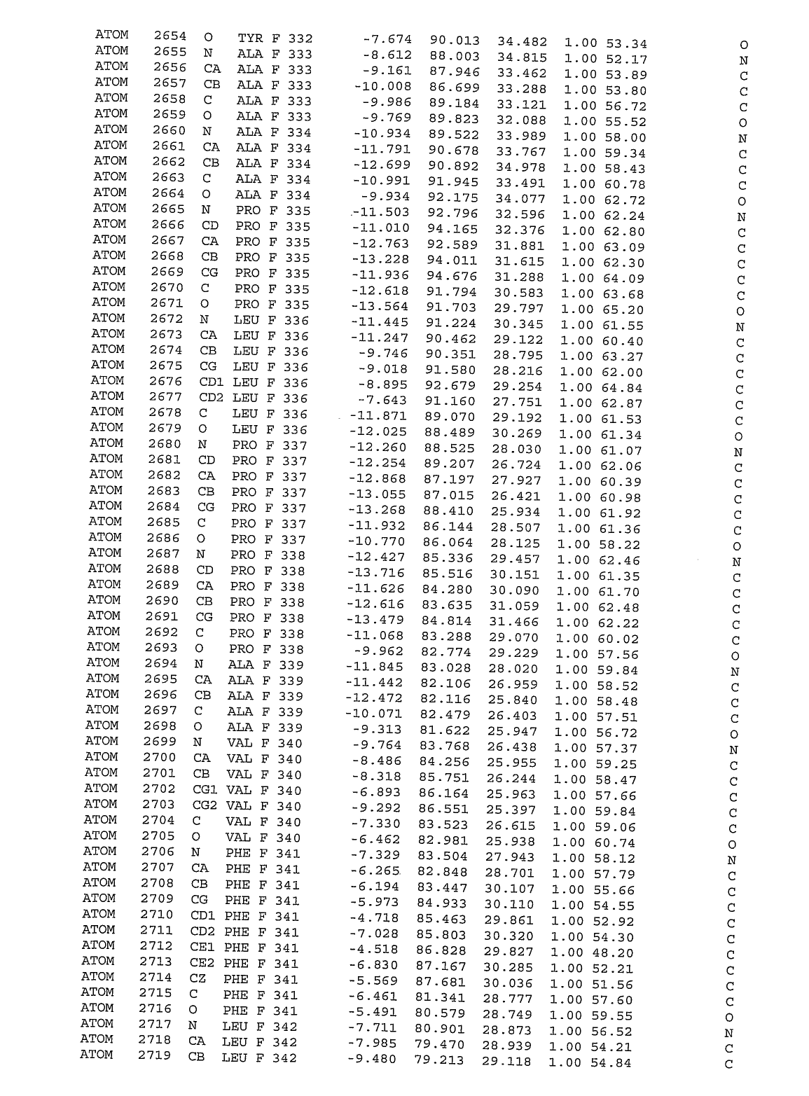

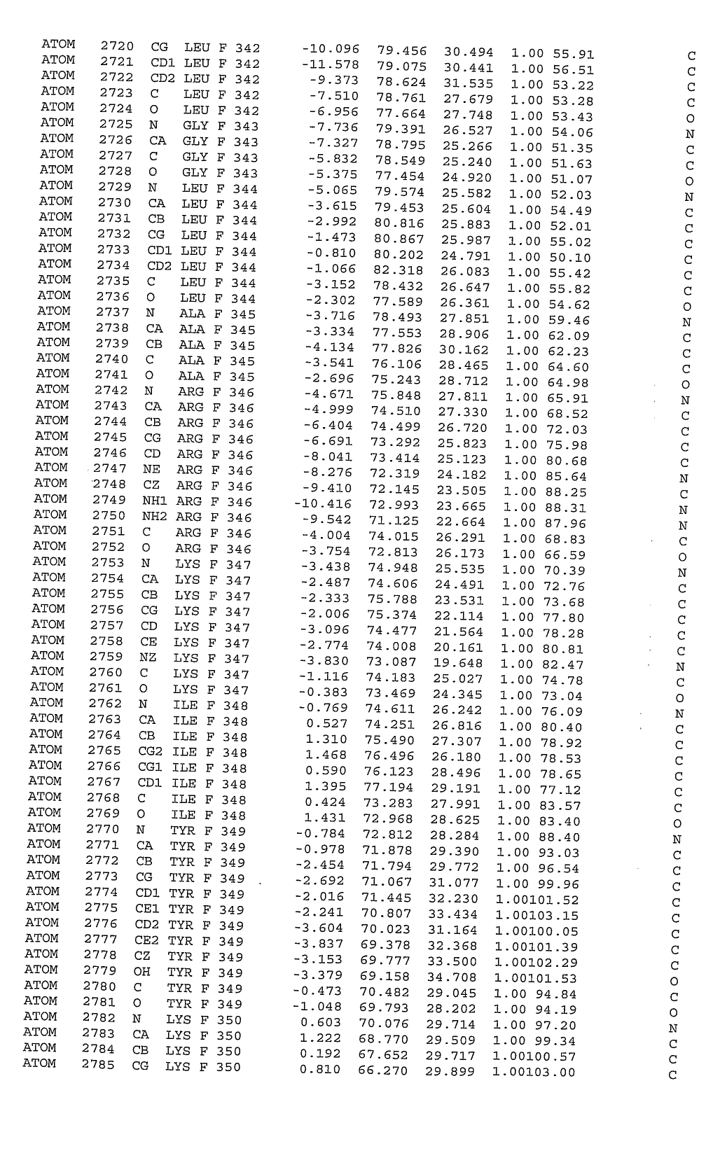

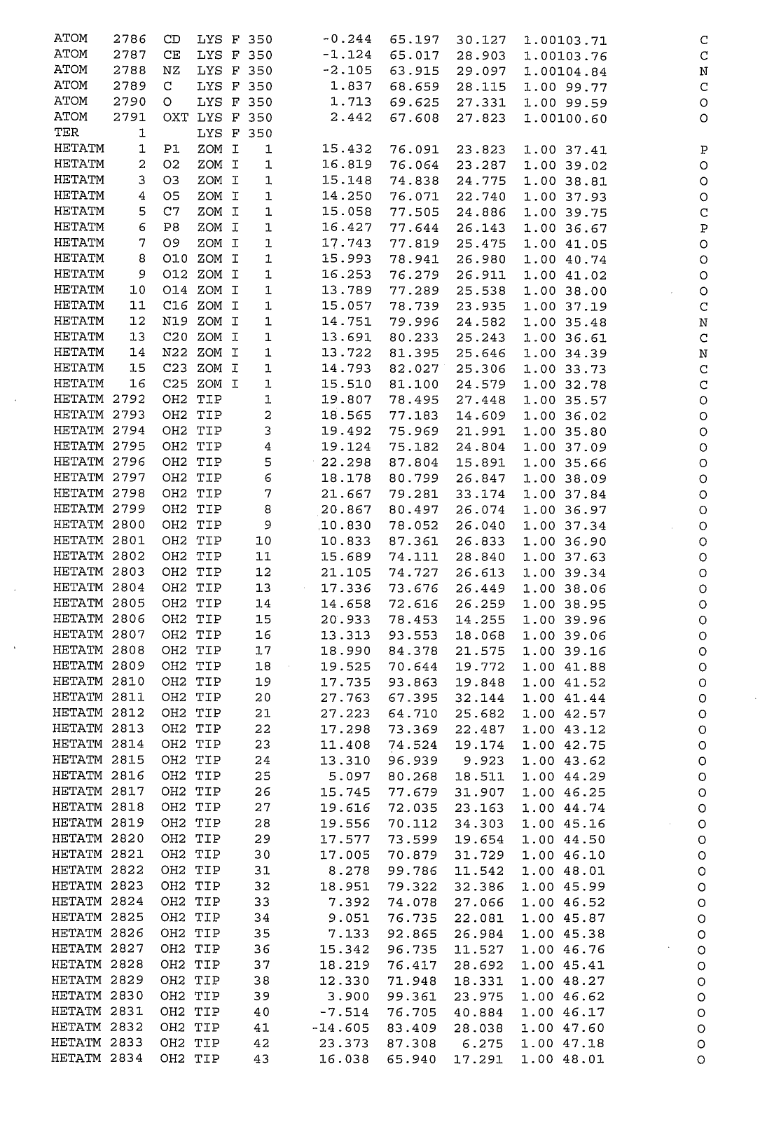

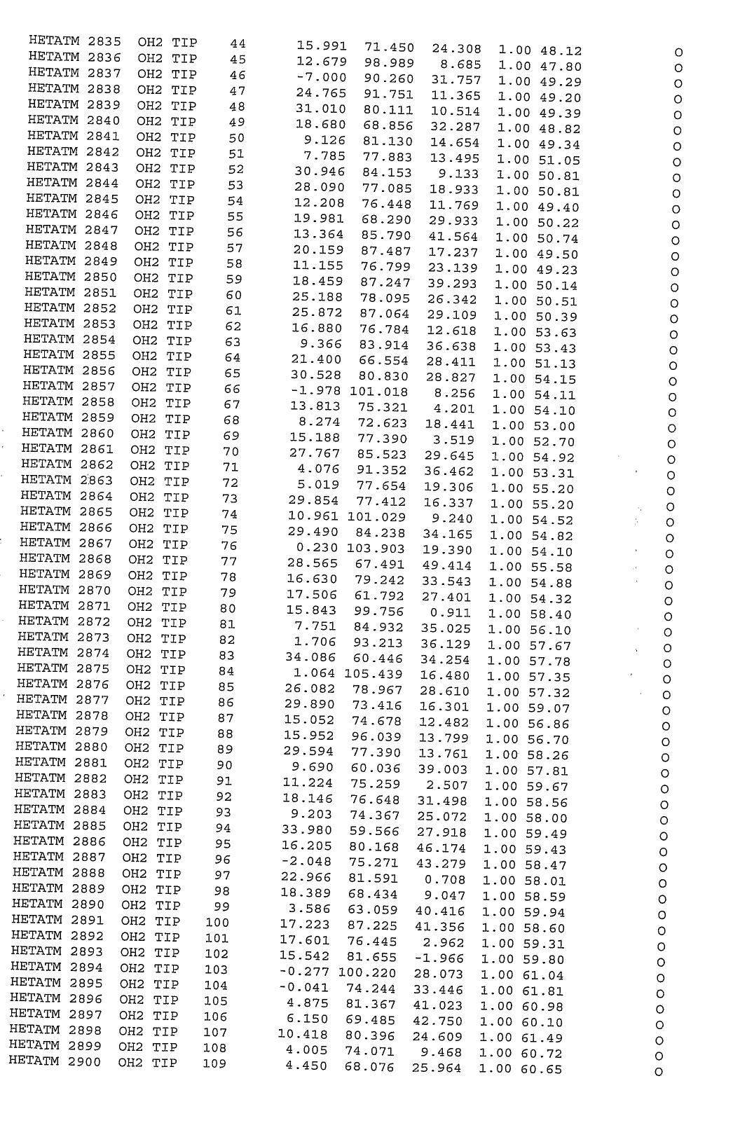

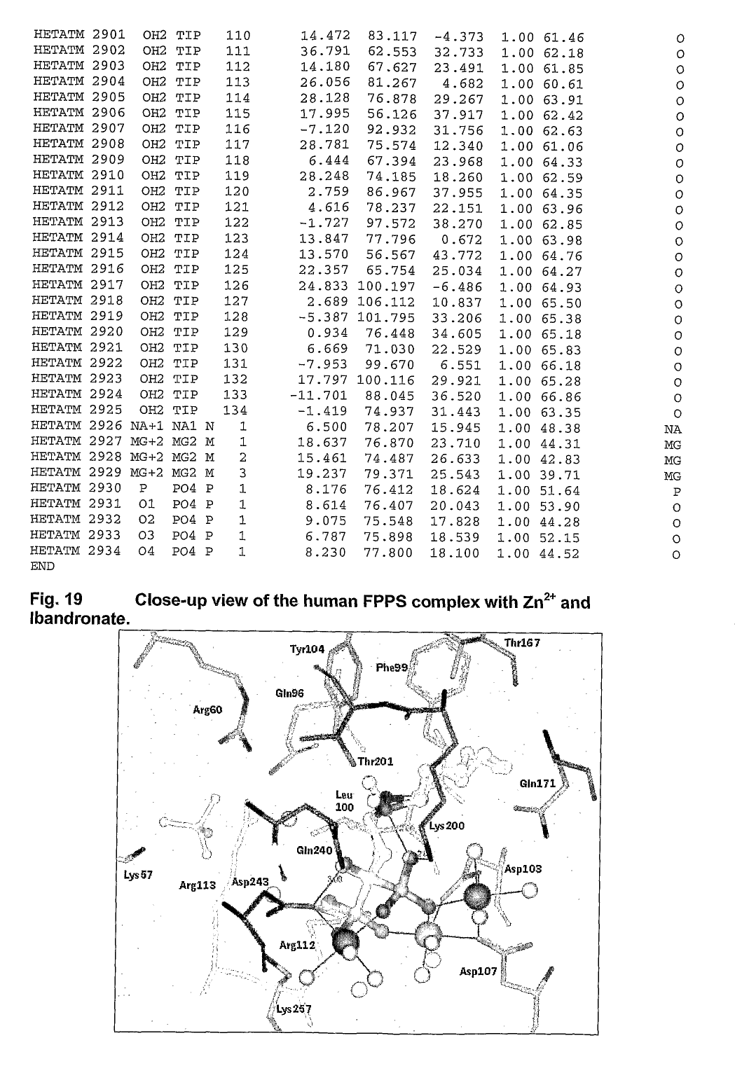

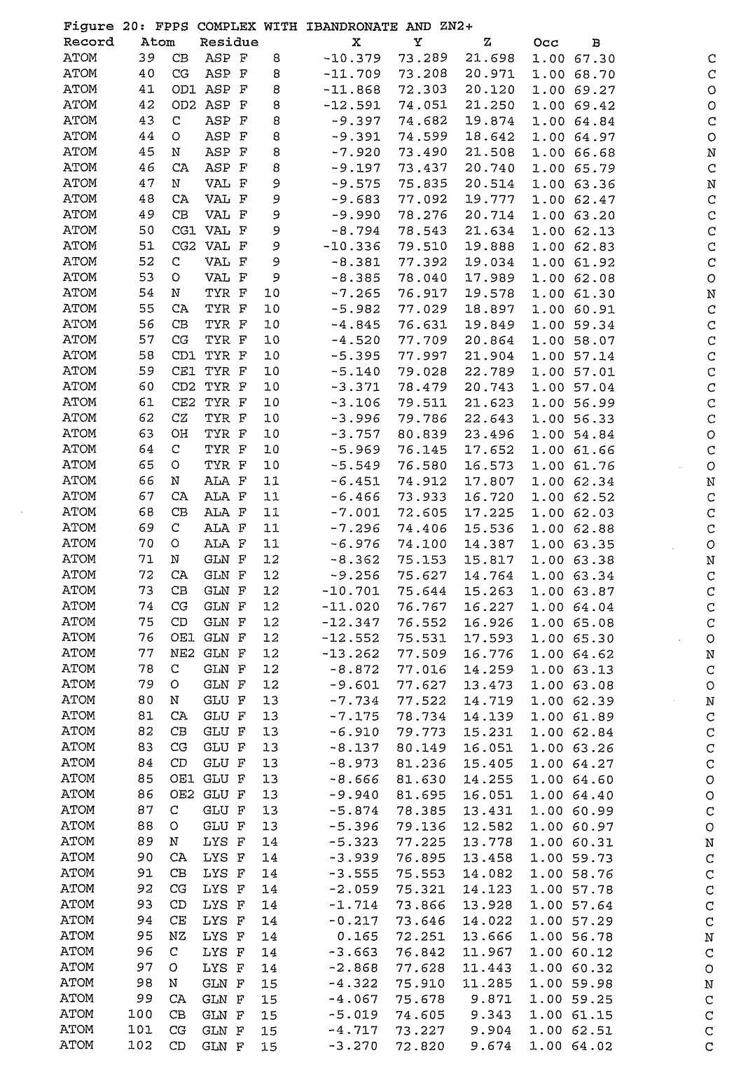

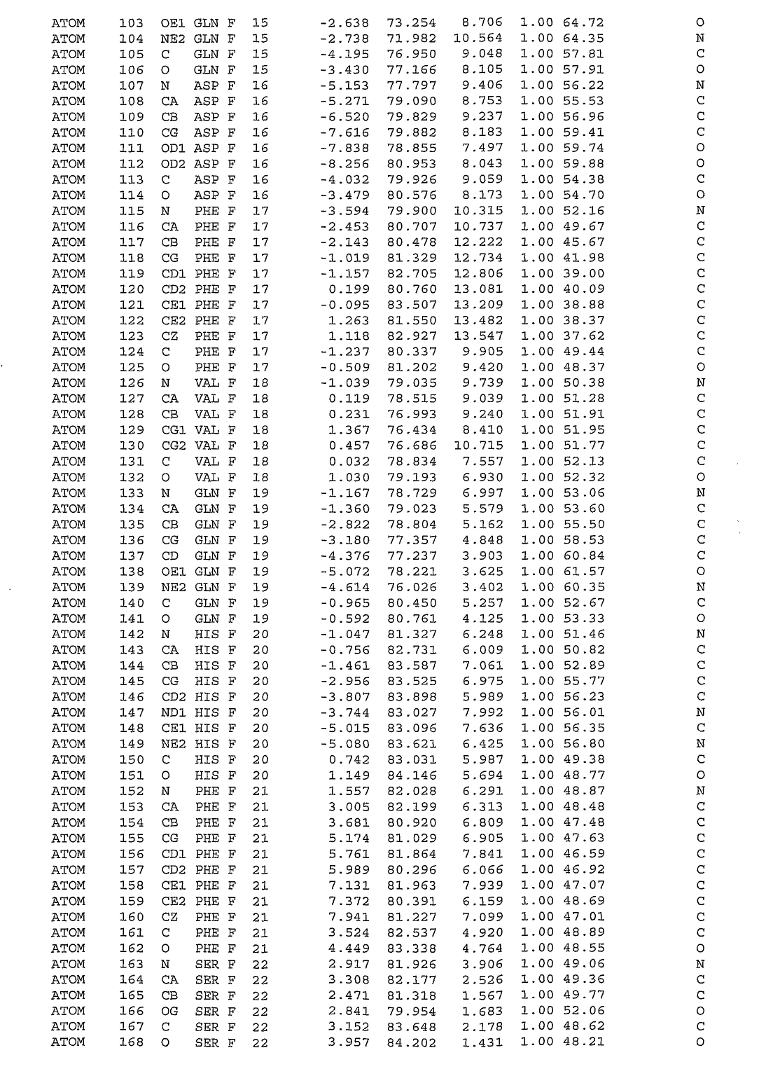

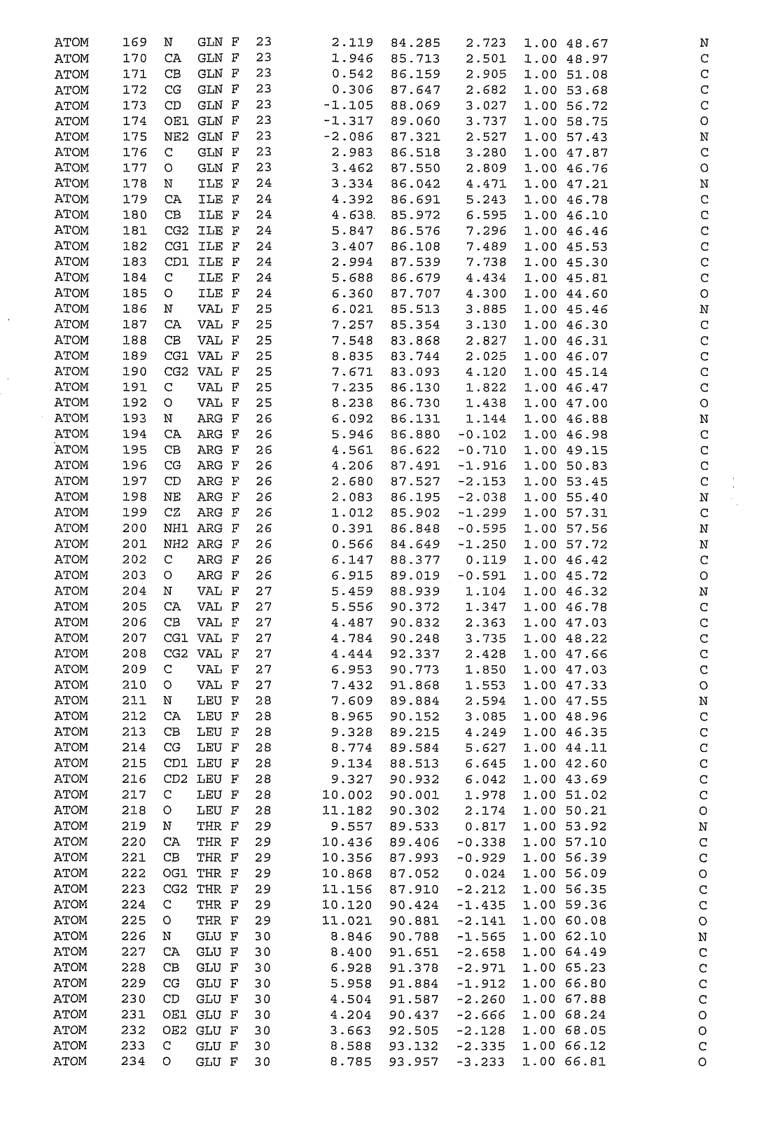

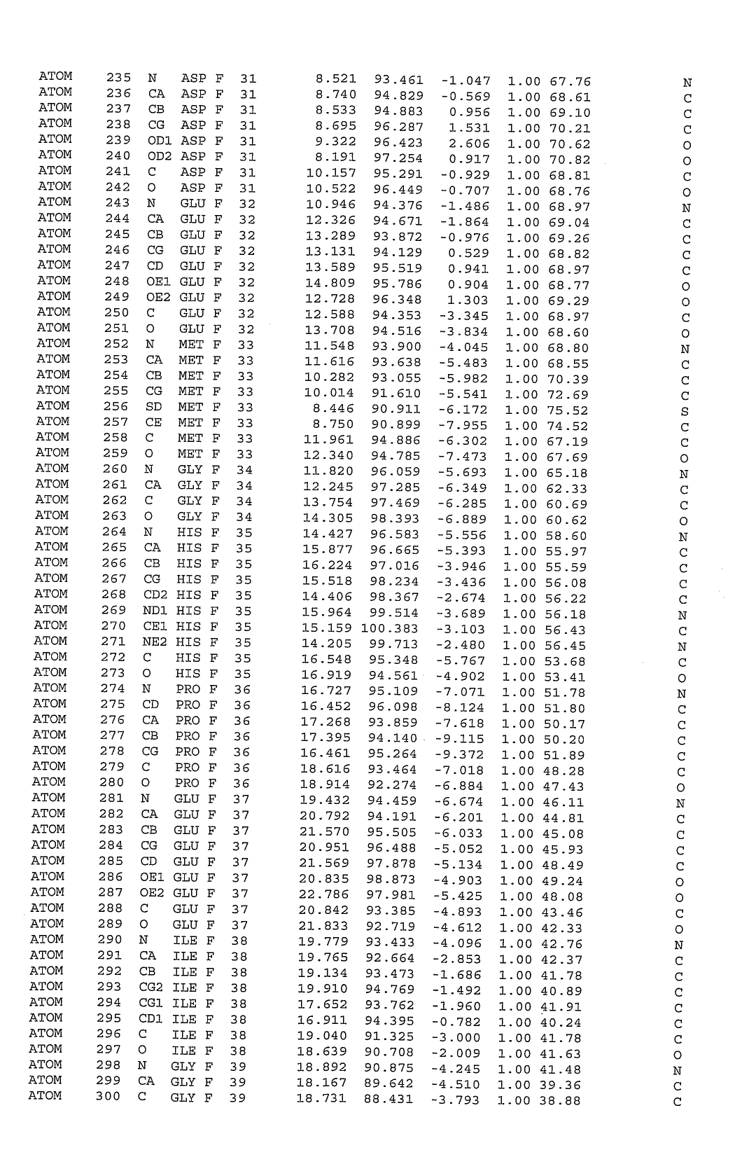

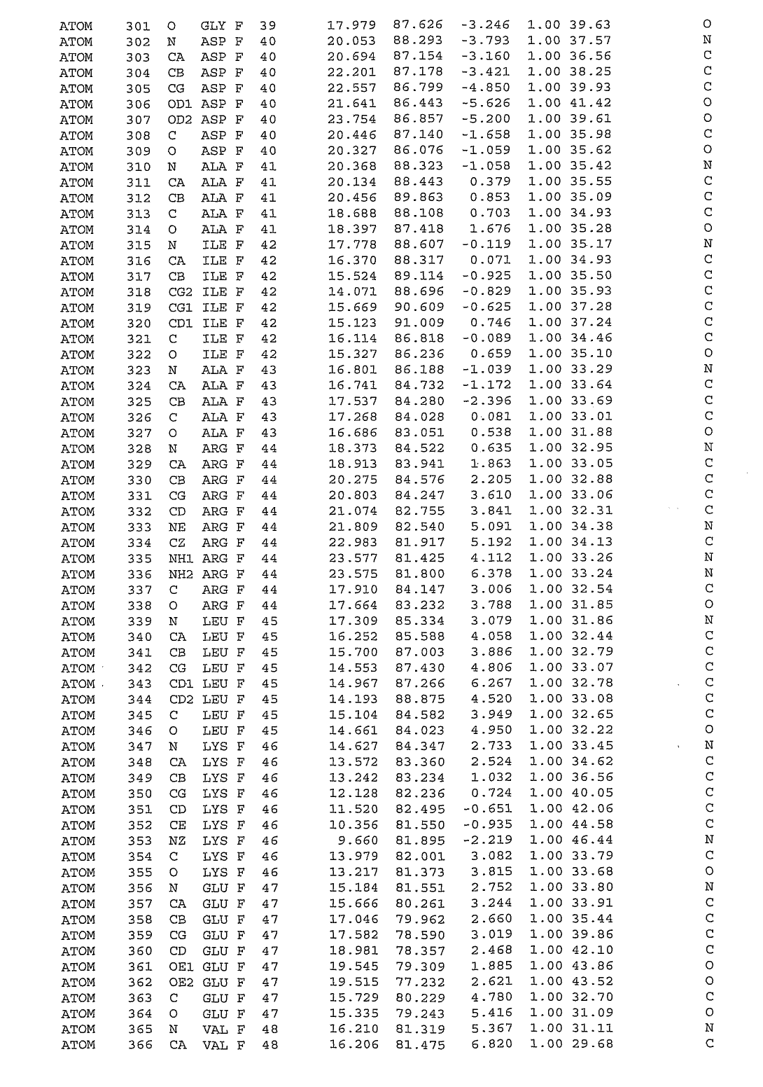

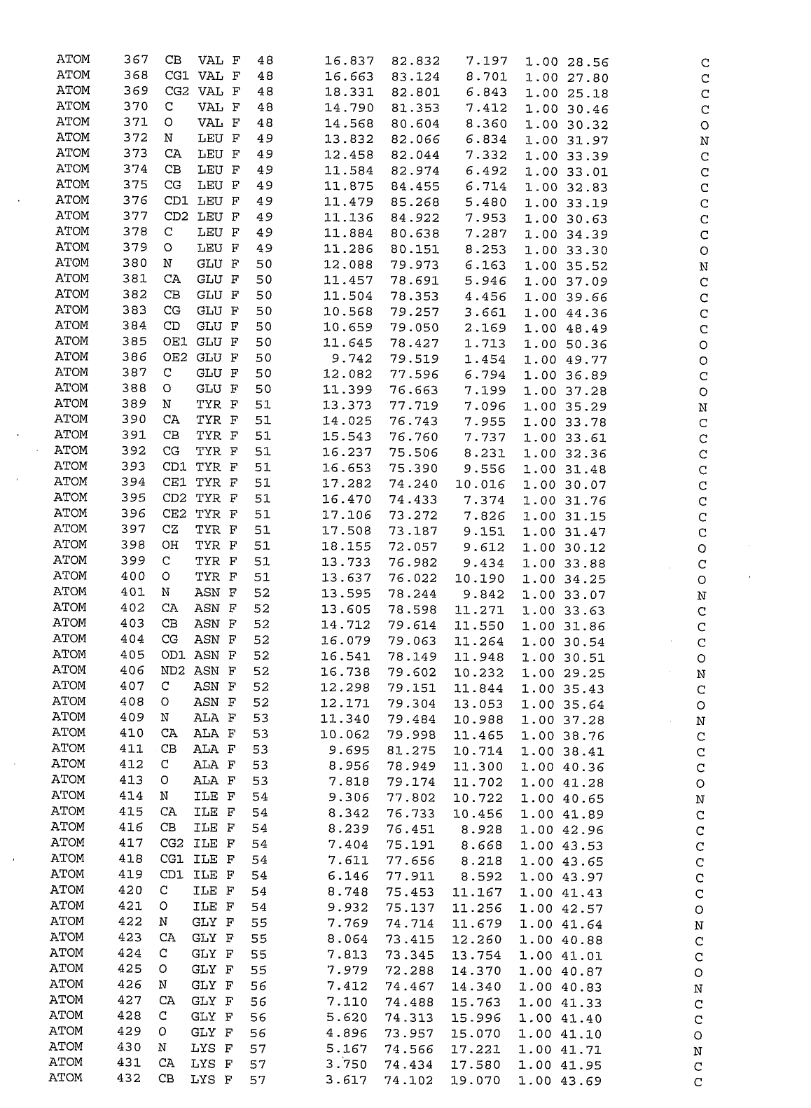

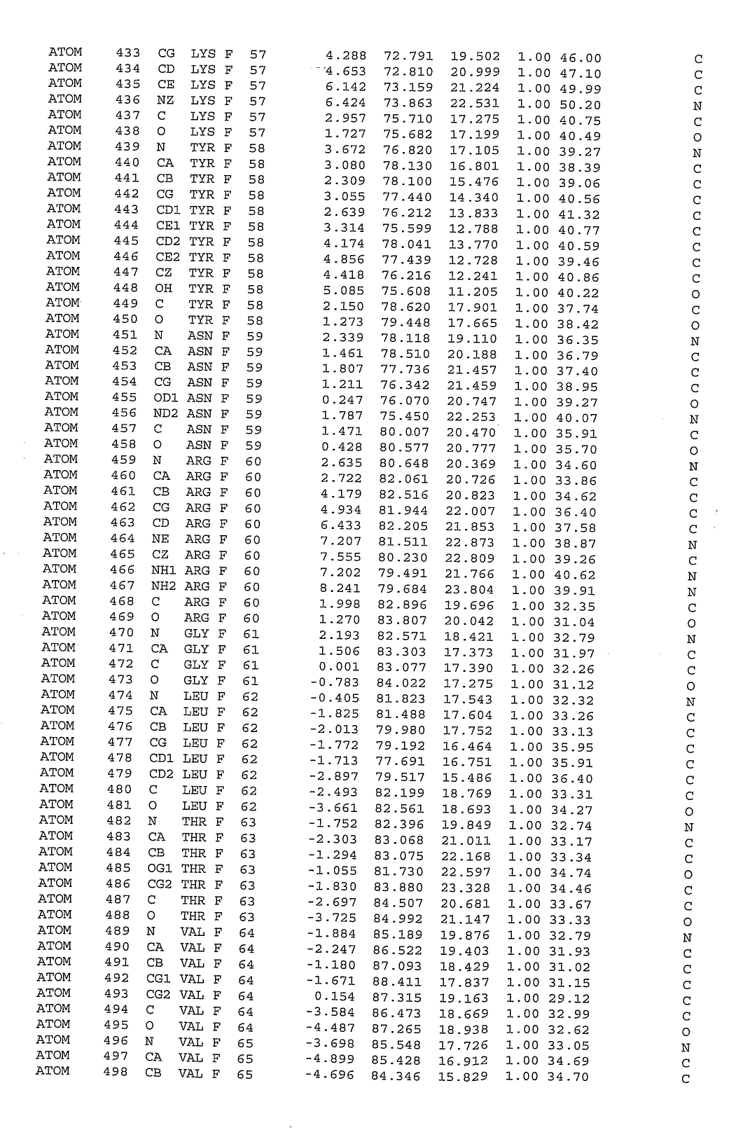

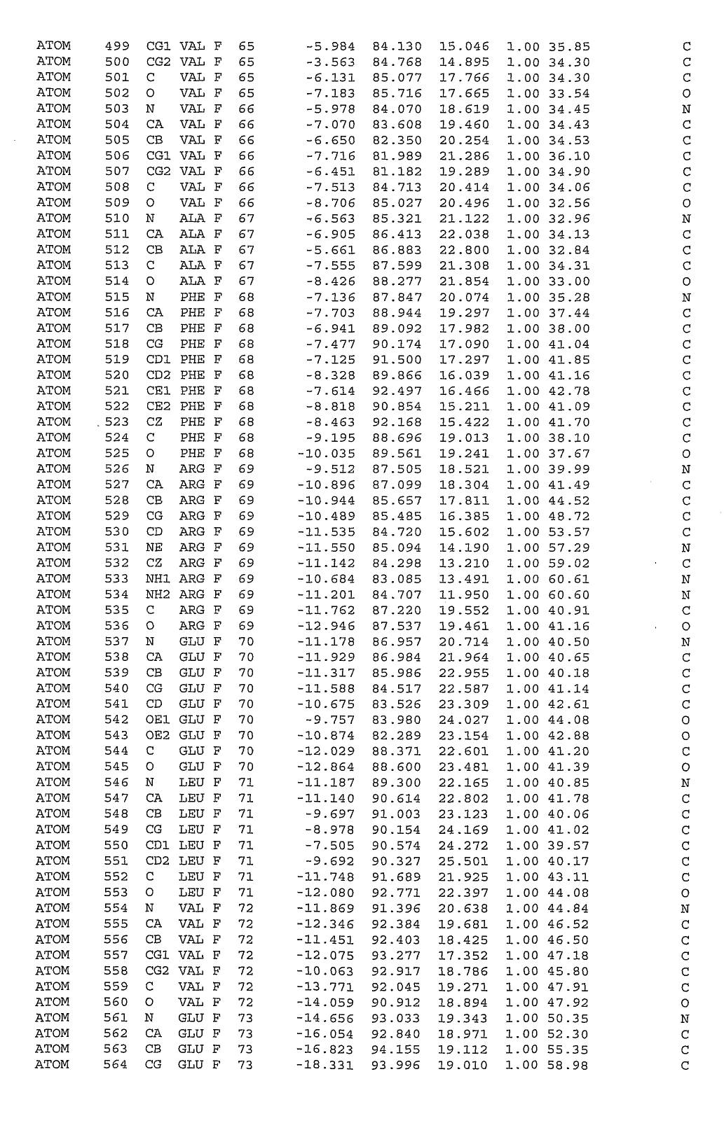

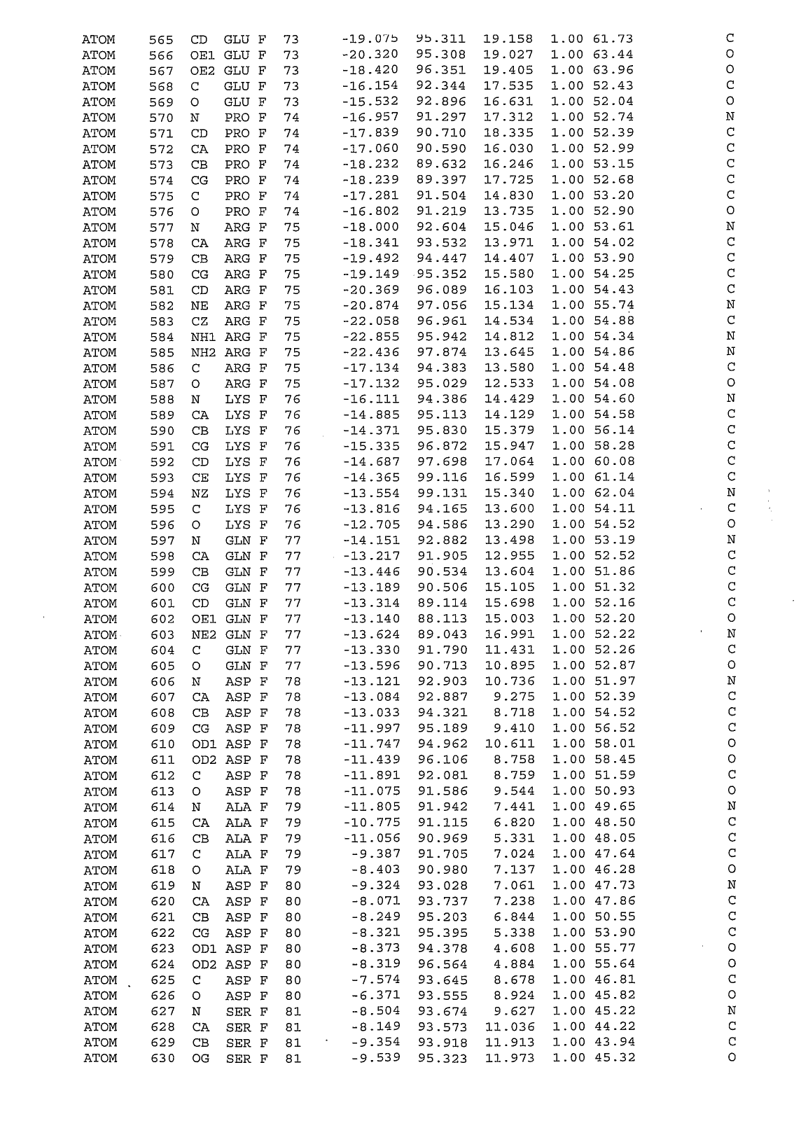

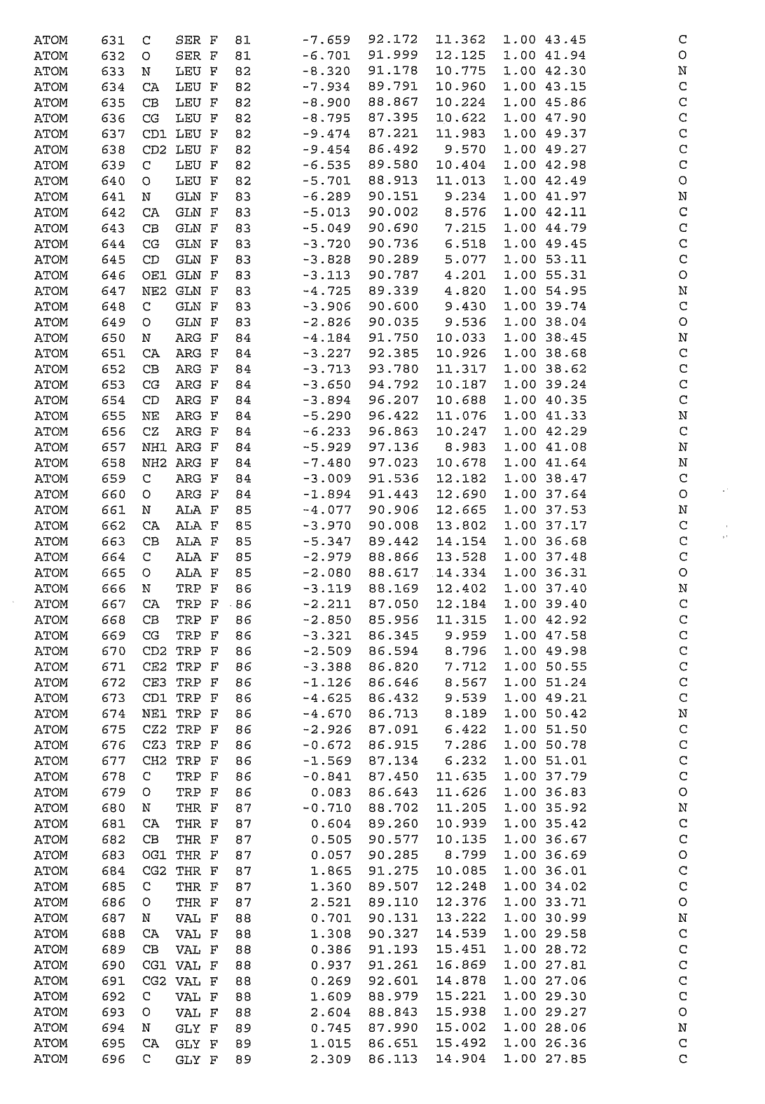

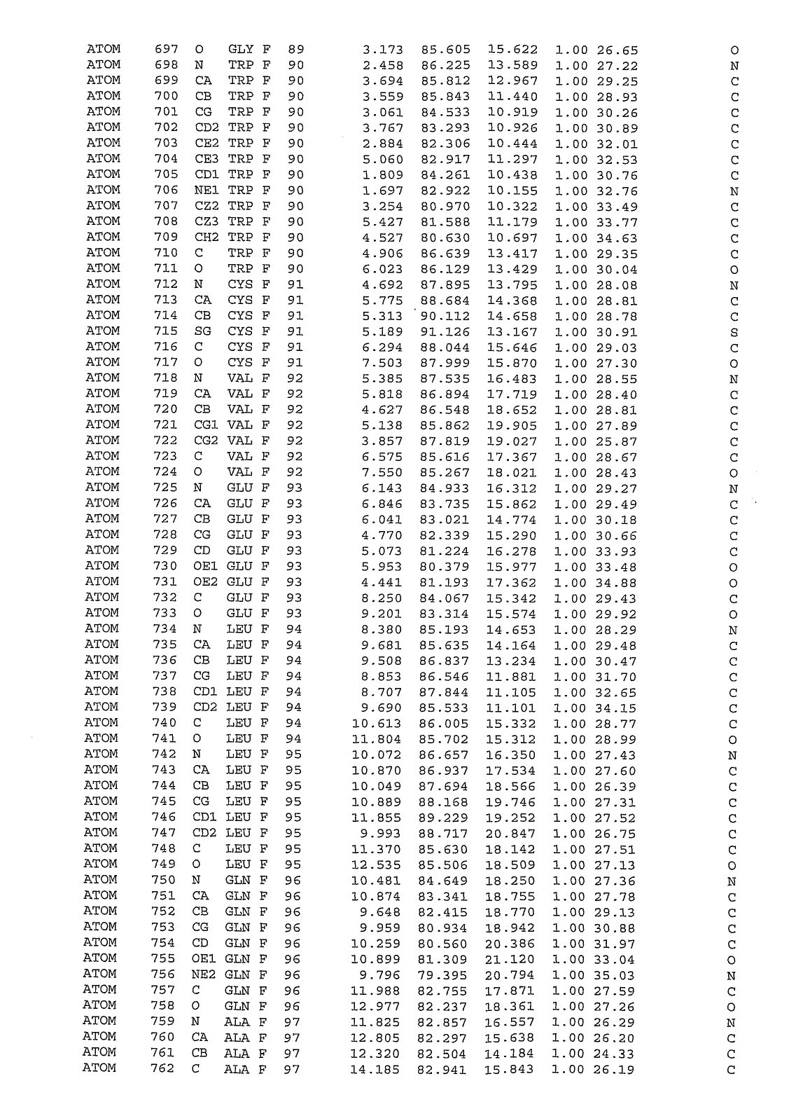

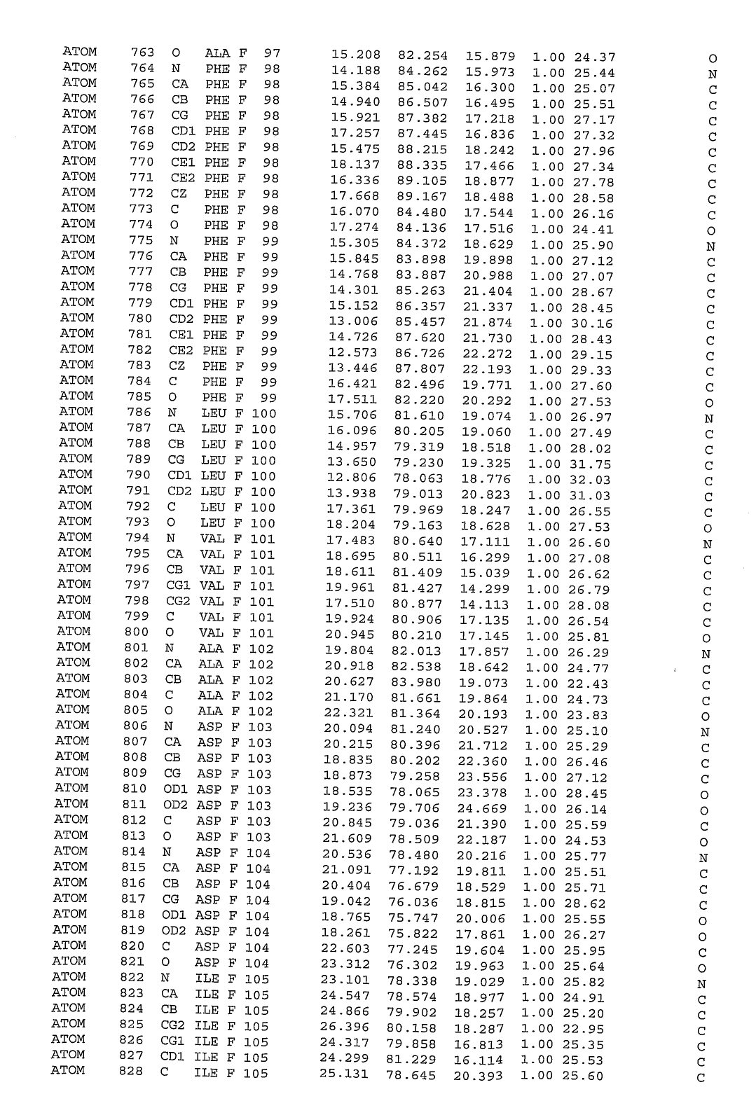

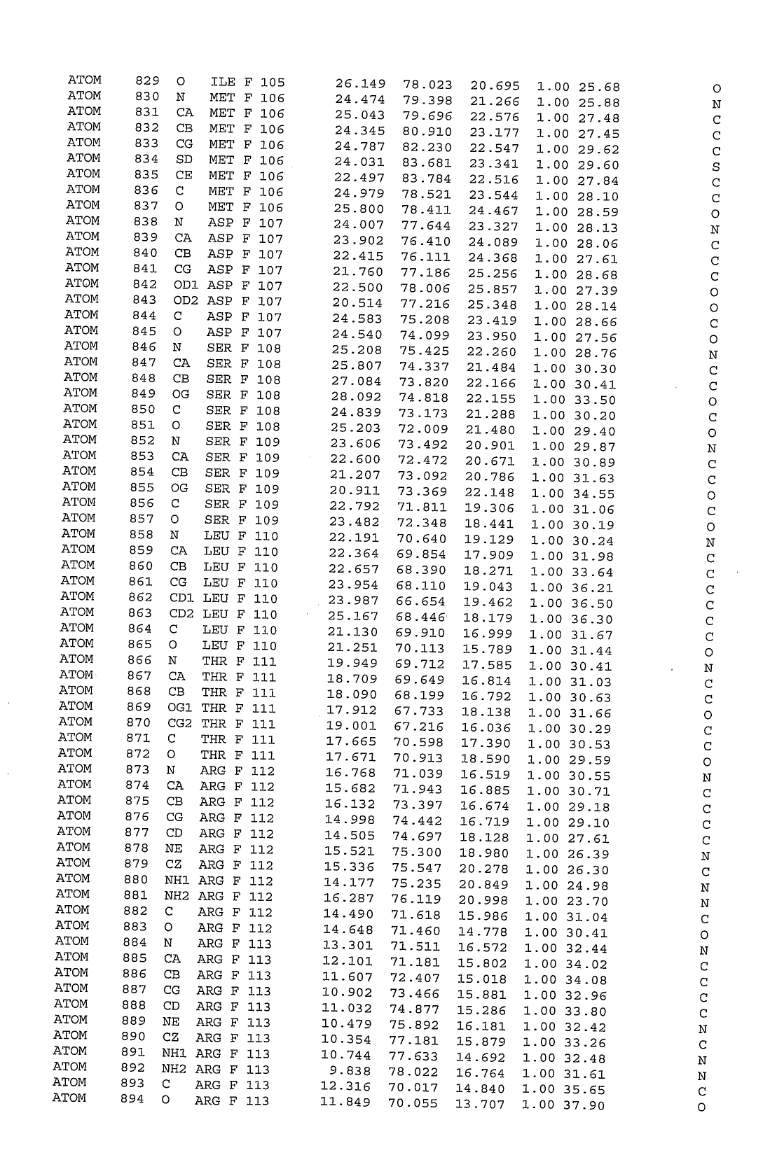

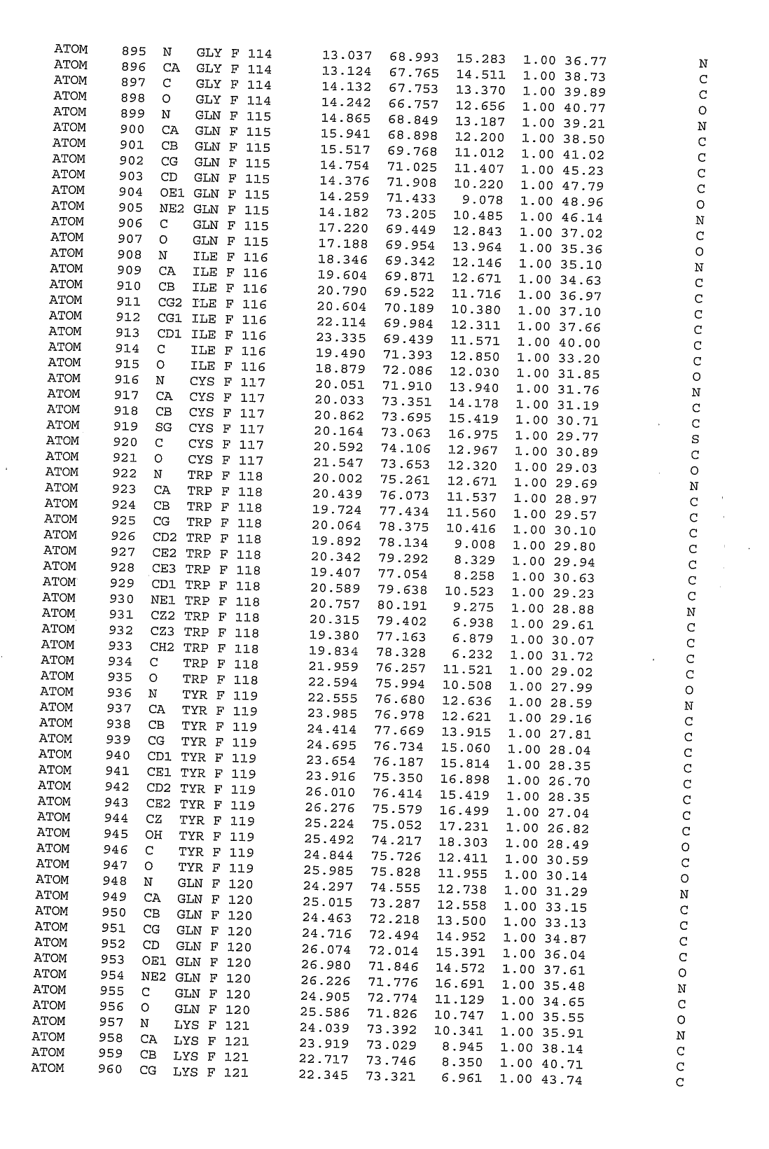

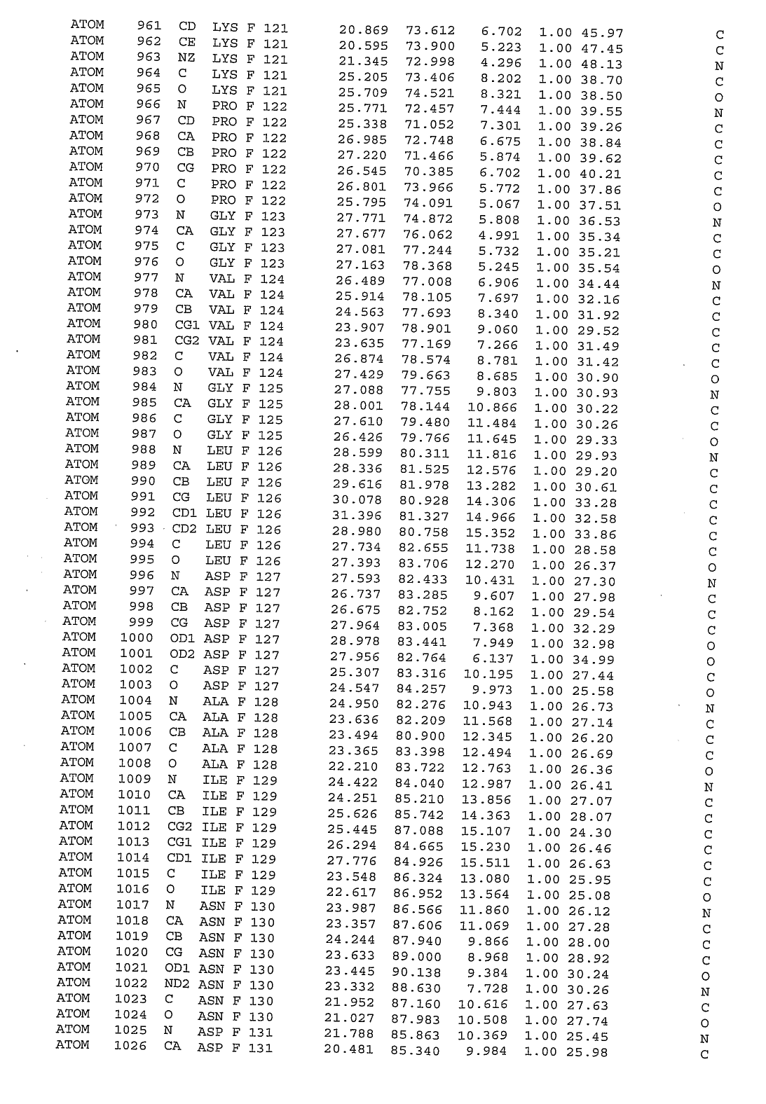

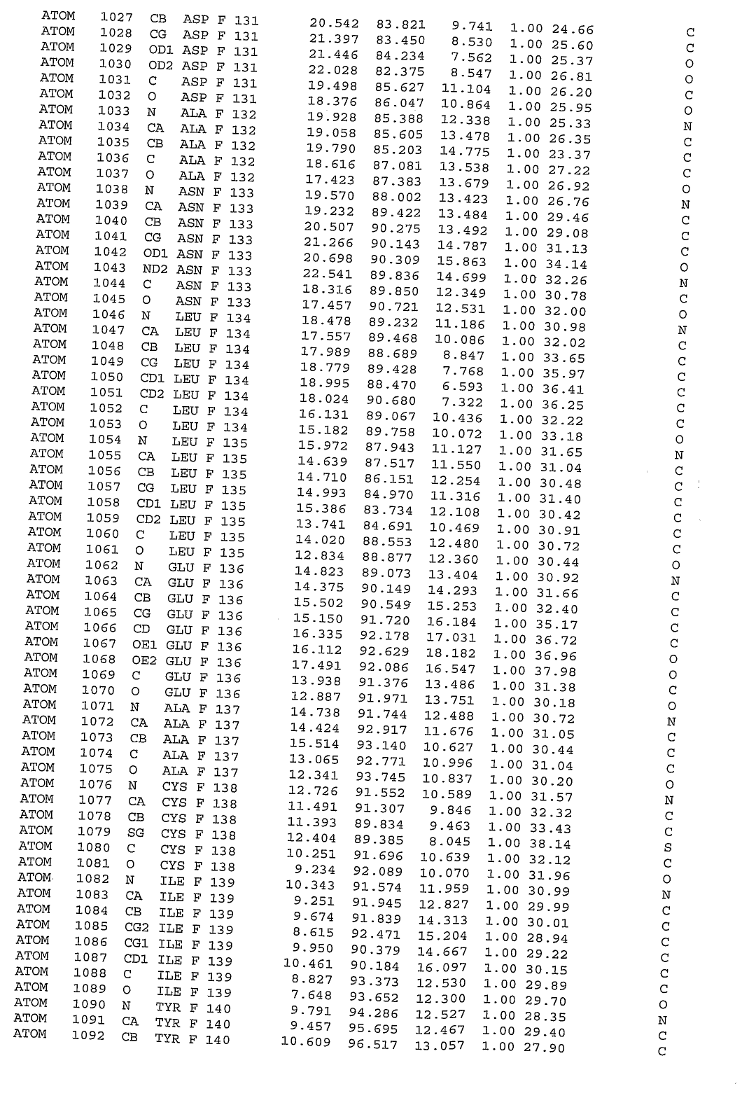

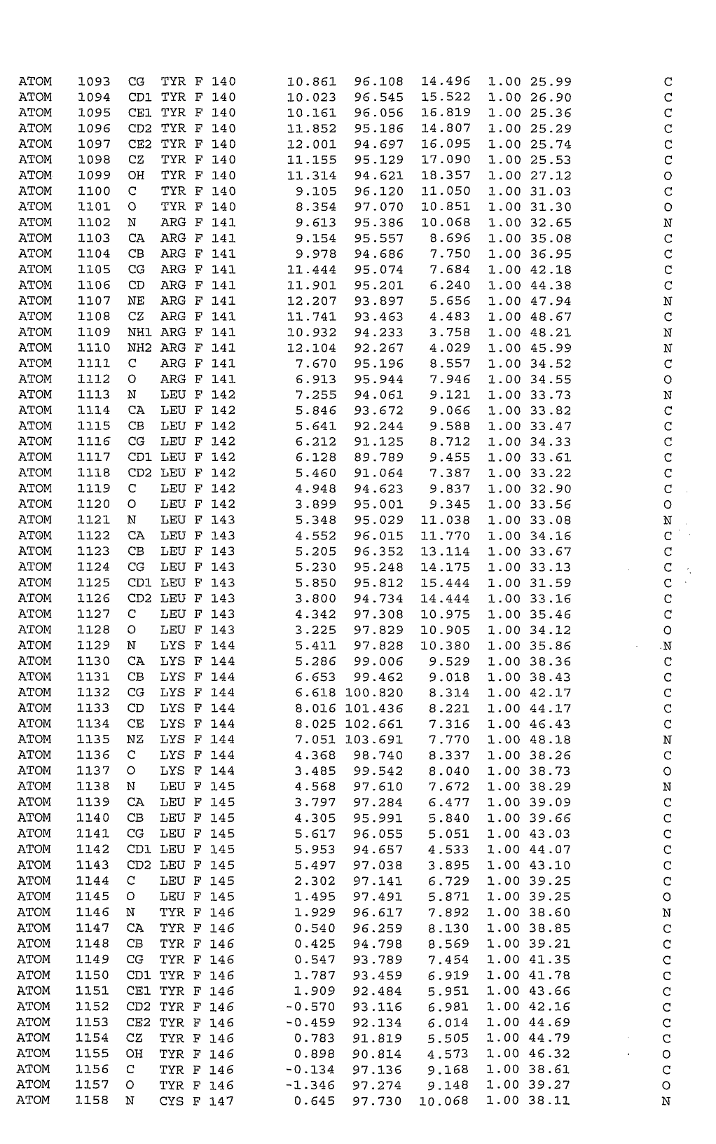

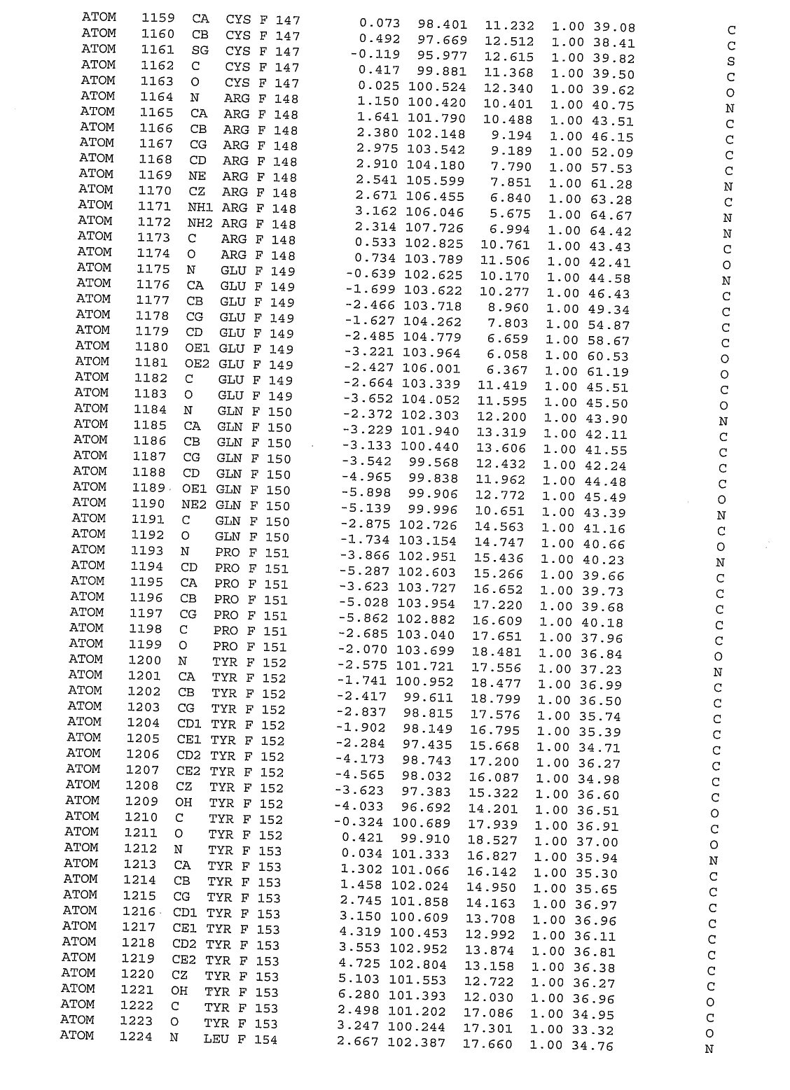

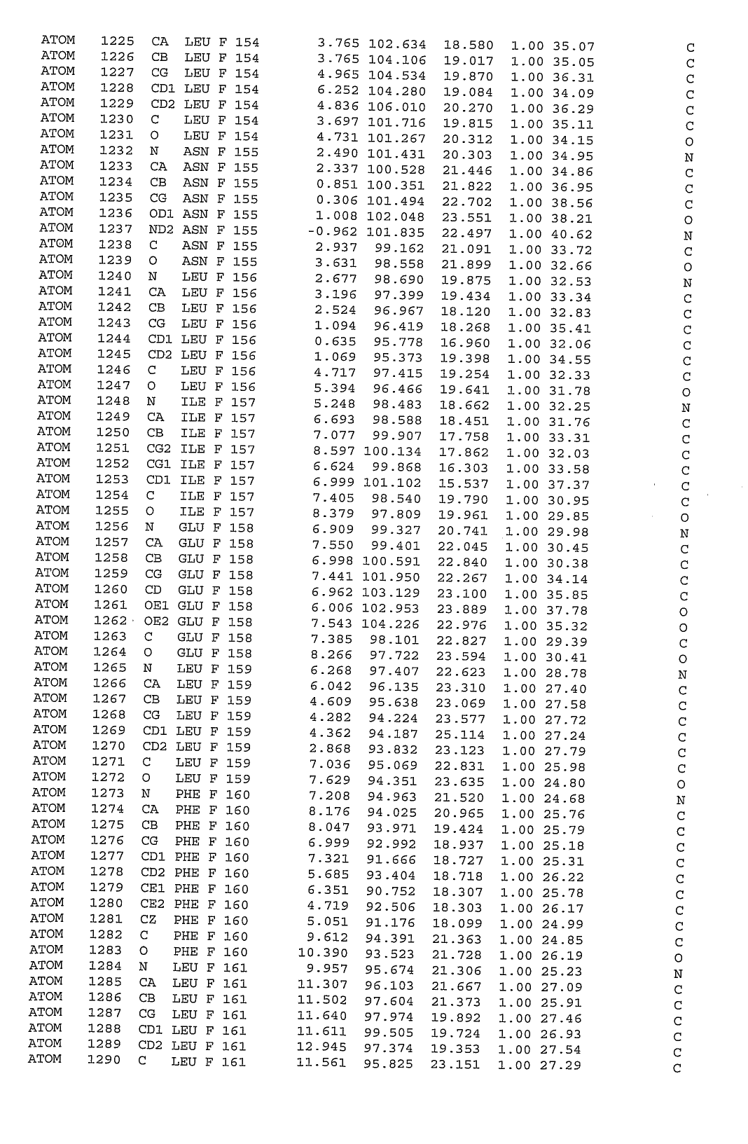

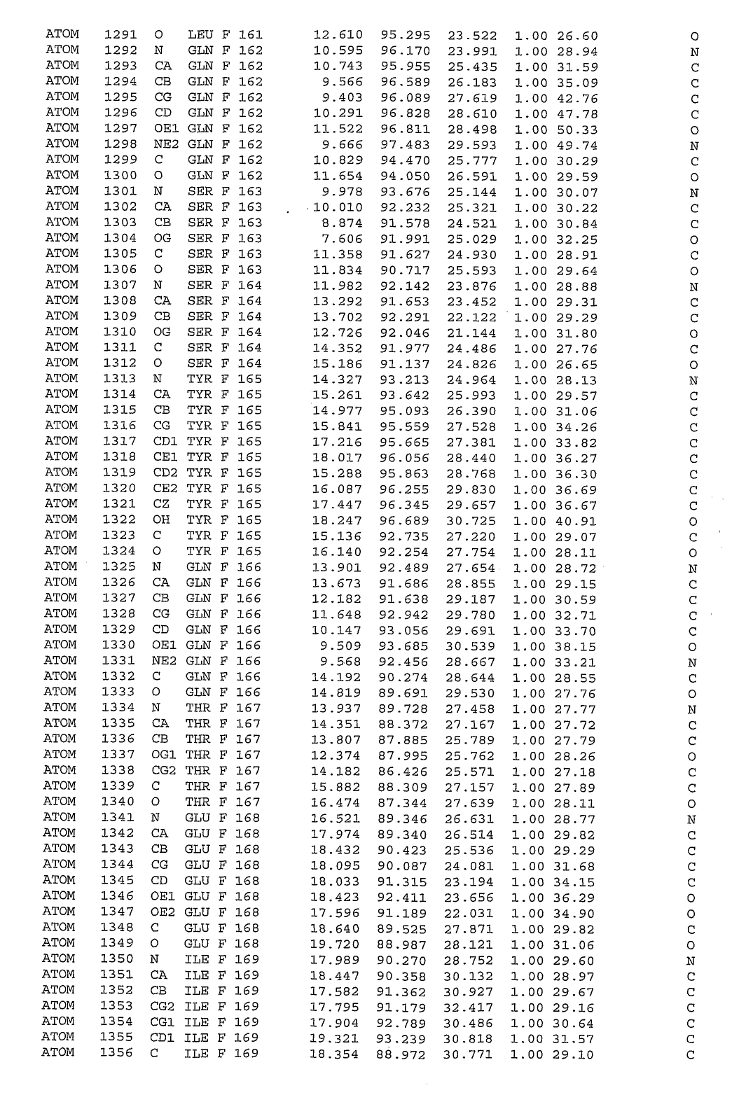

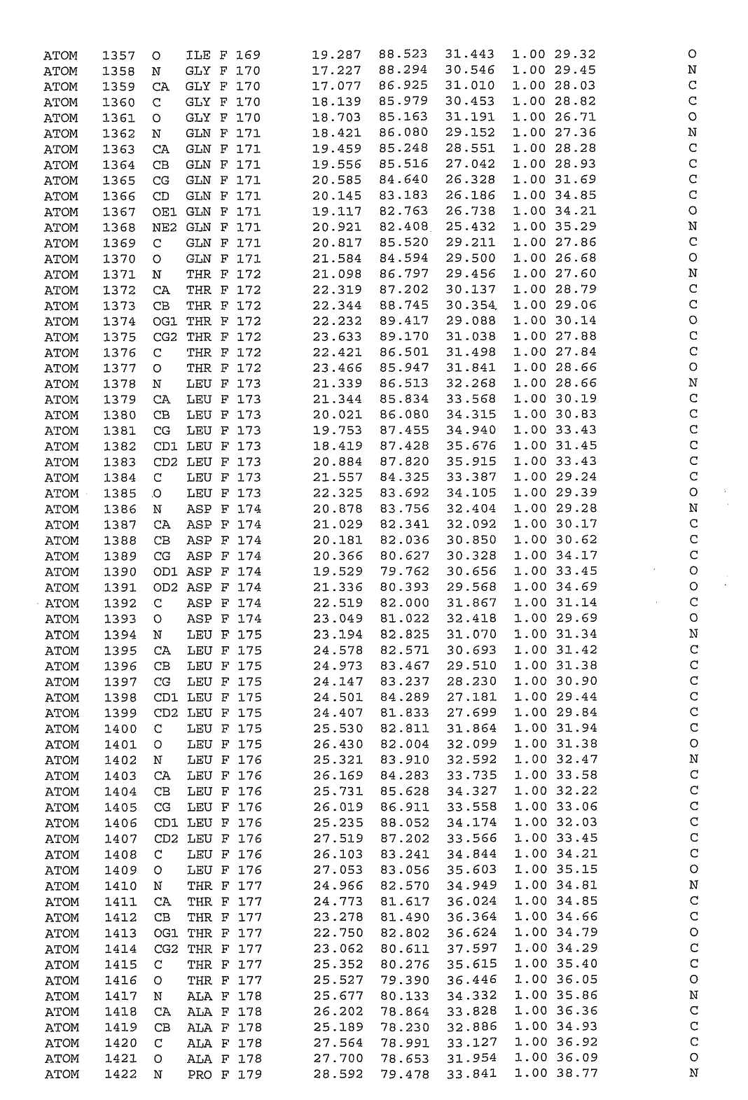

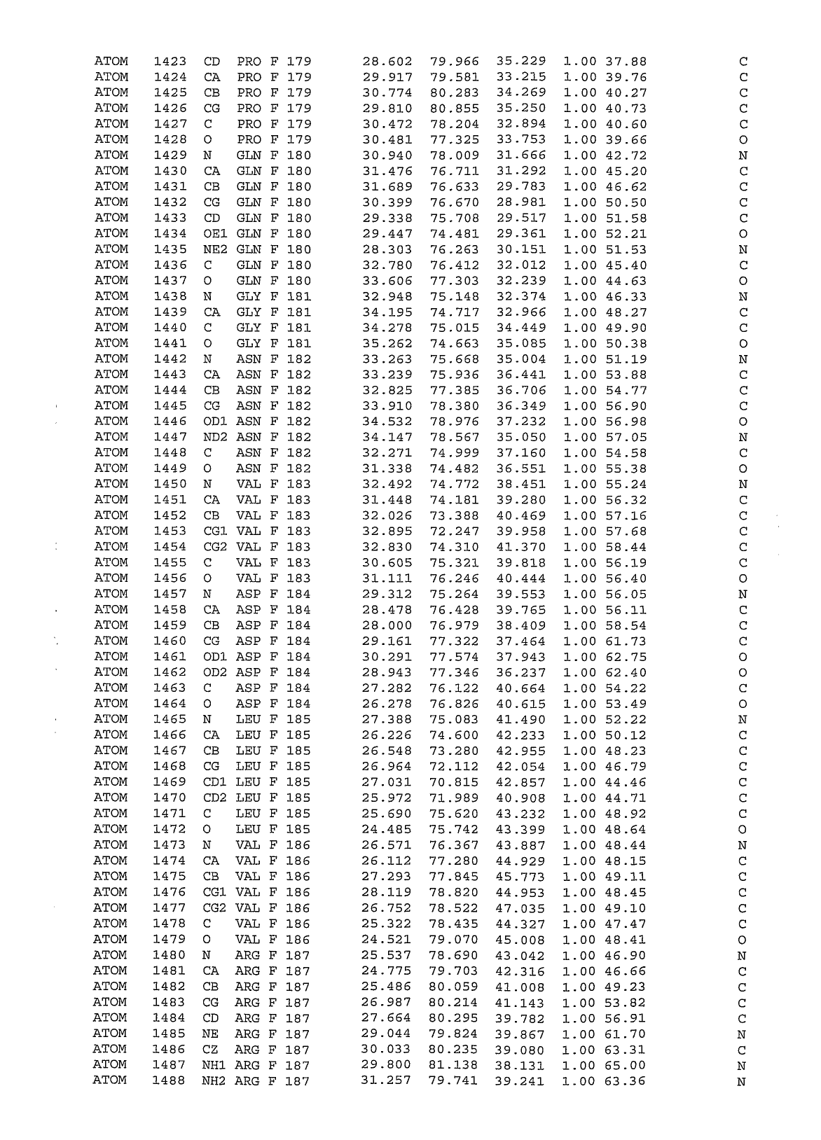

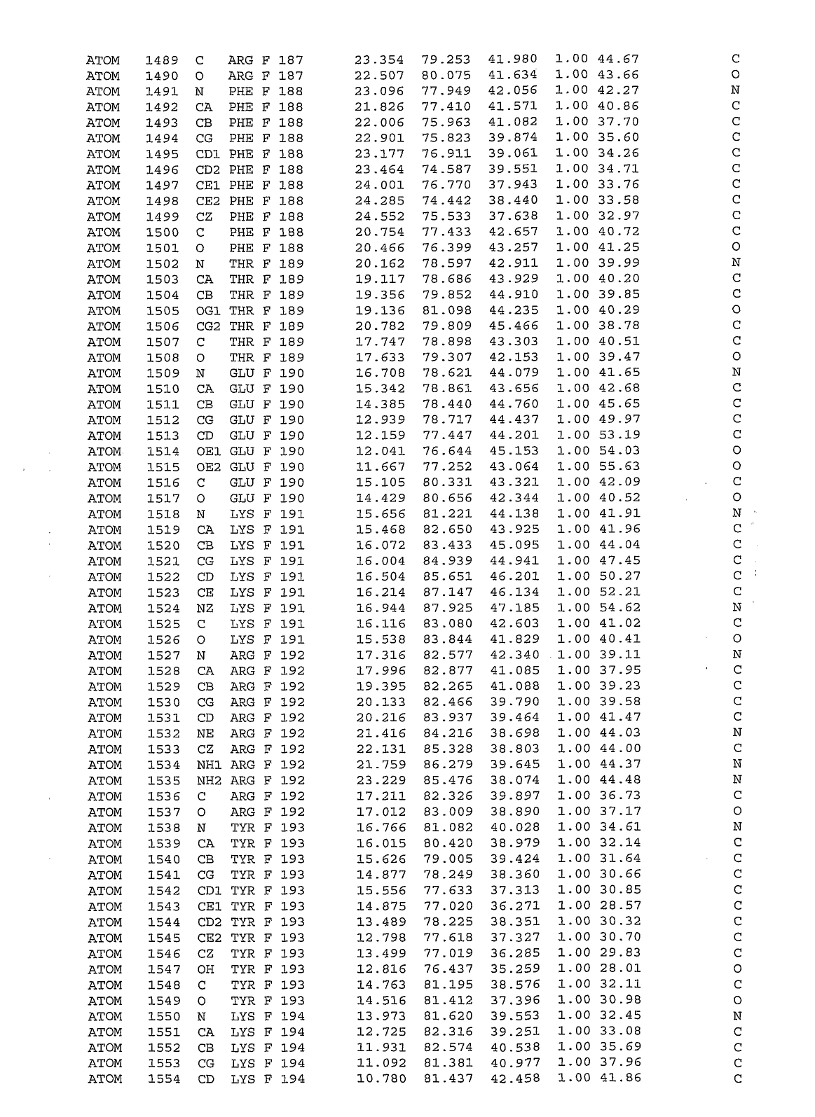

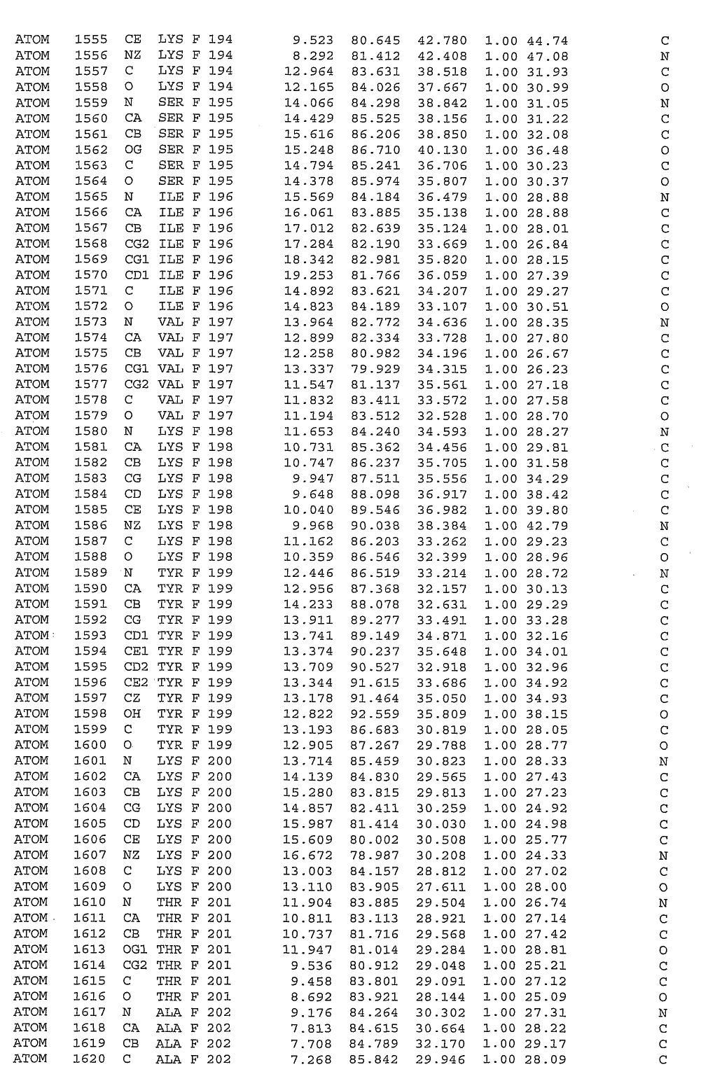

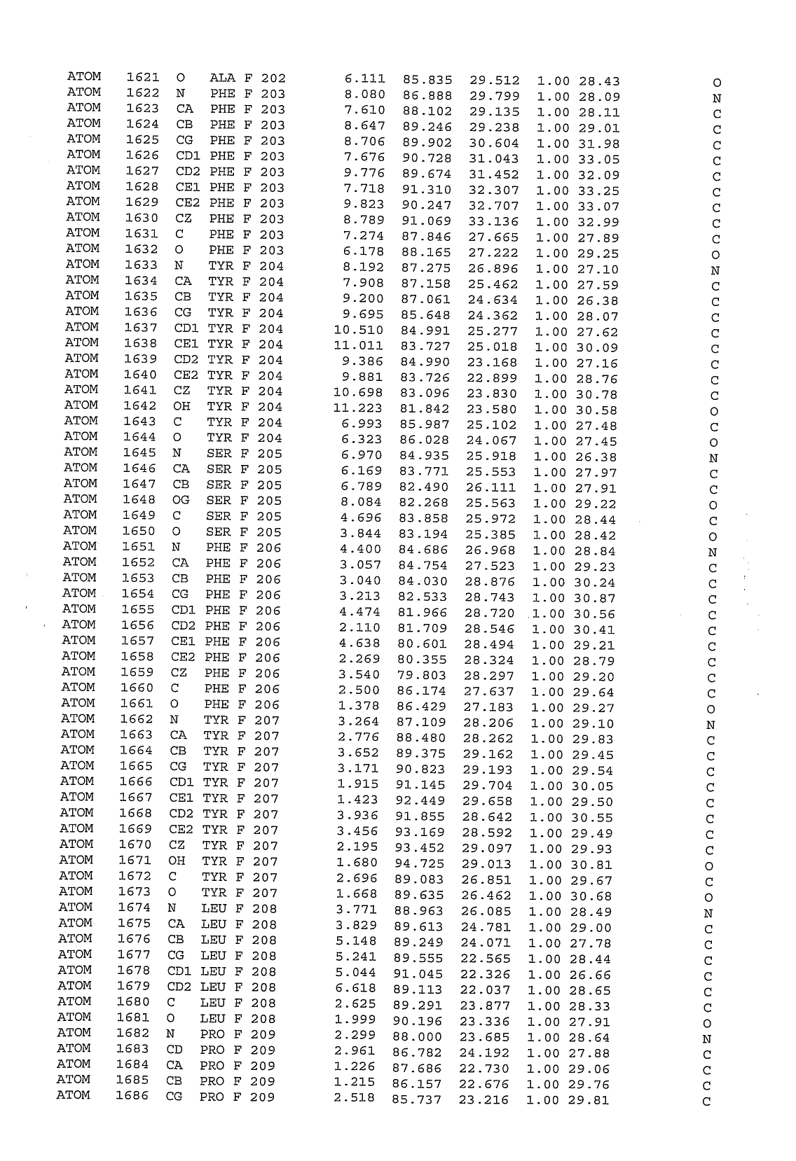

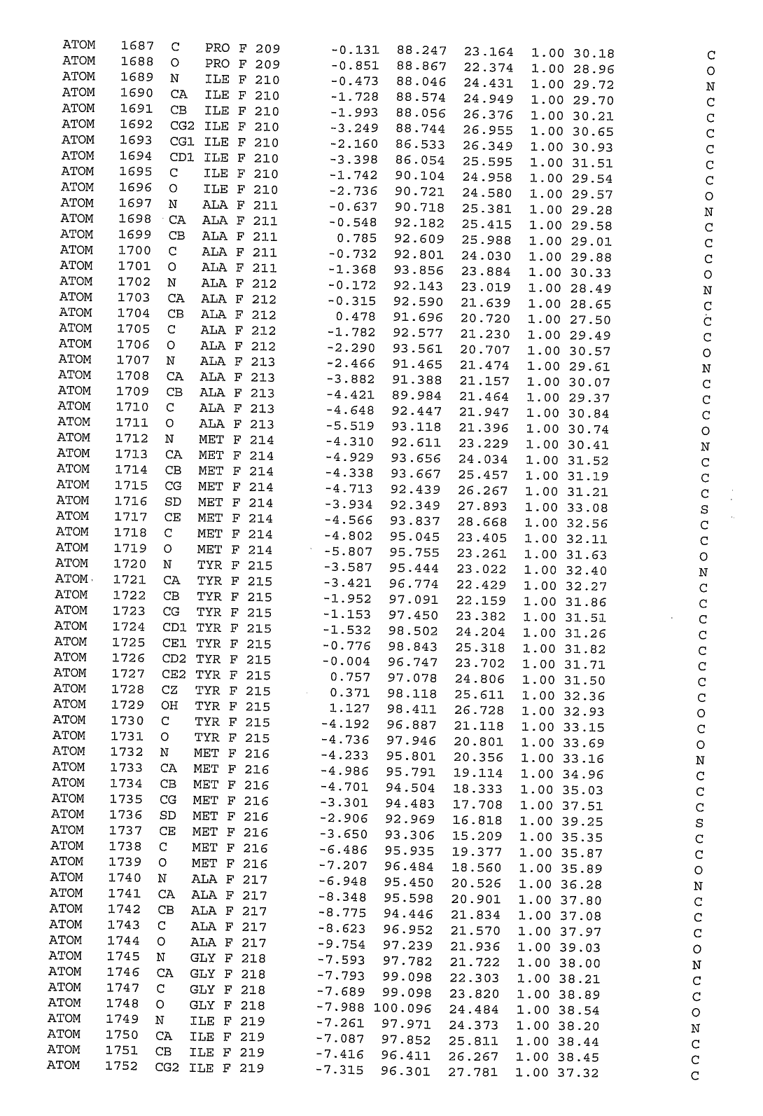

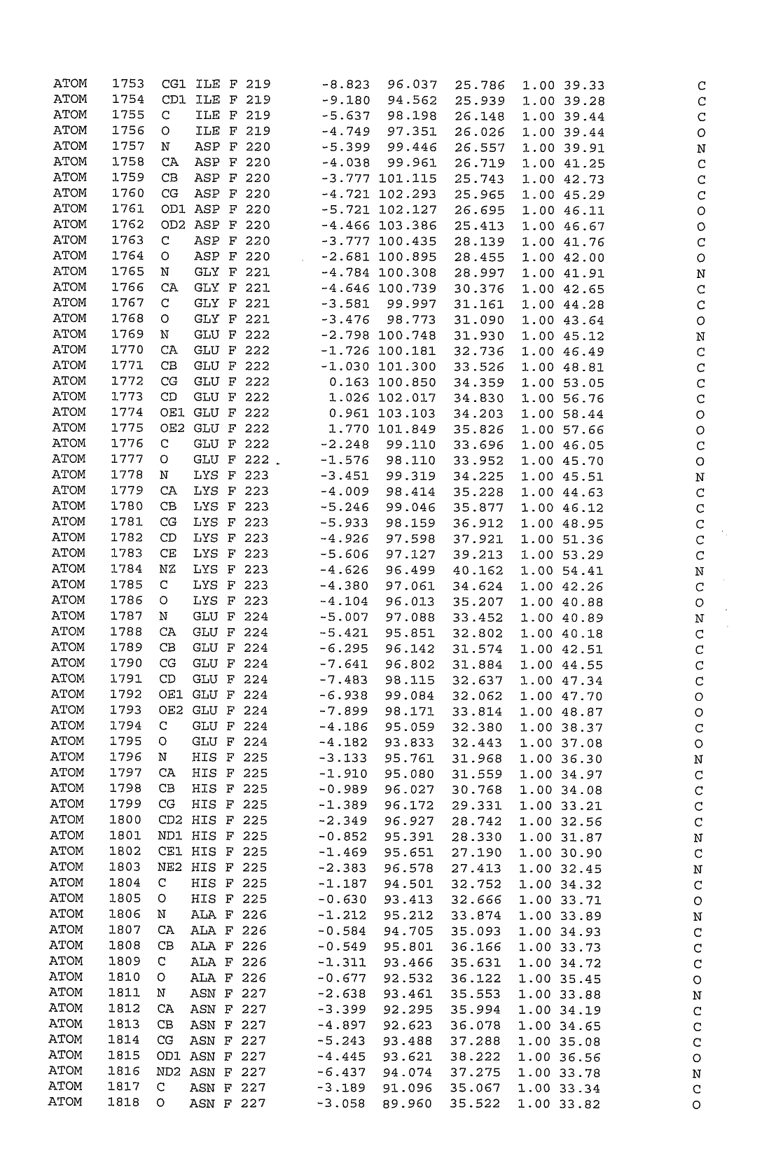

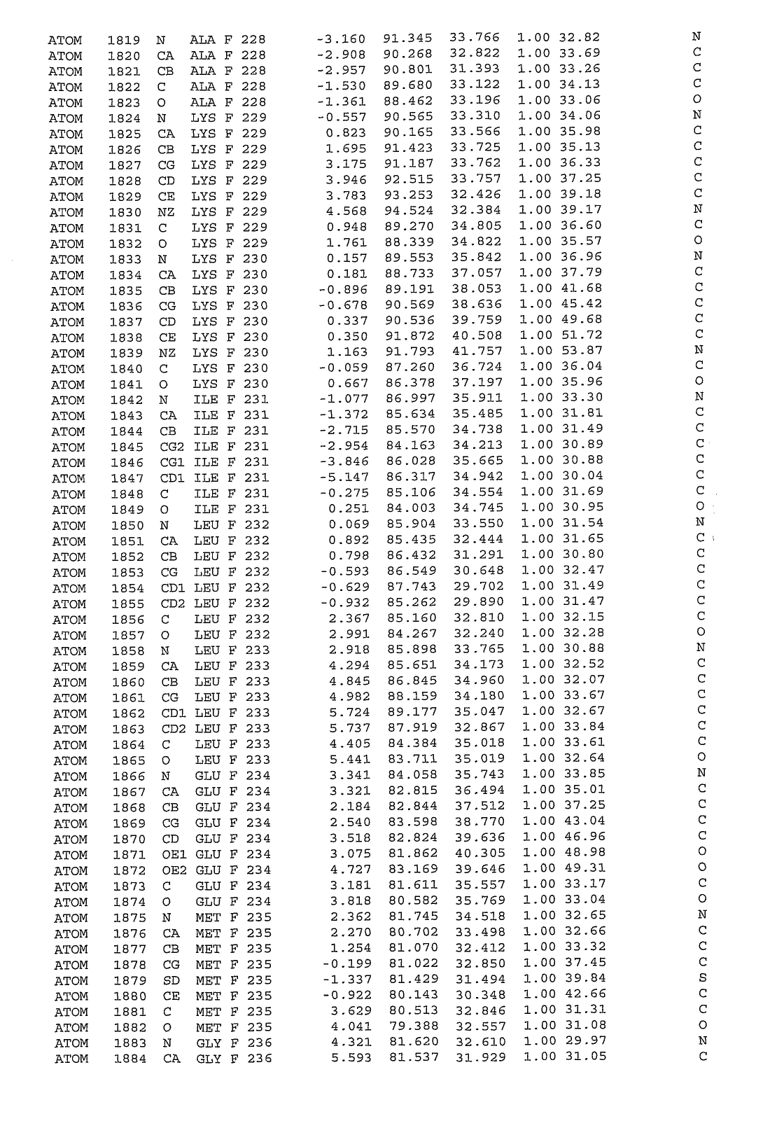

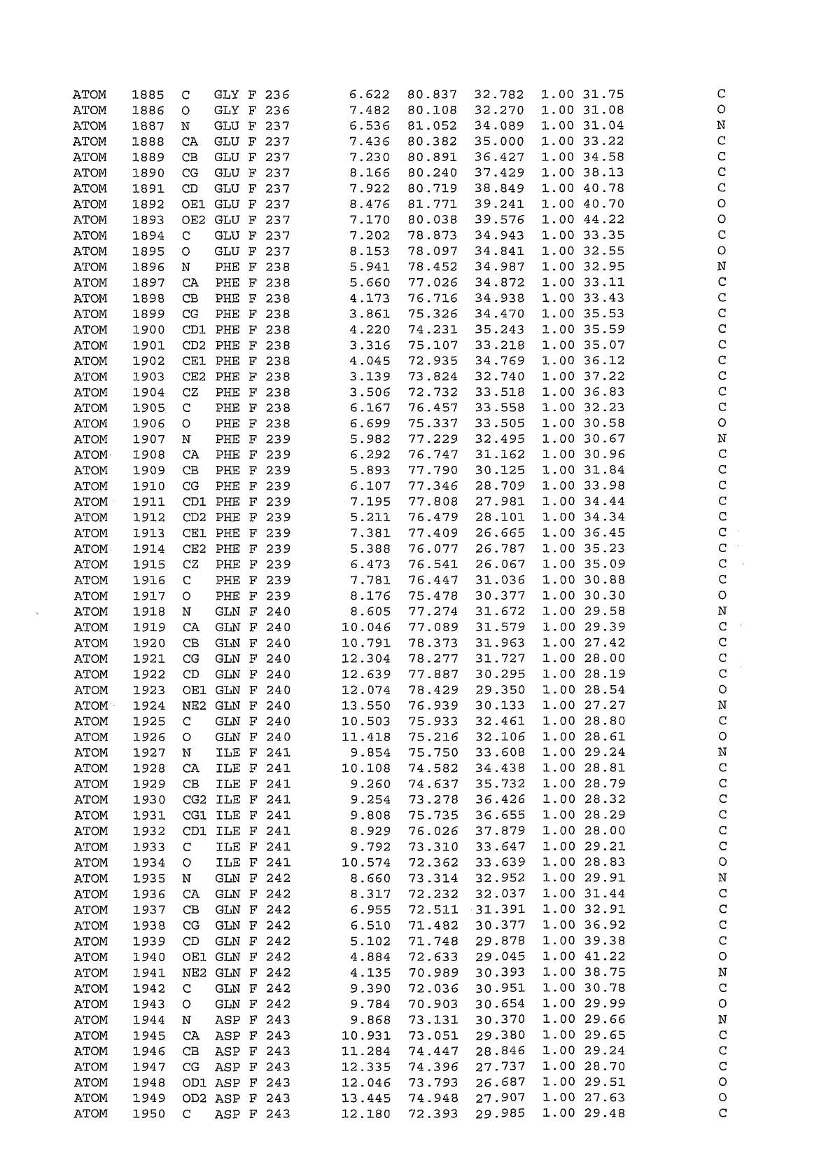

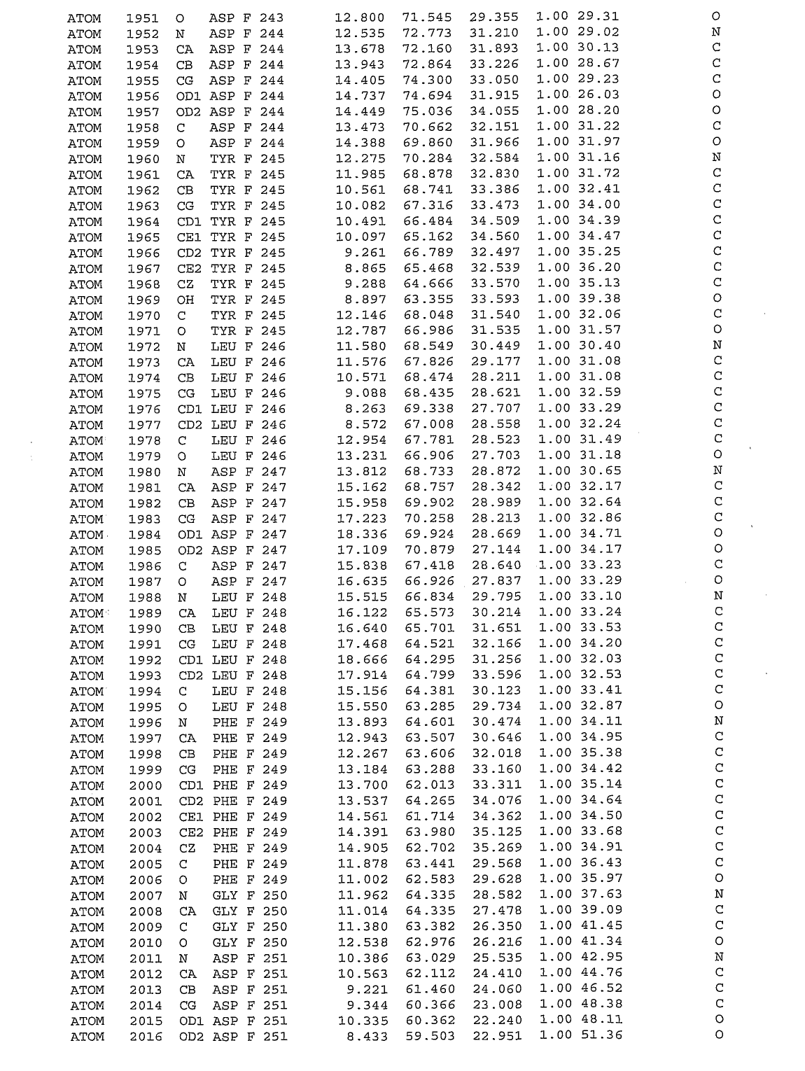

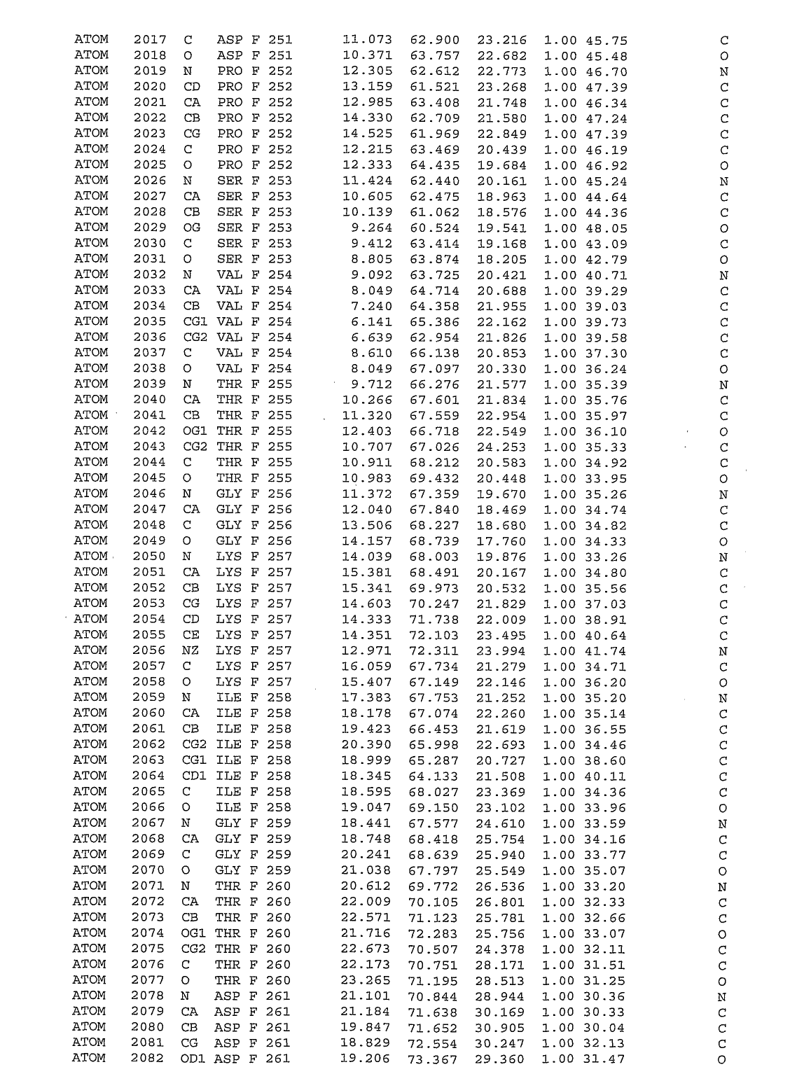

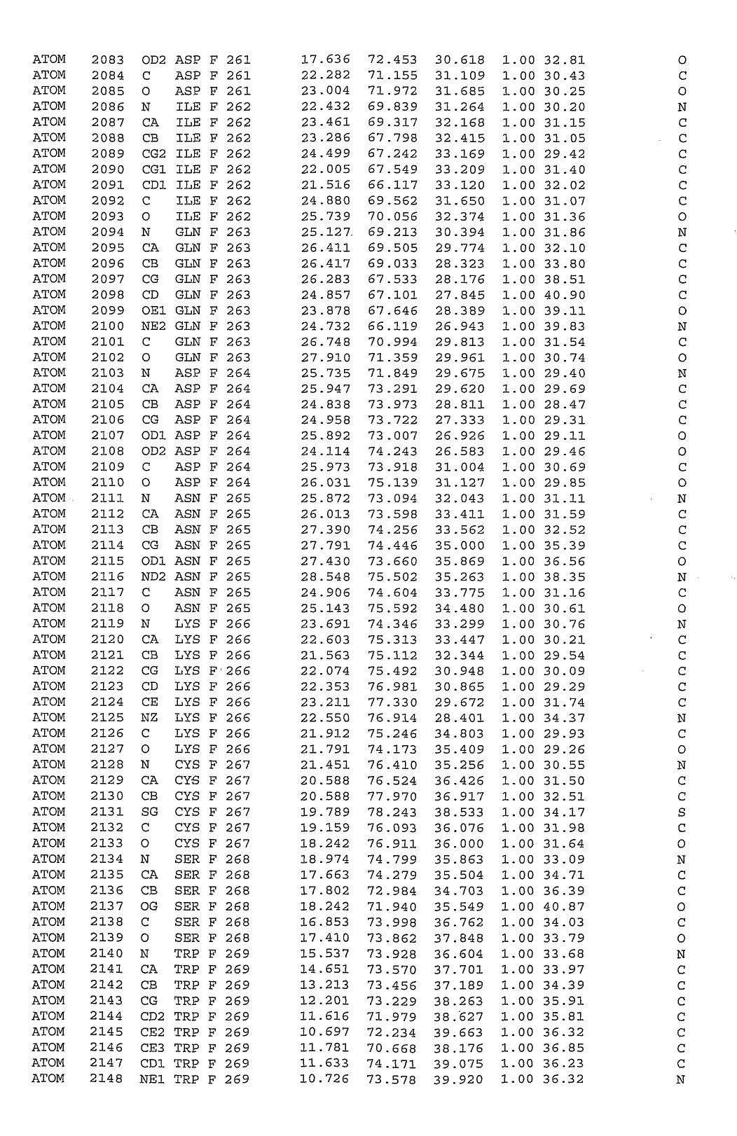

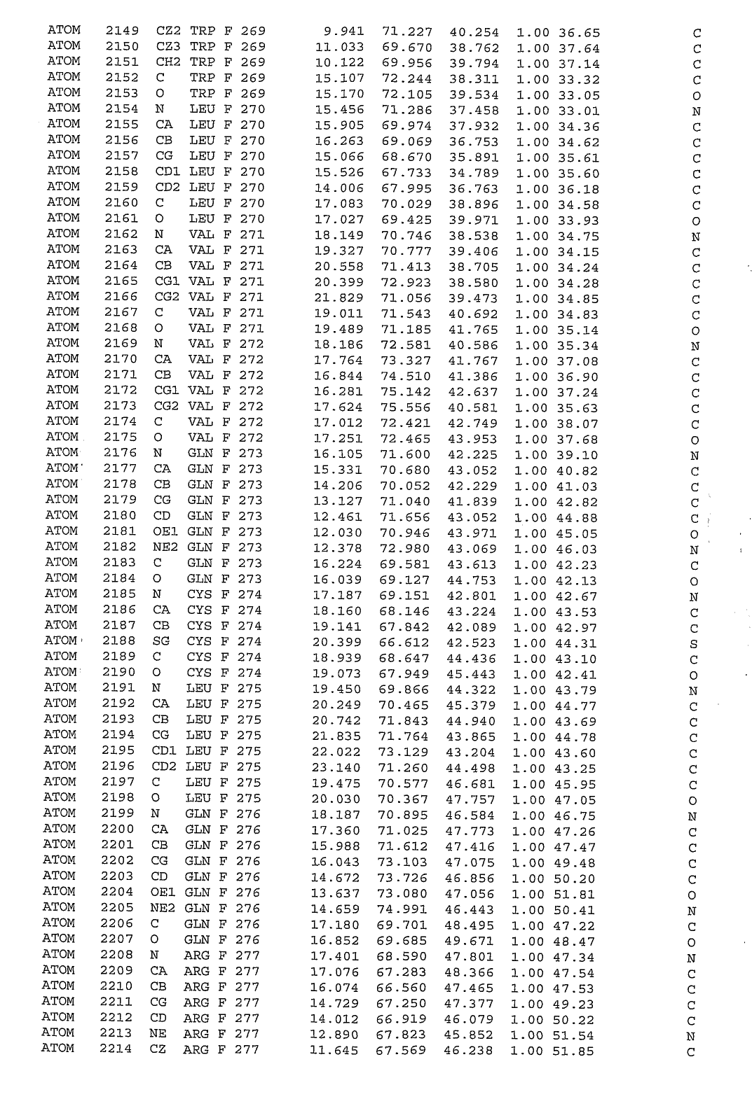

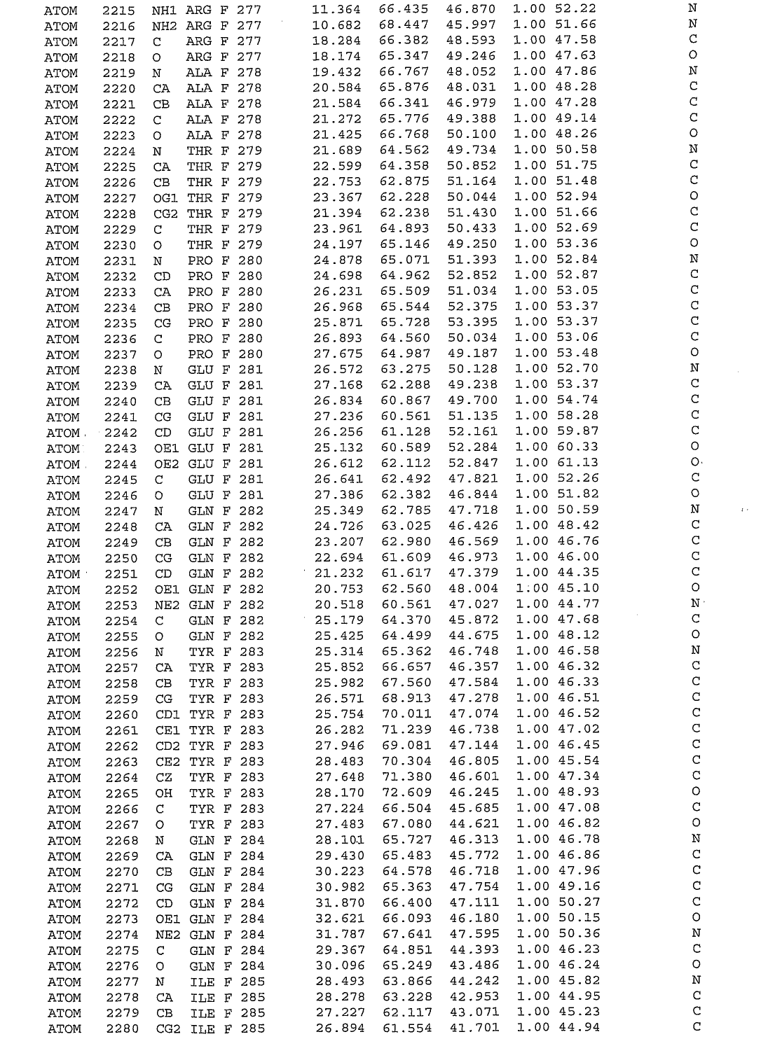

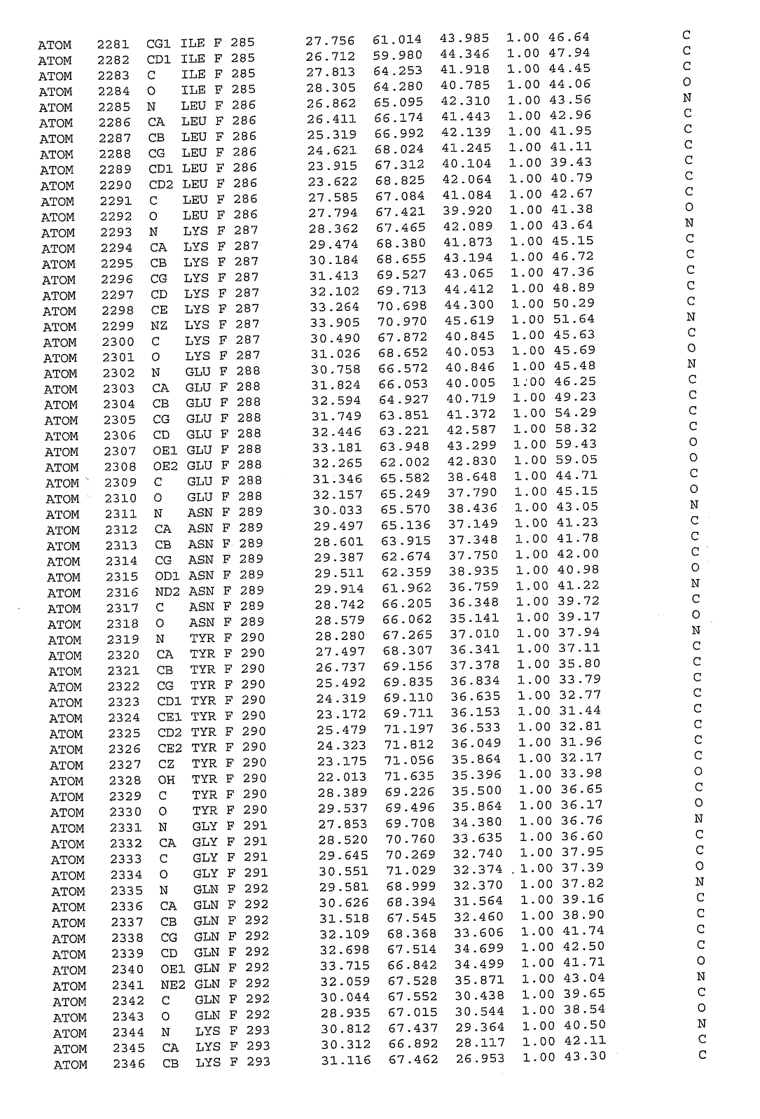

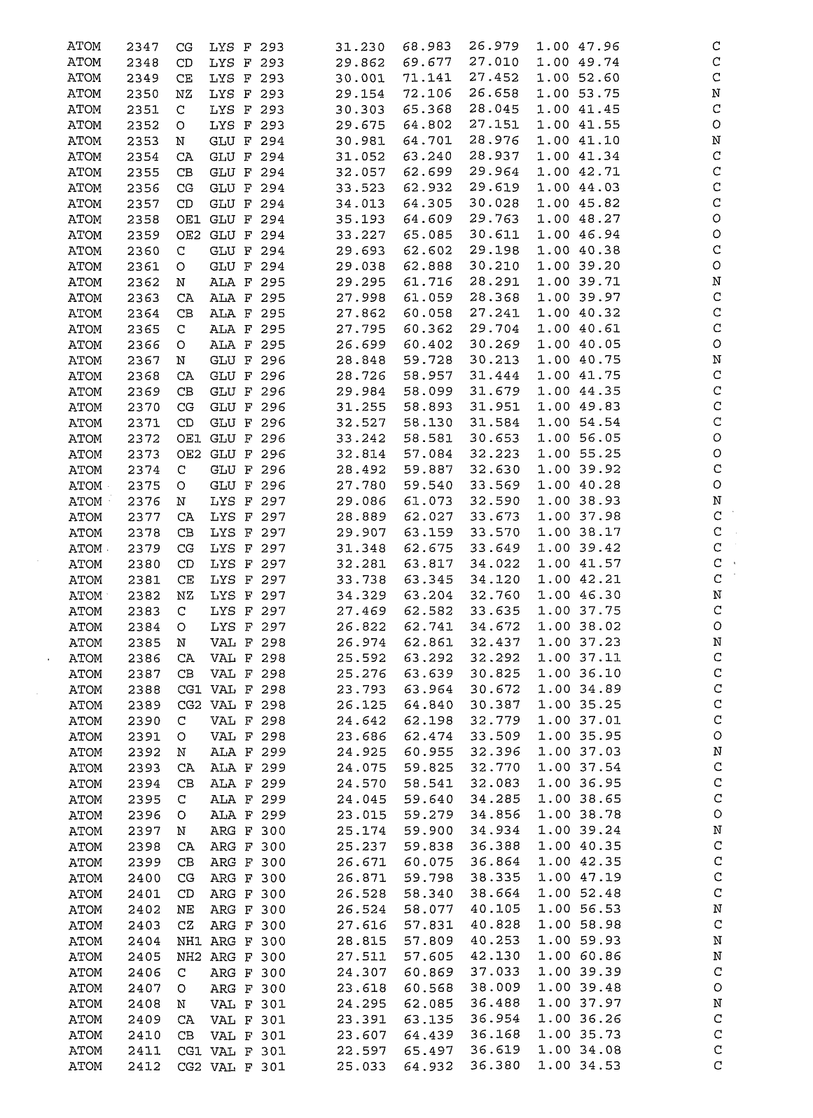

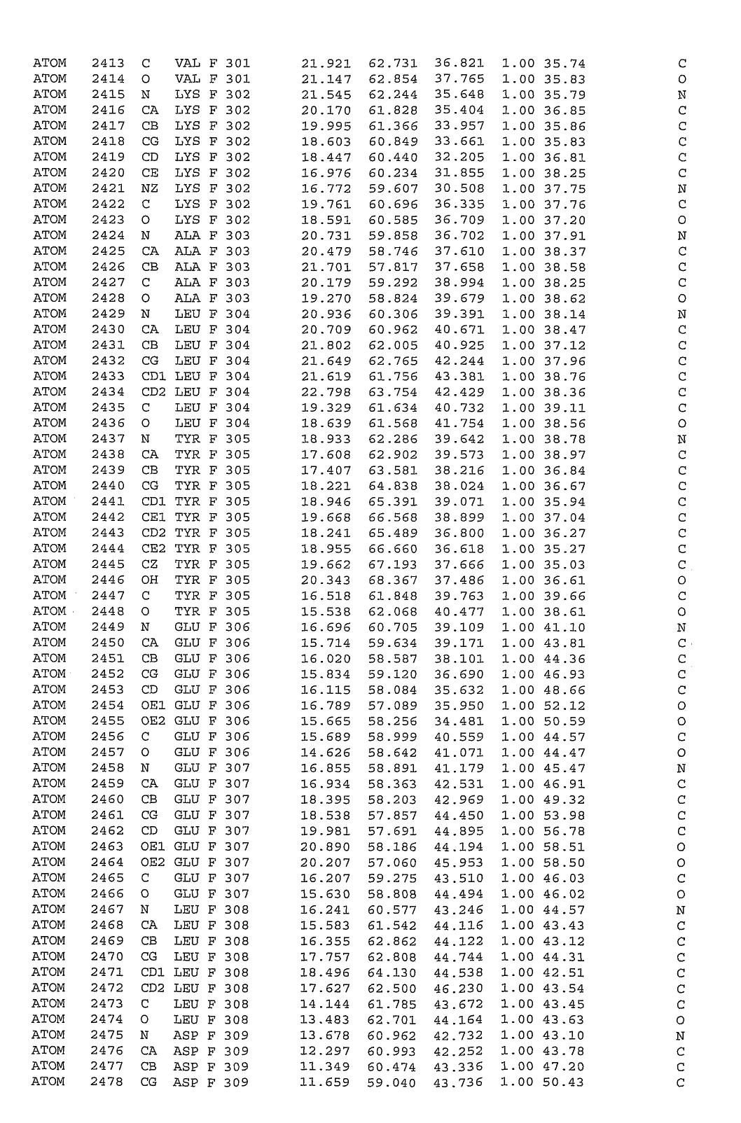

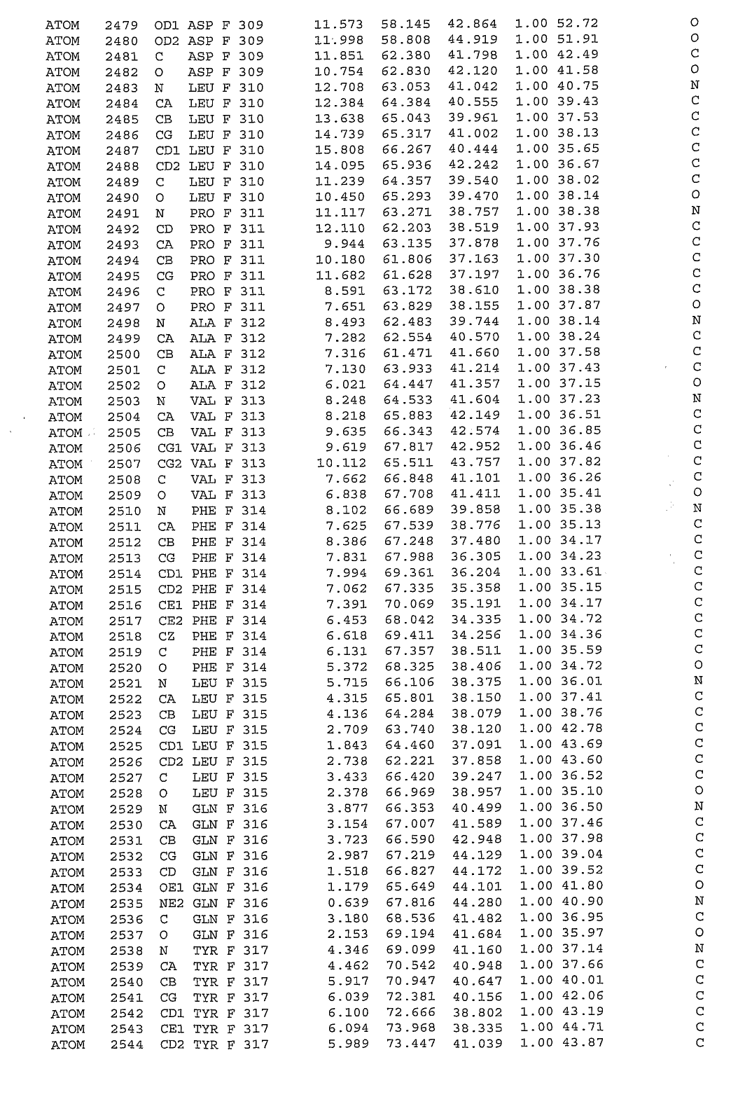

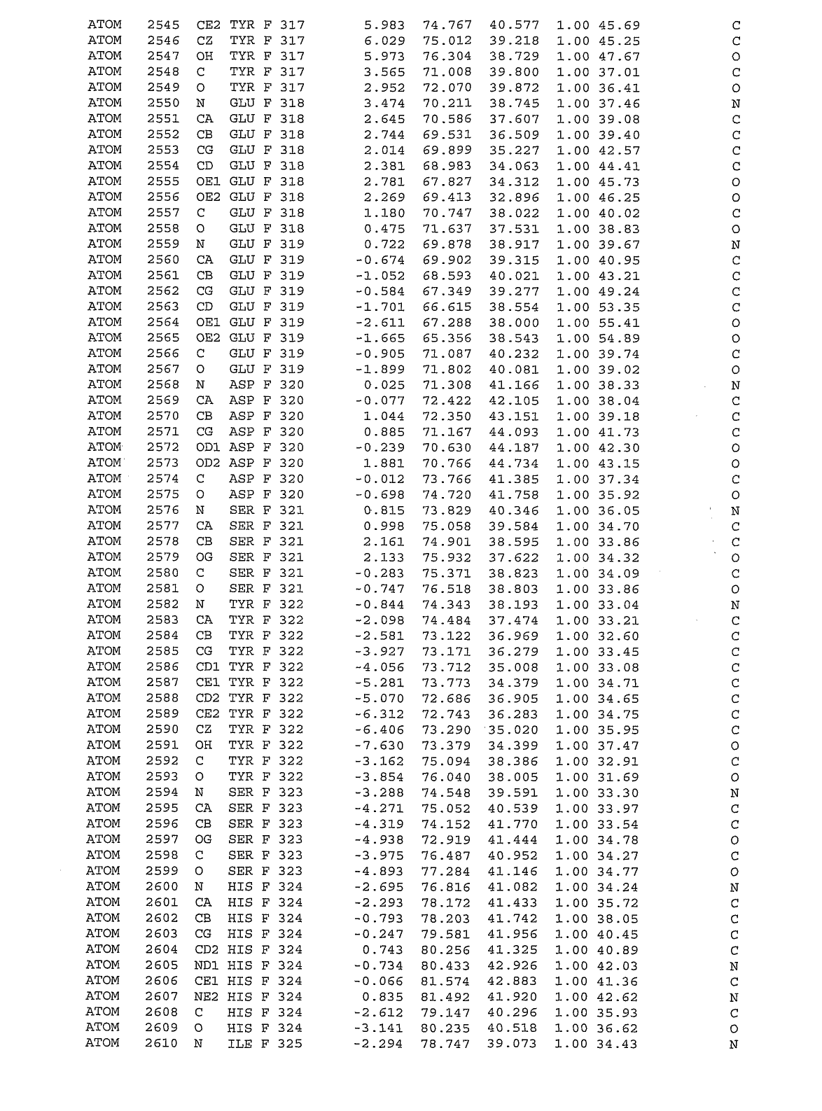

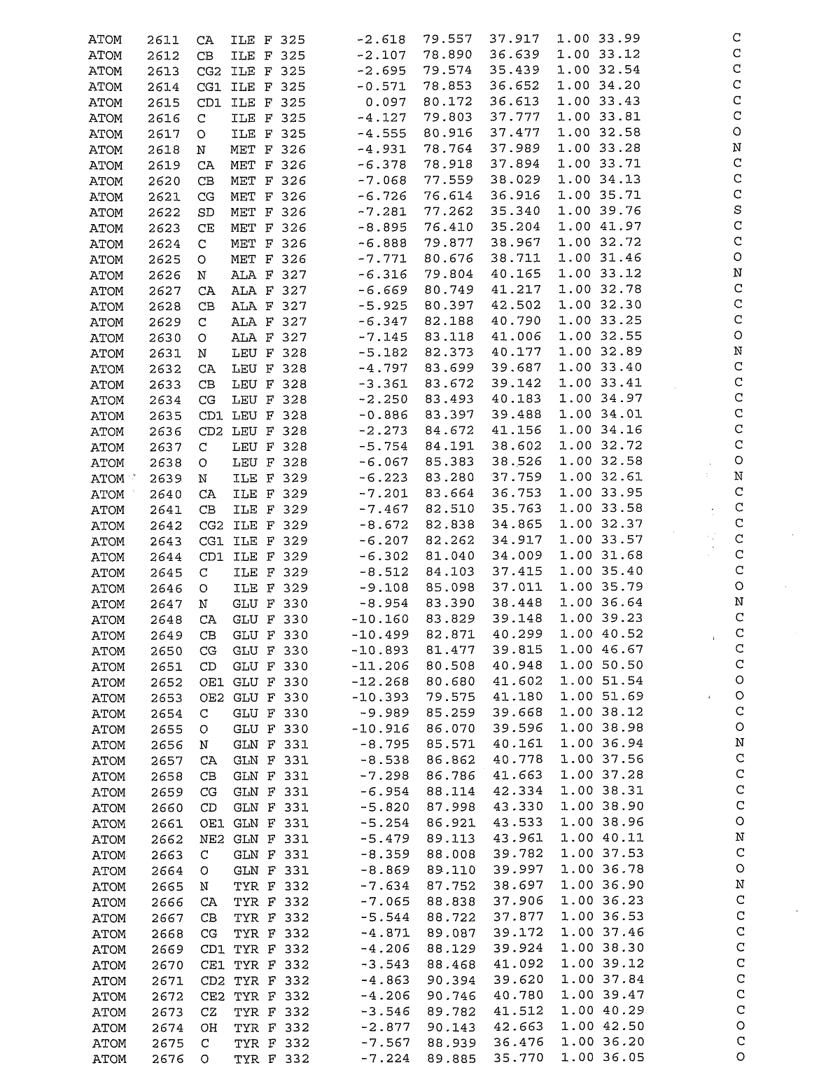

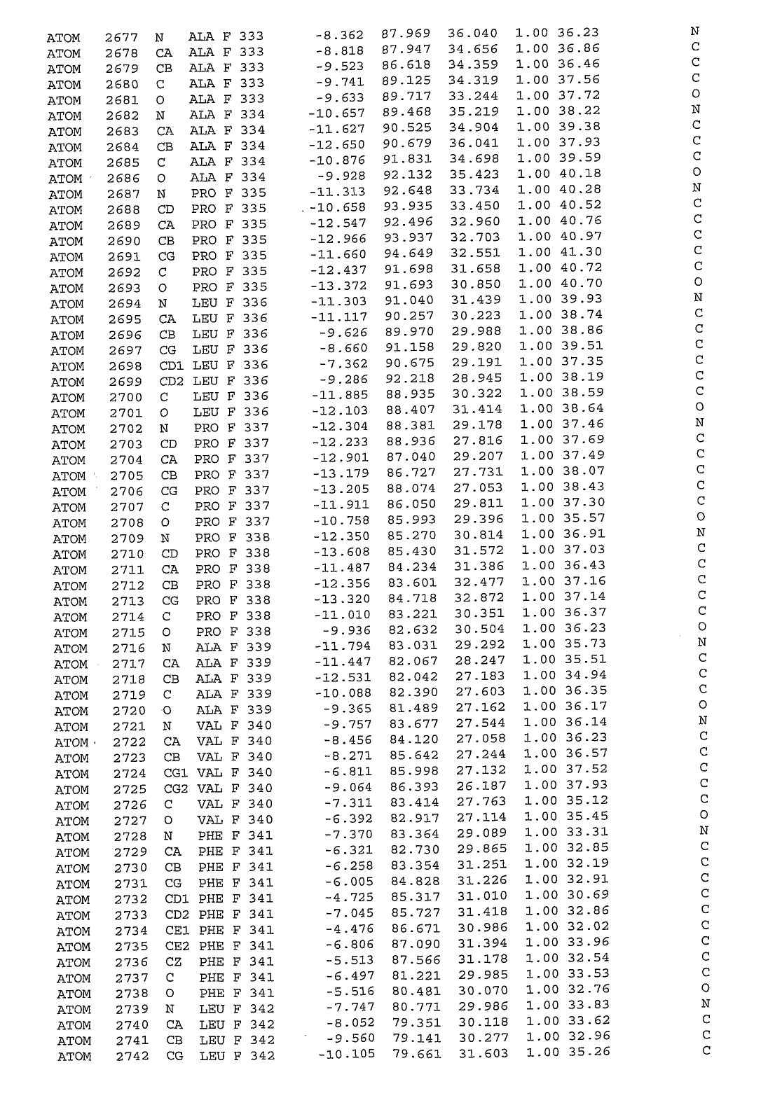

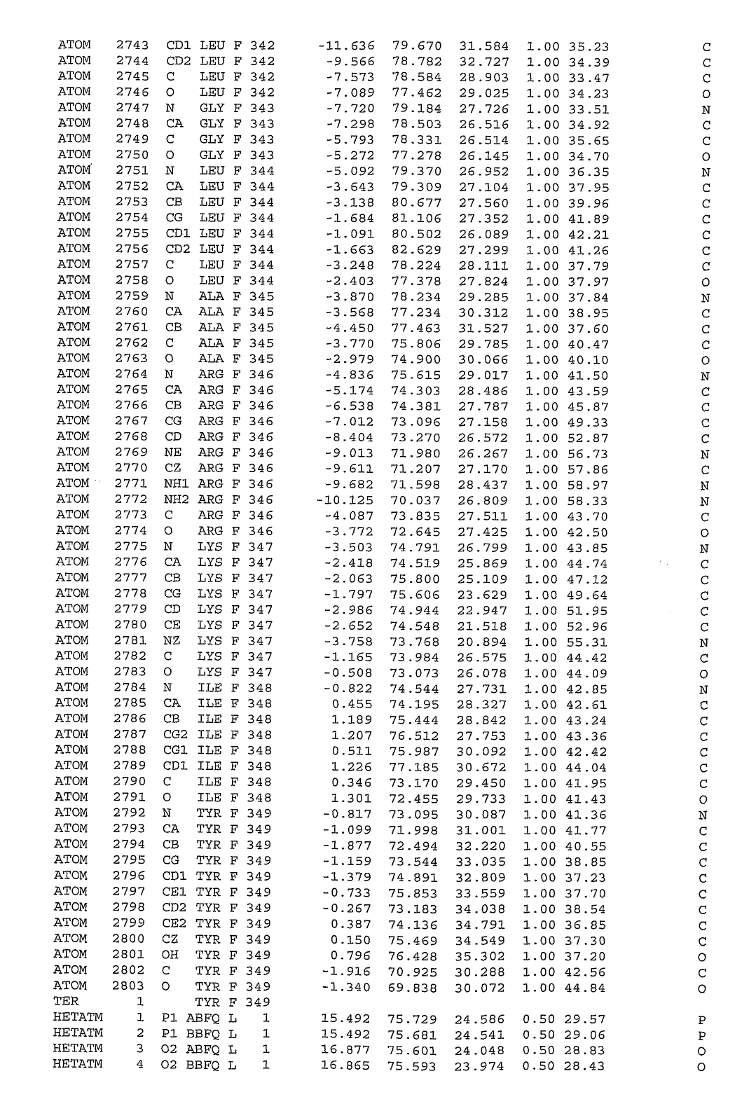

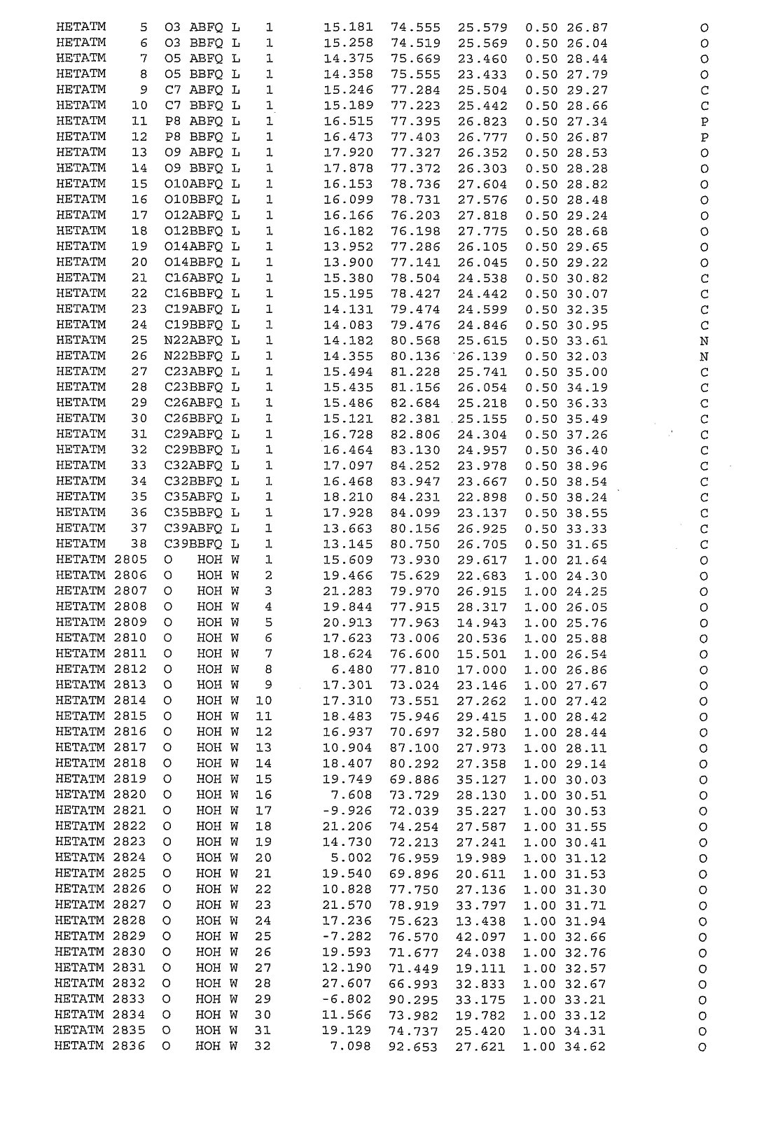

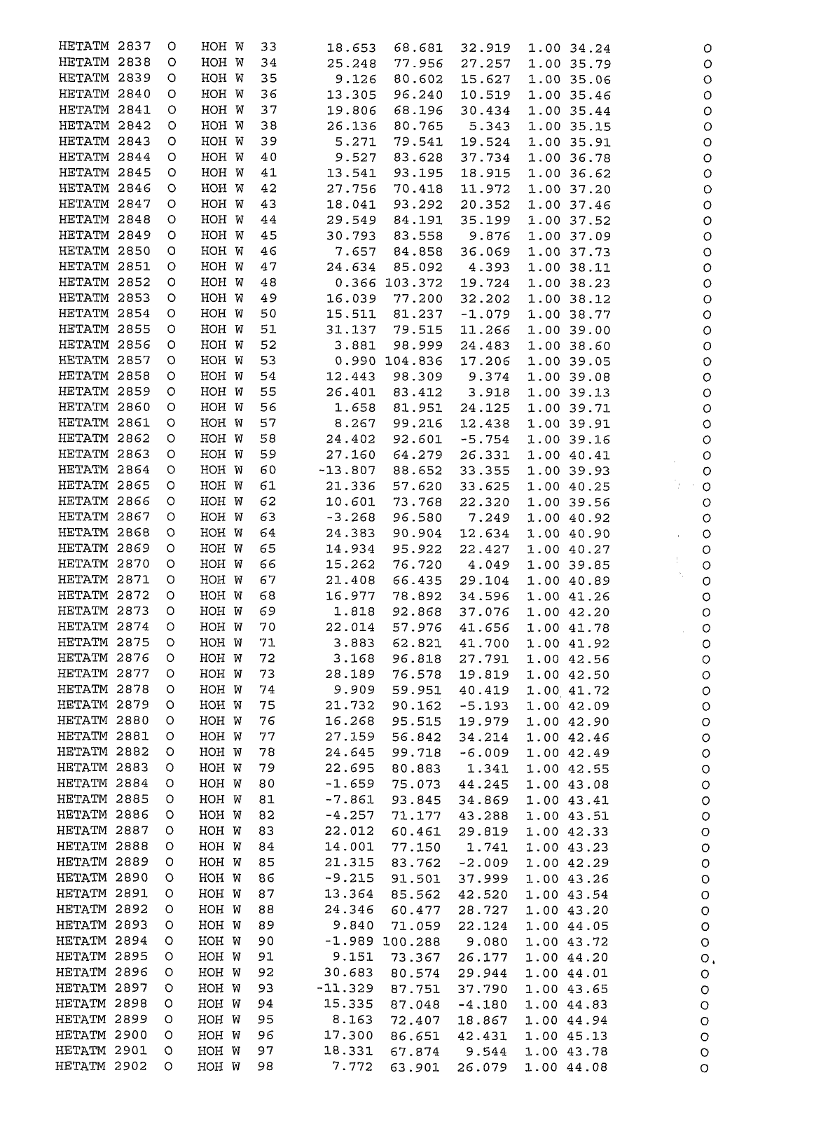

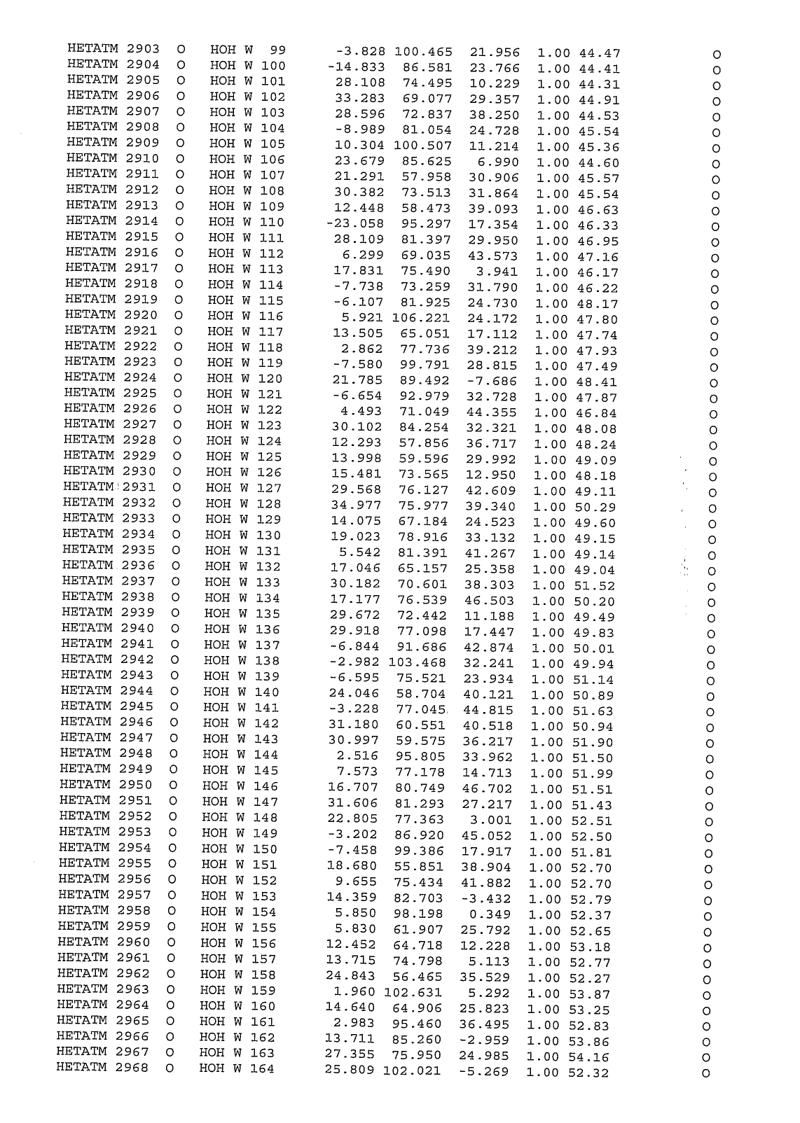

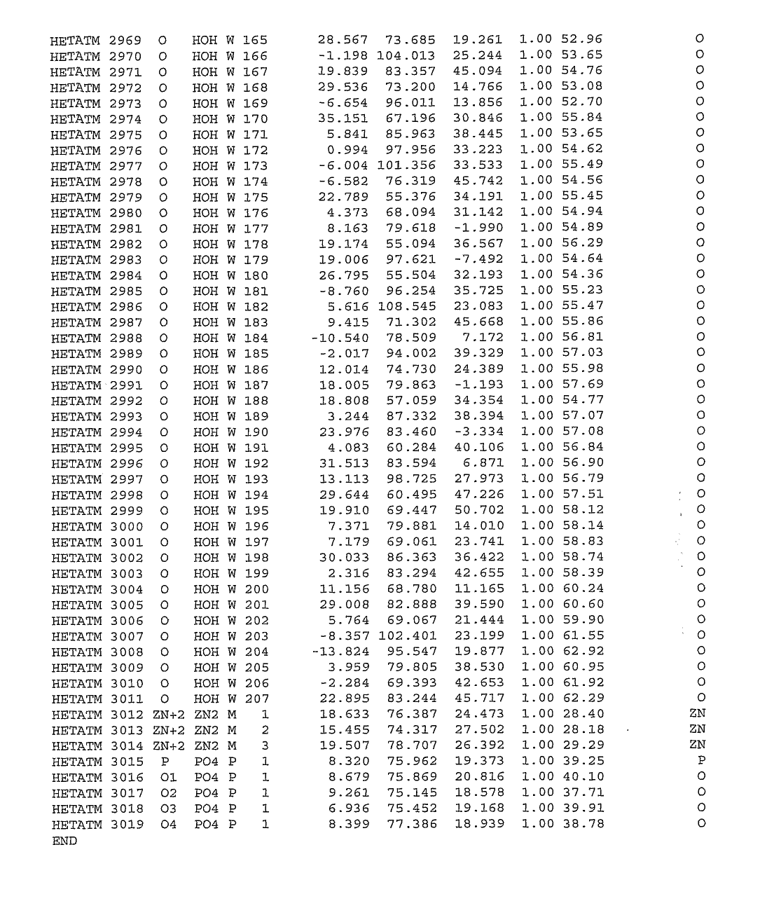

14. A crystal structure of human FPPS defined by all or a selected portion of the structural coordinates shown in FIG. 14, FIG. 15, FIG. 16, FIG. 17 and/or FIG. 18.

15. A crystal structure of human FPPS alone or complexed with a ligand.

16. Use of the crystalline human FPPS according to claim 1 for the design, selection, identification and/or preparation of FPPS ligands.

17. Use according to claim 16, wherein a computer-aided modelling program is used for the selection and/or design of ligand molecules.

18. Use according to claim 16, wherein the ligand has a three-dimensional structure which is complementary to the binding pocket of human FPPS.

19. Use according to claim 18, wherein the ligand is selected and/or designed to interact with one or more amino acids of the binding pocket selected from the group consisting of: Tyr10, Gly56, Lys57, Asn59, Arg60, Thr63, Gln96, Leu100, Asp103, Asp107, Arg112, Arg113, Gln171, Lys200, Thr201, Tyr204, Ser205, Phe206, Phe239, Gln240, Gln242, Asp243, Leu246, Lys257, Leu344, Lys347, Ile348.

20. Use according to claim 18, wherein the ligand is selected and/or designed to interact with one or more amino acids selected from the group consisting of Tyr10, Lys57, Asn59, Arg60, Thr63, Ser205, Phe206, Phe239, Gln242, Leu246, Leu344, Lys347 and Ile348.

21. A method for obtaining a three-dimensional representation of a crystal structure of human FPPS comprising providing all or a selected portion of the structural coordinates shown in FIG. 14, FIG. 15, FIG. 16, FIG. 17 and/or FIG. 18 and using said structural coordinates for constructing a three-dimensional representation of the crystal structure.

22. A computer-based method for the selection, design and/or identification of a ligand capable of binding to human FPPS, comprising the steps of:
a) providing a three-dimensional representation of human FPPS according to claim 21,
b) providing a three-dimensional representation of a candidate compound,
c) selecting the candidate compound whose three dimensional representation is complementary to the binding pocket of human FPPS, and,
d) optionally modifying said compound selected at step c) to maximize physical properties such as solubility, affinity, specificity and/or potency.

23. The computer-based method according to claim 22, wherein said compound is selected among those that interact with one or more amino acids of the binding pocket selected from the group consisting of Tyr10, Gly56, Lys57, Asn59, Arg60, Thr63, Gln96, Leu100, Asp103, Asp107, Arg112, Arg113, Gln171, Lys200, Thr201, Tyr204, Ser205, Phe206, Phe239, Gln240, Gln242, Asp243, Leu246, Lys257, Leu344, Lys347, Ile348.

24. The computer-based method according to claim 22, wherein said compound is selected among those that interact with one or more amino acids selected from the group consisting of Tyr10, Lys57, Asn59, Arg60, Thr63, Ser205, Phe206, Phe239, Gln242, Leu246, Leu344, Lys347 and Ile348.

25. A method for determining the crystal structure of a protein comprising providing all or a selected portion of the structural coordinates shown in FIG. 14, FIG. 15, FIG. 16, FIG. 17 and/or FIG. 18 and using said structural coordinates for molecular replacement to provide a crystal structure for said protein.

26. A computer-readable storage medium comprising a data storage medium with computer-readable data, the data comprising all or a selected portion of the structural coordinates shown in FIG. 14, FIG. 15, FIG. 16, FIG. 17 and/or FIG. 18.

27. A method for selecting a ligand capable of binding to human FPPS, comprising:
a. co-crystallizing or incubating a candidate compound with human FPPS,
b. determining by X-ray or NMR methods the amino acids of human FPPS which interacts with the candidate compound,
c. selecting the compound which interacts with one or more amino acids of the binding pocket selected among the group consisting of Tyr10, Gly56, Lys57, Asn59, Arg60, Thr63, Gln96, Leu100, Asp103, Asp107, Arg112, Arg113, Gln171, Lys200, Thr201, Tyr204, Ser205, Phe206, Phe239, Gln240, Gln242, Asp243, Leu246, Lys257, Leu344, Lys347, Ile348, based on the results of step b.

28. The method of claim 27, wherein said candidate compound is selected from among those that interact with one or more amino acids selected from the group consisting of Tyr10, Lys57, Asn59, Arg60, Thr63, Ser205, Phe206, Phe239, Gln242, Leu246, Leu344, Lys347 and Ile348.

29. The method of claim 27, further comprising the step of:
d. designing analogs of the compound obtained at step c) to maximize physical properties such as solubility, affinity, specificity and/or potency,
e. repeating step a. to c. of claim 27 with the corresponding analogs to select novel compounds capable of binding to human FPPS.

30. A method to design ligand to human FPPS, wherein said method comprises the steps of
a) providing a first ligand that binds to one or more amino acids of a first binding site of human FPPS,
b) providing a second ligand that binds to at least one or more amino acids of a second binding site of human FPPS, and,
c) linking said first ligand to said second ligand to design a ligand that binds to the first and second binding sites of human FPPS.

31. The method of claim 30, further comprising the steps of providing a ligand that binds to one or more amino acids of a third binding site of human FPPS, and linking said third ligand to the ligand obtained to step c) to form a ligand that binds to the first, second and third binding sites.

32. The method according to claim 30, wherein said first ligand at step a) is selected from among the ligands that interact with one or more amino acids selected among the group consisting of: Tyr10, Lys57, Asn59, Arg60, Thr63, Ser205, Phe206, Phe239, Gln242, Leu246, Leu344, Lys347 and Ile348.

33. The method according to any of claims 302, wherein a second ligand at step b) is selected from among the ligands that interact with one or more amino acids selected among the group consisting of: Gly56, Lys57, Arg60, Gln96, Arg113, Thr201, Tyr204, Phe239, Gln240 and Asp243 and/or with one or more amino acids selected among the group consisting of: Leu100, Asp103, Asp107, Arg112, Gln171, Lys200, Thr201, Tyr204, Glu240, Asp243 and Lys257.

34. A ligand for human FPPS, obtained using a crystalline human FPPS according to any of claims 1-8, crystal structure data of human FPPS according to claim 14.

35. Ligand according to claim 34, wherein it is a ligand or inhibitor of FPPS.

36. Pharmaceutical composition comprising a ligand according to claim 34.

37. Pharmaceutical composition according to claim 36 for the treatment and/or prevention of tumor-induced hypercalcemia, Paget's disease of bone, osteolytic metastases, postmenopausal osteoporosis, hypocholesterolemia and/or soft tissue cancer.
